SEQUENCE LISTING (158 sequences)

SEQ ID NO: 1 - DNA, 687 bp, synthetic construct
  misc_feature 1..687: note = DNA sequence of human CMV (hCMV) promoter
  source 1..687: mol_type = other DNA
ttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttacc

SEQ ID NO: 2 - DNA, 1391 bp, synthetic construct
  misc_feature 1..1391: note = DNA sequence of mouse CMV (mCMV) promoter
  source 1..1391: mol_type = other DNA
aactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcag

SEQ ID NO: 3 - DNA, 1662 bp, synthetic construct
  misc_feature 1..1662: note = DNA sequence of strain D26-76 NDV F protein
  source 1..1662: mol_type = other DNA
atgggctccagatcttctaccaggatcccagtacctctgatgctgaccgtccgaatcatgttggcactgagttgcgtctgtccgaccagctcccttgatggcaggcctcttgcagctgcagggattgtggtaacaggagacaaagcagtcaacatatacacctcatctcagacagggtcaatcataatcaagttactcccaaatatgcccaaggataaagaggcgtgtgcaaaagccccattggaagcatacaacaggacattgactactttgctcaccccccttggtgattctatccgtaggatacaagagtctgtgaccacatccggaggagggaaacagggacgtcttataggcgccattatcggtggtgtagctctcggggttgcaaccgctgcacagataacagcagcctcggctctgatacaagccaatcaaaatgctgccaacatcctccggctcaaagagagcattgctgcaaccaatgaggctgtgcacgaggtcactgacggattatcacaactagcagtggcagttgggaagatgcagcaatttgttaatgaccagtttaataaaacagctcaggaattggactgtataaaaattacacagcaggttggtgtagaactcaacctgtacctaactgaattgactacagtattcgggccacaaatcacttcccctgccttaactcagctgactatccaggcgctttacaatctagctggtgggaatatggattacttgttgactaagttaggtgtaggaaacaaccaactcagctcattaattggtagtggcctgattaccggcaaccctatcctgtacgactcacagactcaactcttgggtatacaggtcaccctaccctcagtcgggaatctaaataatatgcgtgccacctacctggaaaccttgtctgtaagtacaaccaaaggatttgcctcagcacttgtcccaaaagtagtgacacaggttggttccgtgatagaagagcttgacacctcgtactgtatcgagaccgatttggacctatattgtacaagaatagtgacattccctatgtctcctggtatttattcctgtttgagtggcaatacatctgcttgcatgtattcaaagactgaaggcgcactcactacgccgtatatgaccctcaaaggctcagttattgccaactgtaagatgacaacatgtagatgtgcagaccccccgggtatcatatcgcagaattatggagaagctgtgtctctaatagataggcaatcatgcaatatcttatccttagacgggataactttgaggctcagtggggaatttgatgcaacttatcaaaagaatatctcaatacaagattctcaagtaatagttacaggcaatcttgacatctcgactgagcttgggaatgtcaacaactcgataagtaatgctttggataagttagaggaaagcaacagcaaactagacaaggtcaatgttaaactgaccagcacatccgctcttattacctatatcgttttaactgtcatatctcttgtatgtggtatacttagcctggttctagcatgctacctgatgtacaagcaaaaggcgcaacagaagaccttgttgtggcttgggaataataccctagaccagatgagggccactacaaaaatgtag

SEQ ID NO: 4 - protein, 553 aa, synthetic construct
  REGION 1..553: note = Amino acid sequence of strain D26-76 NDV F protein
MGSRSSTRIPVPLMLTVRIMLALSCVCPTSSLDGRPLAAAGIVVTGDKAVNIYTSSQTGSIIIKLLPNMPKDKEACAKAPLEAYNRTLTTLLTPLGDSIRRIQESVTTSGGGKQGRLIGAIIGGVALGVATAAQITAASALIQANQNAANILRLKESIAATNEAVHEVTDGLSQLAVAVGKMQQFVNDQFNKTAQELDCIKITQQVGVELNLYLTELTTVFGPQITSPALTQLTIQALYNLAGGNMDYLLTKLGVGNNQLSSLIGSGLITGNPILYDSQTQLLGIQVTLPSVGNLNNMRATYLETLSVSTTKGFASALVPKVVTQVGSVIEELDTSYCIETDLDLYCTRIVTFPMSPGIYSCLSGNTSACMYSKTEGALTTPYMTLKGSVIANCKMTTCRCADPPGIISQNYGEAVSLIDRQSCNILSLDGITLRLSGEFDATYQKNISIQDSQVIVTGNLDISTELGNVNNSISNALDKLEESNSKLDKVNVKLTSTSALITYIVLTVISLVCGILSLVLACYLMYKQKAQQKTLLWLGNNTLDQMRATTKM

SEQ ID NO: 5 - DNA, 1362 bp, synthetic construct
  misc_feature 1..1362: note = DNA Sequence of vIBDV (F52/70) VP2
  source 1..1362: mol_type = other DNA
atgaccaatctccaggaccagacccagcagattgtgccttttattaggagtctcttgatgcctacaaccggccccgccagcatcccggacgacacactggaaaaacatacactgagaagcgagacatctacatacaatttgaccgtgggcgataccggctccgggcttatcgtgttcttcccaggttttcccggatctatcgtaggagcgcactacaccctccaaagtaacggcaattacaaattcgaccagatgctcctgacagcccagaaccttcctgcttcttacaattactgtagacttgtgtccaggtccctgactgtgcggagtagcacgcttccaggaggcgtatacgccctgaacggaactataaacgccgtcaccttccagggctccttgtccgaacttaccgacgtgtcctacaatggcctcatgagcgcaacggccaacataaacgataagatcggcaatgttcttgtgggcgagggggttacagtcctttctctgccaaccagttatgatctgggatacgtgcggcttggcgatcccattcccgctatcggtctcgaccctaaaatggtggctacttgcgactcatctgaccgcccaagggtctatacaattactgcagccgatgactatcagttttccagccaataccagccagggggtgtgacaatcacacttttcagcgccaatattgacgctatcacatccctctcaatcggaggtgagcttgtgttccagacttctgttcagggcttggtattgggcgccactatttacttgatcgggttcgacgggaccgcagtgatcactcgggcagtggctgcggataacggactcactgccggaactgacaaccttatgccttttaatctggtcatccccactaacgagatcacccagcctattacctccataaagctcgaaattgtgaccagcaagagcggagggcaggcaggcgaccaaatgagttggtctgcaagcgggtccctcgccgtgaccatccacggtggcaactatcctggggcgctcagacccgtcaccctggtagcctacgaaagggttgccacaggctcagttgtcacggtggctggagtaagcaatttcgagctcatcccgaatcctgagctcgctaaaaatcttgtgaccgagtatggaaggttcgaccctggcgcaatgaattacacaaagctgattctgtccgaacgggataggctgggtatcaagacagtttggcccacgcgcgaatacacagatttcagggagtactttatggaggtcgcagatttgaatagcccacttaagatcgctggagcatttggctttaaggatattatccgcgcaatcagaaggtag

SEQ ID NO: 6 - DNA, 225 bp, synthetic construct
  misc_feature 1..225: note = DNA sequence of Bovine Growth Hormone Poly A
  source 1..225: mol_type = other DNA
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggt

SEQ ID NO: 7 - DNA, 580 bp, synthetic construct
  misc_feature 1..580: note = DNA Sequence of HVT UL55 Flanking
  source 1..580: mol_type = other DNA
atggcagcgagcatgggatattcatcctcgtcatcgttaacatctctacgggttcagaatgtttggcatgtcgtcgatcctttgcccatcgttgcaaattacaagtccgatcgccatgaccgcgataagcctgtaccatgtggcattagggtgacatctcgatcatacattataagaccaacgtgcgagtcttccaaagacctgcacgccttcttcttcggattgtcaacgggttcttcagaatctatgcccatatctggcgttgagaccattgtgcgtttaatgaacaataaagcggcatgccatggaaaggagggctgcagatctccattttctcacgccactatcctggacgctgtagacgataattataccatgaatatagagggggtatgtttccactgccactgtgatgataagttttctccagattgttggatatctgcattttctgctgccgaacaaacttcatcgctatgcaaagagatgcgtgtgtacacgcgccgttgagtatacgggaaactaaatgttcatagaggtctttgggctatatgttattaaataaaataattgaccagtg

SEQ ID NO: 8 - DNA, 678 bp, synthetic construct
  misc_feature 1..678: note = DNA Sequence of HVT Gene 3 Flanking
  source 1..678: mol_type = other DNA
gtttaatgttagtttattcaatgcattggttgcaaatattcattacttctccaatcccaggtcattctttagcgagatgatgttatgacattgctgtgaaaattactacaggatatatttttaagatgcaggagtaacaatgtgcatagtaggcgtagttatcgcagacgtgcaacgcttcgcatttgagttaccgaagtgcccaacagtgctgcggttatggtttatgcgcacagaatccatgcatgtcctaattgaaccatccgatttttcttttaatcgcgatcgttgtttgggcaactgcgttatttcagatctaaaaaatttaccctttatgaccatcacatctctctggctcataccccgcttggataagatatcatgtagattccgccctaagaaatgcaaactaacattattgtcggttccatatacacttccatcttgtccttcgaaaataacaaactcgcgcaatagaccgtccgtacatgcatggccgatgtgtgtcaacatcattggtctgctagatcccgatgggacgaatcgtacagtcgtcgctccagcattggcaaaaatccccagataccctccatgcggcaaatctaaattgcgaccccgaagagactgcaccaaagtcttatcgacgcacgctgatttttttgaacagcgggagcccat

SEQ ID NO: 9 - DNA, 561 bp, synthetic construct
  misc_feature 1..561: note = DNA Sequence of Pec Promoter
  source 1..561: mol_type = other DNA
agttattaatagtaatcaattacggggtcattagttcatagcccatatatggagttccgcgttacataacttacggtaaatggcccgccggctgaccgcccaacgacccccgcccattgacgtcaataatgacgtatgttcccatagtaacgccaatagggactttccattgacgtcaatgggtggagtatttacggtaaactgcccattggcagtacatcaagtgtatcatatgccaagtacgccccctattgacgtcaatgacggtaaatggatgcagtattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgtctagagg

SEQ ID NO: 10 - DNA, 1362 bp, synthetic construct
  misc_feature 1..1362: note = DNA Sequence of vvIBDV VP2
  source 1..1362: mol_type = other DNA
atgacaaatcttcaggatcagacccagcagatagttccctttattaggtcccttctgatgccaaccacagggcccgctagcattccggacgataccctggagaaacacactttgcggagtgagacaagcacttacaatctgacggtgggagataccggctcaggtcttatcgtgttttttcccggctttcctggatccattgttggcgcgcattacacgctgcagagcaacggcaactataaattcgatcagatgctcctgacggctcagaatctccccgccagttacaattactgccgccttgtaagtaggtccttgactgttagaagctcaacgctgccaggcggagtatatgccctgaatggaaccattaatgctgtaacattccaaggatcactgtccgagctcaccgatgtgtcttacaatggattgatgtctgccacggctaacattaacgacaagatcgggaatgtgctcgtgggcgagggagtgaccgttttgagcctgccgacaagctacgacctcggctacgtaaggctcggggatccaatccccgcgatcggcttggatcccaaaatggttgctacgtgcgacagcagcgatagacccagggtctataccatcaccgctgccgatgattaccagtttagctcccagtaccaggcgggaggggtcacgatcaccctttttagcgccaacatcgacgccataacctcactttctatagggggcgagttggtttttcagaccagtgtccaggggctcatcctcggtgcgacaatctatctgatcggctttgacggaacagctgtcatcacgagggccgtagctgcagataatggcctgactgctgggacagataatctgatgccgttcaacatagtgatccccaccagtgagattacgcaacccatcacgagcatcaaactggagatcgtgacgtcaaaatccggcggtcaggcaggtgaccagatgtcttggtccgcaagcggaagtttggccgtgacaattcacggggggaattaccccggcgcactcaggcccgtgaccctcgtcgcctacgaaagagttgcaacgggaagtgtagtgacagtcgctggagtgagtaacttcgaactcatccctaatcccgagctcgccaaaaatctcgtcacggagtatgggaggtttgatcccggcgccatgaactacacaaaactgatattgtccgaaagggataggttgggcattaaaaccgtgtggcctactagggaatacaccgatttccgcgaatattttatggaggtcgcggatctgaactctcccctgaagatagcaggcgcttttgggttcaaggatattatccgggcgttgcggcggtag

SEQ ID NO: 11 - DNA, 197 bp, synthetic construct
  misc_feature 1..197: note = DNA Sequence SV40 promoter
  source 1..197: mol_type = other DNA
tgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaa

SEQ ID NO: 12 - DNA, 122 bp, synthetic construct
  misc_feature 1..122: note = DNA Sequence SV40 polyA sequence
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 13 - DNA, 531 bp, synthetic construct
  misc_feature 1..531: note = DNA Sequence of HVT UL35 Flanking
  source 1..531: mol_type = other DNA
gccactgtatgggccatttatgtttatcgagtctaaaagtcgtggaagcgcctggccatcgccagtctcgacctgccactgctgagttcggcttcaattccagcatcccagaaatgtcgcagtcaccatcacgccgtgcatcgcttcctacgaggccttttgacgcttctgatttgggcacatacaccctggacatactccaccgctattcgctcgtagatttagtacaactactgaatgacttgccgcgtaacattacctccacgcccgcttctaatgtagaaaccatggcaaaaattaatgttttaagggccatttgcgtaggatttgccgaggtccgtcgccacaacgacgcgcgaactttacagcgaacggcaatgtttgccgccgacgacgtcgcatcacggatcagaccatccattggattaaagcgcacctacccaccgggtatattttccacagctattaccgtatctaattccgaggatgaagagcgaaattcgtgatcgtaaaaataaaaaatacaagata

SEQ ID NO: 14 - DNA, 700 bp, synthetic construct
  misc_feature 1..700: note = DNA Sequence of HVT UL36 Flanking
  source 1..700: mol_type = other DNA
gtacataattcttatttatctttaatccatgaggagcatttttattttaaaaatgtcagccgccagccctataaccctagatcgcaactgatccctagtctgcgttatttgtcttgcaatcttttcgcacgcctttgtgagtgcatacaatgcccccctgctcgcttttctgaaatcgcgtcgggtcattaatgtgtcggctatcacaatgcgagatgtactcgacatgtccgtgtctgtactattgggattgtaaatagtcgaccgcgaatcatcagagtcggaatctgtaaaggatacagattccgactctgagcgcttatgaatgggatccactcggacgttgttgaacttccgttcggattctgcttcagtcaacaccggcccccgatagctactaaggttggggggtttgtgggttgtttgtgaaactgctttgcggtgtgcattaccacggggggtgtggggaagtatctgtttccacgatgcgataacgttcggtggcggagggggcgattcattctctagtgtacgcgtttcaacttcaggaacgtgattatttctttcaggacactctttccaatttccttcttccttcacttcgggtacaggtatattcttaatgtttacatacatgtcgtctgctcgtctcaactgcggggttatgatgggtggtggtgacagtctctccgaatgatcg

SEQ ID NO: 15 - DNA, 1278 bp, synthetic construct
  misc_feature 1..1278: note = DNA Sequence of Chicken Beta-actin Promoter
  source 1..1278: mol_type = other DNA
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggggtttatatcttcccttctctgttcctccgcagccagccatg

SEQ ID NO: 16 - DNA, 2882 bp, synthetic construct
  misc_feature 1..2882: note = DNA Sequence of Modified transfer plasmid for HVT-gfp-A #14*
  source 1..2882: mol_type = other DNA
gccactgtatgggccatttatgtttatcgagtctaaaagtcgtggaagcgcctggccatcgccagtctcgacctgccactgctgagttcggcttcaattccagcatcccagaaatgtcgcagtcaccatcacgccgtgcatcgcttcctacgaggccttttgacgcttctgatttgggcacatacaccctggacatactccaccgctattcgctcgtagatttagtacaactactgaatgacttgccgcgtaacattacctccacgcccgcttctaatgtagaaaccatggcaaaaattaatgttttaagggccatttgcgtaggatttgccgaggtccgtcgccacaacgacgcgcgaactttacagcgaacggcaatgtttgccgccgacgacgtcgcatcacggatcagaccatccattggattaaagcgcacctacccaccgggtatattttccacagctattaccgtatctaattccgaggatgaagagcgaaattcgtgatcgtaaaaataaaaaatacaagatattgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttacctgcaggccaccatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtgacctgcaggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgtacataattcttatttatctttaatccatgaggagcatttttattttaaaaatgtcagccgccagccctataaccctagatcgcaactgatccctagtctgcgttatttgtcttgcaatcttttcgcacgcctttgtgagtgcatacaatgcccccctgctcgcttttctgaaatcgcgtcgggtcattaatgtgtcggctatcacaatgcgagatgtactcgacatgtccgtgtctgtactattgggattgtaaatagtcgaccgcgaatcatcagagtcggaatctgtaaaggatacagattccgactctgagcgcttatgaatgggatccactcggacgttgttgaacttccgttcggattctgcttcagtcaacaccggcccccgatagctactaaggttggggggtttgtgggttgtttgtgaaactgctttgcggtgtgcattaccacggggggtgtggggaagtatctgtttccacgatgcgataacgttcggtggcggagggggcgattcattctctagtgtacgcgtttcaacttcaggaacgtgattatttctttcaggacactctttccaatttccttcttccttcacttcgggtacaggtatattcttaatgtttacatacatgtcgtctgctcgtctcaactgcggggttatgatgggtggtggtgacagtctctccgaatgatcg

SEQ ID NO: 17 - DNA, 2869 bp, synthetic construct
  misc_feature 1..2869: note = DNA sequence Original transfer plasmid for HVT-gfp-A #14
  source 1..2869: mol_type = other DNA
gccactgtatgggccatttatgtttatcgagtctaaaagtcgtggaagcgcctggccatcgccagtctcgacctgccactgctgagttcggcttcaattccagcatcccagaaatgtcgcagtcaccatcacgccgtgcatcgcttcctacgaggccttttgacgcttctgatttgggcacatacaccctggacatactccaccgctattcgctcgtagatttagtacaactactgaatgacttgccgcgtaacattacctccacgcccgcttctaatgtagaaaccatggcaaaaattaatgttttaagggccatttgcgtaggatttgccgaggtccgtcgccacaacgacgcgcgaactttacagcgaacggcaatgtttgccgccgacgacgtcgcatcacggatcagaccatccattggattaaagcgcacctacccaccgggtatattttccacagctattaccgtatctaattccgaggatgaagagcgaaattcgtgatcgtaaaaataaaaaatacaagatattgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccgccaccatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgtacataattcttatttatctttaatccatgaggagcatttttattttaaaaatgtcagccgccagccctataaccctagatcgcaactgatccctagtctgcgttatttgtcttgcaatcttttcgcacgcctttgtgagtgcatacaatgcccccctgctcgcttttctgaaatcgcgtcgggtcattaatgtgtcggctatcacaatgcgagatgtactcgacatgtccgtgtctgtactattgggattgtaaatagtcgaccgcgaatcatcagagtcggaatctgtaaaggatacagattccgactctgagcgcttatgaatgggatccactcggacgttgttgaacttccgttcggattctgcttcagtcaacaccggcccccgatagctactaaggttggggggtttgtgggttgtttgtgaaactgctttgcggtgtgcattaccacggggggtgtggggaagtatctgtttccacgatgcgataacgttcggtggcggagggggcgattcattctctagtgtacgcgtttcaacttcaggaacgtgattatttctttcaggacactctttccaatttccttcttccttcacttcgggtacaggtatattcttaatgtttacatacatgtcgtctgctcgtctcaactgcggggttatgatgggtggtggtgacagtctctccgaatgatcg

SEQ ID NO: 18 - DNA, 2895 bp, synthetic construct
  misc_feature 1..2895: note = DNA Sequence Transfer plasmid for HVT-gfp-B #13
  source 1..2895: mol_type = other DNA
atggcagcgagcatgggatattcatcctcgtcatcgttaacatctctacgggttcagaatgtttggcatgtcgtcgatcctttgcccatcgttgcaaattacaagtccgatcgccatgaccgcgataagcctgtaccatgtggcattagggtgacatctcgatcatacattataagaccaacgtgcgagtcttccaaagacctgcacgccttcttcttcggattgtcaacgggttcttcagaatctatgcccatatctggcgttgagaccattgtgcgtttaatgaacaataaagcggcatgccatggaaaggagggctgcagatctccattttctcacgccactatcctggacgctgtagacgataattataccatgaatatagagggggtatgtttccactgccactgtgatgataagttttctccagattgttggatatctgcattttctgctgccgaacaaacttcatcgctatgcaaagagatgcgtgtgtacacgcgccgttgagtatacgggaaactaaatgttcatagaggtctttgggctatatgttattaaataaaataattgaccagtgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccgccaccatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtgatgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttttaatgttagtttattcaatgcattggttgcaaatattcattacttctccaatcccaggtcattctttagcgagatgatgttatgacattgctgtgaaaattactacaggatatatttttaagatgcaggagtaacaatgtgcatagtaggcgtagttatcgcagacgtgcaacgcttcgcatttgagttaccgaagtgcccaacagtgctgcggttatggtttatgcgcacagaatccatgcatgtcctaattgaaccatccgatttttcttttaatcgcgatcgttgtttgggcaactgcgttatttcagatctaaaaaatttaccctttatgaccatcacatctctctggctcataccccgcttggataagatatcatgtagattccgccctaagaaatgcaaactaacattattgtcggttccatatacacttccatcttgtccttcgaaaataacaaactcgcgcaatagaccgtccgtacatgcatggccgatgtgtgtcaacatcattggtctgctagatcccgatgggacgaatcgtacagtcgtcgctccagcattggcaaaaatccccagataccctccatgcggcaaatctaaattgcgaccccgaagagactgcaccaaagtcttatcgacgcacgctgatttttttgaacagcgggagcccat

SEQ ID NO: 19 - DNA, 2908 bp, synthetic construct
  misc_feature 1..2908: note = DNA sequence Transfer plasmid for HVT-gfp-B #13a
  source 1..2908: mol_type = other DNA
atggcagcgagcatgggatattcatcctcgtcatcgttaacatctctacgggttcagaatgtttggcatgtcgtcgatcctttgcccatcgttgcaaattacaagtccgatcgccatgaccgcgataagcctgtaccatgtggcattagggtgacatctcgatcatacattataagaccaacgtgcgagtcttccaaagacctgcacgccttcttcttcggattgtcaacgggttcttcagaatctatgcccatatctggcgttgagaccattgtgcgtttaatgaacaataaagcggcatgccatggaaaggagggctgcagatctccattttctcacgccactatcctggacgctgtagacgataattataccatgaatatagagggggtatgtttccactgccactgtgatgataagttttctccagattgttggatatctgcattttctgctgccgaacaaacttcatcgctatgcaaagagatgcgtgtgtacacgcgccgttgagtatacgggaaactaaatgttcatagaggtctttgggctatatgttattaaataaaataattgaccagtgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttacctgcaggccaccatggtgagcaagggcgccgagctgttcaccggcatcgtgcccatcctgatcgagctgaatggcgatgtgaatggccacaagttcagcgtgagcggcgagggcgagggcgatgccacctacggcaagctgaccctgaagttcatctgcaccaccggcaagctgcctgtgccctggcccaccctggtgaccaccctgagctacggcgtgcagtgcttctcacgctaccccgatcacatgaagcagcacgacttcttcaagagcgccatgcctgagggctacatccaggagcgcaccatcttcttcgaggatgacggcaactacaagtcgcgcgccgaggtgaagttcgagggcgataccctggtgaatcgcatcgagctgaccggcaccgatttcaaggaggatggcaacatcctgggcaataagatggagtacaactacaacgcccacaatgtgtacatcatgaccgacaaggccaagaatggcatcaaggtgaacttcaagatccgccacaacatcgaggatggcagcgtgcagctggccgaccactaccagcagaatacccccatcggcgatggccctgtgctgctgcccgataaccactacctgtccacccagagcgccctgtccaaggaccccaacgagaagcgcgatcacatgatctacttcggcttcgtgaccgccgccgccatcacccacggcatggatgagctgtacaagtgacctgcaggtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggttttaatgttagtttattcaatgcattggttgcaaatattcattacttctccaatcccaggtcattctttagcgagatgatgttatgacattgctgtgaaaattactacaggatatatttttaagatgcaggagtaacaatgtgcatagtaggcgtagttatcgcagacgtgcaacgcttcgcatttgagttaccgaagtgcccaacagtgctgcggttatggtttatgcgcacagaatccatgcatgtcctaattgaaccatccgatttttcttttaatcgcgatcgttgtttgggcaactgcgttatttcagatctaaaaaatttaccctttatgaccatcacatctctctggctcataccccgcttggataagatatcatgtagattccgccctaagaaatgcaaactaacattattgtcggttccatatacacttccatcttgtccttcgaaaataacaaactcgcgcaatagaccgtccgtacatgcatggccgatgtgtgtcaacatcattggtctgctagatcccgatgggacgaatcgtacagtcgtcgctccagcattggcaaaaatccccagataccctccatgcggcaaatctaaattgcgaccccgaagagactgcaccaaagtcttatcgacgcacgctgatttttttgaacagcgggagcccat

SEQ ID NO: 20 - DNA, 3538 bp, synthetic construct
  misc_feature 1..3538: note = DNA sequence Transfer plasmid for HVT-IBD #1
  source 1..3538: mol_type = other DNA
atggcagcgagcatgggatattcatcctcgtcatcgttaacatctctacgggttcagaatgtttggcatgtcgtcgatcctttgcccatcgttgcaaattacaagtccgatcgccatgaccgcgataagcctgtaccatgtggcattagggtgacatctcgatcatacattataagaccaacgtgcgagtcttccaaagacctgcacgccttcttcttcggattgtcaacgggttcttcagaatctatgcccatatctggcgttgagaccattgtgcgtttaatgaacaataaagcggcatgccatggaaaggagggctgcagatctccattttctcacgccactatcctggacgctgtagacgataattataccatgaatatagagggggtatgtttccactgccactgtgatgataagttttctccagattgttggatatctgcattttctgctgccgaacaaacttcatcgctatgcaaagagatgcgtgtgtacacgcgccgttgagtatacgggaaactaaatgttcatagaggtctttgggctatatgttattaaataaaataattgaccagtgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccgccaccatgacaaatcttcaggatcagacccagcagatagttccctttattaggtcccttctgatgccaaccacagggcccgctagcattccggacgataccctggagaaacacactttgcggagtgagacaagcacttacaatctgacggtgggagataccggctcaggtcttatcgtgttttttcccggctttcctggatccattgttggcgcgcattacacgctgcagagcaacggcaactataaattcgatcagatgctcctgacggctcagaatctccccgccagttacaattactgccgccttgtaagtaggtccttgactgttagaagctcaacgctgccaggcggagtatatgccctgaatggaaccattaatgctgtaacattccaaggatcactgtccgagctcaccgatgtgtcttacaatggattgatgtctgccacggctaacattaacgacaagatcgggaatgtgctcgtgggcgagggagtgaccgttttgagcctgccgacaagctacgacctcggctacgtaaggctcggggatccaatccccgcgatcggcttggatcccaaaatggttgctacgtgcgacagcagcgatagacccagggtctataccatcaccgctgccgatgattaccagtttagctcccagtaccaggcgggaggggtcacgatcaccctttttagcgccaacatcgacgccataacctcactttctatagggggcgagttggtttttcagaccagtgtccaggggctcatcctcggtgcgacaatctatctgatcggctttgacggaacagctgtcatcacgagggccgtagctgcagataatggcctgactgctgggacagataatctgatgccgttcaacatagtgatccccaccagtgagattacgcaacccatcacgagcatcaaactggagatcgtgacgtcaaaatccggcggtcaggcaggtgaccagatgtcttggtccgcaagcggaagtttggccgtgacaattcacggggggaattaccccggcgcactcaggcccgtgaccctcgtcgcctacgaaagagttgcaacgggaagtgtagtgacagtcgctggagtgagtaacttcgaactcatccctaatcccgagctcgccaaaaatctcgtcacggagtatgggaggtttgatcccggcgccatgaactacacaaaactgatattgtccgaaagggataggttgggcattaaaaccgtgtggcctactagggaatacaccgatttccgcgaatattttatggaggtcgcggatctgaactctcccctgaagatagcaggcgcttttgggttcaaggatattatccgggcgttgcggcggtagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgtttaatgttagtttattcaatgcattggttgcaaatattcattacttctccaatcccaggtcattctttagcgagatgatgttatgacattgctgtgaaaattactacaggatatatttttaagatgcaggagtaacaatgtgcatagtaggcgtagttatcgcagacgtgcaacgcttcgcatttgagttaccgaagtgcccaacagtgctgcggttatggtttatgcgcacagaatccatgcatgtcctaattgaaccatccgatttttcttttaatcgcgatcgttgtttgggcaactgcgttatttcagatctaaaaaatttaccctttatgaccatcacatctctctggctcataccccgcttggataagatatcatgtagattccgccctaagaaatgcaaactaacattattgtcggttccatatacacttccatcttgtccttcgaaaataacaaactcgcgcaatagaccgtccgtacatgcatggccgatgtgtgtcaacatcattggtctgctagatcccgatgggacgaatcgtacagtcgtcgctccagcattggcaaaaatccccagataccctccatgcggcaaatctaaattgcgaccccgaagagactgcaccaaagtcttatcgacgcacgctgatttttttgaacagcgggagcccat

SEQ ID NO: 21 - DNA, 3511 bp, synthetic construct
  misc_feature 1..3511: note = DNA sequence Transfer plasmid for HVT-IBD #5
  source 1..3511: mol_type = other DNA
gccactgtatgggccatttatgtttatcgagtctaaaagtcgtggaagcgcctggccatcgccagtctcgacctgccactgctgagttcggcttcaattccagcatcccagaaatgtcgcagtcaccatcacgccgtgcatcgcttcctacgaggccttttgacgcttctgatttgggcacatacaccctggacatactccaccgctattcgctcgtagatttagtacaactactgaatgacttgccgcgtaacattacctccacgcccgcttctaatgtagaaaccatggcaaaaattaatgttttaagggccatttgcgtaggatttgccgaggtccgtcgccacaacgacgcgcgaactttacagcgaacggcaatgtttgccgccgacgacgtcgcatcacggatcagaccatccattggattaaagcgcacctacccaccgggtatattttccacagctattaccgtatctaattccgaggatgaagagcgaaattcgtgatcgtaaaaataaaaaatacaagatattgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccgccaccatgacaaatcttcaggatcagacccagcagatagttccctttattaggtcccttctgatgccaaccacagggcccgctagcattccggacgataccctggagaaacacactttgcggagtgagacaagcacttacaatctgacggtgggagataccggctcaggtcttatcgtgttttttcccggctttcctggatccattgttggcgcgcattacacgctgcagagcaacggcaactataaattcgatcagatgctcctgacggctcagaatctccccgccagttacaattactgccgccttgtaagtaggtccttgactgttagaagctcaacgctgccaggcggagtatatgccctgaatggaaccattaatgctgtaacattccaaggatcactgtccgagctcaccgatgtgtcttacaatggattgatgtctgccacggctaacattaacgacaagatcgggaatgtgctcgtgggcgagggagtgaccgttttgagcctgccgacaagctacgacctcggctacgtaaggctcggggatccaatccccgcgatcggcttggatcccaaaatggttgctacgtgcgacagcagcgatagacccagggtctataccatcaccgctgccgatgattaccagtttagctcccagtaccaggcgggaggggtcacgatcaccctttttagcgccaacatcgacgccataacctcactttctatagggggcgagttggtttttcagaccagtgtccaggggctcatcctcggtgcgacaatctatctgatcggctttgacggaacagctgtcatcacgagggccgtagctgcagataatggcctgactgctgggacagataatctgatgccgttcaacatagtgatccccaccagtgagattacgcaacccatcacgagcatcaaactggagatcgtgacgtcaaaatccggcggtcaggcaggtgaccagatgtcttggtccgcaagcggaagtttggccgtgacaattcacggggggaattaccccggcgcactcaggcccgtgaccctcgtcgcctacgaaagagttgcaacgggaagtgtagtgacagtcgctggagtgagtaacttcgaactcatccctaatcccgagctcgccaaaaatctcgtcacggagtatgggaggtttgatcccggcgccatgaactacacaaaactgatattgtccgaaagggataggttgggcattaaaaccgtgtggcctactagggaatacaccgatttccgcgaatattttatggaggtcgcggatctgaactctcccctgaagatagcaggcgcttttgggttcaaggatattatccgggcgttgcggcggtagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgtacataattcttatttatctttaatccatgaggagcatttttattttaaaaatgtcagccgccagccctataaccctagatcgcaactgatccctagtctgcgttatttgtcttgcaatcttttcgcacgcctttgtgagtgcatacaatgcccccctgctcgcttttctgaaatcgcgtcgggtcattaatgtgtcggctatcacaatgcgagatgtactcgacatgtccgtgtctgtactattgggattgtaaatagtcgaccgcgaatcatcagagtcggaatctgtaaaggatacagattccgactctgagcgcttatgaatgggatccactcggacgttgttgaacttccgttcggattctgcttcagtcaacaccggcccccgatagctactaaggttggggggtttgtgggttgtttgtgaaactgctttgcggtgtgcattaccacggggggtgtggggaagtatctgtttccacgatgcgataacgttcggtggcggagggggcgattcattctctagtgtacgcgtttcaacttcaggaacgtgattatttctttcaggacactctttccaatttccttcttccttcacttcgggtacaggtatattcttaatgtttacatacatgtcgtctgctcgtctcaactgcggggttatgatgggtggtggtgacagtctctccgaatgatcg

SEQ ID NO: 22 - DNA, 3385 bp, synthetic construct
  misc_feature 1..3385: note = DNA Sequence Transfer plasmid for HVT-IBD #6a
  source 1..3385: mol_type = other DNA
gccactgtatgggccatttatgtttatcgagtctaaaagtcgtggaagcgcctggccatcgccagtctcgacctgccactgctgagttcggcttcaattccagcatcccagaaatgtcgcagtcaccatcacgccgtgcatcgcttcctacgaggccttttgacgcttctgatttgggcacatacaccctggacatactccaccgctattcgctcgtagatttagtacaactactgaatgacttgccgcgtaacattacctccacgcccgcttctaatgtagaaaccatggcaaaaattaatgttttaagggccatttgcgtaggatttgccgaggtccgtcgccacaacgacgcgcgaactttacagcgaacggcaatgtttgccgccgacgacgtcgcatcacggatcagaccatccattggattaaagcgcacctacccaccgggtatattttccacagctattaccgtatctaattccgaggatgaagagcgaaattcgtgatcgtaaaaataaaaaatacaagataagttattaatagtaatcaattacggggtcattagttcatagcccatatatggagttccgcgttacataacttacggtaaatggcccgccggctgaccgcccaacgacccccgcccattgacgtcaataatgacgtatgttcccatagtaacgccaatagggactttccattgacgtcaatgggtggagtatttacggtaaactgcccattggcagtacatcaagtgtatcatatgccaagtacgccccctattgacgtcaatgacggtaaatggatgcagtattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgtctagagggccaccatgacaaatcttcaggatcagacccagcagatagttccctttattaggtcccttctgatgccaaccacagggcccgctagcattccggacgataccctggagaaacacactttgcggagtgagacaagcacttacaatctgacggtgggagataccggctcaggtcttatcgtgttttttcccggctttcctggatccattgttggcgcgcattacacgctgcagagcaacggcaactataaattcgatcagatgctcctgacggctcagaatctccccgccagttacaattactgccgccttgtaagtaggtccttgactgttagaagctcaacgctgccaggcggagtatatgccctgaatggaaccattaatgctgtaacattccaaggatcactgtccgagctcaccgatgtgtcttacaatggattgatgtctgccacggctaacattaacgacaagatcgggaatgtgctcgtgggcgagggagtgaccgttttgagcctgccgacaagctacgacctcggctacgtaaggctcggggatccaatccccgcgatcggcttggatcccaaaatggttgctacgtgcgacagcagcgatagacccagggtctataccatcaccgctgccgatgattaccagtttagctcccagtaccaggcgggaggggtcacgatcaccctttttagcgccaacatcgacgccataacctcactttctatagggggcgagttggtttttcagaccagtgtccaggggctcatcctcggtgcgacaatctatctgatcggctttgacggaacagctgtcatcacgagggccgtagctgcagataatggcctgactgctgggacagataatctgatgccgttcaacatagtgatccccaccagtgagattacgcaacccatcacgagcatcaaactggagatcgtgacgtcaaaatccggcggtcaggcaggtgaccagatgtcttggtccgcaagcggaagtttggccgtgacaattcacggggggaattaccccggcgcactcaggcccgtgaccctcgtcgcctacgaaagagttgcaacgggaagtgtagtgacagtcgctggagtgagtaacttcgaactcatccctaatcccgagctcgccaaaaatctcgtcacggagtatgggaggtttgatcccggcgccatgaactacacaaaactgatattgtccgaaagggataggttgggcattaaaaccgtgtggcctactagggaatacaccgatttccgcgaatattttatggaggtcgcggatctgaactctcccctgaagatagcaggcgcttttgggttcaaggatattatccgggcgttgcggcggtagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgtacataattcttatttatctttaatccatgaggagcatttttattttaaaaatgtcagccgccagccctataaccctagatcgcaactgatccctagtctgcgttatttgtcttgcaatcttttcgcacgcctttgtgagtgcatacaatgcccccctgctcgcttttctgaaatcgcgtcgggtcattaatgtgtcggctatcacaatgcgagatgtactcgacatgtccgtgtctgtactattgggattgtaaatagtcgaccgcgaatcatcagagtcggaatctgtaaaggatacagattccgactctgagcgcttatgaatgggatccactcggacgttgttgaacttccgttcggattctgcttcagtcaacaccggcccccgatagctactaaggttggggggtttgtgggttgtttgtgaaactgctttgcggtgtgcattaccacggggggtgtggggaagtatctgtttccacgatgcgataacgttcggtggcggagggggcgattcattctctagtgtacgcgtttcaacttcaggaacgtgattatttctttcaggacactctttccaatttccttcttccttcacttcgggtacaggtatattcttaatgtttacatacatgtcgtctgctcgtctcaactgcggggttatgatgggtggtggtgacagtctctccgaatgatcg

SEQ ID NO: 23 - DNA, 3538 bp, synthetic construct
  misc_feature 1..3538: note = DNA sequence Transfer plasmid for HVT-IBD #9
  source 1..3538: mol_type = other DNA
atggcagcgagcatgggatattcatcctcgtcatcgttaacatctctacgggttcagaatgtttggcatgtcgtcgatcctttgcccatcgttgcaaattacaagtccgatcgccatgaccgcgataagcctgtaccatgtggcattagggtgacatctcgatcatacattataagaccaacgtgcgagtcttccaaagacctgcacgccttcttcttcggattgtcaacgggttcttcagaatctatgcccatatctggcgttgagaccattgtgcgtttaatgaacaataaagcggcatgccatggaaaggagggctgcagatctccattttctcacgccactatcctggacgctgtagacgataattataccatgaatatagagggggtatgtttccactgccactgtgatgataagttttctccagattgttggatatctgcattttctgctgccgaacaaacttcatcgctatgcaaagagatgcgtgtgtacacgcgccgttgagtatacgggaaactaaatgttcatagaggtctttgggctatatgttattaaataaaataattgaccagtgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccgccaccatgaccaatctccaggaccagacccagcagattgtgccttttattaggagtctcttgatgcctacaaccggccccgccagcatcccggacgacacactggaaaaacatacactgagaagcgagacatctacatacaatttgaccgtgggcgataccggctccgggcttatcgtgttcttcccaggttttcccggatctatcgtaggagcgcactacaccctccaaagtaacggcaattacaaattcgaccagatgctcctgacagcccagaaccttcctgcttcttacaattactgtagacttgtgtccaggtccctgactgtgcggagtagcacgcttccaggaggcgtatacgccctgaacggaactataaacgccgtcaccttccagggctccttgtccgaacttaccgacgtgtcctacaatggcctcatgagcgcaacggccaacataaacgataagatcggcaatgttcttgtgggcgagggggttacagtcctttctctgccaaccagttatgatctgggatacgtgcggcttggcgatcccattcccgctatcggtctcgaccctaaaatggtggctacttgcgactcatctgaccgcccaagggtctatacaattactgcagccgatgactatcagttttccagccaataccagccagggggtgtgacaatcacacttttcagcgccaatattgacgctatcacatccctctcaatcggaggtgagcttgtgttccagacttctgttcagggcttggtattgggcgccactatttacttgatcgggttcgacgggaccgcagtgatcactcgggcagtggctgcggataacggactcactgccggaactgacaaccttatgccttttaatctggtcatccccactaacgagatcacccagcctattacctccataaagctcgaaattgtgaccagcaagagcggagggcaggcaggcgaccaaatgagttggtctgcaagcgggtccctcgccgtgaccatccacggtggcaactatcctggggcgctcagacccgtcaccctggtagcctacgaaagggttgccacaggctcagttgtcacggtggctggagtaagcaatttcgagctcatcccgaatcctgagctcgctaaaaatcttgtgaccgagtatggaaggttcgaccctggcgcaatgaattacacaaagctgattctgtccgaacgggataggctgggtatcaagacagtttggcccacgcgcgaatacacagatttcagggagtactttatggaggtcgcagatttgaatagcccacttaagatcgctggagcatttggctttaaggatattatccgcgcaatcagaaggtagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgtttaatgttagtttattcaatgcattggttgcaaatattcattacttctccaatcccaggtcattctttagcgagatgatgttatgacattgctgtgaaaattactacaggatatatttttaagatgcaggagtaacaatgtgcatagtaggcgtagttatcgcagacgtgcaacgcttcgcatttgagttaccgaagtgcccaacagtgctgcggttatggtttatgcgcacagaatccatgcatgtcctaattgaaccatccgatttttcttttaatcgcgatcgttgtttgggcaactgcgttatttcagatctaaaaaatttaccctttatgaccatcacatctctctggctcataccccgcttggataagatatcatgtagattccgccctaagaaatgcaaactaacattattgtcggttccatatacacttccatcttgtccttcgaaaataacaaactcgcgcaatagaccgtccgtacatgcatggccgatgtgtgtcaacatcattggtctgctagatcccgatgggacgaatcgtacagtcgtcgctccagcattggcaaaaatccccagataccctccatgcggcaaatctaaattgcgaccccgaagagactgcaccaaagtcttatcgacgcacgctgatttttttgaacagcgggagcccat

SEQ ID NO: 24 - DNA, 3511 bp, synthetic construct
  misc_feature 1..3511: note = DNA sequence Transfer plasmid for HVT-IBD #30
  source 1..3511: mol_type = other DNA
gccactgtatgggccatttatgtttatcgagtctaaaagtcgtggaagcgcctggccatcgccagtctcgacctgccactgctgagttcggcttcaattccagcatcccagaaatgtcgcagtcaccatcacgccgtgcatcgcttcctacgaggccttttgacgcttctgatttgggcacatacaccctggacatactccaccgctattcgctcgtagatttagtacaactactgaatgacttgccgcgtaacattacctccacgcccgcttctaatgtagaaaccatggcaaaaattaatgttttaagggccatttgcgtaggatttgccgaggtccgtcgccacaacgacgcgcgaactttacagcgaacggcaatgtttgccgccgacgacgtcgcatcacggatcagaccatccattggattaaagcgcacctacccaccgggtatattttccacagctattaccgtatctaattccgaggatgaagagcgaaattcgtgatcgtaaaaataaaaaatacaagatattgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccgccaccatgaccaatctccaggaccagacccagcagattgtgccttttattaggagtctcttgatgcctacaaccggccccgccagcatcccggacgacacactggaaaaacatacactgagaagcgagacatctacatacaatttgaccgtgggcgataccggctccgggcttatcgtgttcttcccaggttttcccggatctatcgtaggagcgcactacaccctccaaagtaacggcaattacaaattcgaccagatgctcctgacagcccagaaccttcctgcttcttacaattactgtagacttgtgtccaggtccctgactgtgcggagtagcacgcttccaggaggcgtatacgccctgaacggaactataaacgccgtcaccttccagggctccttgtccgaacttaccgacgtgtcctacaatggcctcatgagcgcaacggccaacataaacgataagatcggcaatgttcttgtgggcgagggggttacagtcctttctctgccaaccagttatgatctgggatacgtgcggcttggcgatcccattcccgctatcggtctcgaccctaaaatggtggctacttgcgactcatctgaccgcccaagggtctatacaattactgcagccgatgactatcagttttccagccaataccagccagggggtgtgacaatcacacttttcagcgccaatattgacgctatcacatccctctcaatcggaggtgagcttgtgttccagacttctgttcagggcttggtattgggcgccactatttacttgatcgggttcgacgggaccgcagtgatcactcgggcagtggctgcggataacggactcactgccggaactgacaaccttatgccttttaatctggtcatccccactaacgagatcacccagcctattacctccataaagctcgaaattgtgaccagcaagagcggagggcaggcaggcgaccaaatgagttggtctgcaagcgggtccctcgccgtgaccatccacggtggcaactatcctggggcgctcagacccgtcaccctggtagcctacgaaagggttgccacaggctcagttgtcacggtggctggagtaagcaatttcgagctcatcccgaatcctgagctcgctaaaaatcttgtgaccgagtatggaaggttcgaccctggcgcaatgaattacacaaagctgattctgtccgaacgggataggctgggtatcaagacagtttggcccacgcgcgaatacacagatttcagggagtactttatggaggtcgcagatttgaatagcccacttaagatcgctggagcatttggctttaaggatattatccgcgcaatcagaaggtagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgtacataattcttatttatctttaatccatgaggagcatttttattttaaaaatgtcagccgccagccctataaccctagatcgcaactgatccctagtctgcgttatttgtcttgcaatcttttcgcacgcctttgtgagtgcatacaatgcccccctgctcgcttttctgaaatcgcgtcgggtcattaatgtgtcggctatcacaatgcgagatgtactcgacatgtccgtgtctgtactattgggattgtaaatagtcgaccgcgaatcatcagagtcggaatctgtaaaggatacagattccgactctgagcgcttatgaatgggatccactcggacgttgttgaacttccgttcggattctgcttcagtcaacaccggcccccgatagctactaaggttggggggtttgtgggttgtttgtgaaactgctttgcggtgtgcattaccacggggggtgtggggaagtatctgtttccacgatgcgataacgttcggtggcggagggggcgattcattctctagtgtacgcgtttcaacttcaggaacgtgattatttctttcaggacactctttccaatttccttcttccttcacttcgggtacaggtatattcttaatgtttacatacatgtcgtctgctcgtctcaactgcggggttatgatgggtggtggtgacagtctctccgaatgatcg

SEQ ID NO: 25 - DNA, 4102 bp, synthetic construct
  misc_feature 1..4102: note = DNA sequence Transfer plasmid for HVT-IBD #31
  source 1..4102: mol_type = other DNA
gccactgtatgggccatttatgtttatcgagtctaaaagtcgtggaagcgcctggccatcgccagtctcgacctgccactgctgagttcggcttcaattccagcatcccagaaatgtcgcagtcaccatcacgccgtgcatcgcttcctacgaggccttttgacgcttctgatttgggcacatacaccctggacatactccaccgctattcgctcgtagatttagtacaactactgaatgacttgccgcgtaacattacctccacgcccgcttctaatgtagaaaccatggcaaaaattaatgttttaagggccatttgcgtaggatttgccgaggtccgtcgccacaacgacgcgcgaactttacagcgaacggcaatgtttgccgccgacgacgtcgcatcacggatcagaccatccattggattaaagcgcacctacccaccgggtatattttccacagctattaccgtatctaattccgaggatgaagagcgaaattcgtgatcgtaaaaataaaaaatacaagatatcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggggtttatatcttcccttctctgttcctccgcagccagccatggccaccatgaccaatctccaggaccagacccagcagattgtgccttttattaggagtctcttgatgcctacaaccggccccgccagcatcccggacgacacactggaaaaacatacactgagaagcgagacatctacatacaatttgaccgtgggcgataccggctccgggcttatcgtgttcttcccaggttttcccggatctatcgtaggagcgcactacaccctccaaagtaacggcaattacaaattcgaccagatgctcctgacagcccagaaccttcctgcttcttacaattactgtagacttgtgtccaggtccctgactgtgcggagtagcacgcttccaggaggcgtatacgccctgaacggaactataaacgccgtcaccttccagggctccttgtccgaacttaccgacgtgtcctacaatggcctcatgagcgcaacggccaacataaacgataagatcggcaatgttcttgtgggcgagggggttacagtcctttctctgccaaccagttatgatctgggatacgtgcggcttggcgatcccattcccgctatcggtctcgaccctaaaatggtggctacttgcgactcatctgaccgcccaagggtctatacaattactgcagccgatgactatcagttttccagccaataccagccagggggtgtgacaatcacacttttcagcgccaatattgacgctatcacatccctctcaatcggaggtgagcttgtgttccagacttctgttcagggcttggtattgggcgccactatttacttgatcgggttcgacgggaccgcagtgatcactcgggcagtggctgcggataacggactcactgccggaactgacaaccttatgccttttaatctggtcatccccactaacgagatcacccagcctattacctccataaagctcgaaattgtgaccagcaagagcggagggcaggcaggcgaccaaatgagttggtctgcaagcgggtccctcgccgtgaccatccacggtggcaactatcctggggcgctcagacccgtcaccctggtagcctacgaaagggttgccacaggctcagttgtcacggtggctggagtaagcaatttcgagctcatcccgaatcctgagctcgctaaaaatcttgtgaccgagtatggaaggttcgaccctggcgcaatgaattacacaaagctgattctgtccgaacgggataggctgggtatcaagacagtttggcccacgcgcgaatacacagatttcagggagtactttatggaggtcgcagatttgaatagcccacttaagatcgctggagcatttggctttaaggatattatccgcgcaatcagaaggtagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgtacataattcttatttatctttaatccatgaggagcatttttattttaaaaatgtcagccgccagccctataaccctagatcgcaactgatccctagtctgcgttatttgtcttgcaatcttttcgcacgcctttgtgagtgcatacaatgcccccctgctcgcttttctgaaatcgcgtcgggtcattaatgtgtcggctatcacaatgcgagatgtactcgacatgtccgtgtctgtactattgggattgtaaatagtcgaccgcgaatcatcagagtcggaatctgtaaaggatacagattccgactctgagcgcttatgaatgggatccactcggacgttgttgaacttccgttcggattctgcttcagtcaacaccggcccccgatagctactaaggttggggggtttgtgggttgtttgtgaaactgctttgcggtgtgcattaccacggggggtgtggggaagtatctgtttccacgatgcgataacgttcggtggcggagggggcgattcattctctagtgtacgcgtttcaacttcaggaacgtgattatttctttcaggacactctttccaatttccttcttccttcacttcgggtacaggtatattcttaatgtttacatacatgtcgtctgctcgtctcaactgcggggttatgatgggtggtggtgacagtctctccgaatgatcg

SEQ ID NO: 26 - DNA, 3394 bp, synthetic construct
  misc_feature 1..3394: note = DNA sequence Transfer plasmid for HVT-IBD #32
  source 1..3394: mol_type = other DNA
gccactgtatgggccatttatgtttatcgagtctaaaagtcgtggaagcgcctggccatcgccagtctcgacctgccactgctgagttcggcttcaattccagcatcccagaaatgtcgcagtcaccatcacgccgtgcatcgcttcctacgaggccttttgacgcttctgatttgggcacatacaccctggacatactccaccgctattcgctcgtagatttagtacaactactgaatgacttgccgcgtaacattacctccacgcccgcttctaatgtagaaaccatggcaaaaattaatgttttaagggccatttgcgtaggatttgccgaggtccgtcgccacaacgacgcgcgaactttacagcgaacggcaatgtttgccgccgacgacgtcgcatcacggatcagaccatccattggattaaagcgcacctacccaccgggtatattttccacagctattaccgtatctaattccgaggatgaagagcgaaattcgtgatcgtaaaaataaaaaatacaagataactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccagccaccatgaccaatctccaggaccagacccagcagattgtgccttttattaggagtctcttgatgcctacaaccggccccgccagcatcccggacgacacactggaaaaacatacactgagaagcgagacatctacatacaatttgaccgtgggcgataccggctccgggcttatcgtgttcttcccaggttttcccggatctatcgtaggagcgcactacaccctccaaagtaacggcaattacaaattcgaccagatgctcctgacagcccagaaccttcctgcttcttacaattactgtagacttgtgtccaggtccctgactgtgcggagtagcacgcttccaggaggcgtatacgccctgaacggaactataaacgccgtcaccttccagggctccttgtccgaacttaccgacgtgtcctacaatggcctcatgagcgcaacggccaacataaacgataagatcggcaatgttcttgtgggcgagggggttacagtcctttctctgccaaccagttatgatctgggatacgtgcggcttggcgatcccattcccgctatcggtctcgaccctaaaatggtggctacttgcgactcatctgaccgcccaagggtctatacaattactgcagccgatgactatcagttttccagccaataccagccagggggtgtgacaatcacacttttcagcgccaatattgacgctatcacatccctctcaatcggaggtgagcttgtgttccagacttctgttcagggcttggtattgggcgccactatttacttgatcgggttcgacgggaccgcagtgatcactcgggcagtggctgcggataacggactcactgccggaactgacaaccttatgccttttaatctggtcatccccactaacgagatcacccagcctattacctccataaagctcgaaattgtgaccagcaagagcggagggcaggcaggcgaccaaatgagttggtctgcaagcgggtccctcgccgtgaccatccacggtggcaactatcctggggcgctcagacccgtcaccctggtagcctacgaaagggttgccacaggctcagttgtcacggtggctggagtaagcaatttcgagctcatcccgaatcctgagctcgctaaaaatcttgtgaccgagtatggaaggttcgaccctggcgcaatgaattacacaaagctgattctgtccgaacgggataggctgggtatcaagacagtttggcccacgcgcgaatacacagatttcagggagtactttatggaggtcgcagatttgaatagcccacttaagatcgctggagcatttggctttaaggatattatccgcgcaatcagaaggtagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgtacataattcttatttatctttaatccatgaggagcatttttattttaaaaatgtcagccgccagccctataaccctagatcgcaactgatccctagtctgcgttatttgtcttgcaatcttttcgcacgcctttgtgagtgcatacaatgcccccctgctcgcttttctgaaatcgcgtcgggtcattaatgtgtcggctatcacaatgcgagatgtactcgacatgtccgtgtctgtactattgggattgtaaatagtcgaccgcgaatcatcagagtcggaatctgtaaaggatacagattccgactctgagcgcttatgaatgggatccactcggacgttgttgaacttccgttcggattctgcttcagtcaacaccggcccccgatagctactaaggttggggggtttgtgggttgtttgtgaaactgctttgcggtgtgcattaccacggggggtgtggggaagtatctgtttccacgatgcgataacgttcggtggcggagggggcgattcattctctagtgtacgcgtttcaacttcaggaacgtgattatttctttcaggacactctttccaatttccttcttccttcacttcgggtacaggtatattcttaatgtttacatacatgtcgtctgctcgtctcaactgcggggttatgatgggtggtggtgacagtctctccgaatgatcg

SEQ ID NO: 27 - DNA, 3243 bp, synthetic construct
  misc_feature 1..3243: note = DNA sequence Transfer plasmid for HVT-IBD #33
  source 1..3243: mol_type = other DNA
gccactgtatgggccatttatgtttatcgagtctaaaagtcgtggaagcgcctggccatcgccagtctcgacctgccactgctgagttcggcttcaattccagcatcccagaaatgtcgcagtcaccatcacgccgtgcatcgcttcctacgaggccttttgacgcttctgatttgggcacatacaccctggacatactccaccgctattcgctcgtagatttagtacaactactgaatgacttgccgcgtaacattacctccacgcccgcttctaatgtagaaaccatggcaaaaattaatgttttaagggccatttgcgtaggatttgccgaggtccgtcgccacaacgacgcgcgaactttacagcgaacggcaatgtttgccgccgacgacgtcgcatcacggatcagaccatccattggattaaagcgcacctacccaccgggtatattttccacagctattaccgtatctaattccgaggatgaagagcgaaattcgtgatcgtaaaaataaaaaatacaagatagcgcagcaccatggcctgaaataacctctgaaagaggaacttggttagctaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcgccaccatgaccaatctccaggaccagacccagcagattgtgccttttattaggagtctcttgatgcctacaaccggccccgccagcatcccggacgacacactggaaaaacatacactgagaagcgagacatctacatacaatttgaccgtgggcgataccggctccgggcttatcgtgttcttcccaggttttcccggatctatcgtaggagcgcactacaccctccaaagtaacggcaattacaaattcgaccagatgctcctgacagcccagaaccttcctgcttcttacaattactgtagacttgtgtccaggtccctgactgtgcggagtagcacgcttccaggaggcgtatacgccctgaacggaactataaacgccgtcaccttccagggctccttgtccgaacttaccgacgtgtcctacaatggcctcatgagcgcaacggccaacataaacgataagatcggcaatgttcttgtgggcgagggggttacagtcctttctctgccaaccagttatgatctgggatacgtgcggcttggcgatcccattcccgctatcggtctcgaccctaaaatggtggctacttgcgactcatctgaccgcccaagggtctatacaattactgcagccgatgactatcagttttccagccaataccagccagggggtgtgacaatcacacttttcagcgccaatattgacgctatcacatccctctcaatcggaggtgagcttgtgttccagacttctgttcagggcttggtattgggcgccactatttacttgatcgggttcgacgggaccgcagtgatcactcgggcagtggctgcggataacggactcactgccggaactgacaaccttatgccttttaatctggtcatccccactaacgagatcacccagcctattacctccataaagctcgaaattgtgaccagcaagagcggagggcaggcaggcgaccaaatgagttggtctgcaagcgggtccctcgccgtgaccatccacggtggcaactatcctggggcgctcagacccgtcaccctggtagcctacgaaagggttgccacaggctcagttgtcacggtggctggagtaagcaatttcgagctcatcccgaatcctgagctcgctaaaaatcttgtgaccgagtatggaaggttcgaccctggcgcaatgaattacacaaagctgattctgtccgaacgggataggctgggtatcaagacagtttggcccacgcgcgaatacacagatttcagggagtactttatggaggtcgcagatttgaatagcccacttaagatcgctggagcatttggctttaaggatattatccgcgcaatcagaaggtagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgtacataattcttatttatctttaatccatgaggagcatttttattttaaaaatgtcagccgccagccctataaccctagatcgcaactgatccctagtctgcgttatttgtcttgcaatcttttcgcacgcctttgtgagtgcatacaatgcccccctgctcgcttttctgaaatcgcgtcgggtcattaatgtgtcggctatcacaatgcgagatgtactcgacatgtccgtgtctgtactattgggattgtaaatagtcgaccgcgaatcatcagagtcggaatctgtaaaggatacagattccgactctgagcgcttatgaatgggatccactcggacgttgttgaacttccgttcggattctgcttcagtcaacaccggcccccgatagctactaaggttggggggtttgtgggttgtttgtgaaactgctttgcggtgtgcattaccacggggggtgtggggaagtatctgtttccacgatgcgataacgttcggtggcggagggggcgattcattctctagtgtacgcgtttcaacttcaggaacgtgattatttctttcaggacactctttccaatttccttcttccttcacttcgggtacaggtatattcttaatgtttacatacatgtcgtctgctcgtctcaactgcggggttatgatgggtggtggtgacagtctctccgaatgatcg

SEQ ID NO: 28 - DNA, 4124 bp, synthetic construct
  misc_feature 1..4124: note = DNA sequence Transfer plasmid for HVT-IBD #34
  source 1..4124: mol_type = other DNA
atgggctcccgctgttcaaaaaaatcagcgtgcgtcgataagactttggtgcagtctcttcggggtcgcaatttagatttgccgcatggagggtatctggggatttttgccaatgctggagcgacgactgtacgattcgtcccatcgggatctagcagaccaatgatgttgacacacatcggccatgcatgtacggacggtctattgcgcgagtttgttattttcgaaggacaagatggaagtgtatatggaaccgacaataatgttagtttgcatttcttagggcggaatctacatgatatcttatccaagcggggtatgagccagagagatgtgatggtcataaagggtaaattttttagatctgaaataacgcagttgcccaaacaacgatcgcgattaaaagaaaaatcggatggttcaattaggacatgcatggattctgtgcgcataaaccataaccgcagcactgttgggcacttcggtaactcaaatgcgaagcgttgcacgtctgcgataactacgcctactatgcacattgttactcctgcatcttaaaaatatatcctgtagtaattttcacagcaatgtcataacatcatctcgctaaagaatgacctgggattggagaagtaatgaatatttgcaaccaatgcattgaataaactaacattaaactcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggggtttatatcttcccttctctgttcctccgcagccagccatggccaccatgacaaacctgcaagatcaaacccaacagattgttccgttcatacggagccttctgatgccaacaaccggaccggcgtccattccggacgacaccctggagaagcacactctcaggtcagagacctcgacctacaatttgactgtgggggacacagggtcagggctaattgtctttttccctggattccctggctcaattgtgggtgctcactacacactgcagagcaatgggaactacaagttcgatcagatgctcctgactgcccagaacctaccggccagctacaactactgcagactagtgagtcggagtctcacagtgaggtcaagcacactccctggtggcgtttatgcactaaacggcaccataaacgccgtgaccttccaaggaagcctgagtgaactgacagatgttagctacaatgggttgatgtctgcaacagccaacatcaacgacaaaattgggaatgtcctggtaggggaaggggtcactgtcctcagcctacccacatcatatgatcttgggtatgtgaggcttggtgaccccattcccgctatagggcttgacccaaaaatggtagctacatgcgacagcagtgacaggcccagagtctacaccataactgcagccgatgattaccaattctcatcacagtaccaaccaggtggggtaacaatcacactgttctcagccaacattgatgctatcacaagcctcagcattgggggagagctcgtgtttcaaacaagcgtccaaggccttgtactgggcgccaccatctaccttataggctttgatgggactgcggtaatcaccagagctgtagccgcagataatgggctgacggccggcaccgacaatcttatgccattcaatcttgtcattccaaccaatgagataacccagccaatcacatccatcaaactggagatagtgacctccaaaagtggtggtcaggcaggggatcagatgtcatggtcggcaagtgggagcctagcagtgacgatccatggtggcaactatccaggggccctccgtcccgtcacactagtagcctacgaaagagtggcaacaggatccgtcgttacggtcgctggggtgagtaacttcgagctgattccaaatcctgaactagcaaagaacctggttacagaatacggccgatttgacccaggagccatgaactacacaaaattgatactgagtgagagggaccgtcttggcatcaagaccgtctggccaacaagggagtacactgattttcgtgagtacttcatggaggtggccgacctcaactctcccctgaagattgcaggagcatttggcttcaaagacataatccgggctataaggaggtaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtcaattattttatttaataacatatagcccaaagacctctatgaacatttagtttcccgtatactcaacggcgcgtgtacacacgcatctctttgcatagcgatgaagtttgttcggcagcagaaaatgcagatatccaacaatctggagaaaacttatcatcacagtggcagtggaaacataccccctctatattcatggtataattatcgtctacagcgtccaggatagtggcgtgagaaaatggagatctgcagccctcctttccatggcatgccgctttattgttcattaaacgcacaatggtctcaacgccagatatgggcatagattctgaagaacccgttgacaatccgaagaagaaggcgtgcaggtctttggaagactcgcacgttggtcttataatgtatgatcgagatgtcaccctaatgccacatggtacaggcttatcgcggtcatggcgatcggacttgtaatttgcaacgatgggcaaaggatcgacgacatgccaaacattctgaacccgtagagatgttaacgatgacgaggatgaatatcccatgctcgctgccat

SEQ ID NO: 29 - DNA, 4572 bp, synthetic construct
  misc_feature 1..4572: note = DNA sequence of Transfer plasmid for HVT-ND #38
  source 1..4572: mol_type = other DNA
gccactgtatgggccatttatgtttatcgagtctaaaagtcgtggaagcgcctggccatcgccagtctcgacctgccactgctgagttcggcttcaattccagcatcccagaaatgtcgcagtcaccatcacgccgtgcatcgcttcctacgaggccttttgacgcttctgatttgggcacatacaccctggacatactccaccgctattcgctcgtagatttagtacaactactgaatgacttgccgcgtaacattacctccacgcccgcttctaatgtagaaaccatggcaaaaattaatgttttaagggccatttgcgtaggatttgccgaggtccgtcgccacaacgacgcgcgaactttacagcgaacggcaatgtttgccgccgacgacgtcgcatcacggatcagaccatccattggattaaagcgcacctacccaccgggtatattttccacagctattaccgtatctaattccgaggatgaagagcgaaattcgtgatcgtaaaaataaaaaatacaagatagaattcactagtggatcccccaactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcaggcggccgctctagaactcgtcgatcgcagcgatgggctccagatcttctaccaggatcccagtacctctgatgctgaccgtccgaatcatgttggcactgagttgcgtctgtccgaccagctcccttgatggcaggcctcttgcagctgcagggattgtggtaacaggagacaaagcagtcaacatatacacctcatctcagacagggtcaatcataatcaagttactcccaaatatgcccaaggataaagaggcgtgtgcaaaagccccattggaagcatacaacaggacattgactactttgctcaccccccttggtgattctatccgtaggatacaagagtctgtgaccacatccggaggagggaaacagggacgtcttataggcgccattatcggtggtgtagctctcggggttgcaaccgctgcacagataacagcagcctcggctctgatacaagccaatcaaaatgctgccaacatcctccggctcaaagagagcattgctgcaaccaatgaggctgtgcacgaggtcactgacggattatcacaactagcagtggcagttgggaagatgcagcaatttgttaatgaccagtttaataaaacagctcaggaattggactgtataaaaattacacagcaggttggtgtagaactcaacctgtacctaactgaattgactacagtattcgggccacaaatcacttcccctgccttaactcagctgactatccaggcgctttacaatctagctggtgggaatatggattacttgttgactaagttaggtgtaggaaacaaccaactcagctcattaattggtagtggcctgattaccggcaaccctatcctgtacgactcacagactcaactcttgggtatacaggtcaccctaccctcagtcgggaatctaaataatatgcgtgccacctacctggaaaccttgtctgtaagtacaaccaaaggatttgcctcagcacttgtcccaaaagtagtgacacaggttggttccgtgatagaagagcttgacacctcgtactgtatcgagaccgatttggacctatattgtacaagaatagtgacattccctatgtctcctggtatttattcctgtttgagtggcaatacatctgcttgcatgtattcaaagactgaaggcgcactcactacgccgtatatgaccctcaaaggctcagttattgccaactgtaagatgacaacatgtagatgtgcagaccccccgggtatcatatcgcagaattatggagaagctgtgtctctaatagataggcaatcatgcaatatcttatccttagacgggataactttgaggctcagtggggaatttgatgcaacttatcaaaagaatatctcaatacaagattctcaagtaatagttacaggcaatcttgacatctcgactgagcttgggaatgtcaacaactcgataagtaatgctttggataagttagaggaaagcaacagcaaactagacaaggtcaatgttaaactgaccagcacatccgctcttattacctatatcgttttaactgtcatatctcttgtatgtggtatacttagcctggttctagcatgctacctgatgtacaagcaaaaggcgcaacagaagaccttgttgtggcttgggaataataccctagaccagatgagggccactacaaaaatgtagcttgatctagagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgagtacataattcttatttatctttaatccatgaggagcatttttattttaaaaatgtcagccgccagccctataaccctagatcgcaactgatccctagtctgcgttatttgtcttgcaatcttttcgcacgcctttgtgagtgcatacaatgcccccctgctcgcttttctgaaatcgcgtcgggtcattaatgtgtcggctatcacaatgcgagatgtactcgacatgtccgtgtctgtactattgggattgtaaatagtcgaccgcgaatcatcagagtcggaatctgtaaaggatacagattccgactctgagcgcttatgaatgggatccactcggacgttgttgaacttccgttcggattctgcttcagtcaacaccggcccccgatagctactaaggttggggggtttgtgggttgtttgtgaaactgctttgcggtgtgcattaccacggggggtgtggggaagtatctgtttccacgatgcgataacgttcggtggcggagggggcgattcattctctagtgtacgcgtttcaacttcaggaacgtgattatttctttcaggacactctttccaatttccttcttccttcacttcgggtacaggtatattcttaatgtttacatacatgtcgtctgctcgtctcaactgcggggttatgatgggtggtggtgacagtctctccgaatgatcg

SEQ ID NO: 30 - DNA, 4402 bp, synthetic construct
  misc_feature 1..4402: note = DNA sequence Transfer plasmid for HVT-ND #39
  source 1..4402: mol_type = other DNA
gccactgtatgggccatttatgtttatcgagtctaaaagtcgtggaagcgcctggccatcgccagtctcgacctgccactgctgagttcggcttcaattccagcatcccagaaatgtcgcagtcaccatcacgccgtgcatcgcttcctacgaggccttttgacgcttctgatttgggcacatacaccctggacatactccaccgctattcgctcgtagatttagtacaactactgaatgacttgccgcgtaacattacctccacgcccgcttctaatgtagaaaccatggcaaaaattaatgttttaagggccatttgcgtaggatttgccgaggtccgtcgccacaacgacgcgcgaactttacagcgaacggcaatgtttgccgccgacgacgtcgcatcacggatcagaccatccattggattaaagcgcacctacccaccgggtatattttccacagctattaccgtatctaattccgaggatgaagagcgaaattcgtgatcgtaaaaataaaaaatacaagatatcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggggtttatatcttcccttctctgttcctccgcagccagccatggccaccatgggctccagatcttctaccaggatcccagtacctctgatgctgaccgtccgaatcatgttggcactgagttgcgtctgtccgaccagctcccttgatggcaggcctcttgcagctgcagggattgtggtaacaggagacaaagcagtcaacatatacacctcatctcagacagggtcaatcataatcaagttactcccaaatatgcccaaggataaagaggcgtgtgcaaaagccccattggaagcatacaacaggacattgactactttgctcaccccccttggtgattctatccgtaggatacaagagtctgtgaccacatccggaggagggaaacagggacgtcttataggcgccattatcggtggtgtagctctcggggttgcaaccgctgcacagataacagcagcctcggctctgatacaagccaatcaaaatgctgccaacatcctccggctcaaagagagcattgctgcaaccaatgaggctgtgcacgaggtcactgacggattatcacaactagcagtggcagttgggaagatgcagcaatttgttaatgaccagtttaataaaacagctcaggaattggactgtataaaaattacacagcaggttggtgtagaactcaacctgtacctaactgaattgactacagtattcgggccacaaatcacttcccctgccttaactcagctgactatccaggcgctttacaatctagctggtgggaatatggattacttgttgactaagttaggtgtaggaaacaaccaactcagctcattaattggtagtggcctgattaccggcaaccctatcctgtacgactcacagactcaactcttgggtatacaggtcaccctaccctcagtcgggaatctaaataatatgcgtgccacctacctggaaaccttgtctgtaagtacaaccaaaggatttgcctcagcacttgtcccaaaagtagtgacacaggttggttccgtgatagaagagcttgacacctcgtactgtatcgagaccgatttggacctatattgtacaagaatagtgacattccctatgtctcctggtatttattcctgtttgagtggcaatacatctgcttgcatgtattcaaagactgaaggcgcactcactacgccgtatatgaccctcaaaggctcagttattgccaactgtaagatgacaacatgtagatgtgcagaccccccgggtatcatatcgcagaattatggagaagctgtgtctctaatagataggcaatcatgcaatatcttatccttagacgggataactttgaggctcagtggggaatttgatgcaacttatcaaaagaatatctcaatacaagattctcaagtaatagttacaggcaatcttgacatctcgactgagcttgggaatgtcaacaactcgataagtaatgctttggataagttagaggaaagcaacagcaaactagacaaggtcaatgttaaactgaccagcacatccgctcttattacctatatcgttttaactgtcatatctcttgtatgtggtatacttagcctggttctagcatgctacctgatgtacaagcaaaaggcgcaacagaagaccttgttgtggcttgggaataataccctagaccagatgagggccactacaaaaatgtagtgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgtacataattcttatttatctttaatccatgaggagcatttttattttaaaaatgtcagccgccagccctataaccctagatcgcaactgatccctagtctgcgttatttgtcttgcaatcttttcgcacgcctttgtgagtgcatacaatgcccccctgctcgcttttctgaaatcgcgtcgggtcattaatgtgtcggctatcacaatgcgagatgtactcgacatgtccgtgtctgtactattgggattgtaaatagtcgaccgcgaatcatcagagtcggaatctgtaaaggatacagattccgactctgagcgcttatgaatgggatccactcggacgttgttgaacttccgttcggattctgcttcagtcaacaccggcccccgatagctactaaggttggggggtttgtgggttgtttgtgaaactgctttgcggtgtgcattaccacggggggtgtggggaagtatctgtttccacgatgcgataacgttcggtggcggagggggcgattcattctctagtgtacgcgtttcaacttcaggaacgtgattatttctttcaggacactctttccaatttccttcttccttcacttcgggtacaggtatattcttaatgtttacatacatgtcgtctgctcgtctcaactgcggggttatgatgggtggtggtgacagtctctccgaatgatcg

SEQ ID NO: 31 - DNA, 4413 bp, synthetic construct
  misc_feature 1..4413: note = DNA sequence Transfer plasmid for HVT-ND #40
  source 1..4413: mol_type = other DNA
gccactgtatgggccatttatgtttatcgagtctaaaagtcgtggaagcgcctggccatcgccagtctcgacctgccactgctgagttcggcttcaattccagcatcccagaaatgtcgcagtcaccatcacgccgtgcatcgcttcctacgaggccttttgacgcttctgatttgggcacatacaccctggacatactccaccgctattcgctcgtagatttagtacaactactgaatgacttgccgcgtaacattacctccacgcccgcttctaatgtagaaaccatggcaaaaattaatgttttaagggccatttgcgtaggatttgccgaggtccgtcgccacaacgacgcgcgaactttacagcgaacggcaatgtttgccgccgacgacgtcgcatcacggatcagaccatccattggattaaagcgcacctacccaccgggtatattttccacagctattaccgtatctaattccgaggatgaagagcgaaattcgtgatcgtaaaaataaaaaatacaagatatcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggggtttatatcttcccttctctgttcctccgcagccagccatggccaccatgggctccagatcttctaccaggatcccagtacctctgatgctgaccgtccgaatcatgttggcactgagttgcgtctgtccgaccagctcccttgatggcaggcctcttgcagctgcagggattgtggtaacaggagacaaagcagtcaacatatacacctcatctcagacagggtcaatcataatcaagttactcccaaatatgcccaaggataaagaggcgtgtgcaaaagccccattggaagcatacaacaggacattgactactttgctcaccccccttggtgattctatccgtaggatacaagagtctgtgaccacatccggaggagggaaacagggacgtcttataggcgccattatcggtggtgtagctctcggggttgcaaccgctgcacagataacagcagcctcggctctgatacaagccaatcaaaatgctgccaacatcctccggctcaaagagagcattgctgcaaccaatgaggctgtgcacgaggtcactgacggattatcacaactagcagtggcagttgggaagatgcagcaatttgttaatgaccagtttaataaaacagctcaggaattggactgtataaaaattacacagcaggttggtgtagaactcaacctgtacctaactgaattgactacagtattcgggccacaaatcacttcccctgccttaactcagctgactatccaggcgctttacaatctagctggtgggaatatggattacttgttgactaagttaggtgtaggaaacaaccaactcagctcattaattggtagtggcctgattaccggcaaccctatcctgtacgactcacagactcaactcttgggtatacaggtcaccctaccctcagtcgggaatctaaataatatgcgtgccacctacctggaaaccttgtctgtaagtacaaccaaaggatttgcctcagcacttgtcccaaaagtagtgacacaggttggttccgtgatagaagagcttgacacctcgtactgtatcgagaccgatttggacctatattgtacaagaatagtgacattccctatgtctcctggtatttattcctgtttgagtggcaatacatctgcttgcatgtattcaaagactgaaggcgcactcactacgccgtatatgaccctcaaaggctcagttattgccaactgtaagatgacaacatgtagatgtgcagaccccccgggtatcatatcgcagaattatggagaagctgtgtctctaatagataggcaatcatgcaatatcttatccttagacgggataactttgaggctcagtggggaatttgatgcaacttatcaaaagaatatctcaatacaagattctcaagtaatagttacaggcaatcttgacatctcgactgagcttgggaatgtcaacaactcgataagtaatgctttggataagttagaggaaagcaacagcaaactagacaaggtcaatgttaaactgaccagcacatccgctcttattacctatatcgttttaactgtcatatctcttgtatgtggtatacttagcctggttctagcatgctacctgatgtacaagcaaaaggcgcaacagaagaccttgttgtggcttgggaataataccctagaccagatgagggccactacaaaaatgtagcttgatctagagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgagtacataattcttatttatctttaatccatgaggagcatttttattttaaaaatgtcagccgccagccctataaccctagatcgcaactgatccctagtctgcgttatttgtcttgcaatcttttcgcacgcctttgtgagtgcatacaatgcccccctgctcgcttttctgaaatcgcgtcgggtcattaatgtgtcggctatcacaatgcgagatgtactcgacatgtccgtgtctgtactattgggattgtaaatagtcgaccgcgaatcatcagagtcggaatctgtaaaggatacagattccgactctgagcgcttatgaatgggatccactcggacgttgttgaacttccgttcggattctgcttcagtcaacaccggcccccgatagctactaaggttggggggtttgtgggttgtttgtgaaactgctttgcggtgtgcattaccacggggggtgtggggaagtatctgtttccacgatgcgataacgttcggtggcggagggggcgattcattctctagtgtacgcgtttcaacttcaggaacgtgattatttctttcaggacactctttccaatttccttcttccttcacttcgggtacaggtatattcttaatgtttacatacatgtcgtctgctcgtctcaactgcggggttatgatgggtggtggtgacagtctctccgaatgatcg

SEQ ID NO: 32 - DNA, 3849 bp, synthetic construct
  misc_feature 1..3849: note = DNA sequence Transfer plasmid for HVT-ND #41a
  source 1..3849: mol_type = other DNA
atggcagcgagcatgggatattcatcctcgtcatcgttaacatctctacgggttcagaatgtttggcatgtcgtcgatcctttgcccatcgttgcaaattacaagtccgatcgccatgaccgcgataagcctgtaccatgtggcattagggtgacatctcgatcatacattataagaccaacgtgcgagtcttccaaagacctgcacgccttcttcttcggattgtcaacgggttcttcagaatctatgcccatatctggcgttgagaccattgtgcgtttaatgaacaataaagcggcatgccatggaaaggagggctgcagatctccattttctcacgccactatcctggacgctgtagacgataattataccatgaatatagagggggtatgtttccactgccactgtgatgataagttttctccagattgttggatatctgcattttctgctgccgaacaaacttcatcgctatgcaaagagatgcgtgtgtacacgcgccgttgagtatacgggaaactaaatgttcatagaggtctttgggctatatgttattaaataaaataattgaccagtgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccgccaccatgggctccagatcttctaccaggatcccagtacctctgatgctgaccgtccgaatcatgttggcactgagttgcgtctgtccgaccagctcccttgatggcaggcctcttgcagctgcagggattgtggtaacaggagacaaagcagtcaacatatacacctcatctcagacagggtcaatcataatcaagttactcccaaatatgcccaaggataaagaggcgtgtgcaaaagccccattggaagcatacaacaggacattgactactttgctcaccccccttggtgattctatccgtaggatacaagagtctgtgaccacatccggaggagggaaacagggacgtcttataggcgccattatcggtggtgtagctctcggggttgcaaccgctgcacagataacagcagcctcggctctgatacaagccaatcaaaatgctgccaacatcctccggctcaaagagagcattgctgcaaccaatgaggctgtgcacgaggtcactgacggattatcacaactagcagtggcagttgggaagatgcagcaatttgttaatgaccagtttaataaaacagctcaggaattggactgtataaaaattacacagcaggttggtgtagaactcaacctgtacctaactgaattgactacagtattcgggccacaaatcacttcccctgccttaactcagctgactatccaggcgctttacaatctagctggtgggaatatggattacttgttgactaagttaggtgtaggaaacaaccaactcagctcattaattggtagtggcctgattaccggcaaccctatcctgtacgactcacagactcaactcttgggtatacaggtcaccctaccctcagtcgggaatctaaataatatgcgtgccacctacctggaaaccttgtctgtaagtacaaccaaaggatttgcctcagcacttgtcccaaaagtagtgacacaggttggttccgtgatagaagagcttgacacctcgtactgtatcgagaccgatttggacctatattgtacaagaatagtgacattccctatgtctcctggtatttattcctgtttgagtggcaatacatctgcttgcatgtattcaaagactgaaggcgcactcactacgccgtatatgaccctcaaaggctcagttattgccaactgtaagatgacaacatgtagatgtgcagaccccccgggtatcatatcgcagaattatggagaagctgtgtctctaatagataggcaatcatgcaatatcttatccttagacgggataactttgaggctcagtggggaatttgatgcaacttatcaaaagaatatctcaatacaagattctcaagtaatagttacaggcaatcttgacatctcgactgagcttgggaatgtcaacaactcgataagtaatgctttggataagttagaggaaagcaacagcaaactagacaaggtcaatgttaaactgaccagcacatccgctcttattacctatatcgttttaactgtcatatctcttgtatgtggtatacttagcctggttctagcatgctacctgatgtacaagcaaaaggcgcaacagaagaccttgttgtggcttgggaataataccctagaccagatgagggccactacaaaaatgtagcttgatctagagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgagtttaatgttagtttattcaatgcattggttgcaaatattcattacttctccaatcccaggtcattctttagcgagatgatgttatgacattgctgtgaaaattactacaggatatatttttaagatgcaggagtaacaatgtgcatagtaggcgtagttatcgcagacgtgcaacgcttcgcatttgagttaccgaagtgcccaacagtgctgcggttatggtttatgcgcacagaatccatgcatgtcctaattgaaccatccgatttttcttttaatcgcgatcgttgtttgggcaactgcgttatttcagatctaaaaaatttaccctttatgaccatcacatctctctggctcataccccgcttggataagatatcatgtagattccgccctaagaaatgcaaactaacattattgtcggttccatatacacttccatcttgtccttcgaaaataacaaactcgcgcaatagaccgtccgtacatgcatggccgatgtgtgtcaacatcattggtctgctagatcccgatgggacgaatcgtacagtcgtcgctccagcattggcaaaaatccccagataccctccatgcggcaaatctaaattgcgaccccgaagagactgcaccaaagtcttatcgacgcacgctgatttttttgaacagcgggagcccat

SEQ ID NO: 33 - DNA, 4599 bp, synthetic construct
  misc_feature 1..4599: note = DNA sequence Original Transfer plasmid for HVT-ND #42
  source 1..4599: mol_type = other DNA
atggcagcgagcatgggatattcatcctcgtcatcgttaacatctctacgggttcagaatgtttggcatgtcgtcgatcctttgcccatcgttgcaaattacaagtccgatcgccatgaccgcgataagcctgtaccatgtggcattagggtgacatctcgatcatacattataagaccaacgtgcgagtcttccaaagacctgcacgccttcttcttcggattgtcaacgggttcttcagaatctatgcccatatctggcgttgagaccattgtgcgtttaatgaacaataaagcggcatgccatggaaaggagggctgcagatctccattttctcacgccactatcctggacgctgtagacgataattataccatgaatatagagggggtatgtttccactgccactgtgatgataagttttctccagattgttggatatctgcattttctgctgccgaacaaacttcatcgctatgcaaagagatgcgtgtgtacacgcgccgttgagtatacgggaaactaaatgttcatagaggtctttgggctatatgttattaaataaaataattgaccagtggaattcactagtggatcccccaactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcaggcggccgctctagaactcgtcgatcgcagcgatgggctccagatcttctaccaggatcccagtacctctgatgctgaccgtccgaatcatgttggcactgagttgcgtctgtccgaccagctcccttgatggcaggcctcttgcagctgcagggattgtggtaacaggagacaaagcagtcaacatatacacctcatctcagacagggtcaatcataatcaagttactcccaaatatgcccaaggataaagaggcgtgtgcaaaagccccattggaagcatacaacaggacattgactactttgctcaccccccttggtgattctatccgtaggatacaagagtctgtgaccacatccggaggagggaaacagggacgtcttataggcgccattatcggtggtgtagctctcggggttgcaaccgctgcacagataacagcagcctcggctctgatacaagccaatcaaaatgctgccaacatcctccggctcaaagagagcattgctgcaaccaatgaggctgtgcacgaggtcactgacggattatcacaactagcagtggcagttgggaagatgcagcaatttgttaatgaccagtttaataaaacagctcaggaattggactgtataaaaattacacagcaggttggtgtagaactcaacctgtacctaactgaattgactacagtattcgggccacaaatcacttcccctgccttaactcagctgactatccaggcgctttacaatctagctggtgggaatatggattacttgttgactaagttaggtgtaggaaacaaccaactcagctcattaattggtagtggcctgattaccggcaaccctatcctgtacgactcacagactcaactcttgggtatacaggtcaccctaccctcagtcgggaatctaaataatatgcgtgccacctacctggaaaccttgtctgtaagtacaaccaaaggatttgcctcagcacttgtcccaaaagtagtgacacaggttggttccgtgatagaagagcttgacacctcgtactgtatcgagaccgatttggacctatattgtacaagaatagtgacattccctatgtctcctggtatttattcctgtttgagtggcaatacatctgcttgcatgtattcaaagactgaaggcgcactcactacgccgtatatgaccctcaaaggctcagttattgccaactgtaagatgacaacatgtagatgtgcagaccccccgggtatcatatcgcagaattatggagaagctgtgtctctaatagataggcaatcatgcaatatcttatccttagacgggataactttgaggctcagtggggaatttgatgcaacttatcaaaagaatatctcaatacaagattctcaagtaatagttacaggcaatcttgacatctcgactgagcttgggaatgtcaacaactcgataagtaatgctttggataagttagaggaaagcaacagcaaactagacaaggtcaatgttaaactgaccagcacatccgctcttattacctatatcgttttaactgtcatatctcttgtatgtggtatacttagcctggttctagcatgctacctgatgtacaagcaaaaggcgcaacagaagaccttgttgtggcttgggaataataccctagaccagatgagggccactacaaaaatgtagcttgatctagagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgagtttaatgttagtttattcaatgcattggttgcaaatattcattacttctccaatcccaggtcattctttagcgagatgatgttatgacattgctgtgaaaattactacaggatatatttttaagatgcaggagtaacaatgtgcatagtaggcgtagttatcgcagacgtgcaacgcttcgcatttgagttaccgaagtgcccaacagtgctgcggttatggtttatgcgcacagaatccatgcatgtcctaattgaaccatccgatttttcttttaatcgcgatcgttgtttgggcaactgcgttatttcagatctaaaaaatttaccctttatgaccatcacatctctctggctcataccccgcttggataagatatcatgtagattccgccctaagaaatgcaaactaacattattgtcggttccatatacacttccatcttgtccttcgaaaataacaaactcgcgcaatagaccgtccgtacatgcatggccgatgtgtgtcaacatcattggtctgctagatcccgatgggacgaatcgtacagtcgtcgctccagcattggcaaaaatccccagataccctccatgcggcaaatctaaattgcgaccccgaagagactgcaccaaagtcttatcgacgcacgctgatttttttgaacagcgggagcccat

SEQ ID NO: 34 - DNA, 3974 bp, synthetic construct
  misc_feature 1..3974: note = DNA sequence of Cloning plasmid for pSiteB
  source 1..3974: mol_type = other DNA
tctgcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgcagtgcgactaacggcatgaagctcgtcggggctggatcgcttcgtattgcgagctgtgcggctgagttgacgtatctgtgctggatgattactcataacggcaccgctatcaaacgtgccacgttcatgtccgtgtcgcccttcgctgaaactagtctctacacttcttgttaaatggaaagtgcatttgcttgttcttacaatcggcccgagtctcgttcacagcgcctcgttcacacttaaaccacaaatagtctacaggctatatgggagccagactgaaactcacatatgactaatattcgggggtgttagtcacgtgtagcccattgtgtgcatataacgatgttggacgcgtccttattcgcggtgtacttgatactatggcagcgagcatgggatattcatcctcgtcatcgttaacatctctacgggttcagaatgtttggcatgtcgtcgatcctttgcccatcgttgcaaattacaagtccgatcgccatgaccgcgataagcctgtaccatgtggcattagggtgacatctcgatcatacattataagaccaacgtgcgagtcttccaaagacctgcacgccttcttcttcggattgtcaacgggttcttcagaatctatgcccatatctggcgttgagaccattgtgcgtttaatgaacaataaagcggcatgccatggaaaggagggctgcagatctccattttctcacgccactatcctggacgctgtagacgataattataccatgaatatagagggggtatgtttccactgccactgtgatgataagttttctccagattgttggatatctgcattttctgctgccgaacaaacttcatcgctatgcaaagagatgcgtgtgtacacgcgccgttgagtatacgggaaactaaatgttcatagaggtctttgggctatatgttattaaataaaataattgggcgcgccaccggtacgagtcactggatcctctagtcagcctcgagtgactagcgtgctagcagtggccggccgtttaatgttagtttattcaatgcattggttgcaaatattcattacttctccaatcccaggtcattctttagcgagatgatgttatgacattgctgtgaaaattactacaggatatatttttaagatgcaggagtaacaatgtgcatagtaggcgtagttatcgcagacgtgcaacgcttcgcatttgagttaccgaagtgcccaacagtgctgcggttatggtttatgcgcacagaatccatgcatgtcctaattgaaccatccgatttttcttttaatcgcgatcgttgtttgggcaactgcgttatttcagatctaaaaaatttaccctttatgaccatcacatctctctggctcataccccgcttggataagatatcatgtagattccgccctaagaaatgcaaactaacattattgtcggttccatatacacttccatcttgtccttcgaaaataacaaactcgcgcaatagaccgtccgtacatgcatggccgatgtgtgtcaacatcattggtctgctagatcccgatgggacgaatcgtacagtcgtcgctccagcattggcaaaaatccccagataccctccatgcggcaaatctaaattgcgaccccgaagagactgcaccaaagtcttatcgacgcacgctgatttttttgaacagcgggagcccattatcttcagtggagcgtagacgggcgaggctaattatgtgacatagcaacactgcatgtatgtttttataaatcaataagagtacataatttattacgtatcatttccgtttgtaatatactcctgcaggcgtcaaaagggcgacacactgtcattagcaactccttgtccttcgatctcgtcaacaacagcttgcagttcaaatacaagacccagaaggcgactattctggaagcgagcttgaagccagacgctgagtacgaaaaggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgc

SEQ ID NO: 35 - DNA, 7246 bp, synthetic construct
  misc_feature 1..7246: note = DNA sequence Final Transfer plasmid for pSiteB-#42
  source 1..7246: mol_type = other DNA
tctgcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgcagtgcgactaacggcatgaagctcgtcggggctggatcgcttcgtattgcgagctgtgcggctgagttgacgtatctgtgctggatgattactcataacggcaccgctatcaaacgtgccacgttcatgtccgtgtcgcccttcgctgaaactagtctctacacttcttgttaaatggaaagtgcatttgcttgttcttacaatcggcccgagtctcgttcacagcgcctcgttcacacttaaaccacaaatagtctacaggctatatgggagccagactgaaactcacatatgactaatattcgggggtgttagtcacgtgtagcccattgtgtgcatataacgatgttggacgcgtccttattcgcggtgtacttgatactatggcagcgagcatgggatattcatcctcgtcatcgttaacatctctacgggttcagaatgtttggcatgtcgtcgatcctttgcccatcgttgcaaattacaagtccgatcgccatgaccgcgataagcctgtaccatgtggcattagggtgacatctcgatcatacattataagaccaacgtgcgagtcttccaaagacctgcacgccttcttcttcggattgtcaacgggttcttcagaatctatgcccatatctggcgttgagaccattgtgcgtttaatgaacaataaagcggcatgccatggaaaggagggctgcagatctccattttctcacgccactatcctggacgctgtagacgataattataccatgaatatagagggggtatgtttccactgccactgtgatgataagttttctccagattgttggatatctgcattttctgctgccgaacaaacttcatcgctatgcaaagagatgcgtgtgtacacgcgccgttgagtatacgggaaactaaatgttcatagaggtctttgggctatatgttattaaataaaataattgggcgcgccaactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcaggcggccgctctagaactcgtcgatcgcagcgatgggctccagatcttctaccaggatcccagtacctctgatgctgaccgtccgaatcatgttggcactgagttgcgtctgtccgaccagctcccttgatggcaggcctcttgcagctgcagggattgtggtaacaggagacaaagcagtcaacatatacacctcatctcagacagggtcaatcataatcaagttactcccaaatatgcccaaggataaagaggcgtgtgcaaaagccccattggaagcatacaacaggacattgactactttgctcaccccccttggtgattctatccgtaggatacaagagtctgtgaccacatccggaggagggaaacagggacgtcttataggcgccattatcggtggtgtagctctcggggttgcaaccgctgcacagataacagcagcctcggctctgatacaagccaatcaaaatgctgccaacatcctccggctcaaagagagcattgctgcaaccaatgaggctgtgcacgaggtcactgacggattatcacaactagcagtggcagttgggaagatgcagcaatttgttaatgaccagtttaataaaacagctcaggaattggactgtataaaaattacacagcaggttggtgtagaactcaacctgtacctaactgaattgactacagtattcgggccacaaatcacttcccctgccttaactcagctgactatccaggcgctttacaatctagctggtgggaatatggattacttgttgactaagttaggtgtaggaaacaaccaactcagctcattaattggtagtggcctgattaccggcaaccctatcctgtacgactcacagactcaactcttgggtatacaggtcaccctaccctcagtcgggaatctaaataatatgcgtgccacctacctggaaaccttgtctgtaagtacaaccaaaggatttgcctcagcacttgtcccaaaagtagtgacacaggttggttccgtgatagaagagcttgacacctcgtactgtatcgagaccgatttggacctatattgtacaagaatagtgacattccctatgtctcctggtatttattcctgtttgagtggcaatacatctgcttgcatgtattcaaagactgaaggcgcactcactacgccgtatatgaccctcaaaggctcagttattgccaactgtaagatgacaacatgtagatgtgcagaccccccgggtatcatatcgcagaattatggagaagctgtgtctctaatagataggcaatcatgcaatatcttatccttagacgggataactttgaggctcagtggggaatttgatgcaacttatcaaaagaatatctcaatacaagattctcaagtaatagttacaggcaatcttgacatctcgactgagcttgggaatgtcaacaactcgataagtaatgctttggataagttagaggaaagcaacagcaaactagacaaggtcaatgttaaactgaccagcacatccgctcttattacctatatcgttttaactgtcatatctcttgtatgtggtatacttagcctggttctagcatgctacctgatgtacaagcaaaaggcgcaacagaagaccttgttgtggcttgggaataataccctagaccagatgagggccactacaaaaatgtagcttgatctagagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgagctagcagtggccggccgtttaatgttagtttattcaatgcattggttgcaaatattcattacttctccaatcccaggtcattctttagcgagatgatgttatgacattgctgtgaaaattactacaggatatatttttaagatgcaggagtaacaatgtgcatagtaggcgtagttatcgcagacgtgcaacgcttcgcatttgagttaccgaagtgcccaacagtgctgcggttatggtttatgcgcacagaatccatgcatgtcctaattgaaccatccgatttttcttttaatcgcgatcgttgtttgggcaactgcgttatttcagatctaaaaaatttaccctttatgaccatcacatctctctggctcataccccgcttggataagatatcatgtagattccgccctaagaaatgcaaactaacattattgtcggttccatatacacttccatcttgtccttcgaaaataacaaactcgcgcaatagaccgtccgtacatgcatggccgatgtgtgtcaacatcattggtctgctagatcccgatgggacgaatcgtacagtcgtcgctccagcattggcaaaaatccccagataccctccatgcggcaaatctaaattgcgaccccgaagagactgcaccaaagtcttatcgacgcacgctgatttttttgaacagcgggagcccattatcttcagtggagcgtagacgggcgaggctaattatgtgacatagcaacactgcatgtatgtttttataaatcaataagagtacataatttattacgtatcatttccgtttgtaatatactcctgcaggcgtcaaaagggcgacacactgtcattagcaactccttgtccttcgatctcgtcaacaacagcttgcagttcaaatacaagacccagaaggcgactattctggaagcgagcttgaagccagacgctgagtacgaaaaggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgc

SEQ ID NO: 36 - DNA, 4440 bp, synthetic construct
  misc_feature 1..4440: note = DNA sequence Transfer plasmid for HVT-ND #44
  source 1..4440: mol_type = other DNA
atggcagcgagcatgggatattcatcctcgtcatcgttaacatctctacgggttcagaatgtttggcatgtcgtcgatcctttgcccatcgttgcaaattacaagtccgatcgccatgaccgcgataagcctgtaccatgtggcattagggtgacatctcgatcatacattataagaccaacgtgcgagtcttccaaagacctgcacgccttcttcttcggattgtcaacgggttcttcagaatctatgcccatatctggcgttgagaccattgtgcgtttaatgaacaataaagcggcatgccatggaaaggagggctgcagatctccattttctcacgccactatcctggacgctgtagacgataattataccatgaatatagagggggtatgtttccactgccactgtgatgataagttttctccagattgttggatatctgcattttctgctgccgaacaaacttcatcgctatgcaaagagatgcgtgtgtacacgcgccgttgagtatacgggaaactaaatgttcatagaggtctttgggctatatgttattaaataaaataattgaccagtgtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggggtttatatcttcccttctctgttcctccgcagccagccatggccaccatgggctccagatcttctaccaggatcccagtacctctgatgctgaccgtccgaatcatgttggcactgagttgcgtctgtccgaccagctcccttgatggcaggcctcttgcagctgcagggattgtggtaacaggagacaaagcagtcaacatatacacctcatctcagacagggtcaatcataatcaagttactcccaaatatgcccaaggataaagaggcgtgtgcaaaagccccattggaagcatacaacaggacattgactactttgctcaccccccttggtgattctatccgtaggatacaagagtctgtgaccacatccggaggagggaaacagggacgtcttataggcgccattatcggtggtgtagctctcggggttgcaaccgctgcacagataacagcagcctcggctctgatacaagccaatcaaaatgctgccaacatcctccggctcaaagagagcattgctgcaaccaatgaggctgtgcacgaggtcactgacggattatcacaactagcagtggcagttgggaagatgcagcaatttgttaatgaccagtttaataaaacagctcaggaattggactgtataaaaattacacagcaggttggtgtagaactcaacctgtacctaactgaattgactacagtattcgggccacaaatcacttcccctgccttaactcagctgactatccaggcgctttacaatctagctggtgggaatatggattacttgttgactaagttaggtgtaggaaacaaccaactcagctcattaattggtagtggcctgattaccggcaaccctatcctgtacgactcacagactcaactcttgggtatacaggtcaccctaccctcagtcgggaatctaaataatatgcgtgccacctacctggaaaccttgtctgtaagtacaaccaaaggatttgcctcagcacttgtcccaaaagtagtgacacaggttggttccgtgatagaagagcttgacacctcgtactgtatcgagaccgatttggacctatattgtacaagaatagtgacattccctatgtctcctggtatttattcctgtttgagtggcaatacatctgcttgcatgtattcaaagactgaaggcgcactcactacgccgtatatgaccctcaaaggctcagttattgccaactgtaagatgacaacatgtagatgtgcagaccccccgggtatcatatcgcagaattatggagaagctgtgtctctaatagataggcaatcatgcaatatcttatccttagacgggataactttgaggctcagtggggaatttgatgcaacttatcaaaagaatatctcaatacaagattctcaagtaatagttacaggcaatcttgacatctcgactgagcttgggaatgtcaacaactcgataagtaatgctttggataagttagaggaaagcaacagcaaactagacaaggtcaatgttaaactgaccagcacatccgctcttattacctatatcgttttaactgtcatatctcttgtatgtggtatacttagcctggttctagcatgctacctgatgtacaagcaaaaggcgcaacagaagaccttgttgtggcttgggaataataccctagaccagatgagggccactacaaaaatgtagcttgatctagagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgagtttaatgttagtttattcaatgcattggttgcaaatattcattacttctccaatcccaggtcattctttagcgagatgatgttatgacattgctgtgaaaattactacaggatatatttttaagatgcaggagtaacaatgtgcatagtaggcgtagttatcgcagacgtgcaacgcttcgcatttgagttaccgaagtgcccaacagtgctgcggttatggtttatgcgcacagaatccatgcatgtcctaattgaaccatccgatttttcttttaatcgcgatcgttgtttgggcaactgcgttatttcagatctaaaaaatttaccctttatgaccatcacatctctctggctcataccccgcttggataagatatcatgtagattccgccctaagaaatgcaaactaacattattgtcggttccatatacacttccatcttgtccttcgaaaataacaaactcgcgcaatagaccgtccgtacatgcatggccgatgtgtgtcaacatcattggtctgctagatcccgatgggacgaatcgtacagtcgtcgctccagcattggcaaaaatccccagataccctccatgcggcaaatctaaattgcgaccccgaagagactgcaccaaagtcttatcgacgcacgctgatttttttgaacagcgggagcccat

SEQ ID NO: 37 - DNA, 3832 bp, synthetic construct
  misc_feature 1..3832: note = DNA sequence Transfer plasmid for HVT-ND #45
  source 1..3832: mol_type = other DNA
atgggctcccgctgttcaaaaaaatcagcgtgcgtcgataagactttggtgcagtctcttcggggtcgcaatttagatttgccgcatggagggtatctggggatttttgccaatgctggagcgacgactgtacgattcgtcccatcgggatctagcagaccaatgatgttgacacacatcggccatgcatgtacggacggtctattgcgcgagtttgttattttcgaaggacaagatggaagtgtatatggaaccgacaataatgttagtttgcatttcttagggcggaatctacatgatatcttatccaagcggggtatgagccagagagatgtgatggtcataaagggtaaattttttagatctgaaataacgcagttgcccaaacaacgatcgcgattaaaagaaaaatcggatggttcaattaggacatgcatggattctgtgcgcataaaccataaccgcagcactgttgggcacttcggtaactcaaatgcgaagcgttgcacgtctgcgataactacgcctactatgcacattgttactcctgcatcttaaaaatatatcctgtagtaattttcacagcaatgtcataacatcatctcgctaaagaatgacctgggattggagaagtaatgaatatttgcaaccaatgcattgaataaactaacattaaacttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttaccgccaccatgggctccagatcttctaccaggatcccagtacctctgatgctgaccgtccgaatcatgttggcactgagttgcgtctgtccgaccagctcccttgatggcaggcctcttgcagctgcagggattgtggtaacaggagacaaagcagtcaacatatacacctcatctcagacagggtcaatcataatcaagttactcccaaatatgcccaaggataaagaggcgtgtgcaaaagccccattggaagcatacaacaggacattgactactttgctcaccccccttggtgattctatccgtaggatacaagagtctgtgaccacatccggaggagggaaacagggacgtcttataggcgccattatcggtggtgtagctctcggggttgcaaccgctgcacagataacagcagcctcggctctgatacaagccaatcaaaatgctgccaacatcctccggctcaaagagagcattgctgcaaccaatgaggctgtgcacgaggtcactgacggattatcacaactagcagtggcagttgggaagatgcagcaatttgttaatgaccagtttaataaaacagctcaggaattggactgtataaaaattacacagcaggttggtgtagaactcaacctgtacctaactgaattgactacagtattcgggccacaaatcacttcccctgccttaactcagctgactatccaggcgctttacaatctagctggtgggaatatggattacttgttgactaagttaggtgtaggaaacaaccaactcagctcattaattggtagtggcctgattaccggcaaccctatcctgtacgactcacagactcaactcttgggtatacaggtcaccctaccctcagtcgggaatctaaataatatgcgtgccacctacctggaaaccttgtctgtaagtacaaccaaaggatttgcctcagcacttgtcccaaaagtagtgacacaggttggttccgtgatagaagagcttgacacctcgtactgtatcgagaccgatttggacctatattgtacaagaatagtgacattccctatgtctcctggtatttattcctgtttgagtggcaatacatctgcttgcatgtattcaaagactgaaggcgcactcactacgccgtatatgaccctcaaaggctcagttattgccaactgtaagatgacaacatgtagatgtgcagaccccccgggtatcatatcgcagaattatggagaagctgtgtctctaatagataggcaatcatgcaatatcttatccttagacgggataactttgaggctcagtggggaatttgatgcaacttatcaaaagaatatctcaatacaagattctcaagtaatagttacaggcaatcttgacatctcgactgagcttgggaatgtcaacaactcgataagtaatgctttggataagttagaggaaagcaacagcaaactagacaaggtcaatgttaaactgaccagcacatccgctcttattacctatatcgttttaactgtcatatctcttgtatgtggtatacttagcctggttctagcatgctacctgatgtacaagcaaaaggcgcaacagaagaccttgttgtggcttgggaataataccctagaccagatgagggccactacaaaaatgtagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtcaattattttatttaataacatatagcccaaagacctctatgaacatttagtttcccgtatactcaacggcgcgtgtacacacgcatctctttgcatagcgatgaagtttgttcggcagcagaaaatgcagatatccaacaatctggagaaaacttatcatcacagtggcagtggaaacataccccctctatattcatggtataattatcgtctacagcgtccaggatagtggcgtgagaaaatggagatctgcagccctcctttccatggcatgccgctttattgttcattaaacgcacaatggtctcaacgccagatatgggcatagattctgaagaacccgttgacaatccgaagaagaaggcgtgcaggtctttggaagactcgcacgttggtcttataatgtatgatcgagatgtcaccctaatgccacatggtacaggcttatcgcggtcatggcgatcggacttgtaatttgcaacgatgggcaaaggatcgacgacatgccaaacattctgaacccgtagagatgttaacgatgacgaggatgaatatcccatgctcgctgccat

SEQ ID NO: 38 - DNA, 4592 bp, synthetic construct
  misc_feature 1..4592: note = DNA sequence Transfer plasmid for HVT-ND #46
  source 1..4592: mol_type = other DNA
atgggctcccgctgttcaaaaaaatcagcgtgcgtcgataagactttggtgcagtctcttcggggtcgcaatttagatttgccgcatggagggtatctggggatttttgccaatgctggagcgacgactgtacgattcgtcccatcgggatctagcagaccaatgatgttgacacacatcggccatgcatgtacggacggtctattgcgcgagtttgttattttcgaaggacaagatggaagtgtatatggaaccgacaataatgttagtttgcatttcttagggcggaatctacatgatatcttatccaagcggggtatgagccagagagatgtgatggtcataaagggtaaattttttagatctgaaataacgcagttgcccaaacaacgatcgcgattaaaagaaaaatcggatggttcaattaggacatgcatggattctgtgcgcataaaccataaccgcagcactgttgggcacttcggtaactcaaatgcgaagcgttgcacgtctgcgataactacgcctactatgcacattgttactcctgcatcttaaaaatatatcctgtagtaattttcacagcaatgtcataacatcatctcgctaaagaatgacctgggattggagaagtaatgaatatttgcaaccaatgcattgaataaactaacattaaacgaattcactagtggatcccccaactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcaggcggccgctctagaactcgtcgatcgcagcgatgggctccagatcttctaccaggatcccagtacctctgatgctgaccgtccgaatcatgttggcactgagttgcgtctgtccgaccagctcccttgatggcaggcctcttgcagctgcagggattgtggtaacaggagacaaagcagtcaacatatacacctcatctcagacagggtcaatcataatcaagttactcccaaatatgcccaaggataaagaggcgtgtgcaaaagccccattggaagcatacaacaggacattgactactttgctcaccccccttggtgattctatccgtaggatacaagagtctgtgaccacatccggaggagggaaacagggacgtcttataggcgccattatcggtggtgtagctctcggggttgcaaccgctgcacagataacagcagcctcggctctgatacaagccaatcaaaatgctgccaacatcctccggctcaaagagagcattgctgcaaccaatgaggctgtgcacgaggtcactgacggattatcacaactagcagtggcagttgggaagatgcagcaatttgttaatgaccagtttaataaaacagctcaggaattggactgtataaaaattacacagcaggttggtgtagaactcaacctgtacctaactgaattgactacagtattcgggccacaaatcacttcccctgccttaactcagctgactatccaggcgctttacaatctagctggtgggaatatggattacttgttgactaagttaggtgtaggaaacaaccaactcagctcattaattggtagtggcctgattaccggcaaccctatcctgtacgactcacagactcaactcttgggtatacaggtcaccctaccctcagtcgggaatctaaataatatgcgtgccacctacctggaaaccttgtctgtaagtacaaccaaaggatttgcctcagcacttgtcccaaaagtagtgacacaggttggttccgtgatagaagagcttgacacctcgtactgtatcgagaccgatttggacctatattgtacaagaatagtgacattccctatgtctcctggtatttattcctgtttgagtggcaatacatctgcttgcatgtattcaaagactgaaggcgcactcactacgccgtatatgaccctcaaaggctcagttattgccaactgtaagatgacaacatgtagatgtgcagaccccccgggtatcatatcgcagaattatggagaagctgtgtctctaatagataggcaatcatgcaatatcttatccttagacgggataactttgaggctcagtggggaatttgatgcaacttatcaaaagaatatctcaatacaagattctcaagtaatagttacaggcaatcttgacatctcgactgagcttgggaatgtcaacaactcgataagtaatgctttggataagttagaggaaagcaacagcaaactagacaaggtcaatgttaaactgaccagcacatccgctcttattacctatatcgttttaactgtcatatctcttgtatgtggtatacttagcctggttctagcatgctacctgatgtacaagcaaaaggcgcaacagaagaccttgttgtggcttgggaataataccctagaccagatgagggccactacaaaaatgtagcttgatctagagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacaattattttatttaataacatatagcccaaagacctctatgaacatttagtttcccgtatactcaacggcgcgtgtacacacgcatctctttgcatagcgatgaagtttgttcggcagcagaaaatgcagatatccaacaatctggagaaaacttatcatcacagtggcagtggaaacataccccctctatattcatggtataattatcgtctacagcgtccaggatagtggcgtgagaaaatggagatctgcagccctcctttccatggcatgccgctttattgttcattaaacgcacaatggtctcaacgccagatatgggcatagattctgaagaacccgttgacaatccgaagaagaaggcgtgcaggtctttggaagactcgcacgttggtcttataatgtatgatcgagatgtcaccctaatgccacatggtacaggcttatcgcggtcatggcgatcggacttgtaatttgcaacgatgggcaaaggatcgacgacatgccaaacattctgaacccgtagagatgttaacgatgacgaggatgaatatcccatgctcgctgccat

SEQ ID NO: 39 - DNA, 4433 bp, synthetic construct
  misc_feature 1..4433: note = DNA sequence Transfer plasmid for HVT-ND #48
  source 1..4433: mol_type = other DNA
atgggctcccgctgttcaaaaaaatcagcgtgcgtcgataagactttggtgcagtctcttcggggtcgcaatttagatttgccgcatggagggtatctggggatttttgccaatgctggagcgacgactgtacgattcgtcccatcgggatctagcagaccaatgatgttgacacacatcggccatgcatgtacggacggtctattgcgcgagtttgttattttcgaaggacaagatggaagtgtatatggaaccgacaataatgttagtttgcatttcttagggcggaatctacatgatatcttatccaagcggggtatgagccagagagatgtgatggtcataaagggtaaattttttagatctgaaataacgcagttgcccaaacaacgatcgcgattaaaagaaaaatcggatggttcaattaggacatgcatggattctgtgcgcataaaccataaccgcagcactgttgggcacttcggtaactcaaatgcgaagcgttgcacgtctgcgataactacgcctactatgcacattgttactcctgcatcttaaaaatatatcctgtagtaattttcacagcaatgtcataacatcatctcgctaaagaatgacctgggattggagaagtaatgaatatttgcaaccaatgcattgaataaactaacattaaactcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggggtttatatcttcccttctctgttcctccgcagccagccatggccaccatgggctccagatcttctaccaggatcccagtacctctgatgctgaccgtccgaatcatgttggcactgagttgcgtctgtccgaccagctcccttgatggcaggcctcttgcagctgcagggattgtggtaacaggagacaaagcagtcaacatatacacctcatctcagacagggtcaatcataatcaagttactcccaaatatgcccaaggataaagaggcgtgtgcaaaagccccattggaagcatacaacaggacattgactactttgctcaccccccttggtgattctatccgtaggatacaagagtctgtgaccacatccggaggagggaaacagggacgtcttataggcgccattatcggtggtgtagctctcggggttgcaaccgctgcacagataacagcagcctcggctctgatacaagccaatcaaaatgctgccaacatcctccggctcaaagagagcattgctgcaaccaatgaggctgtgcacgaggtcactgacggattatcacaactagcagtggcagttgggaagatgcagcaatttgttaatgaccagtttaataaaacagctcaggaattggactgtataaaaattacacagcaggttggtgtagaactcaacctgtacctaactgaattgactacagtattcgggccacaaatcacttcccctgccttaactcagctgactatccaggcgctttacaatctagctggtgggaatatggattacttgttgactaagttaggtgtaggaaacaaccaactcagctcattaattggtagtggcctgattaccggcaaccctatcctgtacgactcacagactcaactcttgggtatacaggtcaccctaccctcagtcgggaatctaaataatatgcgtgccacctacctggaaaccttgtctgtaagtacaaccaaaggatttgcctcagcacttgtcccaaaagtagtgacacaggttggttccgtgatagaagagcttgacacctcgtactgtatcgagaccgatttggacctatattgtacaagaatagtgacattccctatgtctcctggtatttattcctgtttgagtggcaatacatctgcttgcatgtattcaaagactgaaggcgcactcactacgccgtatatgaccctcaaaggctcagttattgccaactgtaagatgacaacatgtagatgtgcagaccccccgggtatcatatcgcagaattatggagaagctgtgtctctaatagataggcaatcatgcaatatcttatccttagacgggataactttgaggctcagtggggaatttgatgcaacttatcaaaagaatatctcaatacaagattctcaagtaatagttacaggcaatcttgacatctcgactgagcttgggaatgtcaacaactcgataagtaatgctttggataagttagaggaaagcaacagcaaactagacaaggtcaatgttaaactgaccagcacatccgctcttattacctatatcgttttaactgtcatatctcttgtatgtggtatacttagcctggttctagcatgctacctgatgtacaagcaaaaggcgcaacagaagaccttgttgtggcttgggaataataccctagaccagatgagggccactacaaaaatgtagcttgatctagagcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgacaattattttatttaataacatatagcccaaagacctctatgaacatttagtttcccgtatactcaacggcgcgtgtacacacgcatctctttgcatagcgatgaagtttgttcggcagcagaaaatgcagatatccaacaatctggagaaaacttatcatcacagtggcagtggaaacataccccctctatattcatggtataattatcgtctacagcgtccaggatagtggcgtgagaaaatggagatctgcagccctcctttccatggcatgccgctttattgttcattaaacgcacaatggtctcaacgccagatatgggcatagattctgaagaacccgttgacaatccgaagaagaaggcgtgcaggtctttggaagactcgcacgttggtcttataatgtatgatcgagatgtcaccctaatgccacatggtacaggcttatcgcggtcatggcgatcggacttgtaatttgcaacgatgggcaaaggatcgacgacatgccaaacattctgaacccgtagagatgttaacgatgacgaggatgaatatcccatgctcgctgccat

SEQ ID NO: 40 - DNA, 59 bp, synthetic construct
  misc_feature 1..59: note = DNA sequence of 5'Upstream Sbfi gfp gene mutagenesis primer 1
  source 1..59: mol_type = other DNA
ggctagcgtttaaacttaagcttacctgcaggccaccatggtgagcaagggcgccgagc

SEQ ID NO: 41 - DNA, 59 bp, synthetic construct
  misc_feature 1..59: note = DNA sequence of 5'Upstream Sbfi gfp gene mutagenesis primer 2
  source 1..59: mol_type = other DNA
gctcggcgcccttgctcaccatggtggcctgcaggtaagcttaagtttaaacgctagcc

SEQ ID NO: 42 - DNA, 61 bp, synthetic construct
  misc_feature 1..61: note = DNA sequence of 3' downstream Sbfi gfp gene mutagenesis primer 1
  source 1..61: mol_type = other DNA
cacggcatggatgagctgtacaagtgacctgcaggtgtgccttctagttgccagccatctg

SEQ ID NO: 43 - DNA, 61 bp, synthetic construct
  misc_feature 1..61: note = DNA sequence of 3' downstream Sbfi gfp gene mutagenesis primer 2
  source 1..61: mol_type = other DNA
cagatggctggcaactagaaggcacacctgcaggtcacttgtacagctcatccatgccgtg

SEQ ID NO: 44 - DNA, 23 bp, synthetic construct
  misc_feature 1..23: note = DNA sequence of upper UL55-gene 3 primer
  source 1..23: mol_type = other DNA
ccaccgggtatattttccacagc

SEQ ID NO: 45 - DNA, 19 bp, synthetic construct
  misc_feature 1..19: note = DNA sequence of lower UL55-gene 3 primer
  source 1..19: mol_type = other DNA
gacccgacgcgatttcaga

SEQ ID NO: 46 - DNA, 20 bp, synthetic construct
  misc_feature 1..20: note = DNA sequence upper UL55-gene3 PCR primer
  source 1..20: mol_type = other DNA
actgccactgtgatgataag

SEQ ID NO: 47 - DNA, 19 bp, synthetic construct
  misc_feature 1..19: note = DNA sequence lower UL55-gene3 PCR primer
  source 1..19: mol_type = other DNA
ctcgctaaagaatgacctg

SEQ ID NO: 48 - DNA, 24 bp, synthetic construct
  misc_feature 1..24: note = DNA sequence for upper primer localized within IBD VP2 codingregion
  source 1..24: mol_type = other DNA
ggccgacctcaactctccactcaa

SEQ ID NO: 49 - DNA, 24 bp, synthetic construct
  misc_feature 1..24: note = DNA sequence for lower primer downstream within HVT IBD#1
  source 1..24: mol_type = other DNA
ttcacagcgcctcgttcacactta

SEQ ID NO: 50 - DNA, 20 bp, synthetic construct
  misc_feature 1..20: note = DNA sequence for upper primer of the upstream junction of theinsertion site of transfer plasmid HVT IBD#1
  source 1..20: mol_type = other DNA
aatattcgggggtgttagtc

SEQ ID NO: 51 - DNA, 18 bp, synthetic construct
  misc_feature 1..18: note = DNA sequence for lower primer within the IBDV VP2 coding regionof HVT IBD #1
  source 1..18: mol_type = other DNA
tgtggttggcatcagaag

SEQ ID NO: 52 - DNA, 24 bp, synthetic construct
  misc_feature 1..24: note = DNA sequence for upper primer within the IBDV VP2 coding region
  source 1..24: mol_type = other DNA
cggcgccatgaactacacaaaact

SEQ ID NO: 53 - DNA, 24 bp, synthetic construct
  misc_feature 1..24: note = DNA sequence for lower primer downstream of the UL35/36integration site of HVT-IBD #5
  source 1..24: mol_type = other DNA
ggggcggaaacaaataaactctcg

SEQ ID NO: 54 - DNA, 21 bp, synthetic construct
  misc_feature 1..21: note = DNA sequence for upper primer of the UL35/36 insertion site ofHVT IBD #5
  source 1..21: mol_type = other DNA
actcggcggtaggggcatttg

SEQ ID NO: 55 - DNA, 19 bp, synthetic construct
  misc_feature 1..19: note = DNA sequence for lower primer within the hCMV promoter of HVT IBD#5
  source 1..19: mol_type = other DNA
gggtcgttgggcggtcagc

SEQ ID NO: 56 - DNA, 24 bp, synthetic construct
  misc_feature 1..24: note = DNA sequence for upper primer upstream of the integration site ofHVT IBD #6a
  source 1..24: mol_type = other DNA
tagactcggcggtaggggcatttg

SEQ ID NO: 57 - DNA, 21 bp, synthetic construct
  misc_feature 1..21: note = DNA sequence for lower primer localized within the pec promoterof HVT IBD#6a
  source 1..21: mol_type = other DNA
gggggtcgttgggcggtcagc

SEQ ID NO: 58 - DNA, 24 bp, synthetic construct
  misc_feature 1..24: note = DNA sequence for upper primer localized within the IBD VP2 codingregion of HVT IBD #6a
  source 1..24: mol_type = other DNA
cggcgccatgaactacacaaaact

SEQ ID NO: 59 - DNA, 24 bp, synthetic construct
  misc_feature 1..24: note = DNA sequence for lower primer localized downstream of the UL35/36insertion site
  source 1..24: mol_type = other DNA
ggggcggaaacaaataaactctcg

SEQ ID NO: 60 - DNA, 20 bp, synthetic construct
  misc_feature 1..20: note = DNA sequence for upper primer upstream of the integration site ofUL55/Gene3 for HVT IBD#9
  source 1..20: mol_type = other DNA
actgccactgtgatgataag

SEQ ID NO: 61 - DNA, 18 bp, synthetic construct
  misc_feature 1..18: note = DNA sequence for lower primer downstream of the integration siteof UL55/Gene3 for HVT IBD#9
  source 1..18: mol_type = other DNA
ctcgctaaagaatgacct

SEQ ID NO: 62 - DNA, 20 bp, synthetic construct
  misc_feature 1..20: note = DNA sequence for insert orientation upper primer surroundingupstream junction of the HVT IBD#9 VP2 gene insertion
  source 1..20: mol_type = other DNA
aatattcgggggtgttagtc

SEQ ID NO: 63 - DNA, 24 bp, synthetic construct
  misc_feature 1..24: note = DNA sequence for insert orientation lower primer localized withinIBDV VP2 coding region for HVT IBD#9
  source 1..24: mol_type = other DNA
ctcgcttctcagtgtatgtttttc

SEQ ID NO: 64 - DNA, 24 bp, synthetic construct
  misc_feature 1..24: note = DNA sequence for upper primer downstream site determining correctintegration of the IBDV VP2 coding region for HVT IBDV #9
  source 1..24: mol_type = other DNA
atcctgagctcgctaaaaatcttg

SEQ ID NO: 65 - DNA, 22 bp, synthetic construct
  misc_feature 1..22: note = DNA sequence for lower primer downstream site determining correctintegration of the IBDV VP2 coding region for HVT IBDV #9
  source 1..22: mol_type = other DNA
cctcgcccgtctacgctccact

SEQ ID NO: 66 - DNA, 17 bp, synthetic construct
  misc_feature 1..17: note = DNA sequence for upper primer for upstream region of integrationsite of UL55-Gene3 for HVT IBD#30
  source 1..17: mol_type = other DNA
tagcgacccgagtagga

SEQ ID NO: 67 - DNA, 24 bp, synthetic construct
  misc_feature 1..24: note = DNA sequence for lower primer for upstream region of integrationsite of UL55-Gene3 for HVT IBD#30
  source 1..24: mol_type = other DNA
cgcccacggtcaaattgtatgtag

SEQ ID NO: 68 - DNA, 20 bp, synthetic construct
  misc_feature 1..20: note = DNA sequence for upper primer to confirm correct orientation ofVP2 insert surrounding the 3' junction of the insertion site ofHVT IBD#30
  source 1..20: mol_type = other DNA
ttatccgcgcaatcagaagg

SEQ ID NO: 69 - DNA, 22 bp, synthetic construct
  misc_feature 1..22: note = DNA sequence for lower primer to confirm correct orientation ofVP2 insert surrounding the 3' junction of the insertion site ofHVT IBD#30
  source 1..22: mol_type = other DNA
atggggcggaaacaaataaact

SEQ ID NO: 70 - DNA, 23 bp, synthetic construct
  misc_feature 1..23: note = DNAupper primer to confirm VP2 insert outside of the expressioncassette of HVT IBD#30
  source 1..23: mol_type = other DNA
ccaccgggtatattttccacagc

SEQ ID NO: 71 - DNA, 19 bp, synthetic construct
  misc_feature 1..19: note = DNA sequence for lower primer to confirmVP2 insert integrationoutside of the expression cassette of HVT IBD#30
  source 1..19: mol_type = other DNA
gacccgacgcgatttcaga

SEQ ID NO: 72 - DNA, 24 bp, synthetic construct
  misc_feature 1..24: note = DNA sequence for upper primer to confirm correct orientation ofVP2 insert upstream of the UL35/36 integration site of HVT IBD#31
  source 1..24: mol_type = other DNA
ctcggcggtaggggcatttgataa

SEQ ID NO: 73 - DNA, 24 bp, synthetic construct
  misc_feature 1..24: note = DNA sequence for lower primer to confirm correct orientation ofVP2 insert localized within the chicken beta actin promoter ofHVT IBD#31
  source 1..24: mol_type = other DNA
agatggggagagtgaagcagaacg

SEQ ID NO: 74 - DNA, 20 bp, synthetic construct
  misc_feature 1..20: note = DNA sequence for upper primer to confirm confirmation of VP2insert localized within the IBDV VP2 coding region
  source 1..20: mol_type = other DNA
ttatccgcgcaatcagaagg

SEQ ID NO: 75 - DNA, 22 bp, synthetic construct
  misc_feature 1..22: note = DNA sequence for lower primer to confirm correct orientation ofVP2 insert located downstream of UL35/36 integration site of HVTIBD#31
  source 1..22: mol_type = other DNA
atggggcggaaacaaataaact

SEQ ID NO: 76 - DNA, 20 bp, synthetic construct
  misc_feature 1..20: note = DNA sequence for upper primer targeting the downstreamintegration site of the VP2 insert of HVT IBD#31 located withinthe VP2 insert
  source 1..20: mol_type = other DNA
ttatccgcgcaatcagaagg

SEQ ID NO: 77 - DNA, 22 bp, synthetic construct
  misc_feature 1..22: note = DNA sequence for lower primer localized downstream of the UL35/36site of HVT IBD#31
  source 1..22: mol_type = other DNA
atggggcggaaacaaataaact

SEQ ID NO: 78 - DNA, 24 bp, synthetic construct
  misc_feature 1..24: note = DNA sequence for upper primer for upstream region of integrationsite of Gene3-UL55 for HVT-IBD #34
  source 1..24: mol_type = other DNA
cctcgcccgtctacgctccactga

SEQ ID NO: 79 - DNA, 24 bp, synthetic construct
  misc_feature 1..24: note = DNA sequence for lower primer localized within chicken beta-actinpromoter for HVT-IBD #34
  source 1..24: mol_type = other DNA
ccgcccccatcgctgcacaaaata

SEQ ID NO: 80 - DNA, 18 bp, synthetic construct
  misc_feature 1..18: note = DNA sequence for upper primerlocalized within IBDV VP2 codingregion for HVT-IBD #34
  source 1..18: mol_type = other DNA
ggccctccgtcccgtcac

SEQ ID NO: 81 - DNA, 24 bp, synthetic construct
  misc_feature 1..24: note = DNA sequence for lower primer localized downstream of Gene3-UL55insertion site for HVT IBD#34
  source 1..24: mol_type = other DNA
ttcacagcgcctcgttcacactta

SEQ ID NO: 82 - DNA, 19 bp, synthetic construct
  misc_feature 1..19: note = DNA sequence for upper primer localized outside of the VP2expression cassette of HVT IBD #34
  source 1..19: mol_type = other DNA
ctcgctaaagaatgacctg

SEQ ID NO: 83 - DNA, 20 bp, synthetic construct
  misc_feature 1..20: note = DNA sequence for lower primer localized outside of the VP2expression cassette of HVT IBD #34
  source 1..20: mol_type = other DNA
actgccactgtgatgataag

SEQ ID NO: 84 - DNA, 20 bp, synthetic construct
  misc_feature 1..20: note = DNA sequence for upper primer for upstream region of integrationsite of UL35-UL36 of HVT ND#38
  source 1..20: mol_type = other DNA
tgtttaaggcattttcaagt

SEQ ID NO: 85 - DNA, 19 bp, synthetic construct
  misc_feature 1..19: note = DNA sequence for lower primer that localized within NDV F codingregion of HVT ND#38
  source 1..19: mol_type = other DNA
attcggacggtcagcatca

SEQ ID NO: 86 - DNA, 24 bp, synthetic construct
  misc_feature 1..24: note = DNA sequence for upper primer surrounding the 3' junction of theinsertion localized within NDV F coding region of HVT ND#38
  source 1..24: mol_type = other DNA
accagatgagggccactacaaaaa

SEQ ID NO: 87 - DNA, 22 bp, synthetic construct
  misc_feature 1..22: note = DNA sequence for lower primer localized downstream of UL35-UL36insertion site of HVT ND#38
  source 1..22: mol_type = other DNA
atggggcggaaacaaataaact

SEQ ID NO: 88 - DNA, 17 bp, synthetic construct
  misc_feature 1..17: note = DNA sequence for upper primer outside of the expression cassetteof HVT ND#38
  source 1..17: mol_type = other DNA
gaggtccgtcgccacaa

SEQ ID NO: 89 - DNA, 22 bp, synthetic construct
  misc_feature 1..22: note = DNA sequence for lower primer outside of the expression cassetteof HVT ND#38
  source 1..22: mol_type = other DNA
gcaagacaaataacgcagacta

SEQ ID NO: 90 - DNA, 24 bp, synthetic construct
  misc_feature 1..24: note = DNA sequence for upper primer upstream region of integration siteof UL35-UL36 for HVT-ND #39
  source 1..24: mol_type = other DNA
ctcggcggtaggggcatttgataa

SEQ ID NO: 91 - DNA, 24 bp, synthetic construct
  misc_feature 1..24: note = DNA sequence for lower primer localized within chicken beta-actinpromoter HVT-ND #39
  source 1..24: mol_type = other DNA
agatggggagagtgaagcagaacg

SEQ ID NO: 92 - DNA, 20 bp, synthetic construct
  misc_feature 1..20: note = DNA sequence for upper primer surrounding the downstream junctionof the insertion localized within poly A region of HVT-ND #39
  source 1..20: mol_type = other DNA
tggggatgcggtgggctcta

SEQ ID NO: 93 - DNA, 24 bp, synthetic construct
  misc_feature 1..24: note = DNA sequence for lower primer localized downstream of UL35-UL36insertion of HVT-ND #39
  source 1..24: mol_type = other DNA
ggggcggaaacaaataaactctcg

SEQ ID NO: 94 - DNA, 23 bp, synthetic construct
  misc_feature 1..23: note = DNA sequence for upper primer outside of the expression cassetteof HVT-ND #39
  source 1..23: mol_type = other DNA
ccaccgggtatattttccacagc

SEQ ID NO: 95 - DNA, 19 bp, synthetic construct
  misc_feature 1..19: note = DNA sequence for lower primer outside of the expression cassetteHVT-ND #39
  source 1..19: mol_type = other DNA
gacccgacgcgatttcaga

SEQ ID NO: 96 - DNA, 24 bp, synthetic construct
  misc_feature 1..24: note = DNA sequence for upper primer upstream of the UL35/36 integrationsite for HVT ND#40
  source 1..24: mol_type = other DNA
ctcggcggtaggggcatttgataa

SEQ ID NO: 97 - DNA, 24 bp, synthetic construct
  misc_feature 1..24: note = DNA sequence for lower primer localized within chicken beta actinpromoter for HVT ND#40
  source 1..24: mol_type = other DNA
agatggggagagtgaagcagaacg

SEQ ID NO: 98 - DNA, 24 bp, synthetic construct
  misc_feature 1..24: note = DNA sequence for upper primer localized within NDVF coding regionfor HVT ND#40
  source 1..24: mol_type = other DNA
accagatgagggccactacaaaaa

SEQ ID NO: 99 - DNA, 24 bp, synthetic construct
  misc_feature 1..24: note = DNA sequence for lower primer located at the downstream junctionof the insertion site for HVT ND#40
  source 1..24: mol_type = other DNA
atggggcggaaacaaataaactct

SEQ ID NO: 100 - DNA, 23 bp, synthetic construct
  misc_feature 1..23: note = DNA sequence for upper primer located outside of the expressioncassette for HVT ND#40
  source 1..23: mol_type = other DNA
ccaccgggtatattttccacagc

SEQ ID NO: 101 - DNA, 19 bp, synthetic construct
  misc_feature 1..19: note = DNA sequence for lower primer located outside of the expressioncassette for HVT#40
  source 1..19: mol_type = other DNA
gacccgacgcgatttcaga

SEQ ID NO: 102 - DNA, 48 bp, synthetic construct
  misc_feature 1..48: note = DNA sequence for upper primer for PCR amplification of cassettefor HVT ND#42
  source 1..48: mol_type = other DNA
tacttagtcataggcgcgccaactccgcccgttttatgactagaacca

SEQ ID NO: 103 - DNA, 49 bp, synthetic construct
  misc_feature 1..49: note = DNA sequence for lower primer for PCR amplification of cassettefor HVT ND#42
  source 1..49: mol_type = other DNA
tgacagtatctagctagctcgactctagaggatccgaaaaaacctccca

SEQ ID NO: 104 - DNA, 20 bp, synthetic construct
  misc_feature 1..20: note = DNA sequence for upper primer located outside of cassette for HVTND#42
  source 1..20: mol_type = other DNA
actgccactgtgatgataag

SEQ ID NO: 105 - DNA, 19 bp, synthetic construct
  misc_feature 1..19: note = DNA sequence for lower primer located outside of cassette for HVTND #42
  source 1..19: mol_type = other DNA
ctcgctaaagaatgacctg

SEQ ID NO: 106 - DNA, 24 bp, synthetic construct
  misc_feature 1..24: note = DNA sequence for upper primer located upstream and outside of theexpression cassette for HVT ND #42
  source 1..24: mol_type = other DNA
ttcacagcgcctcgttcacactta

SEQ ID NO: 107 - DNA, 24 bp, synthetic construct
  misc_feature 1..24: note = DNA sequence for lower primer located within ND F coding regionfor HVT ND #42
  source 1..24: mol_type = other DNA
ttcggacggtcagcatcagaggta

SEQ ID NO: 108 - DNA, 20 bp, synthetic construct
  misc_feature 1..20: note = DNA sequence for upper primer located upstream and outside of theexpression cassette for HVT ND #42
  source 1..20: mol_type = other DNA
gcctcggcgtggtagttctc

SEQ ID NO: 109 - DNA, 19 bp, synthetic construct
  misc_feature 1..19: note = DNA sequence for lower primer located within ND F coding regionfor HVT ND #42
  source 1..19: mol_type = other DNA
attcggacggtcagcatca

SEQ ID NO: 110 - DNA, 24 bp, synthetic construct
  misc_feature 1..24: note = DNA sequence for upper primer located upstream and outside of theexpression cassette for HVT ND #42
  source 1..24: mol_type = other DNA
ttcacagcgcctcgttcacactta

SEQ ID NO: 111 - DNA, 24 bp, synthetic construct
  misc_feature 1..24: note = DNA sequence for lower primer located within NDV F coding regionfor HVT ND #42
  source 1..24: mol_type = other DNA
gcttttgcacacgcctctttatcc

SEQ ID NO: 112 - DNA, 24 bp, synthetic construct
  misc_feature 1..24: note = DNA sequence for upper primer located upstream and outside of theexpression
  source 1..24: mol_type = other DNA
ttcacagcgcctcgttcacactta

SEQ ID NO: 113 - DNA, 24 bp, synthetic construct
  misc_feature 1..24: note = DNA sequence for lower primer located within ND F coding regionfor HVT ND #42
  source 1..24: mol_type = other DNA
ttcggacggtcagcatcagaggta

SEQ ID NO: 114 - DNA, 24 bp, synthetic construct
  misc_feature 1..24: note = DNA sequence for upper primer located upstream and outside of theexpression cassette for HVT ND #42
  source 1..24: mol_type = other DNA
accagatgagggccactacaaaaa

SEQ ID NO: 115 - DNA, 21 bp, synthetic construct
  misc_feature 1..21: note = DNA sequence for lower primer located within ND F coding regionfor HVT ND #42
  source 1..21: mol_type = other DNA
cgcccgtctacgctccactga

SEQ ID NO: 116 - DNA, 18 bp, synthetic construct
  misc_feature 1..18: note = DNA sequence for upper primer localized upstream of UL55 for HVTND #44
  source 1..18: mol_type = other DNA
aatattcgggggtgttag

SEQ ID NO: 117 - DNA, 20 bp, synthetic construct
  misc_feature 1..20: note = DNA sequence for lower primer localized within chicken beta actinpromoter for HVT ND #44
  source 1..20: mol_type = other DNA
gggggagatggggagagtga

SEQ ID NO: 118 - DNA, 20 bp, synthetic construct
  misc_feature 1..20: note = DNA sequence for upper primer localized upstream of UL55 for HVTND #44
  source 1..20: mol_type = other DNA
gcctcggcgtggtagttctc

SEQ ID NO: 119 - DNA, 24 bp, synthetic construct
  misc_feature 1..24: note = DNA sequence for lower primer localized within chicken beta actinpromoter for HVT ND #44
  source 1..24: mol_type = other DNA
ccgcccccatcgctgcacaaaata

SEQ ID NO: 120 - DNA, 24 bp, synthetic construct
  misc_feature 1..24: note = DNA sequence for upper primer localized within NDV F gene codingsequence for HVT ND#44
  source 1..24: mol_type = other DNA
accagatgagggccactacaaaaa

SEQ ID NO: 121 - DNA, 24 bp, synthetic construct
  misc_feature 1..24: note = DNA sequence for lower primer localized downstream of UL55-Gene3insertion site for HVT ND#44
  source 1..24: mol_type = other DNA
ctcgcccgtctacgctccactgaa

SEQ ID NO: 122 - DNA, 20 bp, synthetic construct
  misc_feature 1..20: note = DNA sequence for upper primer localized outside of the expressioncassette for HVT ND#44
  source 1..20: mol_type = other DNA
actgccactgtgatgataag

SEQ ID NO: 123 - DNA, 19 bp, synthetic construct
  misc_feature 1..19: note = DNA sequence for lower primer localized outside of the expressioncassette for HVT#44
  source 1..19: mol_type = other DNA
ctcgctaaagaatgacctg

SEQ ID NO: 124 - DNA, 20 bp, synthetic construct
  misc_feature 1..20: note = DNA sequence for upper primer localized outside of the expressioncassette for HVT#45
  source 1..20: mol_type = other DNA
actgccactgtgatgataag

SEQ ID NO: 125 - DNA, 19 bp, synthetic construct
  misc_feature 1..19: note = DNA sequence for lower primer localized outside of the expressioncassette for HVT#45
  source 1..19: mol_type = other DNA
ctcgctaaagaatgacctg

SEQ ID NO: 126 - DNA, 24 bp, synthetic construct
  misc_feature 1..24: note = DNA sequence for upper primer located upstream and outside of theexpression cassette for HVT ND#45
  source 1..24: mol_type = other DNA
ctcgcccgtctacgctccactgaa

SEQ ID NO: 127 - DNA, 24 bp, synthetic construct
  misc_feature 1..24: note = DNA sequence for lower primer located within ND F coding regionfor HVT ND#45
  source 1..24: mol_type = other DNA
gcttttgcacacgcctctttatcc

SEQ ID NO: 128 - DNA, 18 bp, synthetic construct
  misc_feature 1..18: note = DNA sequence for upper primer located upstream and outside of theexpression cassette for HVT ND#45
  source 1..18: mol_type = other DNA
cacgctgctattgtaacg

SEQ ID NO: 129 - DNA, 19 bp, synthetic construct
  misc_feature 1..19: note = DNA sequence for lower primer located within the ND F codingregion for HVT ND#45
  source 1..19: mol_type = other DNA
actgggatcctggtagaag

SEQ ID NO: 130 - DNA, 24 bp, synthetic construct
  misc_feature 1..24: note = DNA sequence for upper primer surrounding the downstream junctionof the insertion for HVT ND#45
  source 1..24: mol_type = other DNA
accagatgagggccactacaaaaa

SEQ ID NO: 131 - DNA, 24 bp, synthetic construct
  misc_feature 1..24: note = DNA sequence for lower primer localized downstream of Gene3-UL55insertion site
  source 1..24: mol_type = other DNA
ttcacagcgcctcgttcacactta

SEQ ID NO: 132 - DNA, 23 bp, synthetic construct
  misc_feature 1..23: note = DNA sequence for upper primer surrounding the downstream junctionof the insertion for HVT ND#45
  source 1..23: mol_type = other DNA
gcacatccgctcttattacctat

SEQ ID NO: 133 - DNA, 24 bp, synthetic construct
  misc_feature 1..24: note = DNA sequence for lower primer localized that localized downstreamof Gene3-UL55insertion site
  source 1..24: mol_type = other DNA
ttcacagcgcctcgttcacactta

SEQ ID NO: 134 - DNA, 23 bp, synthetic construct
  misc_feature 1..23: note = DNA sequence for upper primer localized outside of the expressioncassette for HVT ND#46
  source 1..23: mol_type = other DNA
tagagggggtatgtttccactgc

SEQ ID NO: 135 - DNA, 23 bp, synthetic construct
  misc_feature 1..23: note = DNA sequence for lower primer localized outside of the expressioncassette for HVT ND#46
  source 1..23: mol_type = other DNA
gtcataacatcatctcgctaaag

SEQ ID NO: 136 - DNA, 18 bp, synthetic construct
  misc_feature 1..18: note = DNA sequence for upper primer located upstream and outside of theintegration site for HVT ND#46
  source 1..18: mol_type = other DNA
cacgctgctattgtaacg

SEQ ID NO: 137 - DNA, 21 bp, synthetic construct
  misc_feature 1..21: note = DNA sequence for lower primer located within the mCMV promoterfor HVT ND#46
  source 1..21: mol_type = other DNA
ggtgaacgccccctatgtgga

SEQ ID NO: 138 - DNA, 24 bp, synthetic construct
  misc_feature 1..24: note = DNA sequence for upper primer localized within NDV F gene codingsequence for HVT ND#46
  source 1..24: mol_type = other DNA
accagatgagggccactacaaaaa

SEQ ID NO: 139 - DNA, 24 bp, synthetic construct
  misc_feature 1..24: note = DNA sequence for lower primer localized downstream and outside ofexpression cassette for HVT ND#46
  source 1..24: mol_type = other DNA
ttcacagcgcctcgttcacactta

SEQ ID NO: 140 - DNA, 23 bp, synthetic construct
  misc_feature 1..23: note = DNA sequence for upper primer localized within NDV F gene codingsequence for HVT ND#46
  source 1..23: mol_type = other DNA
gcacatccgctcttattacctat

SEQ ID NO: 141 - DNA, 24 bp, synthetic construct
  misc_feature 1..24: note = DNA sequence for lower primer localized downstream and outside ofexpression cassette for HVT ND#46
  source 1..24: mol_type = other DNA
ttcacagcgcctcgttcacactta

SEQ ID NO: 142 - DNA, 23 bp, synthetic construct
  misc_feature 1..23: note = DNA sequence for upper primer localized within NDV F gene codingsequence for HVT ND#46
  source 1..23: mol_type = other DNA
atgtggtatacttagcctggttc

SEQ ID NO: 143 - DNA, 18 bp, synthetic construct
  misc_feature 1..18: note = DNA sequence for lower primer localized downstream and outside ofexpression cassette for HVT ND#46
  source 1..18: mol_type = other DNA
aatattcgggggtgttag

SEQ ID NO: 144 - DNA, 23 bp, synthetic construct
  misc_feature 1..23: note = DNA sequence for upper primer localized within NDV F gene codingsequence for HVT ND#46
  source 1..23: mol_type = other DNA
ggaataataccctagaccagatg

SEQ ID NO: 145 - DNA, 23 bp, synthetic construct
  misc_feature 1..23: note = DNA sequence for lower primer localized downstream and outside ofexpression cassette for HVT ND#46
  source 1..23: mol_type = other DNA
gtcataacatcatctcgctaaag

SEQ ID NO: 146 - DNA, 24 bp, synthetic construct
  misc_feature 1..24: note = DNA sequence for upper primer localized upstream region ofintegration site of Gene 3-UL55 for HVT ND#48
  source 1..24: mol_type = other DNA
cctcgcccgtctacgctccactga

SEQ ID NO: 147 - DNA, 24 bp, synthetic construct
  misc_feature 1..24: note = DNA sequence for lower primer localized within chicken beta-actinpromoter for HVT ND#48
  source 1..24: mol_type = other DNA
ccgcccccatcgctgcacaaaata

SEQ ID NO: 148 - DNA, 24 bp, synthetic construct
  misc_feature 1..24: note = DNA sequence for upper primer surrounding the downstream junctionof the insertion for HVT ND#48
  source 1..24: mol_type = other DNA
accagatgagggccactacaaaaa

SEQ ID NO: 149 - DNA, 24 bp, synthetic construct
  misc_feature 1..24: note = DNA sequence for lower primer localized downstream of Gene 3-UL55insertion site for HVT ND#48
  source 1..24: mol_type = other DNA
ttcacagcgcctcgttcacactta

SEQ ID NO: 150 - DNA, 23 bp, synthetic construct
  misc_feature 1..23: note = DNA sequence for upper primer surrounding the downstream junctionof the insertion for HVT ND#48
  source 1..23: mol_type = other DNA
gcacatccgctcttattacctat

SEQ ID NO: 151 - DNA, 24 bp, synthetic construct
  misc_feature 1..24: note = DNA sequence for lower primer localized downstream of Gene 3-UL55insertion site for HVT ND#48
  source 1..24: mol_type = other DNA
ttcacagcgcctcgttcacactta

SEQ ID NO: 152 - DNA, 20 bp, synthetic construct
  misc_feature 1..20: note = DNA sequence for upper primer located outside of the expressioncassette for HVT ND#48
  source 1..20: mol_type = other DNA
actgccactgtgatgataag

SEQ ID NO: 153 - DNA, 19 bp, synthetic construct
  misc_feature 1..19: note = DNA sequence for lower primer located outside of the expressioncassette for HVT ND#48
  source 1..19: mol_type = other DNA
ctcgctaaagaatgacctg

SEQ ID NO: 154 - DNA, 48 bp, synthetic construct; identical to SEQ ID NO: 102
  misc_feature 1..48: note = DNA sequence for upper primer for amplification of the NDV Fexpression cassette of HVT ND#42 for HVT-IBD-ND #42-#30
  source 1..48: mol_type = other DNA

SEQ ID NO: 155 - DNA, 49 bp, synthetic construct; identical to SEQ ID NO: 103
  misc_feature 1..49: note = DNA sequence for lower primer for amplification of the NDV Fexpression cassette of HVT ND#42 for HVT-IBD-ND #42-#30
  source 1..49: mol_type = other DNA

SEQ ID NO: 156 - DNA, 52 bp, synthetic construct
  misc_feature 1..52: note = DNA sequence for upper primer for amplification of the IBD geneexpression cassette of plasmid #30 for HVT-IBD-ND #42-#30
  source 1..52: mol_type = other DNA
ctgtcagacgagtaccggtttgacattgattattgactagttattaatagta

SEQ ID NO: 157 - DNA, 42 bp, synthetic construct
  misc_feature 1..42: note = DNA sequence for lower primer for amplification of the IBD geneexpression cassette of plasmid #30 for HVT-IBD-ND #42-#30
  source 1..42: mol_type = other DNA
tctagtactgatggtaccaccatagagcccaccgcatcccca

SEQ ID NO: 158 - DNA, 570 bp, synthetic construct
  misc_feature 1..570: note = DNA sequence for RSV LTR promoter
  source 1..570: mol_type = other DNA
actctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctcca

(19) United States
(12) Patent Application Publication
(10) Pub. No.: US 2024/0374702 A1
(43) Pub. Date: Nov. 14, 2024

(54) RECOMBINANT HERPESVIRUS OF TURKEY VECTORS EXPRESSING ANTIGENS OF AVIAN PATHOGENS AND USES THEREOF

(71) Applicant: Zoetis Services LLC, Parsippany, NJ (US)

(72) Inventors: Sing RONG, Kalamazoo, MI (US); Yugang LUO, Kalamazoo, MI (US); Tyler BROWN, Kalamazoo, MI (US)

(21) Appl. No.: 18/542,330

(22) Filed: Dec. 15, 2023

Related U.S. Application Data
(63) Continuation of application No. 17/823,566, filed on Aug. 31, 2022, now Pat. No. 11,883,481.
Application No. 17/823,566 is a continuation of application No. 17/017,342, filed on Sep. 10, 2020, now Pat. No. 11,464,849.
(60) Provisional application No. 62/898,651, filed on Sep. 11, 2019.

(51) Int. Cl. A61K 39/12; A61K 39/00; A61K 39/17; A61P 31/14; A61P 31/20; A61P 37/04; C12N 7/00; C12N 15/86

(52) U.S. Cl. CPC A61K 39/12; A61K 39/17; A61P 31/14; A61P 31/20; A61P 37/04; C12N 7/00; C12N 15/86; A61K 2039/53; A61K 2039/54; A61K 2039/552; C12N 2710/16021; C12N 2710/16034; C12N 2710/16043; C12N 2720/10021; C12N 2720/10034; C12N 2760/18121; C12N 2760/18134; C12N 2830/50

(57) ABSTRACT

The invention relates to recombinant viral vectors for the insertion and expression of foreign genes for use in safe immunizations to protect against a variety of pathogens. The invention also relates to multivalent compositions or vaccine comprising one or more recombinant viral vectors for protection against a variety of pathogens. The present invention relates to methods of making an using said recombinant viral vectors.

### CROSS REFERENCE TO RELATED APPLICATIONS

This application claims the benefit under 35 USC 119(e) to U.S. Provisional Application No. 62/898,651 filed on Sep. 11, 2019, which is hereby incorporated by reference in its entirety.

### FIELD OF THE INVENTION

The invention relates to recombinant viral vectors for the insertion and expression of foreign genes for use in safe immunizations to protect against a variety of pathogens. It also relates to multivalent compositions or vaccine comprising one or more recombinant viral vectors for protection against a variety of pathogens. The present invention relates to methods of making an using said recombinant viral vectors.

### BACKGROUND

Marek's disease, a highly contagious lymphoproliferative disease, is one of the most prevalent avian infections that predominantly affect young chickens. Marek's disease is caused by Marek's Disease virus. Marek's Disease Virus (MDV), a herpesvirus, is a member of the genus Mardivirus, that has three serotypes (species): MDV-1 (Gallid herpesvirus 2), MDV-2 (Gallid herpesvirus 3) and MDV-3 (Meleagrid herpesvirus 1, Turkey Herpesvirus (HVT)). MDV-1 is the most virulent of the three serotypes causing widespread disease in unvaccinated poultry. Birds infected with MDV-1 show neurologic, visceral and cutaneous clinical symptoms such as paralysis of legs, wings and neck; eye lesions and vision impairment, weight loss, cancerous tumors in many organs, such as the thymus, heart, lungs, gonads, muscles and feather follicles. Morbidity of affected birds is 10-50% and mortality can be up to 100%. Even though Marek's disease can affect birds at any age, acute Marek's disease causes death in large number of unvaccinated birds at an early age of four to eight weeks. Marek disease is spread by direct or indirect exposure to chicken dander of infected chicken and the virus is taken in by inhalation. MDV-2 and MDV-3 represent avirulent viral strains and have been used in preparation of vaccination against the related and virulent MDV-1.

In addition to Marek's Disease there are several pathogens that affect poultry and pose a threat to poultry farming. Producers must rely on immunity provided by vaccines to protect flocks from viral, bacterial and other pathogens. Live, killed and recombinant vaccines have been used in vaccinating birds. Live vaccines have the advantage of strong and long-lasting immunity, but they must be handled carefully as they might cause mild to severe reaction. On the other hand, killed vaccines are more stable and safer than live vaccines but generate a weaker immune response thus requiring multiple administrations. Both live and killed vaccines have proven safe and effective however a need remains to develop and continually improve upon multivalent vaccines to provide protection against more than one pathogen in one vaccination.

Recombinant vectored vaccines have been developed to provide immunity to multiple pathogens simultaneously. These vaccines are made by removing some non-essential gene sections within the host genome of a non-pathogenic organism and replacing these with one or more genes coding for antigens that are responsible for producing an immune response against a pathogenic organism. The newly produced vector is then used to infect the host, where it will replicate and express the antigens of the virulent organism(s) to elicit immune response. Recombinant vectored vaccines combine the advantages of live and killed vaccines. Recombinant vectors, similar to live vaccine, provides longer lasting immunity and at the same time causing milder reaction after vaccination as killed vaccines. Additionally, both the vector and the inserted gene(s) can provide immunity protecting the birds from two or more diseases.

Marek Disease Viruses are one of the most efficacious vectors for multivalent vaccines to immunize against poultry diseases since these viruses induce lifetime protection with just one vaccination. Additionally, these viruses are limited to avian hosts, therefore there is no danger of infecting other animals and the people working in poultry farms. Among Marek Disease Viruses, Herpes Virus of Turkeys (HVT) has been used more extensively both as live vaccine and as recombinant vaccine vector against the more virulent MDV-1. HVT was first isolated from turkeys in 1969-1970 and it was soon found to be protective against MDV and licensed as vaccine in 1971. Herpes virus of Turkeys (HVT) has similar antigenic features as Marek's disease virus (MDV-1), but it is not pathogenic to chickens. In addition, HVT is not sensitive to maternally derived antibodies against MDV or HVT therefore live HVT vaccine have been used to effectively vaccinate against MDV-1 in ovo or at an early age before hatching. In addition, the HVT genome has been used as vaccine vector to harbor foreign DNA sequences of other avian pathogens.

### SUMMARY OF THE INVENTION

The invention provides recombinant viral vectors for the insertion and expression of foreign genes for use in safe immunizations to protect avians against a variety of pathogens. The invention also provides multivalent compositions or vaccines comprising one or more recombinant HVT viral vectors for protection against a variety of pathogens. Additionally, the invention provides methods of making and using the recombinant viral vectors alone or in combination with other vaccines or pharmaceutical compositions.

In one aspect the present invention provides a recombinant Herpesvirus of turkey (HVT) genome comprising one or more nucleotide sequence(s) coding for one or more heterologous antigen(s) inserted into the intergenic loci UL 35/UL 36 in the unique long (UL) region of the HVT genome.

In one aspect the present invention provides a recombinant Herpesvirus of Turkey (HVT) genome comprising one or more nucleotide sequences coding for one or more heterologous antigens or antigens inserted into the intergenic loci UL 35/UL 36 in the unique long region of the HVT genome and one or more nucleotide sequences or sequences coding for one or more heterologous antigens inserted at the UL55/Gene 3 site in the unique long region (UL) of the HVT genome.

In one or more embodiments the present invention provides a recombinant HVT wherein the one or more heterologous antigens or antigens are protective against avian pathogens or pathogens selected from the group consisting of: Infectious Bursal Disease Virus (IBDV); Newcastle disease virus (NDV); Infectious Bronchitis Virus (IBV); Infectious Laryngotracheitis Virus (ILTV); Chicken Anemia Virus (CAV); and Avian Influenza Virus (AIV).

In one or more embodiments the present invention provides a recombinant HVT wherein the one or one or more heterologous antigens are selected from the group consisting of: the VP2, VP3 or VP4 proteins of the Infectious Bursal Disease Virus (IBDV); the VP1 or VP2 proteins of the Chicken Anemia Virus (CAV); the F/HN chimera protein or the F, NP, P, M, HN, or L proteins of the Newcastle Disease Virus (NDV); the S1, S2 or M proteins of Infectious Bronchitis Virus (IBV); the gB, gC, gD, gE, gH, gI or gL proteins of the Infectious Laryngotracheitis Virus (ILTV); and any of the HA, NA, NP or M proteins of the Avian Influenza Virus (AIV).

In one or more embodiments the recombinant HVT of the present invention provides that the one or more heterologous antigen is protective against IBDV. In one embodiment the recombinant HVT of the present invention provides the one or more heterologous antigen is the VP2 protein of IBDV. In one embodiment the recombinant HVT of the present invention provides the VP2 protein is encoded by the nucleotide sequence comprising at least 80% sequence identity to the nucleotide sequence comprising SEQ ID NO. 5 or SEQ ID NO. 10. In one embodiment the recombinant HVT of the present invention provides the VP2 protein encoded by the nucleotide sequence comprising either SEQ ID NO. 5 or SEQ ID NO.10.

In one or more embodiments the recombinant HVT of the present invention provides the one or more heterologous antigen or antigen is protective against Newcastle Disease Virus (NDV). In one embodiment the recombinant HVT of the present invention provides the one or more heterologous antigen is the F protein of NDV. In one embodiment the recombinant HVT of the present invention provides that the F protein of NDV is encoded by a nucleotide sequence comprising at least 80% sequence identity to the nucleotide sequence comprising SEQ ID NO. 3. In one embodiment the recombinant HVT of the present invention the F protein of NDV is encoded by the nucleotide sequences comprising SEQ ID NO. 3.

In one or more embodiments the recombinant HVT of the present invention provides the one or more heterologous antigens are protective against NDV and IBDV. In one or more embodiments the recombinant HVT of the present invention provides that the at least one heterologous antigens are the F protein of NDV and the VP2 protein of IBDV.

In one or more embodiments the recombinant HVT of the present invention provides the F protein of NDV encoded by a nucleotide sequence comprising at least 80% sequence identity to the nucleotide sequence comprising SEQ ID NO. 3 and the VP2 protein of IBDV encoded by the nucleotide sequence comprising at least 80% sequence identity to the nucleotide sequence comprising SEQ ID NO. 5 or SEQ ID NO.10.

In one or more embodiments the recombinant HVT of the present invention provides the F protein of NDV encoded by the nucleotide sequence comprising SEQ ID NO. 3 and the VP2 protein of IBDV is encoded by the nucleotide sequence comprising SEQ ID NO. 5 or SEQ ID NO.10.

In one or more embodiments the recombinant HVT of the present invention comprises a genome comprising one or more expression cassette or cassettes comprising one or more nucleotide sequence or sequences that encode one or more heterologous antigen or antigens. In one embodiment the recombinant HVT comprises a recombinant HVT genome an expression cassette that comprises a nucleotide sequence encoding promoters that are operatively linked to one or more nucleotides that encode antigens to be expressed. In one embodiment the antigen to be expressed comprises the F protein of NDV. In one embodiment the antigen to be expressed comprise the VP2 protein of IBDV. In one embodiment the antigens to be expressed comprise both the F protein of NDV and the VP2 protein of IBDV.

In one embodiment the recombinant HVT of the present invention provides the one or more promoters are selected from the group consisting of: immediate early cytomegalovirus human (hCMV) promoter: guinea pig immediate early CMV promoter; murine immediate early CMV promoter; Pec promoter; β-chicken actin promoter; SV40 promoter; Pseudorabies Virus promoters of glycoprotein X promoter; Herpes Simplex Virus-1 alpha 4 promoter; Marek's Disease Virus promoters of glycoproteins gA, gC, gB, gE, or gI promoter; Infectious Laryngotracheitis Virus promoters of glycoprotein gB, gE, gI, gD promoter; and Bovine Herpesvirus 1/1 VP8 promoter. In one embodiment the recombinant HVT comprises the human CMV promoter. In one embodiment the recombinant HVT comprises the murine CMV promoter. In one embodiment the recombinant HVT comprises the hCMV and mCMV promoter.

In one or more embodiments the recombinant HVT comprises a nucleotide sequence encoding a poly adenylation (polyA) signal. In one or more embodiments the recombinant HVT comprises a nucleotide sequence encoding a poly A signal and is selected from BGH poly A (SEQ ID NO.6) or SV40 poly A sequence (SEQ ID NO.12). In one embodiment the poly A signal is a BGH poly A signal. In one embodiment the poly A signal is an SV40 poly A signal.

In one aspect the recombinant HVT of the present invention comprises a CMV promoter operatively linked to a nucleotide sequence encoding a VP2 protein from an IBDV further comprising a nucleotide sequence coding for a polyadenylation signal, all part of a VP2 expression cassette inserted in a non-coding region of the HVT genome. In one embodiment the CMV promoter comprises an hCMV promoter (SEQ ID NO.1). In one embodiment the nucleotide sequence encoding the VP2 protein of IBDV is selected from SEQ ID NO.5 or SEQ ID NO.10. In one embodiment the nucleotide sequence encoding the VP2 protein comprises SEQ ID NO.5. In one embodiment the nucleotide sequence encoding the VP2 protein comprises SEQ ID NO.10. In one embodiment the polyadenylation signal comprises SEQ ID NO. 6. In one embodiment the polyadenylation signal comprises SEQ IDNO.12. In one embodiment the promoter, the nucleotide sequence encoding the VP2 protein and the poly A signal comprise an expression cassette. In one embodiment the expression cassette is inserted into the HVT genome at the UL55/gene 3 site. In one embodiment the expression cassette is inserted into the HVT genome at the UL35/36 site within the genome. In one embodiment the expression cassette comprises, in order, SEQ ID NO.1, SEQ ID NO.5 or SEQ ID NO.10 and SEQ ID NO.6 inserted into the HVT genome at the UL55/gene 3 site.

In one aspect the recombinant HVT of the present invention comprises a CMV promoter operatively linked to a nucleotide sequence encoding an F protein of an NDV further comprising a nucleotide sequence coding for a polyadenylation signal all part of an NDV F cassette inserted into a non-coding location within the HVT genome. In one embodiment the CMV promoter comprises an mCMV (SEQ ID NO.2) promoter. In one embodiment the nucleotide sequence encoding the F protein of an NDV which comprises SEQ ID NO.3. In one embodiment the polyadenylation signal is coded for by nucleotide sequence comprising SEQ ID NO.12. In one embodiment the promoter, the nucleotide sequence encoding the F protein and the poly A signal comprise an expression cassette. In one embodiment the expression cassette is inserted into the HVT genome at the UL55/gene 3 site. In one embodiment the expression cassette is inserted into the HVT genome at the UL35/36 site within the genome. In one embodiment the expression cassette comprises, in order, SEQ ID NO.2, SEQ ID NO.3 and SEQ ID NO.12 inserted into the HVT genome at the UL55/gene 3 site.

In one aspect the recombinant HVT of the present invention comprises a CMV promoter operatively linked to a nucleotide sequence encoding a VP2 protein of an IBDV further comprising a nucleotide sequence coding for a polyadenylation signal, all comprising a VP2 expression cassette inserted into a non-coding location within the HVT genome. In one embodiment the recombinant HVT of the present invention further comprises a CMV promoter operatively linked to a nucleotide sequence encoding the F protein of an NDV further comprising a nucleotide sequence coding for a polyadenylation signal as part of an NDV F expression cassette inserted into the same insertion site as the VP2 cassette. In one embodiment the recombinant HVT of the present further comprises a CMV promoter operatively linked to a nucleotide sequence encoding an F protein of NDV further comprising a nucleotide sequence coding for a polyadenylation signal as part of an NDV F expression cassette inserted into a different site as the VP2 cassette.

In one embodiment the recombinant HVT of the present invention provides a VP2 expression cassette comprising, in order, a nucleotide sequence encoding hCMV promoter (SEQ ID NO. 1), nucleotide sequence encoding IBDV VP2 (selected from SEQ ID NO.5 or SEQ ID NO.10) and nucleotide sequence encoding BGH polyadenylation signal (SEQ ID NO.6) inserted into the HVT genome in the UL35/36 non-coding region and, in order, nucleotide sequence encoding mCMV promoter (SEQ ID NO.2), a nucleotide sequence encoding the F protein from NDV (SEQ ID NO.3) and a nucleotide sequence encoding an SV40 polyadenylation signal (SEQ ID NO.12) inserted into the HVT genome in the UL55/gene 3 non-coding region. In one aspect the recombinant HVT of the present invention comprises a promoter operatively linked to a nucleotide sequence encoding an Infectious Laryngotracheitis Virus antigen further comprising a nucleotide sequence coding for a polyadenylation signal. In one embodiment the ILT antigen comprises one or more antigens selected from the group consisting of: the gB, gC, gD, gE, gH, gI, gL or chimeric proteins of one or more of the ILT antigens of the Infectious Laryngotracheitis Virus (ILTV). In one embodiment the recombinant HVT of the present invention further comprises a nucleotide sequence that encodes one or more antigens selected from the group consisting of Infectious Bursal Disease Virus, Chicken Anemia Virus, Newcastle Disease Virus, Infectious Bronchitis Virus and Avian Influenza Virus. In one embodiment the recombinant HVT of the present invention further provides a promoter operatively linked to a nucleotide sequence encoding antigens selected from the group consisting of: a VP1, VP2, VP3 or VP4 antigen of the Infectious Bursal Disease Virus (IBDV); the VP1 or VP2 proteins of the Chicken Anemia Virus (CAV); the F/HN chimera protein or the F, NP, P, M, HN, or L proteins of the Newcastle Disease Virus (NDV); the S1, S2 or M proteins of Infectious Bronchitis Virus (IBV); and any of the HA, NA, NP or M proteins of the Avian Influenza Virus (AIV).

In one embodiment the recombinant HVT of the present invention comprises one or more ILT antigens as part of an expression cassette comprising a promoter that is operatively linked to the nucleotide encoding the ILT antigen and further comprising a nucleotide sequence encoding a polyadenylation signal. In one embodiment the recombinant HVT of the present invention comprises a second and a third expression cassette each comprising nucleotide sequences encoding a promoter operatively linked to a nucleotide sequence encoding avian antigens selected from the group consisting of a VP1, VP2, VP3 or VP4 antigen of the Infectious Bursal Disease Virus (IBDV); the VP1 or VP2 proteins of the Chicken Anemia Virus (CAV); the F/HN chimera protein or the F, NP, P, M, HN, or L proteins of the Newcastle Disease Virus (NDV); the S1, S2 or M proteins of Infectious Bronchitis Virus (IBV); and any of the HA, NA, NP or M proteins of the Avian Influenza Virus (AIV) and further comprising a nucleotide sequence encoding a polyadenylation signal.

In one or more aspects the present invention provides a recombinant DNA encoding the recombinant HVT genome of the present invention.

In one or more aspects the present invention provides an immunogenic composition comprising the recombinant HVT of the present invention and further comprising a pharmaceutically acceptable carrier, excipient or adjuvant.

In one or more aspects the present invention provides a vaccine composition comprising the recombinant HVT of the invention and further comprising a pharmaceutically acceptable carrier, excipient or adjuvant.

In one embodiment the vaccine of the present invention further comprises an additional Marek's disease Virus (MDV) selected from the group consisting of: naturally attenuated MDV-1 strain Rispens (CVI-988); or a Gallid Herpesvirus 3 strain SB-1 virus. In one embodiment the vaccine of the present invention provides that the additional MDV comprises a recombinant genome. In one embodiment the vaccine of the present invention provides that the additional recombinant MDV genome comprises one or more nucleotide sequence(s) encoding one or more heterologous antigen(s) that are protective against one or more avian pathogen(s).

In one embodiment the vaccine of the present invention provides for use in vaccinating an avian against one or more diseases caused by one or more avian pathogen(s). In one or more embodiments the vaccine of the present invention provides for use in protecting an avian against clinical symptoms caused by one or more avian pathogen(s). In one or more embodiments the vaccine of the present invention provides for use in protecting an avian against clinical symptoms caused by Marek's Disease Virus and clinical symptoms caused by one or more avian pathogen(s). In one or more embodiments the vaccine of the present invention provides the one or more avian pathogen(s) selected from a group consisting of: Infectious Bursal Disease Virus (IBDV); Newcastle disease virus (NDV); Infectious Bronchitis Virus (IBV); Infectious Laryngotracheitis Virus (ILTV); Chicken Anemia Virus (CAV); and Avian Influenza Virus (AIV). In one embodiment the vaccine of the present invention provides that the one or more avian pathogen comprises the Newcastle Disease Virus. In one embodiment the vaccine of the present invention provides that the one or more avian pathogen comprises the Infectious Bursal Disease Virus (IBDV). In one embodiment the vaccine of the present invention provides that the one or more avian pathogen comprises the Newcastle Disease Virus and the Infectious Bursal Disease Virus.

In one or more embodiments the vaccine of the present invention provides for use in vaccinating an avian wherein the vaccine is administered by at least one administration of the vaccine by spray administration, in ovo administration, subcutaneous administration, intramuscular administration, oral administration, nasal administration or combination thereof. In one embodiment the vaccine of the present invention provides that the vaccine is administered by in ovo administration. In one embodiment the vaccine of the present invention provides that the in ovo administration occurs in an embryonated egg between about 16-22 days of development. In one or more embodiments the vaccine of the present invention provides that the in ovo administration occurs in an embryonated egg at about 18 days of development. In one embodiment the vaccine of the present invention provides that the administration of the vaccine comprises in ovo administration followed by spray administration. In one embodiment the vaccine of the present invention provides that the administration of the vaccine comprises spray administration.

In one aspect the present invention provides a method of vaccinating an avian to treat or prevent Marek's disease and one or more avian diseases caused by one or more avian pathogens comprises the step of administering an effective amount of the vaccine composition the present invention. In one embodiment the method of the present invention provides that the one or more avian pathogens are selected from a group consisting of Infectious Bursal Disease Virus (IBDV); Newcastle disease virus (NDV); Infectious Bronchitis Virus (IBV); Infectious Laryngotracheitis Virus (ILTV); Chicken Anemia Virus (CAV); and Avian Influenza Virus (AIV). In one embodiment the method of the present invention provides that the one or more avian pathogen comprises the Infectious Bursal Disease Virus (IBDV). In one embodiment the method of the present invention provides that the one or more avian pathogen comprises the Newcastle Disease Virus (NDV). In one embodiment the method of the present invention provides that the one or more avian pathogens comprises the Infectious Bursal Disease Virus (IBDV) and the Newcastle Disease Virus (NDV).

An aspect of the invention provides a method of inducing an immune response in an avian animal to Marek's Disease Virus and one or more avian pathogen(s), comprising the step of administering to an avian an effective amount of an immunogenic or vaccine composition of the invention. In one embodiment the method of the present invention provides that the one or more avian pathogen(s) are selected from a group consisting of Infectious Bursal Disease Virus (IBDV); Newcastle disease virus (NDV); Infectious Bronchitis Virus (IBV); Infectious Laryngotracheitis Virus (ILTV); Chicken Anemia Virus (CAV); and Avian Influenza Virus (AIV). In one embodiment the method of the present invention provides that the one or more avian pathogen(s) comprises the Infectious Bursal Disease Virus (IBDV). In one embodiment the method of the present invention provides that the one or more avian pathogen(s) comprises the Newcastle Disease Virus (NDV). In one embodiment the method of the present invention provides that the one or more avian pathogens comprise the Infectious Bursal Disease Virus (IBDV) and the Newcastle Disease Virus (NDV). In one or more embodiments the method of the present invention provides that the administration is performed by spray administration, in ovo administration, subcutaneous administration, intramuscular administration, oral administration or nasal administration. In one embodiment the method comprises in ovo administration. In one embodiment the method provides that the in ovo administration occurs in an embryonated egg between about 16-22 days of development. In one or more embodiments the method provides that the in ovo administration occurs in an embryonated egg at about 18 days of development. In one or more embodiments the method provides that the administration route comprises in ovo administration followed by spray administration. In one embodiment the method provides that the administration route comprises spray administration. In one or more embodiments the method provides that the avian is selected from the group consisting of chicken, turkey, goose, duck, pheasant, ostrich, pigeon and quail. In one embodiment the method provides that the avian comprises a chicken.

An aspect of the present invention provides a vaccine composition comprising the recombinant HVT of the invention which comprises a nucleotide sequence encoding the F protein from the Newcastle Disease Virus further comprising a composition comprising an attenuated Infectious Bursal Virus and an antibody that specifically binds to the Infectious Bursal Virus. In one or more embodiments the composition comprising the IBDV is the attenuated IBD strain 2512 and comprises the Bursaplex™ vaccine. In one or more embodiments the composition comprising the IBDV is the attenuated IBD strain V877 comprises the Magniplex™ vaccine.

### BRIEF DESCRIPTION OF THE FIGURES

FIG. 1 is a representation of a PCR reaction demonstrating the correct insertion of a gfp gene at the UL55/Gene 3 site of the HVT genome.

FIG. 2 is a representation of a PCR reaction demonstration the integration site of a gfp gene at the UL35/36 integration site of the HVT genome.

FIGS. 3A, 3B, 3C are representations of PCR reactions demonstrating the correct integration of the VP2 gene into the HVT genome for HVT IBD 1.

FIGS. 4A and 4B are representations of transfected/infected JBJ-1 cells staining for IBDV VP2 (panel A) and HVT infection (panel B) for HVT IBD 5.

FIG. 5 is a representation of PCR reactions performed to confirm correct orientation of the VP2 insert into the UL35/36 integration site of the HVT genome for HVT IBD 5.

FIG. 6 is a representation of a Western blot analysis of infected cell lysate using a monoclonal antibody against IBDVR63 showing a protein band of about 50 KD for HVT IBD 6a.

FIGS. 7A and 7B is a representation of a PCR reactions demonstrating correct VP2 gene integration at the UL35/36 site in the HVT genome for HVT IBD 6a.

FIG. 8A is a representation of PCR reactions demonstrating the correct VP2 gene integration at the UL55/gene3 site in the HVT genome for HVT IBD 9.

FIG. 8B is a representation of PCR reactions demonstrating insertions of the IBD insert in HVT-IBD (TB03-24-1) and HVT-IBD (TB03-24-6) constructs.

FIGS. 9A, 9B and 9C is a are representations of PCR reactions demonstrating the correct VP2 gene integration at the UL55/gene 3 site in the HVT genome for HVT IBD 30.

FIGS. 10A and 10B are representations of Western blot analysis of transfected/infected cell lysates using a monoclonal antibody against IBDVR63 for HVT IBD 31.

FIGS. 11A and 11B are representations of PCR reactions demonstrating correct VP2 gene integration at the UL35/36 integration site in the HVT genome for HVT IBD 31.

FIGS. 12A, 12B, and 12C are representations of PCR reactions demonstration correct VP2 gene integration at the UL55/gene 3 integration site in the HVT genome for HVT IBD 34.

FIG. 13 is a graphical representation of the IBDV serology responses of HVT-IBD 1, 5, 9 and 15.

FIG. 14 is a graphical representation of the IBDV serology responses of HVT IBD 6a, 30, 31.

FIGS. 15A and 15B are representations of PCR reactions demonstration correct orientation of the NDVF insert for HVT ND #38.

FIGS. 16A and 16B are representations of PCR reactions demonstration correct orientation for the NDVF insert for HVT ND #39.

FIGS. 17A, 17B, 17C are representations of PCR reactions demonstrating correct orientation for the NDV F insert for HVT ND #40.

FIG. 18 is a representation of multiple PCR reactions demonstration correct orientation for the NDVF insert for HVT NDV 42.

FIG. 19 is a representation of PCR reactions demonstrating the correct orientation of the NDVF insert for HVT NDV 45.

FIGS. 20A, 20B, 20C is a representation of PCR reactions demonstrating the correct orientation of the NDVF insert for HVT NDV 46.

FIG. 21 is a linear representation of the HVT genome denoting insertion site A, UL35 (HVT043)-UL36 (HVT044).

FIG. 22 is a linear representation of the HVT genome denoting insertion site B, UL55 (HVT065)-Gene 3 (HVT066).

FIG. 23 is a representation of the IBDV VP2 Faragher strain F52/70.

FIG. 24 is a representation of the synthesis of plasmid pHVT-IBD #30.

FIG. 25 is a representation of the synthesis of plasmid pHVT-ND #42.

FIG. 26 is a representation of the circular map of Transfer Plasmid pSiteA #30.

FIG. 27 is a representation of the circular map of Transfer Plasmid pSite B #42.

FIG. 28 is a representation of the production of the intermediate recombinant HVT-ND #42.

FIG. 29 is a representation of the production of HVT-IBD #30-ND #42.

FIG. 30 is a representation of the construct characterization of HVT-IBD #30-ND #42 based on PCR and restriction endonuclease digestion of site A.

FIG. 31 is a representation of the construct characterization of HVT-IBD #30-ND #42 based on PCR and restriction endonuclease digestion of site B.

FIG. 32 is a representation of a western blot analysis of HVT-IBD #30-ND #42 target protein expression of IBD VP2.

FIG. 33 is a representation of western blot analysis of HVT-IBD #30-ND #42 target protein expression of NDV F.

### BRIEF DESCRIPTION OF THE SEQUENCES

| | |
|---|---|
| SEQ ID NO. 1 | DNA sequence of human CMV (hCMV) promoter |
| SEQ ID NO. 2 | DNA sequence of mouse CMV (mCMV) promoter |
| SEQ ID NO. 3 | DNA sequence of strain D26-76 NDV F protein |
| SEQ ID NO. 4 | Amino acid sequence of strain D26-76 NDV F protein |
| SEQ ID NO. 5 | DNA Sequence of vIBDV (F52/70) VP2 |
| SEQ ID NO. 6 | DNA sequence of Bovine Growth Hormone Poly A |
| SEQ ID NO. 7 | DNA Sequence of HVT UL55 Flanking |
| SEQ ID NO. 8 | DNA Sequence of HVT Gene 3 Flanking |
| SEQ ID NO. 9 | DNA Sequence of Pec Promoter |
| SEQ ID NO. 10 | DNA Sequence of vvIBDV VP2 |
| SEQ ID NO. 11 | DNA Sequence SV40 promoter |
| SEQ ID NO. 12 | DNA Sequence SV40 polyA sequence |
| SEQ ID NO. 13 | DNA Sequence of HVT UL35 Flanking |
| SEQ ID NO. 14 | DNA Sequence of HVT UL36 Flanking |
| SEQ ID NO. 15 | DNA Sequence of Chicken Beta-actin Promoter |
| SEQ ID NO. 16 | DNA Sequence of Modified transfer plasmid for HVT-gfp-A #14* |
| SEQ ID NO. 17 | DNA sequence Original transfer plasmid for HVT-gfp-A #14 |
| SEQ ID NO. 18 | DNA Sequence Transfer plasmid for HVT-gfp-B #13 |
| SEQ ID NO. 19 | DNA sequence Transfer plasmid for HVT-gfp-B #13a |
| SEQ ID NO. 20 | DNA sequence Transfer plasmid for HVT-IBD #1 |
| SEQ ID NO. 21 | DNA sequence Transfer plasmid for HVT-IBD #5 |
| SEQ ID NO. 22 | DNA Sequence Transfer plasmid for HVT-IBD #6a |
| SEQ ID NO. 23 | DNA sequence Transfer plasmid for HVT-IBD #9 |
| SEQ ID NO. 24 | DNA sequence Transfer plasmid for HVT-IBD #30 |
| SEQ ID NO. 25 | DNA sequence Transfer plasmid for HVT-IBD #31 |
| SEQ ID NO. 26 | DNA sequence Transfer plasmid for HVT-IBD #32 |
| SEQ ID NO. 27 | DNA sequence Transfer plasmid for HVT-IBD #33 |
| SEQ ID NO. 28 | DNA sequence Transfer plasmid for HVT-IBD #34 |
| SEQ ID NO. 29 | DNA sequence of Transfer plasmid for HVT-ND #38 |
| SEQ ID NO. 30 | DNA sequence Transfer plasmid for HVT-ND #39 |
| SEQ ID NO. 31 | DNA sequence Transfer plasmid for HVT-ND #40 |
| SEQ ID NO. 32 | DNA sequence Transfer plasmid for HVT-ND #41a |
| SEQ ID NO. 33 | DNA sequence Original Transfer plasmid for HVT-ND #42 |
| SEQ ID NO. 34 | DNA sequence of Cloning plasmid for pSiteB |
| SEQ ID NO. 35 | DNA sequence Final Transfer plasmid for pSiteB-#42 |
| SEQ ID NO. 36 | DNA sequence Transfer plasmid for HVT-ND #44 |
| SEQ ID NO. 37 | DNA sequence Transfer plasmid for HVT-ND #45 |
| SEQ ID NO. 38 | DNA sequence Transfer plasmid for HVT-ND #46 |
| SEQ ID NO. 39 | DNA sequence Transfer plasmid for HVT-ND #48 |
| SEQ ID NO. 40 | DNA sequence of 5′Upstream Sbfi gfp gene mutagenesis primer 1 |
| SEQ ID NO. 41 | DNA sequence of 5′Upstream Sbfi gfp gene mutagenesis primer 2 |
| SEQ ID NO. 42 | DNA sequence of 3′ downstream Sbfi gfp gene mutagenesis primer 1 |
| SEQ ID NO. 43 | DNA sequence of 3′ downstream Sbfi gfp gene mutagenesis primer 2 |
| SEQ ID NO. 44 | DNA sequence of upper UL55-gene 3 primer |
| SEQ ID NO. 45 | DNA sequence of lower UL55-gene 3 primer |
| SEQ ID NO. 46 | DNA sequence upper UL55-gene3 PCR primer |
| SEQ ID NO. 47 | DNA sequence lower UL55-gene3 PCR primer |
| SEQ ID NO. 48 | DNA sequence for upper primer localized within IBD VP2 coding |
| | region |
| SEQ ID NO. 49 | DNA sequence for lower primer downstream within HVT IBD#1 |
| SEQ ID NO. 50 | DNA sequence for upper primer of the upstream junction of the |
| | insertion site of transfer plasmid HVT IBD#1 |
| SEQ ID NO. 51 | DNA sequence for lower primer within the IBDV VP2 coding region |
| | of HVT IBD #1 |
| SEQ ID NO. 52 | DNA sequence for upper primer within the IBDV VP2 coding region |
| SEQ ID NO. 53 | DNA sequence for lower primer downstream of the UL35/36 |
| | integration site of HVT-IBD #5 |
| SEQ ID NO. 54 | DNA sequence for upper primer of the UL35/36 insertion site of HVT |
| | IBD #5 |
| SEQ ID NO. 55 | DNA sequence for lower primer within the hCMV promoter of HVT |
| | IBD #5 |
| SEQ ID NO. 56 | DNA sequence for upper primer upstream of the integration site of |
| | HVT IBD #6a |
| SEQ ID NO. 57 | DNA sequence for lower primer localized within the pec promoter of |
| | HVT IBD#6a |
| SEQ ID NO. 58 | DNA sequence for upper primer localized within the IBD VP2 coding |
| | region of HVT IBD #6a |
| SEQ ID NO. 59 | DNA sequence for lower primer localized downstream of the |
| | UL35/36 insertion site |
| SEQ ID NO. 60 | DNA sequence for upper primer upstream of the integration site of |
| | UL55/Gene3 for HVT IBD#9 |
| SEQ ID NO. 61 | DNA sequence for lower primer downstream of the integration site of |
| | UL55/Gene3 for HVT IBD#9 |
| SEQ ID NO. 62 | DNA sequence for insert orientation upper primer surrounding |
| | upstream junction of the HVT IBD#9 VP2 gene insertion |
| SEQ ID NO. 63 | DNA sequence for insert orientation lower primer localized within |
| | IBDV VP2 coding region for HVT IBD#9 |
| SEQ ID NO. 64 | DNA sequence for upper primer downstream site determining correct |
| | integration of the IBDV VP2 coding region for HVT IBDV #9 |
| SEQ ID NO. 65 | DNA sequence for lower primer downstream site determining correct |
| | integration of the IBDV VP2 coding region for HVT IBDV #9 |
| SEQ ID NO. 66 | DNA sequence for upper primer for upstream region of integration |
| | site of UL55-Gene3 for HVT IBD#30 |
| SEQ ID NO. 67 | DNA sequence for lower primer for upstream region of integration |
| | site of UL55-Gene3 for HVT IBD#30 |
| SEQ ID NO. 68 | DNA sequence for upper primer to confirm correct orientation of VP2 |
| | insert surrounding the 3′ junction of the insertion site of HVT |
| | IBD#30 |
| SEQ ID NO. 69 | DNA sequence for lower primer to confirm correct orientation of VP2 |
| | insert surrounding the 3′ junction of the insertion site of HVT |
| | IBD#30 |
| SEQ ID NO. 70 | DNA sequence for upper primer to confirm correct orientation of VP2 |
| | insert integration outside of the expression cassette of HVT IBD#30 |
| SEQ ID NO. 71 | DNA sequence for lower primer to confirm correct orientation of VP2 |
| | insert integration outside of the expression cassette of HVT IBD#30 |
| SEQ ID NO. 72 | DNA sequence for upper primer to confirm correct orientation of VP2 |
| | insert upstream of the UL35/36 integration site of HVT IBD #31 |
| SEQ ID NO. 73 | DNA sequence for lower primer to confirm correct orientation of VP2 |
| | insert localized within the chicken beta actin promoter of HVT |
| | IBD#31 |
| SEQ ID NO. 74 | DNA sequence for upper primer to confirm confirmation of VP2 |
| | insert localized within the IBDV VP2 coding region |
| SEQ ID NO. 75 | DNA sequence for lower primer to confirm correct orientation of VP2 |
| | insert located downstream of UL35/36 integration site of HVT |
| | IBD#31 |
| SEQ ID NO. 76 | DNA sequence for upper primer targeting the downstream integration |
| | site of the VP2 insert of HVT IBD#31 located within the VP2 insert |
| SEQ ID NO. 77 | DNA sequence for lower primer localized downstream of the |
| | UL35/36 site of HVT IBD#31 |
| SEQ ID NO. 78 | DNA sequence for upper primer for upstream region of integration |
| | site of Gene3-UL55 for HVT-IBD #34 |
| SEQ ID NO. 79 | DNA sequence for lower primer localized within chicken beta-actin |
| | promoter for HVT-IBD #34 |
| SEQ ID NO. 80 | DNA sequence for upper primer localized within IBDV VP2 coding |
| | region for HVT-IBD #34 |
| SEQ ID NO. 81 | DNA sequence for lower primer localized downstream of Gene3- |
| | UL55 insertion site for HVT IBD#34 |
| SEQ ID NO. 82 | DNA sequence for upper primer localized outside of the VP2 |
| | expression cassette of HVT IBD #34 |
| SEQ ID NO. 83 | DNA sequence for lower primer localized outside of the VP2 |
| | expression cassette of HVT IBD #34 |
| SEQ ID NO. 84 | DNA sequence for upper primer for upstream region of integration |
| | site of UL35-UL36 of HVT ND#38 |
| SEQ ID NO. 85 | DNA sequence for lower primer that localized within NDV F coding |
| | region of HVT ND#38 |
| SEQ ID NO. 86 | DNA sequence for upper primer surrounding the 3′ junction of the |
| | insertion localized within NDV F coding region of HVT ND#38 |
| SEQ ID NO. 87 | DNA sequence for lower primer localized downstream of UL35- |
| | UL36 insertion site of HVT ND#38 |
| SEQ ID NO. 88 | DNA sequence for upper primer outside of the expression cassette of |
| | HVT ND#38 |
| SEQ ID NO. 89 | DNA sequence for lower primer outside of the expression cassette of |
| | HVT ND#38 |
| SEQ ID NO. 90 | DNA sequence for upper primer upstream region of integration site of |
| | UL35-UL36 for HVT-ND #39 |
| SEQ ID NO. 91 | DNA sequence for lower primer localized within chicken beta-actin |
| | promoter HVT-ND #39 |
| SEQ ID NO. 92 | DNA sequence for upper primer surrounding the downstream junction |
| | of the insertion localized within poly A region of HVT-ND #39 |
| SEQ ID NO. 93 | DNA sequence for lower primer localized downstream of UL35- |
| | UL36 insertion of HVT-ND #39 |
| SEQ ID NO. 94 | DNA sequence for upper primer outside of the expression cassette of |
| | HVT-ND #39 |
| SEQ ID NO. 95 | DNA sequence for lower primer outside of the expression cassette |
| | HVT-ND #39 |
| SEQ ID NO. 96 | DNA sequence for upper primer upstream of the UL35/36 integration |
| | site for HVT ND#40 |
| SEQ ID NO. 97 | DNA sequence for lower primer localized within chicken beta actin |
| | promoter for HVT ND#40 |
| SEQ ID NO. 98 | DNA sequence for upper primer localized within NDVF coding |
| | region for HVT ND#40 |
| SEQ ID NO. 99 | DNA sequence for lower primer located at the downstream junction |
| | of the insertion site for HVT ND#40 |
| SEQ ID NO. 100 | DNA sequence for upper primer located outside of the expression |
| | cassette for HVT ND#40 |
| SEQ ID NO. 101 | DNA sequence for lower primer located outside of the expression |
| | cassette for HVT#40 |
| SEQ ID NO. 102 | DNA sequence for upper primer for PCR amplification of cassette for |
| | HVT ND#42 |
| SEQ ID NO. 103 | DNA sequence for lower primer for PCR amplification of cassette for |
| | HVT ND#42 |
| SEQ ID NO. 104 | DNA sequence for upper primer located outside of cassette for HVT |
| | ND#42 |
| SEQ ID NO. 105 | DNA sequence for lower primer located outside of cassette for HVT |
| | ND #42 |
| SEQ ID NO. 106 | DNA sequence for upper primer located upstream and outside of the |
| | expression cassette for HVT ND #42 |
| SEQ ID NO. 107 | DNA sequence for lower primer located within ND F coding region |
| | for HVT ND #42 |
| SEQ ID NO. 108 | DNA sequence for upper primer located upstream and outside of the |
| | expression cassette for HVT ND #42 |
| SEQ ID NO. 109 | DNA sequence for lower primer located within ND F coding region |
| | for HVT ND #42 |
| SEQ ID NO. 110 | DNA sequence for upper primer located upstream and outside of the |
| | expression cassette for HVT ND #42 |
| SEQ ID NO. 111 | DNA sequence for lower primer located within NDV F coding region |
| | for HVT ND #42 |
| SEQ ID NO. 112 | DNA sequence for upper primer located upstream and outside of the |
| | expression |
| SEQ ID NO. 113 | DNA sequence for lower primer located within ND F coding region |
| | for HVT ND #42 |
| SEQ ID NO. 114 | DNA sequence for upper primer located upstream and outside of the |
| | expression cassette for HVT ND #42 |
| SEQ ID NO. 115 | DNA sequence for lower primer located within ND F coding region |
| | for HVT ND #42 |
| SEQ ID NO. 116 | DNA sequence for upper primer localized upstream of UL55 for HVT |
| | ND #44 |
| SEQ ID NO. 117 | DNA sequence for lower primer localized within chicken beta actin |
| | promoter for HVT ND #44 |
| SEQ ID NO. 118 | DNA sequence for upper primer localized upstream of UL55 for HVT |
| | ND #44 |
| SEQ ID NO. 119 | DNA sequence for lower primer localized within chicken beta actin |
| | promoter for HVT ND #44 |
| SEQ ID NO. 120 | DNA sequence for upper primer localized within NDV F gene coding |
| | sequence for HVT ND#44 |
| SEQ ID NO. 121 | DNA sequence for lower primer localized downstream of UL55- |
| | Gene3 insertion site for HVT ND#44 |
| SEQ ID NO. 122 | DNA sequence for upper primer localized outside of the expression |
| | cassette for HVT ND#44 |
| SEQ ID NO. 123 | DNA sequence for lower primer localized outside of the expression |
| | cassette for HVT#44 |
| SEQ ID NO. 124 | DNA sequence for upper primer localized outside of the expression |
| | cassette for HVT#45 |
| SEQ ID NO. 125 | DNA sequence for lower primer localized outside of the expression |
| | cassette for HVT#45 |
| SEQ ID NO. 126 | DNA sequence for upper primer located upstream and outside of the |
| | expression cassette for HVT ND#45 |
| SEQ ID NO. 127 | DNA sequence for lower primer located within ND F coding region |
| | for HVT ND#45 |
| SEQ ID NO. 128 | DNA sequence for upper primer located upstream and outside of the |
| | expression cassette for HVT ND#45 |
| SEQ ID NO. 129 | DNA sequence for lower primer located within the ND F coding |
| | region for HVT ND#45 |
| SEQ ID NO. 130 | DNA sequence for upper primer surrounding the downstream junction |
| | of the insertion for HVT ND#45 |
| SEQ ID NO. 131 | DNA sequence for lower primer localized downstream of Gene3- |
| | UL55 insertion site |
| SEQ ID NO. 132 | DNA sequence for upper primer surrounding the downstream junction |
| | of the insertion for HVT ND#45 |
| SEQ ID NO. 133 | DNA sequence for lower primer localized that localized downstream |
| | of Gene3-UL55 insertion site |
| SEQ ID NO. 134 | DNA sequence for upper primer localized outside of the expression |
| | cassette for HVT ND#46 |
| SEQ ID NO. 135 | DNA sequence for lower primer localized outside of the expression |
| | cassette for HVT ND#46 |
| SEQ ID NO. 136 | DNA sequence for upper primer located upstream and outside of the |
| | integration site for HVT ND#46 |
| SEQ ID NO. 137 | DNA sequence for lower primer located within the mCMV promoter |
| | for HVT ND#46 |
| SEQ ID NO. 138 | DNA sequence for upper primer localized within NDV F gene coding |
| | sequence for HVT ND#46 |
| SEQ ID NO. 139 | DNA sequence for lower primer localized downstream and outside of |
| | expression cassette for HVT ND#46 |
| SEQ ID NO. 140 | DNA sequence for upper primer localized within NDV F gene coding |
| | sequence for HVT ND#46 |
| SEQ ID NO. 141 | DNA sequence for lower primer localized downstream and outside of |
| | expression cassette for HVT ND#46 |
| SEQ ID NO. 142 | DNA sequence for upper primer localized within NDV F gene coding |
| | sequence for HVT ND#46 |
| SEQ ID NO. 143 | DNA sequence for lower primer localized downstream and outside of |
| | expression cassette for HVT ND#46 |
| SEQ ID NO. 144 | DNA sequence for upper primer localized within NDV F gene coding |
| | sequence for HVT ND#46 |
| SEQ ID NO. 145 | DNA sequence for lower primer localized downstream and outside of |
| | expression cassette for HVT ND#46 |
| SEQ ID NO. 146 | DNA sequence for upper primer localized upstream region of |
| | integration site of Gene 3-UL55 for HVT ND#48 |
| SEQ ID NO. 147 | DNA sequence for lower primer localized within chicken beta-actin |
| | promoter for HVT ND#48 |
| SEQ ID NO. 148 | DNA sequence for upper primer surrounding the downstream junction |
| | of the insertion for HVT ND#48 |
| SEQ ID NO. 149 | DNA sequence for lower primer localized downstream of Gene 3- |
| | UL55 insertion site for HVT ND#48 |
| SEQ ID NO. 150 | DNA sequence for upper primer surrounding the downstream junction |
| | of the insertion for HVT ND#48 |
| SEQ ID NO. 151 | DNA sequence for lower primer localized downstream of Gene 3- |
| | UL55 insertion site for HVT ND#48 |
| SEQ ID NO. 152 | DNA sequence for upper primer located outside of the expression |
| | cassette for HVT ND#48 |
| SEQ ID NO. 153 | DNA sequence for lower primer located outside of the expression |
| | cassette for HVT ND#48 |
| SEQ ID NO. 154 | DNA sequence for upper primer for amplification of the NDV F |
| | expression cassette of HVT ND#42 for HVT-IBD-ND #42-#30 |
| SEQ ID NO. 155 | DNA sequence for lower primer for amplification of the NDV F |
| | expression cassette of HVT ND#42 for HVT-IBD-ND #42-#30 |
| SEQ ID NO. 156 | DNA sequence for upper primer for amplification of the IBD gene |
| | expression cassette of plasmid #30 for HVT-IBD-ND #42-#30 |
| SEQ ID NO. 157 | DNA sequence for lower primer for amplification of the IBD gene |
| | expression cassette of plasmid #30 for HVT-IBD-ND #42-#30 |
| SEQ ID NO. 158 | DNA sequence for RSV LTR promoter |

### DETAILED DESCRIPTION OF THE INVENTION

The following is a detailed description provided to aid those skilled in the art. Those of ordinary skill in the art may make modifications and variations in the embodiments described herein without departing from the spirit or scope of the present invention.

The present invention relates to vaccines for avian use based on live recombinant avian herpesviruses, namely, in particular, Marek's disease virus (MDV) and more especially on HVT virus (herpesvirus of turkeys), into which has been inserted one or more nucleotide sequence(s) coding for and expressing an antigenic polypeptide of a pathogenic agent, under conditions affording an immunization leading to an effective protection of the vaccinated animal against the said pathogenic agent or agents. Marek's Disease (MD) is a common lymphoproliferative disease of chickens, caused by Marek's Disease virus (MDV), which can result in significant losses in the poultry industry. Currently, MD is controlled in poultry using vaccines using serotype 3 of MDV, which is the related Herpesvirus of Turkeys (HVT). By introducing genes from poultry viruses other than MDV into the HVT genome at particular genetic positions, the inventors have been able to develop novel recombinant viral vaccines that enable simultaneous protection in poultry against MD and one or more additional diseases through administration of a single viral vaccine.

The invention provides recombinant viral vectors for the insertion and expression of foreign genes for use in safe immunizations to protect avians against a variety of pathogens. The invention also provides multivalent compositions or vaccines comprising one or more recombinant HVT viral vectors for protection against a variety of pathogens. Additionally, the invention provides methods of making and using the recombinant viral vectors alone or in combination with other vaccines or pharmaceutical compositions.

In one aspect the present invention provides a recombinant Herpesvirus of turkey (HVT) genome comprising one or more nucleotide sequence(s) coding for one or more heterologous antigen(s) inserted into the intergenic loci UL 35/UL 36 in the unique long (UL) region of the HVT genome.

In one aspect the present invention provides a recombinant Herpesvirus of Turkey (HVT) genome comprising one or more nucleotide sequences coding for one or more heterologous antigens or antigens inserted into the intergenic loci UL 35/UL 36 in the unique long region of the HVT genome and one or more nucleotide sequences or sequences coding for one or more heterologous antigens inserted at the UL55/Gene 3 site in the unique long region (UL) of the HVT genome.

In one or more embodiments the present invention provides a recombinant HVT wherein the one or more heterologous antigens or antigens are protective against avian pathogens or pathogens selected from the group consisting of: Infectious Bursal Disease Virus (IBDV); Newcastle disease virus (NDV); Infectious Bronchitis Virus (IBV); Infectious Laryngotracheitis Virus (ILTV); Chicken Anemia Virus (CAV); and Avian Influenza Virus (AIV).

In one or more embodiments the present invention provides a recombinant HVT wherein the one or one or more heterologous antigens are selected from the group consisting of: the VP2, VP3 or VP4 proteins of the Infectious Bursal Disease Virus (IBDV); the VP1 or VP2 proteins of the Chicken Anemia Virus (CAV); the F/HN chimera protein or the F, NP, P, M, HN, or L proteins of the Newcastle Disease Virus (NDV); the S1, S2 or M proteins of Infectious Bronchitis Virus (IBV); the gB, gC, gD, gE, gH, gI or gL proteins of the Infectious Laryngotracheitis Virus (ILTV); and any of the HA, NA, NP or M proteins of the Avian Influenza Virus (AIV).

In one or more embodiments the recombinant HVT of the present invention provides that the one or more heterologous antigen is protective against IBDV. In one embodiment the recombinant HVT of the present invention provides the one or more heterologous antigen is the VP2 protein of IBDV. In one embodiment the recombinant HVT of the present invention provides the VP2 protein is encoded by the nucleotide sequence comprising at least 80% sequence identity to the nucleotide sequence comprising SEQ ID NO. 5 or SEQ ID NO. 10. In one embodiment the recombinant HVT of the present invention provides the VP2 protein encoded by the nucleotide sequence comprising either SEQ ID NO. 5 or SEQ ID NO.10.

In one or more embodiments the recombinant HVT of the present invention provides the one or more heterologous antigen or antigen is protective against Newcastle Disease Virus (NDV). In one embodiment the recombinant HVT of the present invention provides the one or more heterologous antigen is the F protein of NDV. In one embodiment the recombinant HVT of the present invention provides that the F protein of NDV is encoded by a nucleotide sequence comprising at least 80% sequence identity to the nucleotide sequence comprising SEQ ID NO. 3. In one embodiment the recombinant HVT of the present invention the F protein of NDV is encoded by the nucleotide sequences comprising SEQ ID NO. 3.

In one or more embodiments the recombinant HVT of the present invention provides the one or more heterologous antigens are protective against NDV and IBDV. In one or more embodiments the recombinant HVT of the present invention provides the at least one heterologous antigens are the F protein of NDV and the VP2 protein of IBDV.

In one or more embodiments the recombinant HVT of the present invention provides the F protein of NDV encoded by a nucleotide sequence comprising at least 80% sequence identity to the nucleotide sequence comprising SEQ ID NO. 3 and the VP2 protein of IBDV encoded by the nucleotide sequence comprising at least 80% sequence identity to the nucleotide sequence comprising SEQ ID NO. 5 or SEQ ID NO.10.

In one or more embodiments the recombinant HVT of the present invention provides the F protein of NDV encoded by the nucleotide sequence comprising SEQ ID NO. 3 and the VP2 protein of IBDV is encoded by the nucleotide sequence comprising SEQ ID NO. 5 or SEQ ID NO.10.

In one or more embodiments the recombinant HVT of the present invention comprises a genome comprising one or more expression cassette or cassettes comprising one or more nucleotide sequence or sequences that encode one or more heterologous antigen or antigens. In one embodiment the recombinant HVT comprises a recombinant HVT genome an expression cassette that comprises a nucleotide sequence encoding promoters that are operatively linked to one or more nucleotides that encode antigens to be expressed. In one embodiment the antigen to be expressed comprises the F protein of NDV. In one embodiment the antigen to be expressed comprise the VP2 protein of IBDV. In one embodiment the antigens to be expressed comprise both the F protein of NDV and the VP2 protein of IBDV.

In one embodiment the recombinant HVT of the present invention provides the one or more promoters are selected from the group consisting of: immediate early cytomegalovirus human (hCMV) promoter: guinea pig immediate early CMV promoter; murine immediate early CMV promoter; Pec promoter; β-chicken actin promoter; SV40 promoter; Pseudorabies Virus promoters of glycoprotein X promoter; Herpes Simplex Virus-1 alpha 4 promoter; Marek's Disease Virus promoters of glycoproteins gA, gC, gB, gE, or gI promoter; Infectious Laryngotracheitis Virus promoters of glycoprotein gB, gE, gI, gD promoter; and Bovine Herspesvirus 1/1 VP8 promoter. In one embodiment the recombinant HVT comprises the human CMV promoter. In one embodiment the recombinant HVT comprises the murine CMV promoter. In one embodiment the recombinant HVT comprises the hCMV and mCMV promoter.

In one or more embodiments the recombinant HVT comprises a nucleotide sequence encoding a poly adenylation (polyA) signal. In one or more embodiments the recombinant HVT comprises a nucleotide sequence encoding a poly A signal and is selected from BGH poly A (SEQ ID NO.6) or SV40 poly A sequence (SEQ ID NO.12). In one embodiment the poly A signal is a BGH poly A signal. In one embodiment the poly A signal is an SV40 poly A signal.

In one aspect the recombinant HVT of the present invention comprises a CMV promoter operatively linked to a nucleotide sequence encoding a VP2 protein from a IBDV further comprising a nucleotide sequence coding for a polyadenylation signal, all part of a VP2 expression cassette inserted in a non-coding region of the HVT genome. In one embodiment the CMV promoter comprises an hCMV promoter (SEQ ID NO.1). In one embodiment the nucleotide sequence encoding the VP2 protein of IBDV is selected from SEQ ID NO.5 or SEQ ID NO.10. In one embodiment the nucleotide sequence encoding the VP2 protein comprises SEQ ID NO.5. In one embodiment the nucleotide sequence encoding the VP2 protein comprises SEQ ID NO.10. In one embodiment the polyadenylation signal comprises SEQ ID NO. 6. In one embodiment the polyadenylation signal comprises SEQ IDNO.12. In one embodiment the promoter, the nucleotide sequence encoding the VP2 protein and the poly A signal comprise an expression cassette. In one embodiment the expression cassette is inserted into the HVT genome at the UL55/gene 3 site. In one embodiment the expression cassette is inserted into the HVT genome at the UL35/36 site within the genome. In one embodiment the expression cassette comprises, in order, SEQ ID NO.1, SEQ ID NO.5 or SEQ ID NO.10 and SEQ ID NO.6 inserted into the HVT genome at the UL55/gene 3 site.

In one aspect the recombinant HVT of the present invention comprises a CMV promoter operatively linked to a nucleotide sequence encoding an F protein of an NDV further comprising a nucleotide sequence coding for a polyadenylation signal all part of an NDV F cassette inserted into a non-coding location within the HVT genome. In one embodiment the CMV promoter comprises an mCMV (SEQ ID NO.2) promoter. In one embodiment the nucleotide sequence encoding the F protein of an NDV which comprises SEQ ID NO.3. In one embodiment the polyadenylation signal is coded for by nucleotide sequence comprising SEQ ID NO.12. In one embodiment the promoter, the nucleotide sequence encoding the F protein and the poly A signal comprise an expression cassette. In one embodiment the expression cassette is inserted into the HVT genome at the UL55/gene 3 site. In one embodiment the expression cassette is inserted into the HVT genome at the UL35/36 site within the genome. In one embodiment the expression cassette comprises, in order, SEQ ID NO.2, SEQ ID NO.3 and SEQ ID NO.12 inserted into the HVT genome at the UL55/gene 3 site.

In one aspect the recombinant HVT of the present invention comprises a CMV promoter operatively linked to a nucleotide sequence encoding a VP2 protein of an IBDV further comprising a nucleotide sequence coding for a polyadenylation signal, all comprising a VP2 expression cassette inserted into a non-coding location within the HVT genome. In one embodiment the recombinant HVT of the present invention further comprises a CMV promoter operatively linked to a nucleotide sequence encoding the F protein of an NDV further comprising a nucleotide sequence coding for a polyadenylation signal as part of an NDV F expression cassette inserted into the same insertion site as the VP2 cassette. In one embodiment the recombinant HVT of the present further comprises a CMV promoter operatively linked to a nucleotide sequence encoding an F protein of NDV further comprising a nucleotide sequence coding for a polyadenylation signal as part of an NDV F expression cassette inserted into a different site as the VP2 cassette.

In one embodiment the recombinant HVT of the present invention provides a VP2 expression cassette comprising, in order, a nucleotide sequence encoding hCMV promoter (SEQ ID NO. 1), nucleotide sequence encoding IBDV VP2 (selected from SEQ ID NO.5 or SEQ ID NO.10) and nucleotide sequence encoding BGH polyadenylation signal (SEQ ID NO.6) inserted into the HVT genome in the UL35/36 non-coding region and, in order, nucleotide sequence encoding mCMV promoter (SEQ ID NO.2), a nucleotide sequence encoding the F protein from NDV (SEQ ID NO.3) and a nucleotide sequence encoding an SV40 polyadenylation signal (SEQ ID NO.12) inserted into the HVT genome in the UL55/gene 3 non-coding region. In one embodiment the recombinant HVT of the present invention further comprises a nucleotide sequence that encodes one or more antigens selected from the group consisting of Infectious Bursal Disease Virus, Chicken Anemia Virus, Newcastle Disease Virus, Infectious Bronchitis Virus, Infectious Laryngotracheitis Virus and Avian Influenza Virus. In one embodiment the recombinant HVT of the present invention further provides a promoter operatively linked to a nucleotide sequence encoding antigens selected from the group consisting of: a VP1, VP2, VP3 or VP4 antigen of the Infectious Bursal Disease Virus (IBDV);the VP1 or VP2 proteins of the Chicken Anemia Virus (CAV); the F/HN chimera protein or the F, NP, P, M, HN, or L proteins of the Newcastle Disease Virus (NDV); the S1, S2 or M proteins of Infectious Bronchitis Virus (IBV); and any of the HA, NA, NP or M proteins of the Avian Influenza Virus (AIV).

In one embodiment the recombinant HVT of the present invention comprises one or more ILT antigens as part of an expression cassette comprising a promoter that is operatively linked to the nucleotide encoding the ILT antigen and further comprising a nucleotide sequence encoding a polyadenylation signal. In one embodiment the recombinant HVT of the present invention comprises a second and a third expression cassette each comprising nucleotide sequences encoding a promoter operatively linked to a nucleotide sequence encoding avian antigens selected from the group consisting of a VP1, VP2, VP3 or VP4 antigen of the Infectious Bursal Disease Virus (IBDV); the VP1 or VP2 proteins of the Chicken Anemia Virus (CAV); the F/HN chimera protein or the F, NP, P, M, HN, or L proteins of the Newcastle Disease Virus (NDV); the S1, S2 or M proteins of Infectious Bronchitis Virus (IBV); and any of the HA, NA, NP or M proteins of the Avian Influenza Virus (AIV) and further comprising a nucleotide sequence encoding a polyadenylation signal.

In one or more aspects the present invention provides a recombinant DNA encoding the recombinant HVT genome of the present invention.

In one or more aspects the present invention provides an immunogenic composition comprising the recombinant HVT of the present invention and further comprising a pharmaceutically acceptable carrier, excipient or adjuvant.

In one or more aspects the present invention provides a vaccine composition comprising the recombinant HVT of the invention and further comprising a pharmaceutically acceptable carrier, excipient or adjuvant.

In one embodiment the vaccine of the present invention further comprises an additional Marek's disease Virus (MDV) selected from the group consisting of: naturally attenuated MDV-1 strain Rispens (CVI-988); or a Gallid Herpesvirus 3 strain SB-1 virus. In one embodiment the vaccine of the present invention provides that the additional MDV comprises a recombinant genome. In one embodiment the vaccine of the present invention provides that the additional recombinant MDV genome comprises one or more nucleotide sequence(s) encoding one or more heterologous antigen(s) that are protective against one or more avian pathogen(s).

In one embodiment the vaccine of the present invention provides for use in vaccinating an avian against one or more diseases caused by one or more avian pathogen(s). In one or more embodiments the vaccine of the present invention provides for use in protecting an avian against clinical symptoms caused by one or more avian pathogen(s). In one or more embodiments the vaccine of the present invention provides for use in protecting an avian against clinical symptoms caused by Marek's Disease Virus and clinical symptoms caused by one or more avian pathogen(s). In one or more embodiments the vaccine of the present invention provides the one or more avian pathogen(s) selected from a group consisting of: Infectious Bursal Disease Virus (IBDV); Newcastle disease virus (NDV); Infectious Bronchitis Virus (IBV); Infectious Laryngotracheitis Virus (ILTV); Chicken Anemia Virus (CAV); and Avian Influenza Virus (AIV). In one embodiment the vaccine of the present invention provides that the one or more avian pathogen comprises the Newcastle Disease Virus. In one embodiment the vaccine of the present invention provides that the one or more avian pathogen comprises the Infectious Bursal Disease Virus (IBDV). In one embodiment the vaccine of the present invention provides that the one or more avian pathogen comprise the Newcastle Disease Virus and the Infectious Bursal Disease Virus.

In one or more embodiments the vaccine of the present invention provides for use in vaccinating an avian wherein the vaccine is administered by at least one administration of the vaccine by spray administration, in ovo administration, subcutaneous administration, intramuscular administration, oral administration, nasal administration or combination thereof. In one embodiment the vaccine of the present invention provides that the vaccine is administered by in ovo administration. In one embodiment the vaccine of the present invention provides that the in ovo administration occurs in an embryonated egg between about 16-22 days of development. In one or more embodiments the vaccine of the present invention provides that the in ovo administration occurs in an embryonated egg at about 18 days of development. In one embodiment the vaccine of the present invention provides that the administration of the vaccine comprises in ovo administration followed by spray administration. In one embodiment the vaccine of the present invention provides that the administration of the vaccine comprises spray administration.

In one aspect the present invention provides a method of vaccinating an avian to treat or prevent Marek's disease and one or more avian diseases caused by one or more avian pathogens comprises the step of administering an effective amount of the vaccine composition the present invention. In one embodiment the method of the present invention provides that the one or more avian pathogens are selected from a group consisting of Infectious Bursal Disease Virus (IBDV); Newcastle disease virus (NDV); Infectious Bronchitis Virus (IBV); Infectious Laryngotracheitis Virus (ILTV); Chicken Anemia Virus (CAV); and Avian Influenza Virus (AIV). In one embodiment the method of the present invention provides that the one or more avian pathogen comprises the Infectious Bursal Disease Virus (IBDV). In one embodiment the method of the present invention provides that the one or more avian pathogen comprises the Newcastle Disease Virus (NDV). In one embodiment the method of the present invention provides that the one or more avian pathogens comprises the Infectious Bursal Disease Virus (IBDV) and the Newcastle Disease Virus (NDV).

An aspect of the invention provides a method of inducing an immune response in an avian animal to Marek's Disease Virus and one or more avian pathogen(s), comprising the step of administering to an avian an effective amount of an immunogenic or vaccine composition of the invention. In one embodiment the method of the present invention provides that the one or more avian pathogen(s) are selected from a group consisting of Infectious Bursal Disease Virus (IBDV); Newcastle disease virus (NDV); Infectious Bronchitis Virus (IBV); Infectious Laryngotracheitis Virus (ILTV); Chicken Anemia Virus (CAV); and Avian Influenza Virus (AIV). In one embodiment the method of the present invention provides that the one or more avian pathogen(s) comprises the Infectious Bursal Disease Virus (IBDV). In one embodiment the method of the present invention provides that the one or more avian pathogen(s) comprises the Newcastle Disease Virus (NDV). In one embodiment the method of the present invention provides that the one or more avian pathogens comprise the Infectious Bursal Disease Virus (IBDV) and the Newcastle Disease Virus (NDV).In one or more embodiments the method of the present invention provides that the administration is performed by spray administration, in ovo administration, subcutaneous administration, intramuscular administration, oral administration or nasal administration. In one embodiment the method comprises in ovo administration. In one embodiment the method provides that the in ovo administration occurs in an embryonated egg between about 16-22 days of development. In one or more embodiments the method provides that the in ovo administration occurs in an embryonated egg at about 18 days of development. In one or more embodiments the method provides that the administration route comprises in ovo administration followed by spray administration. In one embodiment the method provides that the administration route comprises spray administration. In one or more embodiments the method provides that the avian is selected from the group consisting of chicken, turkey, goose, duck, pheasant, ostrich, pigeon and quail. In one embodiment the method provides that the avian comprises a chicken.

### General Methodologies

It should be understood that this invention is not limited to the particular methodology, protocols, and reagents, etc. described herein and as such may vary. The terminology used herein is for the purpose of describing particular embodiments only and is not intended to limit the scope of the present invention.

Unless otherwise defined, scientific and technical terms used in connection with the invention described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo-or polynucleotide chemistry and hybridization described herein are those well-known and commonly used in the art.

Standard techniques are used for recombinant DNA, oligonucleotide synthesis, and tissue culture and transfection that are well known to those of skill in the art. Enzymatic reactions and purification techniques are performed according to manufacturer's specifications or as commonly accomplished in the art or as described herein. The foregoing techniques and procedures are generally performed according to conventional methods well known in the art and as described, but not limited to the various general and more specific references that are cited and discussed throughout the present specification, See ex. Sambrook et al. MOLECULAR CLONING: LAB. MANUAL (3^{rd} ed., Cold Spring Harbor Lab. Press, Cold Spring Harbor, N.Y., 2001) and Ausubel et al. Current Protocols in Molecular Biology (New York: Greene Publishing Association JWiley Interscience), Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. 1. Freshney, ed.1987); Introduction to Cell and Tissue Culture (1. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach(P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (Y. T. DeVita et al., eds., J.B. Lippincott Company, 1993).

Other than in the operating examples, or where otherwise indicated, all numbers expressing quantities of ingredients or reaction conditions used herein should be understood as modified in all instances by the term “about”.

All patents and other publications identified are expressly incorporated herein by reference for the purpose of describing and disclosing, for example, the methodologies described in such publications that might be used in connection with the present invention. These publications are provided solely for their disclosure prior to the filing date of the present application.

### Definitions

Before describing the present invention in detail, several terms used in the context of the present invention will be defined. In addition to these terms, others are defined elsewhere in the specification as necessary. Unless otherwise expressly defined herein, terms of art used in this specification will have their art-recognized meanings.

It is noted that in this disclosure, terms such as “comprises”, “comprised”, “comprising”, “contains”, “containing”, “consisting”, “consisted”, “consisting essentially of”, “includes”, “included” and the like are defined according to standard United States and international patent law practice

The term “about” is used herein to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When not used in conjunction closed wording in the claims or specifically noted otherwise, the words “a” and “an” denote “one or more.” The term “conferred by a transgene,” for example, thus encompasses one or more transgene(s).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, ex. hydroxyproline, γ-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, ex. homoserine, norleucine, methionine sulfoxide, methionine methyl sulfonium. Such analogs have modified R groups (ex. norleucine) or modified peptide backbones but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. Macromolecular structures such as polypeptide structures may be described in terms of various levels of organization. “Primary structure” refers to the amino acid sequence of a particular peptide. “Secondary structure” refers to locally ordered, three dimensional structures within a polypeptide. These structures are commonly known as domains, for example enzymatic domains, extracellular domains, transmembrane domains, pore domains, or cytoplasmic tail domains. Domains are portions of a polypeptide that form a compact unit of the polypeptide. Exemplary domains include domains with enzymatic activity. A domain may be made up of sections of lesser organization such as stretches of β-sheet and α-helices. “Tertiary structure” refers to the complete three-dimensional structure of a polypeptide monomer. “Quaternary structure” refers to the three-dimensional structure formed by the noncovalent association of independent tertiary units. Anisotropic terms are also known as energy terms.

As used herein, an “antibody” refers to a polypeptide comprising a framework region from an immunoglobulin gene or fragments thereof that specifically binds and recognizes an antigen. The recognized immunoglobulin genes may include the kappa, lambda, alpha, gamma, delta, epsilon, and mu constant region genes, as well as the myriad immunoglobulin variable region genes. Light chains may be classified as either kappa or lambda. Heavy chains may be classified as gamma, mu, alpha, delta, or epsilon, which in turn define the immunoglobulin classes, IgY, IgG, IgM, IgA, IgD, and IgE, respectively.

An exemplary immunoglobulin (antibody) structural unit may comprise a tetramer, with each tetramer composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain and variable heavy chain refer to these light and heavy chains. Antibodies exist, ex. as intact immunoglobulins or as several well-characterized fragments produced by digestion with various peptidases. While various antibody fragments are defined in terms of the digestion of an intact antibody, one of skill will appreciate that such fragments may be synthesized de novo either chemically or by using recombinant DNA methodology. Thus, the term “antibody,” as used herein, also includes antibody fragments either produced by the modification of whole antibodies, or those synthesized de novo using recombinant DNA methodologies or those identified using other methods known in the art.

For preparation of antibodies, ex. recombinant, monoclonal, or polyclonal antibodies, many techniques known in the art may be used. The genes encoding the heavy and light chains of an antibody of interest can be cloned from a cell and used to produce a recombinant monoclonal antibody. Gene libraries encoding heavy and light chains of monoclonal antibodies may also be used. Random combinations of the heavy and light chain gene products generate a large pool of antibodies with different antigenic specificity. Techniques to produce single chain antibodies or recombinant antibodies are found in the art and may be adapted to produce antibodies to polypeptides according to the invention. Phage display technology may also be used to identify antibodies and heteromeric fragments that specifically bind to selected antigens. Antibodies may also be made bispecific, i.e., able to recognize two different antigens, or heteroconjugates, ex. two covalently joined antibodies, or immunotoxins.

As used herein, an “antigen” refers to a viral protein or polypeptide, such as a viral polypeptide, as well as viral particles. In some embodiments, an antigen in accordance with the invention may also be a viral nucleic acid. An antigen is a molecule that is recognized by the immune system and is capable of inducing an immune response in a host organism. The antigen may comprise a whole, attenuated, killed or live organism or a subunit or portion of an organism. It can also be a piece or fragment of DNA, a polypeptide, an epitope, a hapten or any combination of these that can induce immune response.

The term “avian” as used herein, includes poultry such as members of the order Galliformes. More particularly a class of birds more with economical and/or agronomical interest, such as chicken, turkeys, goose, duck, pheasant, ostrich, pigeon and quail and the like.

As used herein, a “biological sample” or “sample” may include blood and blood parts including, but not limited to serum, plasma, platelets, or red blood cells; sputum, cloacal swabs, mucosa, tissue, cultured cells, including primary cultures, explants, and transformed cells; biological fluids, stool, and urine. A biological sample may also include sections of tissues such as biopsy and autopsy samples, and frozen sections taken for histologic purposes. A biological sample may be obtained from a eukaryotic organism, such as a bird, including, but not limited to, a bird from the order Galliformes, such as chickens, quails and turkeys. Any tissue appropriate for use in accordance with the invention may be used, for instance, skin, brain, spinal cord, adrenals, pectoral muscle, lung, heart, liver, crop, proventriculus, ventriculus, duodenum, small intestine, large intestine, cloaca, kidney, bursa of fabricus, spleen, pancreas, adrenal gland, bone marrow, lumbosacral spinal cord, or blood.

The term ‘conservative amino acid substitution” indicates any amino acid substitution for a given amino acid residue, where the substitute residue is so chemically similar to that of the given residue that no substantial decrease in polypeptide function (e.g., enzymatic activity) results. Conservative amino acid substitutions are commonly known in the art and examples thereof are described, e.g., in U.S. Pat. Nos. 6,790,639, 6,774,107, 6,194,167, or 5,350,576. In a preferred embodiment, a conservative amino acid substitution will be anyone that occurs within one of the following six groups:
1. Small aliphatic, substantially non-polar residues: Ala, Gly, Pro, Ser, and Thr;
2. Large aliphatic, non-polar residues: lie, Leu, and Val; Met;
3. Polar, negatively charged residues and their amides: Asp and Glu;
4. Amides of polar, negatively charged residues: Asn and Gin; His;
5. Polar, positively charged residues: Arg and Lys; His; and
6. Large aromatic residues: Trp and Tyr; Phe.

In a preferred embodiment, a conservative amino acid substitution will be any one of the following, which are listed as Native Residue (Conservative Substitutions) pairs: Ala (Ser); Arg (Lys); Asn (Gin; His); Asp (Glu); Gin (Asn); Glu (Asp); Gly (Pro); His (Asn; Gln); lie (Leu; Val); Leu (lie; Val); Lys (Arg; Gin; Glu); Met (Leu; lie); Phe (Met; Leu; Tyr); Ser (Thr); Thr (Ser); Trp (Tyr); Tyr (Trp; Phe); and Val (lie; Leu).

The phrase “functional effects” in the context of assays for testing compounds that modulate activity of a virus as described herein includes the determination of a parameter that is indirectly or directly under the influence of such a virus, ex. a phenotypic or chemical effect. “Functional effects” may include in vitro, in vivo, and ex vivo activities and may be measured by any means known to those skilled in the art, such as changes in spectroscopic characteristics, shape, chromatographic, or solubility properties for a protein, measuring inducible markers or transcriptional activation of a protein; measuring binding activity or binding assays, e.g. binding to antibodies; measuring changes in ligand or substrate binding activity, measuring viral replication, measuring cell surface marker expression, measurement of changes in protein levels, measurement of RNA stability, identification of downstream or reporter gene expression via, for example, chemiluminescence, fluorescence, colorimetric reactions, antibody binding, and/or inducible markers.

The term “gene” refers to components that comprise viral DNA or RNA, cDNA, viral intron and exon DNA, artificial viral DNA polynucleotide, or other DNA that encodes a viral peptide, viral polypeptide, viral protein, or viral RNA transcript molecule, and the genetic elements that may flank the coding sequence that are involved in the regulation of expression, such as, promoter regions, 5′ leader regions, 3′ untranslated region that may exist as native genes or transgenes in a viral genome. The gene or a fragment thereof can be subjected to polynucleotide sequencing methods that determines the order of the nucleotides that comprise the gene.

The term “Herpesvirus of Turkey (HVT)” is defined as a nonpathogenic virus of domestic turkeys and it is classified as the third serotype within the Marek's disease virus group of antigenically and genetically related lymphotropic avian herpes viruses

The term “heterologous” when used with reference to portions of a nucleic acid indicates that the nucleic acid comprises two or more sequences that are not found in the same relationship to each other in nature. For instance, the nucleic acid is typically recombinantly produced, having two or more sequences from unrelated genes arranged to make a new functional nucleic acid, ex. a promoter from one source and a coding region from another source. Similarly, a heterologous protein indicates that the protein comprises two or more subsequences that are not found in the same relationship to each other in nature (ex. a fusion protein). Heterologous may also refer to a viral sequence, such as a gene or transgene, or a portion thereof, being inserted into a viral genome in which it is not typically found, or a gene introduced into an organism in which it is not typically found.

The term “host cell” means any cell of any organism that is selected, modified, transformed, grown, or used or manipulated in any way, to produce a substance by the cell, for example the expression by the cell of a gene, a DNA or RNA sequence, a protein or an enzyme. A host cell is intended to include any individual cell or cell culture which can be or has been a recipient for vectors or for the incorporation of exogenous nucleic acid molecules, polynucleotides, and/or proteins. It also is intended to include progeny of a single cell. The progeny may not necessarily be completely identical (in morphology or in genomic or total DNA complement) to the original parent cell due to natural, accidental, or deliberate mutation. The cells may be prokaryotic or eukaryotic.

As used herein, the term “host,” “subject,” “patient,” or “organism” may include animals, particularly birds, especially poultry. For veterinary applications, birds may be from the order Galliformes, which includes chickens, quails and turkeys, and the like. The term “living host” refers to a host as noted above or another organism that is alive. The term may also refer to the entire host or organism and not just a part excised (ex. a brain or other organ) from the living host. These terms also include an individual in all stages of development, including embryonic and fetal stages.

The terms “identical” or “percent identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, ex. the NCBI web site found at ncbi.nlm.nih.gov/BLAST/or the like). Such sequences are then referred to as “substantially identical.” This definition also refers to, or applies to, the compliment of a particular sequence. The definition may also include sequences that have deletions, additions, and/or substitutions.

For sequence comparison, one sequence typically serves as a reference sequence, to which other sequences are compared. When using a sequence comparison algorithm, reference and comparison sequences may be entered into a computer, and sequence algorithm program parameters are selected as desired. Percent sequence identities are then generated for the comparison sequences relative to the reference sequence, based on the parameters selected. An example of an algorithm that may be suitable for determining percent sequence identity and sequence similarity are the BLAST and BLAST 2.0 algorithms, which are described in Altschul et al., (Nuc Acids Res 25:3389-3402, 1977) and Altschul et al., (J Mol Biol 215:403-410, 1990), respectively. BLAST and BLAST 2.0 are well known in the art and may be used to determine percent sequence identity for any nucleic acids or proteins, such as those described herein.

As used herein, an “immunogenic composition” or “pharmaceutical composition” or “vaccine” is meant to encompass a composition comprising an antigen suitable for administration to a subject, such as an avian subject. Said composition is generally meant to elicit an immune response in a subject. The immune response can include a T cell response, a B cell response, or both a T cell and B cell response. The composition may serve to sensitize the subject patient by the presentation of antigen in association with MHC molecules at the cell surface. In addition, antigen-specific T-lymphocytes or antibodies can be generated to allow for the future protection of an immunized host. An “immunogenic composition” may contain a live, attenuated, or killed/inactivated vaccine comprising a whole microorganism or an immunogenic portion derived therefrom that induces either a cell-mediated (T cell) immune response or an antibody-mediated (B cell) immune response, or both, and may protect the animal from one or more symptoms associated with infection by the microorganism, or may protect the animal from death due to the infection with the microorganism. In general, an “immunogenic composition” is sterile, and preferably free of contaminants that can elicit an undesirable response within the subject (ex. the compound(s) in the immunogenic composition is pharmaceutical grade). Immunogenic compositions may be designed for administration to subjects in need thereof via a number of different routes of administration including in ovo, oral, intravenous, buccal, rectal, parenteral, intraperitoneal, intradermal, intracheal, intramuscular, subcutaneous, inhalational, and the like.

The term “immunogenic” protein or peptide as used herein includes polypeptides that are immunologically active in the sense that once administered to the host, it is able to evoke an immune response of the humoral and/or cellular type directed against the protein. Preferably the protein fragment is such that it has substantially the same immunological activity as the full-length protein. Thus, a protein fragment according to the invention comprises or consists essentially of or consists of at least one epitope or antigenic determinant. An “immunogenic” protein or polypeptide, as used herein, includes the full-length sequence of the protein, analogs thereof, or immunogenic fragments thereof. By “immunogenic fragment” is meant a fragment of a protein which includes one or more epitopes and thus elicits the immunological response described above.

The term “immunogenic protein or peptide” further contemplates deletions, additions and substitutions to the sequence, as long as the polypeptide functions to produce an immunological response as defined herein. The term “conservative variation” denotes the replacement of an amino acid residue by another biologically similar residue, or the replacement of a nucleotide in a nucleic acid sequence such that the encoded amino acid residue does not change or is another biologically similar residue. In this regard, particularly preferred substitutions will generally be conservative in nature, i.e., those substitutions that take place within a family of amino acids. For example, amino acids are generally divided into four families: (1) acidic—aspartate and glutamate: (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine, threonine, tyrosine. Phenylalanine, tryptophan, and tyrosine are sometimes classified as aromatic amino acids. Examples of conservative variations include the substitution of one hydrophobic residue such as isoleucine, valine, leucine or methionine for another hydrophobic residue, or the substitution of one polar residue for another polar residue, such as the substitution of arginine for lysine, glutamic acid for aspartic acid, or glutamine for asparagine, and the like; or a similar conservative replacement of an amino acid with a structurally related amino acid that will not have a major effect on the biological activity. Proteins having substantially the same amino acid sequence as the reference molecule but possessing minor amino acid substitutions that do not substantially affect the immunogenicity of the protein are, therefore, within the definition of the reference polypeptide. All of the polypeptides produced by these modifications are included herein. The term “conservative variation” also includes the use of a substituted amino acid in place of an unsubstituted parent amino acid provided that antibodies raised to the substituted polypeptide also immunoreact with the unsubstituted polypeptide.

An “immunologically effective amount” as used herein refers to the amount of antigen or vaccine sufficient to elicit an immune response, either a cellular (T cell) or humoral (B cell or antibody) response, as measured by standard assays known to one skilled in the art. For example, with respect to the present invention, an “immunologically effective amount” is a minimal protection dose (titer). The effectiveness of an antigen as an immunogen, can be measured either by proliferation assays, by cytolytic assays, such as chromium release assays to measure the ability of a T cell to lyse its specific target cell, or by measuring the levels of B cell activity by measuring the levels of circulating antibodies specific for the antigen in serum or other assays which are known and used by those of skill in the art. Furthermore, the level of protection of the immune response may be measured by challenging the immunized host with the antigen that has been injected. For example, if the antigen to which an immune response is desired is a virus or a tumor cell, the level of protection induced by the “immunologically effective amount” of the antigen is measured by detecting the percent survival or the percent mortality after virus or tumor cell challenge of the animals.

Determination of what is an immunologically effective amount of the vaccine according to the invention is well within reach of the skilled person, for instance by monitoring the immunological response following vaccination, or after a challenge infection, e.g. by re-isolation of the pathogen, or by monitoring the targets' clinical signs of disease, or serological parameters, and comparing these to responses seen in mock-vaccinated animals. The dosing scheme for applying the vaccine according to the invention to a target organism can be in single or multiple doses, which may be given at the same time or sequentially, in a manner compatible with the formulation of the vaccine, and in such an amount as will be immunologically effective.

The terms “inhibitors,” activators,” and “modulators” of viral nucleic acid and polypeptide sequences are used to refer to activating, inhibitory, or modulating molecules identified using in vitro and in vivo assays of the viral nucleic acid and polypeptide sequences. Inhibitors are compounds that may bind to, partially or totally block activity, decrease, prevent, delay activation, inactivate, desensitize, or down regulate the activity or expression of a virus. Activators refer to compounds that increase, open, activate, facilitate, enhance activation, sensitize, agonize, or up regulate viral activity. Inhibitors, activators, or modulators also include genetically modified versions of a virus as described herein, ex. versions with altered activity, as well as naturally occurring and synthetic ligands, substrates, antagonists, agonists, antibodies, peptides, cyclic peptides, nucleic acids, antisense molecules, ribozymes, small chemical molecules and the like. Assays for inhibitors and activators include, ex. expressing a virus is the invention in vitro, in cells, or cell membranes, applying putative modulator compounds, and then determining the functional effects on activity, as described herein.

Test samples or assays comprising a virus of the invention that are treated with a potential activator, inhibitor, or modulator may be compared to a control sample lacking the inhibitor, activator, or modulator in order to determine the extent of inhibition. Control samples to which a test sample or assay is compared may be assigned a relative protein activity value of 100%. Inhibition of virus is achieved when the activity value of the test sample relative to the control sample is less than about 80%, including about 75%, about 70%, about 65%, about 60%, about 55%, about 50%, about 45%, about 40%, about 35%, about 30%, about 25%, about 20%, about 15%, about 10%, about 5%, and about 0%.

Intergenic loci, as described herein, is defined as a region of DNA sequence located between genes, including untranslated regions, 5′ and 3′ flanking regions, introns, etc. Intergenic regions are part of the noncoding DNA that may contain gene control elements such as promoters and enhancers.

The term “isolated” means a substance that has been substantially separated from, or enriched relative to, other substances with which it occurs in nature. Isolated substances are usually at least about 80%, at least 90% pure, at least 98% pure, or at least about 99% pure, by weight.

A “label” or a “detectable moiety” is a composition detectable by spectroscopic, photochemical, biochemical, immunochemical, chemical, or other physical means. For example, useful labels include ³²P, fluorescent dyes, electron-dense reagents, enzymes (ex. as commonly used in an ELISA), biotin, digoxigenin, or haptens and proteins which can be made detectable, ex. by incorporating a radiolabel into the peptide or used to detect antibodies specifically reactive with the peptide.

As used herein “Marek's Disease Virus” or “MDV” refers to any alphaherpesvirus of the genus Mardivirus, which includes the Herpesvirus of Turkeys (HVT), as described herein. In a specific embodiment, the invention relates to the Marek's disease virus, its genetic components, genes, and proteins produced thereby. As used herein, such a virus may include the genetic components of the virus, i.e., the genome and transcripts thereof, proteins encoded by the genome (including structural and nonstructural proteins), and functional or nonfunctional viral particles. The polynucleotide and polypeptide sequences encoding such viruses are well known in the art and would be easily found by one of skill in the art.

The terms “mutant” and “mutation” mean any detectable change in genetic material, ex. DNA, or any process, mechanism, or result of such a change. This includes gene mutations, in which the structure (ex. DNA sequence) of a gene is altered, any gene or DNA arising from any mutation process, and any expression product (ex. protein or enzyme) expressed by a modified gene or DNA sequence. The term “variant” may also be used to indicate a modified or altered gene, DNA sequence, enzyme, cell, etc., i.e., any kind of mutant.

As used herein, the term “nucleic acid” refers to a single or double-stranded polymer of deoxyribonucleotide bases or ribonucleotide bases read from the 5′ to the 3′ end. A “nucleic acid” may also optionally contain non-naturally occurring or altered nucleotide bases that permit correct read through by a polymerase and do not reduce expression of a polypeptide encoded by that nucleic acid. The term “nucleotide sequence” or “nucleic acid sequence” refers to both the sense and antisense strands of a nucleic acid as either individual single strands or in the duplex. The term “ribonucleic acid” (RNA) is inclusive of RNAi (inhibitory RNA), dsRNA (double stranded RNA), siRNA (small interfering RNA), mRNA (messenger RNA), miRNA (micro-RNA), tRNA (transfer RNA, whether charged or discharged with a corresponding acylated amino acid), and cRNA (complementary RNA). The terms “nucleic acid segment,” “nucleotide sequence segment,” or more generally, “segment,” will be understood by those in the art as a functional term that includes genomic sequences, ribosomal RNA sequences, transfer RNA sequences, messenger RNA sequences, operon sequences, and smaller engineered nucleotide sequences that express or may be adapted to express, proteins, polypeptides or peptides. The nomenclature used herein is that required by Title 37 of the United States Code of Federal Regulations § 1.822 and set forth in the tables in WIPO Standard ST.25 (1998), Appendix 2, Tables 1 and 3.

The term “operably linked” is used herein to refer to an arrangement of flanking sequences wherein the flanking sequences so described are configured or assembled to perform their usual function. Thus, a flanking sequence operably linked to a coding sequence may be capable of effecting the replication, transcription and/or translation of the coding sequence. For example, a promoter or enhancer is operably linked to a coding sequence if it affects the transcription of the sequence. A flanking sequence need not be contiguous with the coding sequence, so long as it functions correctly. Thus, for example, intervening untranslated yet transcribed sequences can be present between a promoter sequence and the coding sequence and the promoter sequence can still be considered “operably linked” to the coding sequence.

The term “pharmaceutically acceptable carrier” refers to an ingredient in a pharmaceutical formulation, other than an active ingredient, that is physiologically compatible to administer to a subject. Pharmaceutically acceptable carrier includes, but is not limited to a buffer, excipient, stabilizer, adjuvant, preservative, diluent, aqueous or non-aqueous vehicle and other additives. Additionally, this term refers to an element of an immunogenic composition or vaccine that is generally approved by a regulatory agency of a Federal, a state government, or other regulatory agency, or listed in the U.S. Pharmacopeia or other generally recognized pharmacopeia for use in both human and non-human animals. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. The formulation should suit the mode of administration.

As used herein, “poultry” refers to a domestic or commercial bird kept for the eggs they produce, as well as their meat and feathers. In some embodiments, poultry may include a bird from the order Galliformes, which includes chickens, quails, and turkeys, and may also include geese, ducks, swan, guinea, pigeons, and the like.

Polynucleotides as described herein may be complementary to all or a portion of a viral gene sequence, including a promoter, intron, coding sequence, exon, 5′ untranslated region, and 3′ untranslated region.

A particular nucleic acid sequence may also encompass “splice variants.” Similarly, a particular protein encoded by a nucleic acid implicitly encompasses any protein encoded by a splice variant of that nucleic acid. Splice variants are products of alternative splicing of a gene. After transcription, an initial nucleic acid transcript may be spliced such that different (alternate) nucleic acid splice products encode different polypeptides. Mechanisms to produce splice variants vary but include alternate splicing of exons. Alternate polypeptides derived from the same nucleic acid by read-through transcription are also encompassed by this definition. Any products of a splicing reaction, including recombinant forms of the splice products, are included in this definition.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The terms “polyvalent vaccine”, “combination or combo vaccine” and “multivalent vaccine” are used interchangeably to refer to a vaccine containing more than one antigen. The polyvalent vaccine may contain two, three, four or more antigens. The polyvalent vaccine may comprise recombinant viral vectors, active or attenuated or killed wild-type viruses, or a mixture of recombinant viral vectors and wild-type viruses in active or attenuated or killed forms.

“Promoters”, as used herein, refer to DNA sequences that define where transcription of a gene by RNA polymerase begins. Promoters are typically located upstream of the transcription initiation site. A promoter can also comprise a distal enhancer or repressor elements, which can be located as much as several thousand nucleotides from transcription start site. Promoters define the direction of transcription and indicate which DNA strand will be transcribed. A promoter can be derived from sources including viral, bacterial, fungal, plants, insects, and animals. A promoter can regulate the expression of a gene component constitutively or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, RSV-LTR promoter, CMV IE promoter, human CMV promoter; murine CMV promoter; Pec promoter; β-chicken actin promoter; a guinea pig CMV promoter, a Pseudorabies Virus promoter; a glycoprotein X promoter, a Herpes Simplex Virus-1 promoter; a Marek's Disease Virus promoter; and an SV40 promoter.

As used herein, the terms “prophylactically treat” or “prophylactically treating” refers to completely or partially preventing a disease or symptom thereof and/or may be therapeutic in terms of a partial or complete cure for a disease and/or adverse effect attributable to the disease.

The term “recombinant” when used with reference, ex. to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. In some embodiments, recombinant sequences may also include nucleic acids, proteins, or recombinant genomes, such as viral genomes. Recombinant viral vectors as described herein may contain transgenes that are operatively linked to a heterologous promoter in order to effect transcription of the transgene.

The phrase “stringent hybridization conditions” refers to conditions under which a probe will hybridize to its target subsequence, typically in a complex mixture of nucleic acids, but to no other sequences. Stringent conditions may be sequence-dependent and will be different in different circumstances. Longer sequences hybridize specifically at higher temperatures. Stringent conditions may be achieved with the addition of destabilizing agents such as formamide.

Appropriate stringency conditions that promote DNA hybridization are well known to one of skill in the art and may include, for example, 6× sodium chloride/sodium citrate (SSC) at about 45° C., followed by a wash of 2× SSC at 50° C. The salt concentration in the wash step may be selected from a low stringency of approximately 2× SSC at 50° C. to a high stringency of about 0.2× SSC at 50° C. The temperature in the wash step may be increased from low stringency conditions at room temperature, about 22° C., to high stringency conditions at about 65° C. The temperature and/or salt conditions may be varied as appropriate for optimum results. In accordance with the invention, a nucleic acid may exhibit at least from about 80% to about 100% sequence identity with one or more nucleic acid molecules as described herein, for example at least from about 80%, about 85%, about 90%, about 95%, about 98%, about 99%, or about 100% sequence identity.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions.

The term “therapeutically effective amount,” “effective amount,” or “therapeutically effective dose” as used herein refers to a dose that produces an effect for which it is administered. Such a dose or amount may also refer to the amount of an embodiment of the agent being administered that will relieve to some extent one or more of the symptoms of the disease, i.e., infection, being treated, and/or that amount that will prevent, to some extent, one or more of the symptoms of the disease, i.e., infection, that the host being treated has or is at risk of developing. The exact dose will depend on the purpose of the treatment, and one of skill in the art will be able to determine such a dose using techniques known in the art.

As used herein, a “transgene” refers to a segment of DNA containing a heterologous coding sequence or other genetic material for introduction from one organism into another. For instance, in certain embodiments, a transgene according to the present invention may comprise an antigenic coding sequence, such as a viral gene, or a sequence encoding a viral protein.

As used herein, the terms “treatment,” “treating,” and “treat” are defined as acting upon a disease, disorder, or condition with an agent to reduce or ameliorate the pharmacologic and/or physiologic effects of the disease, disorder, or condition and/or its symptoms. “Treatment,” as used herein, covers any treatment of a disease in a subject or host (ex. an animal of veterinary interest), and includes: (a) reducing the risk of occurrence of the disease in a subject determined to be predisposed to the disease but not yet diagnosed as infected with the disease, (b) impeding the development of the disease, and (c) relieving the disease, i.e., causing regression of the disease and/or relieving one or more disease symptoms. “Treatment” is also meant to encompass delivery of an inhibiting agent to provide a pharmacologic effect, even in the absence of a disease or condition. For example, “treatment” encompasses delivery of a disease or pathogen inhibiting agent that provides for enhanced or desirable effects in the subject (ex. reduction of pathogen load, reduction of disease symptoms, etc.).

The term “unit dosage form,” as used herein, refers to physically discrete units suitable as unitary dosages for animal subjects, each unit containing a predetermined quantity of a compound (ex. an antiviral compound, as described herein) calculated in an amount sufficient to produce the desired effect in association with a pharmaceutically acceptable diluent, carrier, or vehicle. The specifications for unit dosage forms depend on the particular compound employed, the route and frequency of administration, the effect to be achieved, and the pharmacodynamics associated with each compound in the host.

The terms “vaccine” or “vaccine composition”, which are herein used interchangeably, refer to pharmaceutical compositions comprising at least one immunogenic composition of the invention that induces an immune response in subject. A vaccine or vaccine composition may protect the subject from disease or possible death and may or may not include one or more additional components that enhance the immunological activity of the active component. The composition of the invention that induces a protective immune response comprises a recombinant HVT virus having one or more heterologous antigen encoding genes inserted into the HVT genome at intergenic region UL 35/36. In some embodiments the composition of the invention comprises a recombinant HVT virus having one or more heterologous antigen encoding genes inserted into the HVT genome at UL 35/36 and one or more antigen encoding genes inserted into the HVT genome at UL55. In some embodiments the antigen encoding genes are antigens derived from poultry pathogens such as Newcastle Disease Virus, Infectious Bursal Disease Virus, Infectious Bronchitis Virus, Avian Influenza Virus, Infectious Laryngotracheitis Virus and/or Chicken Anemia Virus. In some embodiments the recombinant HVT is combined with another recombinant Marek's Disease Virus vaccine that causes a protective immune response in poultry. The vaccine or vaccine composition of the invention may additionally comprise further components typical to vaccines or vaccine compositions, including, for example, an adjuvant or an immunomodulator. A vaccine may comprise one or simultaneously more than one of the elements described above.

The vaccine of the invention may further comprise a suitable pharmaceutical carrier. The term “pharmaceutically acceptable carrier” is intended to include any and all solvents, dispersion media, coatings, antibacterial and antifungal agents, isotonic and absorption delaying agents, to hosts. The term “carrier” refers to a diluent, adjuvant, excipient, or vehicle with which the pharmaceutical composition is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained release formulations and the like. The composition can be formulated with traditional binders and carriers such as triglycerides depending on the method of administration. Particular formulations can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. The formulation should suit the mode of administration. The appropriate carrier is evident to those skilled in the art and will depend in large part upon the route of administration. Additional components that may be present in this invention are adjuvants, preservatives, surface active agents, chemical stabilizers, suspending or dispersing agents. Typically, stabilizers, adjuvants and preservatives are optimized to determine the best formulation for efficacy in the target subject

A “variant” peptide refers herein to a peptide which differs in amino acid sequence from a “parent” vaccine peptide amino acid sequence by virtue of addition, deletion, and/or substitution of one or more amino acid residue(s) in the parent peptide sequence and retains at least one desired activity of the parent vaccine peptide. For example, the variant may comprise at least one, ex. from about one to about ten, and preferably from about two to about five, substitutions in one or more amino acid sequences of the peptide to be used as part of the vaccine of the present invention. Ordinarily, the variant will have an amino acid sequence having at least 50% amino acid sequence identity with the parent amino acid sequences, preferably at least 65%, more preferably at least 70%, more preferably at least 75%, more preferably at least 80%, more preferably at least 85%, more preferably at least 90%, and most preferably at least 95% sequence identity. Identity or homology with respect to this sequence is defined herein as the percentage of amino acid residues in the candidate sequence that are identical with the parent peptide residues, after aligning the sequences and introducing gaps, if necessary, to achieve the maximum percent sequence identity. None of N-terminal, C-terminal, or internal extensions, deletions, or insertions into the peptide sequence shall be construed as affecting sequence identity or homology. The variant retains the ability to elicit an immune response and preferably has desired activities which are superior to those of the parent peptide.

Variant peptides may be fully functional or may lack function in one or more activities. Fully functional variants typically contain only conservative variations or variations in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region.

Moreover, polypeptides often contain amino acids other than the twenty “naturally occurring” amino acids. Further, many amino acids, including the terminal amino acids, may be modified by natural processes, such as processing and other post-translational modifications, or by chemical modification techniques well known in the art. Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Such modifications are well known to those of skill in the art and have been described in great detail in the scientific literature. Several particularly common modifications, glycosylation, lipid attachment, sulfation, gamma-carboxylation of glutamic acid residues, hydroxylation and ADP ribosylation, for instance, are described in most basic texts, such as Proteins-Structure and Molecular Properties (2nd ed., T. E. Creighton, W.H. Freeman & Co., NY, 1993). Many detailed reviews are available on this subject, such as by Wold, Posttranslational Covalent Modification of proteins, 1-12 (Johnson, ed., Academic Press, NY, 1983); Seifter et al. 182 Meth. Enzymol. 626-46 (1990); and Rattan et al. 663 Ann. NY Acad. Sci. 48-62 (1992).

Accordingly, the peptides of the present invention also encompass derivatives or analogs in which a substituted amino acid residue is not one encoded by the genetic code. Similarly, the additions and substitutions in the amino acid sequence as well as variations, and modifications just described may be equally applicable to the amino acid sequence of antigen and/or epitope or peptides thereof and are thus encompassed by the present invention.

A “variant” nucleic acid refers herein to a molecule which differs in sequence from a “parent” nucleic acid. Polynucleotide sequence divergence may result from mutational changes such as deletions, substitutions, or additions of one or more nucleotides. Each of these changes may occur alone or in combination, one or more times in a given sequence.

Just as a polypeptide may contain conservative amino acid substitution(s), a polynucleotide thereof may contain conservative codon substitution(s). A codon substitution is considered conservative if, when expressed, it produces a conservative amino acid substitution, as described above. Degenerate codon substitution, which results in no amino acid substitution, is also useful in polynucleotides according to the present invention. Thus, for example, a polynucleotide encoding a selected polypeptide useful in an embodiment of the present invention may be mutated by degenerate codon substitution in order to approximate the codon usage frequency exhibited by an expression host cell to be transformed therewith, or to otherwise improve the expression thereof.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells. Vectors, as described herein, have expression control sequences meaning that a nucleic acid sequence that directs transcription of a nucleic acid. An expression control sequence can be a promoter, such as a constitutive or an inducible promoter, or an enhancer. The expression control sequence is ‘operably linked’ to the nucleic acid sequence to be transcribed. A nucleic acid is “operably linked” when it is placed into a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, “viral proteins” or “viral polypeptides” refers to a protein encoded by a virus described herein, including structural and non-structural proteins. Such proteins may include naturally occurring or non-naturally occurring viral proteins from MDV, NDV, and/or IBDV, including VP2, F, and/or HN, NP, P, M, or L proteins. Such proteins may also include naturally occurring or non-naturally occurring viral proteins from ILTV such as gB, gC, gD, gE, gH, gI or gL, the S1, S2 or M proteins from Infectious Bronchitis Virus (IBV) the VP1 or VP2 proteins of the Chicken Anemia Virus (CAV) and/or any of the HA, NA, NP or M proteins of the Avian Influenza Virus (AIV)

In accordance with the invention, recombinant viral vectors as described herein may enable protection of poultry against two or more different viral pathogens by providing recombinant viral vectors that express genes from such viral pathogens. In some embodiments, the recombinant viral vectors of the present invention may be provided to poultry in an immunogenic composition as described herein. Genes from any viral pathogen suitable for use with a recombinant viral vector as described herein may be used. For example, in some embodiments, the recombinant viral vector may express genes from Newcastle disease Virus (NDV), infectious bursal disease virus (IBDV), avian influenza virus (AIV), Chicken Anemia Virus (CAV), Infectious Bronchitis Virus (IBV), and Infectious Laryngotracheitis Virus (ILTV) or the like.

In accordance with the invention, a transgene conferring protection from or resistance to a particular virus or viruses may be inserted into the viral genome at a specific location. For example, in some embodiments, a transgene as described herein may be inserted into the viral genome in an intergenic region flanked by HVT UL 35/UL 36 in the unique long region of the genome. In another embodiment of the present invention the transgene is described herein as comprising one or more heterologous genes inserted into the viral genome in an intergenic region flanked by HVT UL 35/UL 36 of the HVT genome in addition to a second site used wherein one or more heterologous genes are inserted into the UL55 site within the HVT genome. In other embodiments, more than one transgene may be inserted into one or both of these regions.

In some embodiments, the recombinant viral vector may express multiple genes from a single virus species or may express genes from more than one virus species in order to obtain resistance to multiple viruses. For instance, in one embodiment, the invention provides a recombinant viral vector comprising the HVT genome and at least one transgene from a different viral pathogen, thus providing protection in a bird such as poultry against Marek's disease, and at least one other viral disease. For example, in one embodiment, a recombinant viral vector in accordance with the invention may provide protection in poultry against MDV and NDV, or may provide protection against MDV and IBDV, or may provide protection against MDV, NDV, and IBDV.

Viral antigens for expression in poultry by a recombinant viral vector of the present invention may be encoded by a viral gene, such as a viral gene as described herein. One of skill in the art will appreciate in this regard that it may not be required to incorporate the entirety of a particular viral gene in order to obtain a desired viral resistance. Rather, a portion of such a gene may be used. It may be desirable to choose a particular portion of a desired gene that is specific to any given targeted virus or viruses. Optimization of a desired viral protein or sequence encoding such a protein regardless of the length of the protein may be readily carried out using the methodologies known in the art that are appropriate for use with the present invention. One of skill in the art will appreciate that modifications may be made to a viral gene or genes, or the proteins encoded thereby, to increase the activity of the viral protein when introduced into the subject. Modifications made to viral genes or proteins may increase or decrease the response in a host to a specific virus.

In certain embodiments, a recombinant Marek's disease virus or recombinant viral vector of the invention may have a transgene encoding an IBDV viral protein or gene product, such as an IBDV VP2 protein or gene product. In another embodiment, such a recombinant virus or viral vector may have a transgene encoding an NDV viral protein or gene product, such as an NDV F or HN protein or gene product. In another embodiment, such a recombinant virus or viral vector may have a transgene encoding an Avian Influenza Virus (AIV) viral protein or gene product, such as an AIV HA or N protein or gene product. In another embodiment, such a recombinant virus or viral vector may have a transgene encoding an Infectious Bronchitis Virus (IBV) viral protein or gene product, such as IBV S1 or S2 protein or gene product. A transgene of the invention may have more than one gene, including a gene-fusion protein or gene product, such as an NDV F-HN fusion protein, chimera, or gene product. In some embodiments, the complete coding sequence of such a gene may be used such that a full-length or fully functional protein or polypeptide is produced. Alternatively, a portion or fragment of a viral protein or polypeptide may be sufficient to provide protection from or resistance to a particular virus or viruses.

In certain embodiments, a recombinant Marek's disease virus or recombinant viral vector of the invention may have a transgene encoding an immunomodulator such as a cytokine protein or gene product. In accordance with the invention, a cytokine may be an interleukin (IL) including, but not limited to, IL2, IL6, IL7, IL8, IL12, IL 18, or the like. Such a transgene encoding a cytokine may be inserted into one or both genomic sites as described herein. In some embodiments, a transgene encoding may be inserted into one site described herein and a transgene encoding a viral protein inserted into the other site. Other immunomodulators may be useful, such as interferons, chemokines, glucans, granulocyte colony stimulating factors, oligodeoxynucleotides may also be used in accordance with the invention.

### Isolation of Viral Genes or Proteins

In embodiments of the invention, a viral gene as described herein may be isolated using nucleic acid probes and/or oligonucleotides under stringent hybridization conditions, PCR or microarray, screening DNA libraries, or using any other methods known in the art. One of skill in the art will readily understand how to isolate viral genes or proteins for use according to the invention. Alternatively, expression libraries may be used to clone a virus, polymorphic variants thereof, orthologs, or alleles by detecting homologs immunologically with antisera or purified antibodies directed against a virus from another species or portions thereof.

Methods for making and screening cDNA libraries are well known in the art. For example, to make acDNA library to clone viral genes expressed by the genome, mRNA may be reverse-transcribed into cDNA using reverse transcriptase. The cDNA may then be ligated into a vector, such as recombinant vector, and introduced into a host cell or organism for propagation, screening, and cloning.

For a genomic library, DNA may be extracted from a desired tissue and may be digested using biological enzymes or may be mechanically sheared. The resulting DNA fragments may then be isolated from undesired DNA fragments and constructed into an appropriate vector, which may then be packaged in vitro. Recombinant vectors may be analyzed by any method known in the art.

Methods such as polymerase chain reaction (PCR and RT-PCR) and ligase chain reaction (LCR) may be used to amplify nucleic acid sequences directly from mRNA, from cDNA, from genomic libraries or cDNA libraries. Degenerate oligonucleotides can be designed to amplify homologs using the sequences provided herein. Restriction endonuclease sites may be incorporated into the primers. Polymerase chain reaction or other in vitro amplification methods may also be useful, for example, to clone nucleic acid sequences that code for proteins to be expressed, to make nucleic acids to use as probes for detecting the presence of the disease to be targeted, such as MDV, NDV, and/or IBDV, encoding mRNA in biological samples, for nucleic acid sequencing, or for other purposes. Genes amplified by PCR may be purified from agarose and cloned into an appropriate vector.

Expression of viral genes may also be analyzed by techniques known in the art, ex. reverse transcription and amplification of mRNA, isolation of total RNA or polyA RNA, northern blotting, dot blotting, in situ hybridization, RNase protection, high density polynucleotide array technology, and the like.

Nucleic acids encoding a viral genome or protein may be used with high density oligonucleotide array technology (ex. GeneChip™) to identify viral genes, orthologs, alleles, variants thereof, and polymorphic variants in this invention. The gene of choice may be cloned into an intermediate vector before transformation into prokaryotic or eukaryotic cells for replication and/or expression. These intermediate vectors may be prokaryote vectors, ex. plasmids, or shuttle vectors.

### Modification of Nucleic Acids

Any number of methods well known to those skilled in the art can be used to isolate and manipulate a DNA molecule. For example, polymerase chain reaction (PCR) technology may be used to amplify a particular starting DNA molecule and/or to produce variants of the starting DNA molecule. DNA molecules, or fragments thereof, can also be obtained by any techniques known in the art, including directly synthesizing a fragment by chemical means. Thus, all or a portion of a nucleic acid as described herein may be synthesized.

As used herein, the term “complementary nucleic acids” refers to two nucleic acid molecules that are capable of specifically hybridizing to one another, wherein the two molecules can form an anti-parallel, double-stranded nucleic acid structure. In this regard, a nucleic acid molecule is said to be the complement of another nucleic acid molecule if they exhibit complete complementarity. Two molecules are said to be “minimally complementary” if they can hybridize to one another with sufficient stability to permit them to remain annealed to one another under at least conventional low-stringency conditions. Similarly, the molecules are said to be complementary if they can hybridize to one another with sufficient stability to permit them to remain annealed to one another under conventional high-stringency conditions. Conventional stringency conditions are described by Sambrook, et al. (1989), and by Haymes et al. (1985).

Departures from complete complementarity are permissible, as long as the capacity of the molecules to form a double-stranded structure remains. Thus, in order for a nucleic acid molecule or a fragment of the nucleic acid molecule to serve as a primer or probe such a molecule or fragment need only be sufficiently complementary in sequence to be able to form a stable double-stranded structure under the particular solvent and salt concentrations employed.

As used herein, the terms “sequence identity,” “sequence similarity,” or “homology” are used to describe sequence relationships between two or more nucleotide sequences. The percentage of “sequence identity” between two sequences is determined by comparing two optimally aligned sequences over a specific number of nucleotides, wherein the portion of the sequence in the comparison window may comprise additions or deletions (i.e., gaps) as compared to a reference sequence. Two sequences are said to be identical if nucleotide at every position is the same. A nucleotide sequence when observed in the 5′ to 3′ direction is said to be a “complement” of, or complementary to, a second nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence.

### Recombinant Vectors and Host Cells

A recombinant DNA vector may be, for example, a linear or circular plasmid. The vector system may be a single vector or plasmid or two or more vectors or plasmids that together contain the total DNA to be introduced into the genome of a host cell. A recombinant vector as described herein may be an expression vector, for example to enable production of a desired protein in a host cell such as a bacterial cell. Nucleic acid molecules as described herein, or complements or fragments thereof, may be inserted into a vector under the control of a suitable promoter that functions in one or more microbial hosts to drive expression of a linked coding sequence or other DNA sequence. Many vectors are available and known in the art for this purpose, and selection of the appropriate vector depends on the size of the nucleic acid to be inserted into the vector and the host cell to be transformed with the vector. Each vector may contain various components depending on its function (ex. amplification of DNA or expression of DNA) and the particular host cell with which it is compatible. Vector components for bacterial transformation generally include, but are not limited to, one or more of the following: a signal sequence, an origin of replication, one or more selectable marker genes, and an inducible promoter allowing the expression of exogenous DNA.

As used herein, a “recombinant Marek's Disease Virus” or “recombinant HVT” or “recombinant virus” denotes an infective virus or viral particle that has been genetically modified by the incorporation into the viral genome of one or more heterologous nucleic acid sequences, i.e. DNA coding for a viral gene or fragment or portion thereof not identical to the nucleic acid sequence of a gene naturally present in the virus. On infection of a cell by the recombinant Marek's disease virus, the recombinant virus expresses the heterologous gene in the form of a heterologous polypeptide.

A “recombinant viral vector” or “viral vector” as used herein refers to a recombinant construct that is inserted into a virus for introduction into a host cell. Such a vector according to the invention may be derived from any HVT strain. As appropriate, viral genes or protein-coding sequences may be incorporated into such a recombinant viral vector as described herein for introduction into a chicken or other poultry for protection from one or more viral diseases.

As used herein, an “insertion site” refers to a region in a viral genome into which a transgene or exogenous DNA is inserted. The insertion sites of the present invention may be intergenic regions. An intergenic region in accordance with the invention may be flanked by HVT UL35 and HVT UL36 in the unique long region of the genome. In some embodiments of the present invention one or more heterologous nucleotide encoding antigens may also be inserted into the regions defined by the UL55 locus of the HVT genome. In some embodiments, the insertion sites of the present invention may include all or a portion of a flanking gene on either side of the intergenic region. Insertion of one or more transgenes into one of these regions enables the production of a recombinant viral vector that can then be introduced into a chicken or other poultry for protection against one or more diseases.

As used herein, the term “operably linked” when used in reference to a regulatory sequence and a nucleotide sequence, means that the regulatory sequence causes regulated expression of the linked structural nucleotide sequence. The terms “regulatory sequences,” “regulatory elements,” or “control elements” refer to nucleotide sequences located upstream (5′ sequences), within, or downstream (3′ sequences) of a structural nucleotide sequence. Such sequences influence the timing and level or amount of transcription, RNA processing or stability, or translation of an associated structural nucleotide sequence. Regulatory sequences may include but are not limited to promoters, leader sequences, introns, enhancers, stem-loop structures, repressor binding sequences, and polyadenylation recognition sequences, including, but not limited to, a bovine growth hormone polyA signal, a Simian virus 40 (SV40) polyA signal, an Autographa californica nuclear polyhedrosis virus (AcNPV) 1629 ORF poly (A) signal, and a herpes simplex virus (HSV) thymidine kinase (TK) polyA signal. One of skill in the art will recognize that different combinations of promoters and/or regulatory elements may be used to increase or decrease expression of a transgene as described herein.

Promoters that function in different species are also well known in the art. Promoters useful for expression of polypeptides include those that are inducible, viral, synthetic, or constitutive, and/or promoters that are tissue-specific, temporally regulated, spatially regulated, and spatial-temporally regulated. For example, a promoters useful in accordance with the invention may include, but is not limited to, an immediate early (IE) cytomegalovirus (CMV) promoter, guinea pig CMV promoter, an SV40 promoter, Pseudorabies Virus promoters such as that of glycoprotein X promoter, Herpes Simplex Virus-1, such as the alpha 4 promoter, Marek's disease viruses promoters, including any isolate or strain of MDV, such as MDV-1, MDV-2, and HVT, for example a promoter controlling expression of glycoproteins such as gC, gB, gE, or gI, Infectious Laryngotracheitis Virus promoters such as those of glycoprotein gB, gE, gI, gD genes, or any other suitable promoters. One of skill in the art would be aware of how to identify a promoter useful in accordance with the invention.

In accordance with the invention, a recombinant Marek's disease virus or recombinant viral vector as described herein may comprise one or more transgenes operatively linked to one or more promoters for expression of one or more viral proteins or peptides or fragments or portions thereof. In some embodiments, a single transgene may be operatively linked to a single promoter, or more than one transgene may be operatively linked to a single promoter. In other embodiments, more than one transgene may be present in a recombinant vector wherein a first transgene is operatively linked to a first promoter, a second transgene is operatively linked to a second promoter.

### Construction and Selection of Vectors

Construction of vectors containing one or more components as described herein useful for inserting genes or transgenes, or portions thereof, into a target site is known to one of skill in the art and may employ standard recombinant DNA techniques. A recombinant DNA vector or construct may comprise a selectable marker that confers a selectable phenotype to a cell. Selectable markers may also be used to select for cells that contain the exogenous nucleic acids encoding polypeptides or proteins as described herein. Such a marker may encode for example, biocide resistance, or antibiotic resistance (ex. kanamycin, G418, bleomycin, hygromycin, etc.). Selectable markers are well known to one of skill in the art and may include any markers suitable for use in accordance with the invention.

A recombinant vector or construct may also include a screenable marker, which may be used to monitor expression but which may not result in death of a cell. Suitable screenable markers may include for example, a β-glucuronidase or uidA gene (GUS), one or more of the various fluorescent protein genes, such as green fluorescent protein (GFP), red fluorescent protein (RFP), or any one of a large family of proteins which fluoresce at characteristic wavelengths, a gene that encodes an enzyme for which various chromogenic substrates are known, a luciferase gene, a xylE gene, which encodes a catechol dioxygenase that converts chromogenic catechols, an β-amylase gene, a tyrosinase gene, which encodes an enzyme capable of oxidizing tyrosine to DOPA and dopaquinone, which in turn condense to melanin, or an α-galactosidase, which catalyzes a chromogenic α-galactose substrate.

### Expression of Proteins in Host Cells

To obtain high level expression of a cloned viral gene as described herein, a nucleic acid may be subcloned into an expression vector that contains a strong promoter to direct transcription, and a transcription/translation terminator. For encoded proteins, a ribosome binding site for translation initiation may also be included. Suitable promoters for use in expression vectors are well known in the art, such as a bacterial promoter, a viral promoter, or the like. Expression systems for expressing a protein are available in several prokaryotic and eukaryotic species known in the art. Commercial kits for such expression systems are also readily available. Eukaryotic expression systems for mammalian cells, yeast, and insect cells are well known in the art and are also commercially available.

Selection of an appropriate promoter to direct expression of a heterologous nucleic acid will depend on the particular application. Such a promoter may be positioned a distance from the heterologous transcription start site that is similar to the distance in its natural setting, although one of skill in the art will understand that some variation in this distance may be permitted without loss of promoter function.

In addition to a promoter, an expression vector typically contains a transcriptional or expression cassette that contains all elements required for expression of a nucleic acid in a host cell. Any conventional vectors known in the art that may be used for expression in eukaryotic or prokaryotic cells may be used to transport genetic information into a cell. A typical expression cassette thus contains a promoter operably linked to a nucleic acid sequence encoding the nucleic acid of choice and corresponding signals required for efficient processing, ex. ribosome binding sites, polyadenylation, and translation termination. Additional elements may include enhancers and, for the case of genomic DNA as the structural gene, introns with functional splice donor and acceptor sites.

In addition to a promoter sequence, such as a promoter set forth herein, an expression cassette may also contain a transcription termination region downstream of the structural gene in order to provide for efficient termination of transcription. The termination region may be from the same gene as the promoter sequence, or it may be from a different gene. Markers such as fluorescent proteins, green or red fluorescent protein, β-gal, CAT, and the like can be included in the vectors as markers for vector transduction. Epitope tags or sequence tags may also be added to recombinant proteins to provide convenient methods of isolation.

Expression vectors containing regulatory elements from eukaryotic viruses are typically used in eukaryotic expression vectors, ex. SV40 vectors, papilloma virus vectors, retroviral vectors, and vectors derived from Epstein-Barr virus. Other exemplary eukaryotic vectors include pMSG, pAV009/A+, pMTO10/A+, pMAMneo-5, baculovirus pDSVE, and any other vector allowing expression of proteins under the direction of the CMV promoter, S V40 early promoter, SV40 late promoter, metallothionein promoter, murine mammary tumor virus promoter, Rous sarcoma virus promoter, polyhedrin promoter, or other promoters known in the art that may be effective for expression in eukaryotic cells.

Expression of proteins from eukaryotic vectors can be also be regulated using inducible promoters. With inducible promoters, expression levels are tied to the concentration of inducing agents, such as tetracycline or ecdysone, by the incorporation of response elements for these agents into the promoter. High levels of expression may be obtained from inducible promoters in the presence of an inducing agent. Some expression systems have markers such as thymidine kinase and dihydrofolate reductase, which provide gene amplification.

An expression vector may also include a replicon that functions in E. coli, an antibiotic resistance gene for selection of bacteria harboring recombinant plasmids, and unique restriction sites in nonessential regions of the plasmid to allow insertion of eukaryotic sequences. Any antibiotic resistance gene suitable for use with the present invention may be employed.

Standard transfection methods known in the art may be used to produce bacterial, mammalian, yeast, or insect cell lines that express large quantities of protein. Such cell lines may then be purified using standard techniques known in the art, and prokaryotic and/or eukaryotic cells may be transformed according to any method known in the art for introducing cloned genomic DNA, cDNA, synthetic DNA or other foreign genetic material into a host cell. Such methods may include, but are not limited to plasmid or viral vectors, calcium phosphate transfection, protoplast fusion, electroporation, biolistics, liposomes, microinjection, or any methods available in the art.

After an expression vector or transgene is introduced into a host cell, the cell may then be cultured under conditions optimum for expression of the desired protein, which may be recovered using standard techniques known in the art. Viral pathogens or viral proteins such as those described herein may then be purified for use in diagnostic assays, for making antibodies and immunogenic compositions, and for identification of antiviral compounds. Naturally occurring proteins may be purified from biological samples, such as a tissue sample from a bird infected with a virus as described herein, while recombinant proteins may be purified using any suitable methods or expression systems known in the art.

A number of procedures for purifying recombinant protein are available in the art. For example, proteins having established molecular adhesion properties can be reversibly fused to another protein. Additionally, a specific protein may be selectively adsorbed to a purification column and then freed from the column in a relatively pure form using appropriate ligands or substrates. The fused protein may then be removed by enzymatic activity. Protein may also be purified using affinity columns. Recombinant protein can be purified from any suitable source.

### Purification of Protein From Recombinant Bacteria

Recombinant proteins may be expressed by bacteria in large amounts, for example using an inducible or constitutive promoter. Promoter induction using IPTG is an example of an inducible promoter system. Bacteria may be grown from fresh or frozen culture according to standard procedures known in the art.

Proteins expressed in bacteria may form insoluble aggregates called inclusion bodies. Suitable protocols for purification of protein inclusion bodies are known in the art. Lysing of bacterial for recovery of expressed proteins may be performed using any methods known in the art, which may include introduction of chemical buffers, sonication, mechanical disruption, and the like. Inclusion bodies may also be solubilized, and the lysed cell suspension may be centrifuged to remove unwanted cellular debris. Inclusion body proteins may be renatured by dilution or dialysis with an appropriate buffer.

Recombinant proteins may also be obtained from bacteria periplasm. After lysis of bacterial cells, the periplasmic fraction of the bacteria may be isolated by any methods known in the art. Recombinant proteins present in the supernatant may be separated from host proteins by standard separation techniques well known to those of skill in the art.

Proteins may be separated using any techniques known in the art, for example, solubility fractionation or size differential filtration, which isolates a protein on the basis of molecular weight using filtration through membranes of different pore size. Column chromatography may be used for isolation of a protein from other proteins on the basis of size, net surface charge, hydrophobicity, or affinity for ligands or substrates. In addition, antibodies raised against a protein of interest may be conjugated to a column and the proteins immunopurified. All of these methods are well known in the art. It will be apparent to one of skill that chromatographic techniques may be performed on any scale and using any appropriate commercial equipment.

### Antibody Production

Methods of producing polyclonal and monoclonal antibodies that react specifically with viral proteins, virus particles, and/or nucleic acids are known in the art. Such techniques may include antibody preparation by selection of antibodies from recombinant antibody libraries in phage or other vectors, as well as preparation of polyclonal and monoclonal antibodies by immunizing rabbits or mice.

A number of antigens or antigenic regions comprising a viral protein or portions thereof, virus particles, and/or nucleic acids may be used to produce antibodies specifically reactive to a desired viral pathogen. For example, a recombinant viral protein or an antigenic fragment thereof, may be isolated using any methods described herein or known in the art. Recombinant proteins may be expressed in prokaryotic or eukaryotic cells and purified as described herein. Monoclonal and/or polyclonal antibodies may be produced using naturally occurring (in pure or impure form) or recombinant proteins using methods known in the art. Synthetic peptides derived from a viral sequence may also be used to generate antibodies and may be conjugated to a carrier protein and injected into an animal capable of producing antibodies (ex. rabbit).

Methods of production of polyclonal antibodies are known to those of skill in the art. For example, an inbred strain of mice or rabbits may be immunized with a protein using a standard adjuvant, such as an adjuvant described herein, using a standard immunization protocol known in the art. When appropriately high titers of antibody to the protein are obtained, antisera may be prepared, and enrichment performed to obtain antibodies reactive to the protein.

Monoclonal antibodies may also be obtained by various methods known in the art. For example, spleen cells from an animal immunized with a desired antigen may be immortalized, commonly by fusion with a myeloma cell or through transformation with Epstein Barr Virus (EBV), oncogenes, or retroviruses, or other methods well known in the art. The immortalized cells may then be screened for production of antibodies of the desired specificity and affinity for the antigen. Yield of the monoclonal antibodies produced by such cells may be enhanced by various techniques known in the art, for example by injection into the peritoneal cavity of a vertebrate host.

Monoclonal antibodies and polyclonal sera may be collected and titered against the desired antigen or protein in an immunoassay, for example, a solid phase immunoassay with the protein immobilized on a solid support. Antibodies specific only for a particular viral protein may also be made by subtracting out other cross-reacting proteins. In this manner, antibodies that bind only to the protein of choice may be obtained.

Once the specific antibodies against the desired viral antigen, such as protein, virus, and/or nucleic acid are available, the desired antigen may be detected using a variety of immunoassay methods. The antibody may also be used therapeutically.

Protein either associated with or distinct from a viral particle as described herein may be detected and/or quantified using any of a number of well recognized immunological binding assays. Viral particles may be detected based on an epitope defined by the viral proteins as presented in a viral particle and/or an epitope defined by a viral protein that is separate from a viral particle (ex. such as may be present in an infected cell). Immunological assays may use an antibody that specifically binds to a protein or antigen of choice. The antibody may be produced by any of a number of methods well known to those of skill in the art. Immunoassays may also use a labeling agent to specifically bind to the complex formed by the antibody and antigen for detection purposes. The labeling agent may itself be one of the moieties comprising the antibody/antigen complex. Thus, the labeling agent may be a labeled viral protein nucleic acid or a labeled antiviral antibody. Alternatively, the labeling agent may be a third moiety, such as a secondary antibody, that specifically binds to the antibody/antigen complex. A secondary antibody may be specific to antibodies of the species from which the first antibody is derived. A labeling agent can be modified with a detectable moiety, such as biotin, to which another molecule can specifically bind, such as streptavidin. A variety of detectable moieties are well known to those skilled in the art.

Immunoassays for detecting viral protein, virus, and/or nucleic acid in samples are well known in the art. Such assays may be either competitive or noncompetitive and may be either quantitative or non-quantitative. Noncompetitive immunoassays are assays in which antigen may be directly detected and, in some instances, the amount of antigen directly measured. In competitive assays, viral antigen present in a sample is detected indirectly by a detectable signal associated with a known, added (exogenous) viral antigen displaced from an antiviral antigen antibody by the viral antigen present in a sample. In this manner, such assays can also be adapted to provide for an indirect measurement of the amount of viral antigen present in the sample. Competitive binding immunoassays may also be used to determine cross-reactivity, in which any cross-reacting antibodies may be removed from pooled antisera. Additional assay types, including but not limited to western blot or liposome immunoassays may also be used in accordance with the present invention.

One of skill in the art will appreciate that it is often desirable to minimize nonspecific binding in immunoassays. Particularly, where the assay involves an antigen or antibody immobilized on a solid substrate it is desirable to minimize the amount of nonspecific binding to the substrate. Means of reducing such non-specific binding are well known to those of skill in the art.

An assay as described herein may include a label or detectable group that does not significantly interfere with the specific binding of the antibody used in the assay. A detectable group may be any material having a detectable physical or chemical property. Such detectable labels are known in the art and generally, any label useful in such methods may be applied to the present invention. Thus, a “label” as used herein may be any composition detectable by spectroscopic, photochemical, biochemical, immunochemical, electrical, optical or chemical means. Useful labels in the present invention may include magnetic beads (ex. DYNABEADS™), fluorescent dyes (ex. fluorescein isothiocyanate, Texas red, rhodamine, and the like), radiolabels (ex. ³H, ¹²⁵I, ³⁵S, ¹⁴C, or ³²P), enzymes (ex. horse radish peroxidase, alkaline phosphatase, and/or any others known in the art and used in ELISA), and colorimetric labels such as colloidal gold or colored glass or plastic beads (ex. polystyrene, polypropylene, latex, etc.).

A label in accordance with the invention may be coupled directly or indirectly to the desired component of the assay according to methods well known in the art. As described above, a wide variety of labels may be used, with the choice of label depending on sensitivity, ease of conjugation with the compound, stability requirements, or available instrumentation, among others.

Non-radioactive labels may be attached by indirect means. Generally, a ligand molecule (ex. biotin) is covalently bound to the molecule. The ligand may then bind to another molecule (ex. streptavidin), which may be either inherently detectable or covalently bound to a signal system, such as a detectable enzyme, a fluorescent compound, or a chemiluminescent compound. The ligands and their corresponding targets may be used in any suitable combination with antibodies that recognize a viral antigen, or secondary antibodies that recognize an antiviral antigen. The molecules may also be conjugated directly to signal generating compounds, ex. by conjugation to an enzyme or fluorophore. Enzymes of interest to be used as labels may be hydrolases, for example phosphatases, esterases and glycosidases, or oxidotases, such as peroxidases. Fluorescent compounds may include fluorescein and its derivatives, rhodamine and its derivatives, dansyl, umbelliferone, and the like. Chemiluminescent compounds may include luciferin, 2,3-dihydrophthalazinediones, ex. luminol, or others known in the art.

Means of detecting labels are well known to those of skill in the art and will depend on the type of label used. For example, autoradiography may be used to detect a radioactive label, or fluorochromes may be used to detect a fluorescent label. Fluorescence may be detected visually, for example by electronic detectors such as charge coupled devices (CCDs) or photomultipliers, and the like. Similarly, enzymatic labels may be detected by providing the appropriate substrates for the enzyme and detecting the resulting reaction product. Colorimetric or chemiluminescent labels may be detected by observing a color associated with a particular label. In some embodiments, an assay formats may not require the use of a labeled component but rather may be detected by simple visual inspection.

### Pharmaceutical/Immunogenic Compositions and Administration Thereof

In some aspects recombinant vectors comprising one or more transgenes expressing one or more viral proteins or peptides or fragments thereof as described herein may be used as pharmaceutical compositions or immunogenic compositions for administering to a subject such as a chicken or other poultry in order to provide protection from one or more viruses. For example, an immunogenic composition as described herein comprise a recombinant vector with one or more transgenes as described herein which are inserted into the viral genome, for example in an intergenic region flanked by the intergenic loci UL 35/UL 36 in the unique long (UL) region of the HVT genome. In one aspect the present invention provides a recombinant Herpesvirus of Turkey (HVT) genome comprising one or more nucleotide sequence(s) coding for one or more heterologous antigen(s) inserted into the intergenic loci UL 35/UL 36 in the unique long region of the HVT genome and one or more nucleotide sequence(s) coding for one or more heterologous antigens inserted at the UL55 site in the unique long region (UL) of the HVT genome.

In other aspects, proteins or peptides and immunogenic fragments thereof, and/or polynucleotides, as well as antiviral antibodies and/or T cells, may be incorporated into pharmaceutical compositions or immunogenic compositions (ex. vaccines). In another embodiment, an immunogenic composition according to the invention may comprise at least a third transgene, a fourth transgene, or the like, which may encode additional viral proteins. In such a way, it is possible to provide an immunogenic composition to a subject such as poultry that provides protection from any desired number of viruses. Whole virus vaccine (live and attenuated, or replication incompetent, or killed) or subunit vaccines, such as structural or non-structural viral proteins or immunogenic fragments thereof, can be used to treat or prevent viral infections by eliciting an immune response in a subject. Alternatively, a pharmaceutical composition may comprise an antigen-presenting cell transfected with a viral polynucleotide such that the antigen-presenting cell expresses a viral peptide.

Immunogenic compositions in accordance with the invention may be designed to generate antibody immunity and/or cellular immunity in a subject. Such compositions may comprise one or more such compounds along with a non-naturally occurring pharmaceutically acceptable carrier. In other embodiments, an immunogenic composition in accordance with the invention may include more than one adjuvants or pharmaceutically acceptable carriers such that at least one is non-naturally occurring. A pharmaceutically acceptable carrier or adjuvant may be any substance that enhances an immune response in a subject to an exogenous antigen, including but not limited to, adjuvants, liposomes, biodegradable microspheres. A pharmaceutically acceptable carrier or adjuvant may contain a substance designed to protect the antigen from rapid catabolismsuch as aluminum hydroxide or mineral oil, or a stimulator of immune responses, such as proteins derived from Bortadella pertussis or Mycobacterium tuberculosis. Commercially available adjuvants may include for example, Freund's Incomplete Adjuvant and Complete Adjuvant, Merck Adjuvant 65, aluminum salts such as aluminum hydroxide gel (alum) or aluminum phosphate; CpG oligonucleotides, salts of calcium, iron or zinc; an insoluble suspension of acylated tyrosine; acylated sugars; cationically or anionically derivatized polysaccharides; polyphosphazenes; biodegradable microspheres; and monophosphoryl lipid A. One of skill in the art will be able to identify appropriate pharmaceutically acceptable carriers for use with the present invention.

Pharmaceutical or immunogenic compositions and/or vaccines within the scope of the present invention may also contain other compounds, which may be biologically active or inactive. For example, one or more immunogenic portions of other antigens may be present, either incorporated into a fusion polypeptide or as a separate compound, within a composition or vaccine according to the invention. In some embodiments, polypeptides useful with the present invention may be conjugated to other macromolecules. Pharmaceutical or immunogenic compositions and vaccines may generally be used for prophylactic and/or therapeutic purposes. For example, in accordance with the invention, a composition as described herein may be provided to a subject, such as a bird, prior to infection with or exposure to a virus in order to provide protection against infection with one or more viruses or development of symptoms of infection. In other embodiments, such a composition may be provided to a subject, such as a bird, after infection with or exposure to one or more viruses in order to provide treatment of the viruses in the subject, such as by reducing or eliminating infection in the subject.

Nucleic acid vaccines encoding a genome, structural or non-structural protein, or a fragment thereof of a virus described herein may also be used to elicit an immune response to treat or prevent viral infection. Numerous gene delivery techniques are well known in the art. Appropriate nucleic acid expression systems may contain the necessary DNA sequences for expression in a subject (such as a suitable promoter and termination signal). In some embodiments, a DNA as described herein may be introduced using a viral expression system (ex. Marek's disease virus or HVT), which may involve the use of a non-pathogenic, replication competent virus.

Pharmaceutical or immunogenic compositions may be provided in single-dose or multi-dose containers, such as sealed ampoules or vials. Such containers may be sealed to preserve sterility of the composition until use. In general, compositions as described herein may be stored as suspensions, solutions, or emulsions in oily or aqueous vehicles. Alternatively, such a composition may be stored in a freeze-dried condition requiring only the addition of a sterile liquid carrier immediately prior to use.

As described herein, an immunogenic composition may be combined with a pharmaceutically acceptable carrier. The selection of a suitable carrier may be determined in part by the particular composition being administered (ex. nucleic acid, protein, modulatory compounds, or transduced cell), as well as by the particular method used to administer the composition. Accordingly, a wide variety of suitable formulations of pharmaceutical or immunogenic compositions are available that may of use in the present invention. Administration may be in any convenient manner, ex. by injection, oral administration, inhalation, transdermal application, or rectal administration. Injection of a recombinant vector or an immunogenic composition as described herein may be provided to a subject such as poultry in a single administration or dose, or may be administered more than once, such as repeated doses.

Formulations suitable for parenteral administration, such as, for example, by intraarticular (in the joints), intravenous, intramuscular, intradermal, intraperitoneal, in ovo, and subcutaneous routes, include aqueous and non-aqueous, isotonic sterile injection solutions, which can contain antioxidants, buffers, bacteriostats, and solutes that render the formulation isotonic with the blood of the intended subject, and aqueous and nonaqueous sterile suspensions that can include suspending agents, solubilizers, thickening agents, stabilizers, and preservatives. In the practice of this invention, compositions may be administered, for example, by intravenous infusion, orally, topically, intraperitoneally, intravesically or intrathecally.

Such compositions may also comprise buffers (ex. neutral buffered saline or phosphate buffered saline), carbohydrates (ex. glucose, mannose, sucrose or dextrans), mannitol, proteins, polypeptides or amino acids such as glycine, antioxidants, bacteriostats, chelating agents such as EDTA or glutathione, adjuvants (ex. aluminum hydroxide), solutes that render the formulation isotonic, hypotonic, or weakly hypertonic with the blood of a subject, suspending agents, thickening agents, and/or preservatives. Alternatively, compositions of the present invention may be formulated as a lyophilizate. Compounds may also be encapsulated within liposomes using methods known in the art.

Injection solutions and suspensions may be prepared from sterile powders, granules, and tablets as described herein. Cells transduced by nucleic acids for ex vivo therapy may also be administered intravenously or parenterally as described above. An injection as described herein may involve a suspension of one or more of a killed, inactivated, attenuated, or otherwise non-virulent virus culture, purified or non-purified solution of a viral protein, or a nucleic acid as described herein. An injection solution may also contain a pharmaceutically acceptable carrier as described herein.

Formulations suitable for oral administration may consist of (a) liquid solutions, such as an effective amount of the packaged viral protein or nucleic acid suspended in diluents, such as water, saline or PEG 400; (b) capsules or tablets, each containing a predetermined amount of the active ingredient, as liquids, solids, granules or gelatin; (c) suspensions in an appropriate liquid; or (d) suitable emulsions. Tablet forms may include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms may comprise the active ingredient in a flavor, ex. sucrose, as well as pastilles comprising the active ingredient in an inert base, such as gelatin and glycerin or sucrose and acacia emulsions, gels, and the like containing, in addition to the active ingredient, carriers known in the art.

The compound of choice, alone or in combination with other suitable components, may be made into aerosol formulations to be administered via inhalation. Aerosol formulations may be placed into pressurized acceptable propellants, such as dichlorodifluoromethane, propane, nitrogen, and the like.

The dose administered to a subject in the context of the present invention should be sufficient to affect a beneficial therapeutic response in the subject over time. The dose will be determined by the efficacy of the particular vector employed and the condition of the subject, as well as the body weight and/or surface area of the patient to be treated. The size of the dose also may be determined by the existence, nature, and extent of any adverse side-effects that accompany the administration of a particular vector, or transduced cell type in a particular patient. For compositions comprising a vector as described herein, the effective amount of the vector to be administered may be determined in part based on circulating plasma levels of the vector, vector toxicities, health of the subject, and production of anti-vector antibodies.

For administration, compounds and transduced cells of the present invention can be administered at a rate determined by the LD-50 of the inhibitor, vector, or transduced cell type, and the side-effects of the inhibitor, vector or cell type at various concentrations, as applied to the mass and overall health of the subject. Administration may be accomplished via single, multiple, or divided doses.

### Immunological Detection of Polypeptides and Nucleic Acids

Immunoassays may be used to detect viral proteins, virus particles, and/or nucleic acids. Such assays may be useful for therapeutic and/or diagnostic applications, such as those described herein. Immunoassays are well known in the art and may be used to qualitatively or quantitatively analyze proteins, virus particles, and/or nucleic acids.

### Assays for Viral Proteins and Antibodies to Viral Antigens

In one embodiment of the present invention, the presence of a virus as described herein, a viral nucleic acid, or a viral protein in a sample may be determined by an immunoassay. Enzyme-mediated immunoassays such as immunofluorescence assays (IFA), enzyme linked immunosorbent assays (ELISA), capture assays, micro-agglutination tests, and immunoblotting assays (ex. western blot) can be readily adapted to accomplish detection of a virus or viral proteins. An ELISA method may be effective for detection of a virus or viral protein as described herein. Such an ELISA may, for example, have steps such as: (1) bind an antiviral antibody or antigen to a substrate; (2) contact the bound receptor with a biological sample containing a virus, a viral antigen, a viral protein, or antibodies to the virus; (3) contact the biological sample with an antibody bound to a detectable moiety (ex. horseradish peroxidase enzyme or alkaline phosphatase enzyme); (4) contact the biological sample with the substrate for the enzyme: (5) contact the biological sample with a detecting reagent, such as a color reagent; (6) observe a detectable result. In some embodiments, a biological sample suitable for use in such an ELISA may be blood or other fluids. In another embodiment, an ELISA as described herein may detect a virus or viral protein in a tissue sample. Such methods may be readily modified by those of skill to detect the presence of an antiviral antibody in a sample, or a specific viral protein, as well as the virus. In certain embodiments, an ELISA according to the invention may detect the presence of an antiviral antibody.

ELISA assays as described herein may include a nitrocellulose strip impregnated with a viral protein as described herein. The nitrocellulose strip may produce a visual result when contacted with a test sample containing antiviral nucleoprotein antibodies. Such a test may identify a subject already having antibodies against a viral protein and thus the subject may have immunity to the virus. Administration of an immunogenic composition to prevent viral infection such as described herein may be unnecessary in such a subject and therefore, identification of subjects already having immunogenic antibodies may prevent unnecessary administration of an immunogenic compound to such a subject. In this regard, an embodiment of the present invention may involve identifying a subject lacking antiviral antibodies using an assay as described herein, such as an ELISA assay, and then providing an immunogenic composition as described herein to that subject in order to prevent viral infection. In another embodiment, a nitrocellulose strip for use in an ELISA according to the invention may be impregnated with an antibody, such as antiviral antibody, and may produce a visual result when contacted with a test sample containing a viral protein. Such a test may identify a subject infected with a virus as described herein.

Another immunologic technique that can be useful in the detection of a virus is a competitive inhibition assay. Such an assay utilizes monoclonal antibodies (MABs) reactive with a specific virus. A biological fluid (ex. blood) from a subject may be contacted with a first antibody bound to a substrate, and a labeled monoclonal antibody contacted with the first antibody-virus complex. The amount of inhibition of monoclonal antibody binding is measured relative to a control.

As will be readily understood by one of skill in the art, a biological sample for use in the above assays may be taken directly from a subject or may be in a partially purified form. An antibody specific for a particular virus will react by binding to the virus as a primary reaction. Thereafter, a secondary reaction with an antibody bound to or labeled with a detectable moiety may also be added in order to enhance the detection of the primary reaction. Generally, in the secondary reaction, an antibody or other ligand which is reactive, either specifically or nonspecifically with a different binding site (epitope) of the virus will be selected for its ability to react with multiple sites on the complex of antibody and virus. Thus, for example, several molecules of the antibody in the secondary reaction can react with each complex formed by the primary reaction, making the primary reaction more detectable.

The detectable moiety can allow visual detection of a precipitate or a color change, visual detection by microscopy, or automated detection by spectrometry, radiometric measurement or the like. Examples of detectable moieties include fluorescein and rhodamine (for fluorescence microscopy), horseradish peroxidase (for either light or electron microscopy and biochemical detection), biotin-streptavidin (for light or electron microscopy) and alkaline phosphatase (for biochemical detection by color change). The detection methods and moieties used can be selected, for example, from any disclosed herein or available in the art.

### Detecting the Presence of a Viral Nucleic Acid

In some embodiments, a viral infection as described herein may be detected based on the level of a particular RNA or DNA in a biological sample. Primers from a particular virus or viral pathogen may be used for detection, diagnosis, and determination of the presence of a virus. Any suitable primer may be used to detect genomic DNA or any sequence therein, an open reading frame or gene, or a protein of choice, using any appropriate methods known in the art. A suitable nucleic acid sequence may be used as single-or double-stranded probes or primers for detection of viral mRNA or cDNA generated therefrom, as may be present in a biological sample. Viral polynucleotides as described herein may also be used to generate additional copies of the polynucleotides, in order to generate antisense oligonucleotides, or as triple-strand forming oligonucleotides. For example, two oligonucleotide primers may be used in a PCR-based assay to amplify a portion of a viral cDNA derived from a biological sample, wherein at least one of the oligonucleotide primers is specific for (i.e., hybridizes to) the viral polynucleotide. Such primers may be any length sufficient to hybridize to and enable amplification of a viral nucleic acid as described herein, including at least or about 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, 30 nucleotides, 35 nucleotides, 40 nucleotides, 45 nucleotides, or 50 nucleotides; or from about 12 to about 50 nucleotides in length, 15 to 30 nucleotides in length, 15 to 25 nucleotides in length, or 20 to 30 nucleotides in length. DNA primers suitable for use with the present invention may be any primers described herein, such as those set forth as SEQ ID NOs: 40-157

An amplified nucleotide, for example a cDNA, may then be separated and detected using techniques well known in the art, such as gel electrophoresis. Similarly, oligonucleotide probes that specifically hybridize to a viral polynucleotide may be used in a hybridization assay to detect the presence of a viral polynucleotide in a biological sample.

Nucleic acid probes or primers specific to a virus as described herein may be generated using the polynucleotide sequences disclosed herein. The probes are preferably at least about 12, 15, 16, 18, 20, 22, 24, or 25 nucleotide fragments or other polynucleotide sequence encoding a viral nucleic acid or polypeptide. Nucleic acid probes can be less than about 200 bp, 150 bp, 100 bp. 75 bp, 50 bp, 60 bp, 40 bp, 30 bp, 25 bp 2 kb, 1.5 kb, 1 kb, 0.5 kb, 0.25 kb, 0.1 kb, or 0.05 kb in length. The probes can be produced by, for example, chemical synthesis, PCR amplification, generation from longer polynucleotides using restriction enzymes, or other methods well known in the art. The polynucleotides described herein may also be used in methods or assays that involve the use of solid substrates, such as arrays. Such an array may have one or more different polynucleotides, which may be immobilized on the arrays using methods known in the art.

In some embodiments, a polynucleotide of the invention may be detectably labeled. Detectable labels may include, but are not limited to, radiolabels, fluorochromes, including fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein, 6-carboxy-X-rhodamine (ROX), 6-carboxy-2′,4′,7′,4,7-hexachlorofluorescein (HEX), 5-carboxy fluorescein (5-FAM) or N,N,N′,N′-tetramethyl-6-carboxyrho-damine (TAMRA); radioactive labels such as ³²P, ³⁵S, and ³H), and the like. In some embodiments, a detectable label may involve multiple steps (ex. biotin-avidin, hapten-anti-hapten antibody, and the like).

In accordance with the invention, any suitable qualitative or quantitative methods known in the art for detecting specific viral nucleic acids (ex. RNA or DNA) may be used. A viral nucleic acid as described herein may be detected by, for example, in situ hybridization in tissue sections, using methods that detect single base pair differences between hybridizing nucleic acid, by reverse transcriptase-PCR, or in northern blots containing poly A mRNA, or other methods well known in the art. For detection of viral polynucleotides in blood or blood-derived samples, methods that allow for detection of single base pair mismatches may be employed.

A viral nucleic acid sequence may be present in a biological sample obtained from an infected individual at relatively low levels, and thus amplification techniques known in the art (ex. PCR) may be used to amplify the sequence prior to performing a hybridization assays.

Nucleic acid probes may be prepared using a viral genome as described herein. Such a probe may include at least about 8 nucleotides or more and may be prepared synthetically or by excision from recombinant polynucleotides. A probe as described herein may hybridize with a viral nucleic acid, and thus such a probe may be useful for detection of a particular virus in a biological sample. Probes as described herein may also be useful for identification of infected subjects, as well as for further characterization of viral genomes. A probe for detecting viral polynucleotides (natural or derived) may be of a specific length or have a sequence which allows the detection of unique viral sequences by hybridization. While about 6-8 nucleotides may be useful, longer sequences may be preferred, ex. sequences of about 10-12 nucleotides, or about 20 nucleotides or more. One of skill in the art will be aware how to make and use a probe as described herein.

Nucleic acid probes may be prepared using routine methods, including, but not limited to, automated oligonucleotide synthetic methods. A sequence useful for preparing such a probe may include a complement to any unique portion of a viral genome, for example a portion of the viral genome that allows for distinguishing a particular virus from other viruses that may be present in the sample. A probe as described herein may have complete complementarity to the target sequence of interest or may have one or more mismatches. A probe useful in accordance with the invention having one of more mismatches will still hybridize to the target sequence of interest. For use of such probes as diagnostics, the biological sample to be analyzed may be treated prior to analysis, if desired, to extract the nucleic acids contained therein. The resulting nucleic acids from the sample may be subjected to gel electrophoresis or other size separation techniques. A probe may be labeled with a detectable label as described herein. Suitable labels, and methods for labeling probes are known in the art and may include any labels described herein or others useful with the present invention.

A probe may be completely complementary to a viral genome or portion thereof (ex. to all or a portion of a sequence encoding a viral protein as described herein). High stringency conditions may be desirable in order to prevent or at least minimize false positive results. The stringency of hybridization may be determined by several factors during hybridization and washing, including temperature, ionic strength, length of time, and concentration of reagents. A probe or nucleic acid from a sample may be provided in solution for such assays or may be affixed to a support (ex. solid or semi-solid support). Examples of supports that may be used include but are not limited to nitrocellulose (ex. membrane or microtiter well form), polyvinyl chloride (ex. sheets or microtiter wells), polystyrene latex (ex. beads or microtiter plates, polyvinylidine fluoride, diazotized paper, nylon membranes, activated beads, and Protein A beads).

In one embodiment, a probe or sample nucleic acid may be provided on an array for detection. Arrays may be created by, for example, spotting polynucleotide probes onto a substrate (ex. glass, nitrocellulose, and the like) in a two-dimensional matrix or array. The probes may be bound to the substrate by either covalent bonds or by non-specific interactions, such as hydrophobic interactions. Samples of polynucleotides can be detectably labeled (ex. using radioactive or fluorescent labels) and then hybridized to the probes. Double stranded polynucleotides, comprising the labeled sample polynucleotides bound to probe polynucleotides, may be detected once the unbound portion of a sample is removed. Techniques for constructing arrays and methods of using these arrays are known in the art. Arrays may be used for a single sample to be analyzed for the presence of two or more nucleic acid target regions. In such a case, the probes for each of the target regions, as well as controls (both positive and negative) may be provided on a single array. Arrays thus facilitate rapid and convenience analysis.

### Diagnostic Tests and Kits

The invention further provides diagnostic reagents and kits comprising one or more such reagents for use in a variety of diagnostic assays, including for example, immunoassays such as ELISA and “sandwich”-type immunoassays, as well as nucleic acid assays, ex. PCR assays. In a related embodiment, an assay may be performed in a flow-through or strip test format, wherein the binding agent is immobilized on a membrane, such as nitrocellulose. Such kits may preferably include at least a first peptide, or a first antibody or antigen binding fragment of the invention, a functional fragment thereof, or a cocktail thereof, or a first oligonucleotide pair, and means for signal generation. In some embodiments, a kit may comprise an immunogenic composition, such as a recombinant virus as described herein. Reagents and other compounds, such as a pharmaceutically acceptable carrier may be included in the kit. An immunogenic composition when provided in such a kit may be in a solution such as in a pre-measured dose or amount, or may be a dry composition, such as in desiccated or lyophilized form suitable for rehydration or resuspension. The kit components may be pre-attached to a solid support or may be applied to the surface of a solid support when the kit is used. The signal generating means may come pre-associated with an antibody or nucleic acid of the invention or may require combination with one or more components, ex. buffers, nucleic acids, antibody-enzyme conjugates, enzyme substrates, or the like, prior to use.

Kits may also include additional reagents, ex. blocking reagents for reducing nonspecific binding to the solid phase surface, washing reagents, enzyme substrates, enzymes, and the like. The solid phase surface may be in the form of microtiter plates, microspheres, or other materials suitable for immobilizing nucleic acids, proteins, peptides, or polypeptides. An enzyme that catalyzes the formation of a chemiluminescent or chromogenic product or the reduction of a chemiluminescent or chromogenic substrate is one such component of the signal generating means. Such enzymes are well known in the art. Where a radiolabel, chromogenic, fluorigenic, or other type of detectable label or detecting means is included within the kit, the labeling agent may be provided either in the same container as the diagnostic or therapeutic composition itself or may alternatively be placed in a second distinct container into which this second composition may be placed and suitably aliquoted. Alternatively, the detection reagent and the label may be prepared in a single container means.

All patents and other publications identified are expressly incorporated herein by reference for the purpose of describing and disclosing, for example, the methodologies described in such publications that might be used in connection with the present invention. These publications are provided solely for their disclosure prior to the filing date of the present application.

The present invention is further illustrated and supported by the following examples. However, these examples should in no way be considered to further limit the scope of the invention. To the contrary, one having ordinary skill in the art would readily understand that there are other embodiments, modifications, and equivalents of the present invention without departing from the spirit of the present invention and/or the scope of the appended claims.

### EXAMPLES

### Example 1

### Construction of HVT-gfp Plasmids

HVT-Green Fluorescent Protein (gfp)-B Transfer Plasmid Construction

HVT-gfp-B transfer plasmid (SEQ ID NO.18) was chemically synthesized by GeneArt, ThermoFisher). 2.5 ug of the plasmid was transfected into secondary CEF cells using LTX transfection reagent (Invitrogen) in 6-well plate. About 4-6 hours later, the transfected cells were infected with HVT at 0.006 moi (1.5×10⁴ pfu/2.5×10⁶ cells). Three days later, the cells were passaged 1:15 to T75 with fresh CEF (1×10⁷ cells/T75). The cells were then plated 1:50 onto 24 well-plates three days later. Cells from wells that contain green fluorescent foci were plated onto 96-well plate at 1:200, 1:500 and 1:1000 dilutions with fresh cells (6×10\hat{ }4 cells/well). The wells that contain single green foci were purified 3 rounds by limiting dilution method using 96-well plates. The purified virus was expanded using CEF cells and frozen stock made. It was designated as “HVT-gfp-B”.

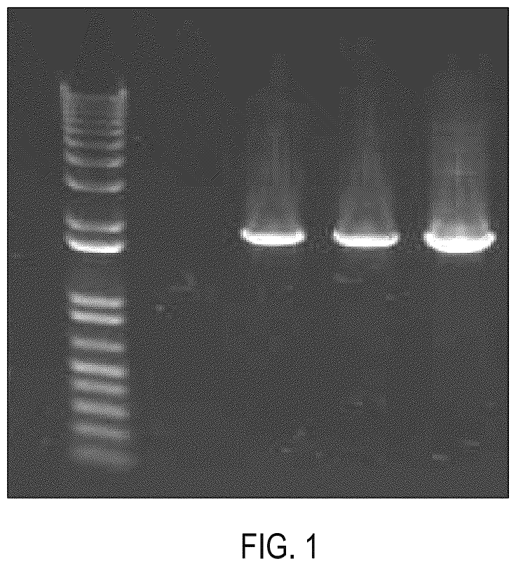

PCR analysis of three purified clone using primers just outside of integration site of UL55-Gene3 (upper primer: SEQ ID NO. 49; lower primer: 5′-SEQ ID NO. 50) gave a band of 1.893 kb as predicted. HVT gave a band of 0.15 kb as expected. Please refer to FIG. 1.

### HVT-gfp-A Modified Transfer Plasmid Construction

Modified transfer plasmid HVT-gfp-A (SEQ ID NO.16) was created by applying site-specific mutagenesis using two pairs of primers (upper primer pairs to generate SbfI upstream of gfp gene: SEQ ID NO.40 and SEQ ID NO 41; lower primers to generate SbfI downstream of gfp gene:—SEQ ID NO. 42 and SEQ ID NO.43 for original transfer plasmid HVT-gfp-A (SEQ ID NO. 17) that was chemically synthesized by GeneArt, ThermoFisher. 0.01 ug of modified transfer plasmid HVT-gfp-A was co-transfected with 2.5 ug HVT DNA using 7.5 uL PEI (Polyethylenimine) onto secondary CEF cells on 6-well plate. Green fluorescent foci became apparent at passage 1. After three rounds of purification by limiting dilution method, 1 clone of HVT-gfp-A was further expanded and frozen stocks made.

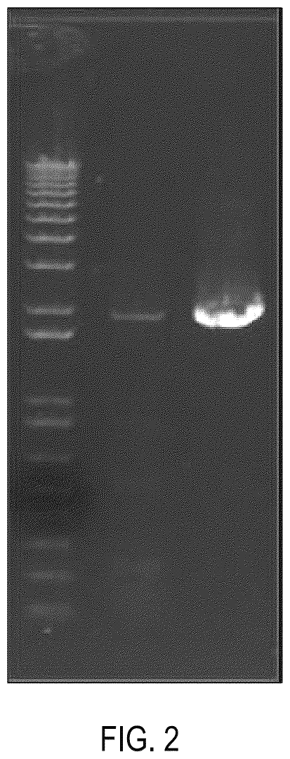

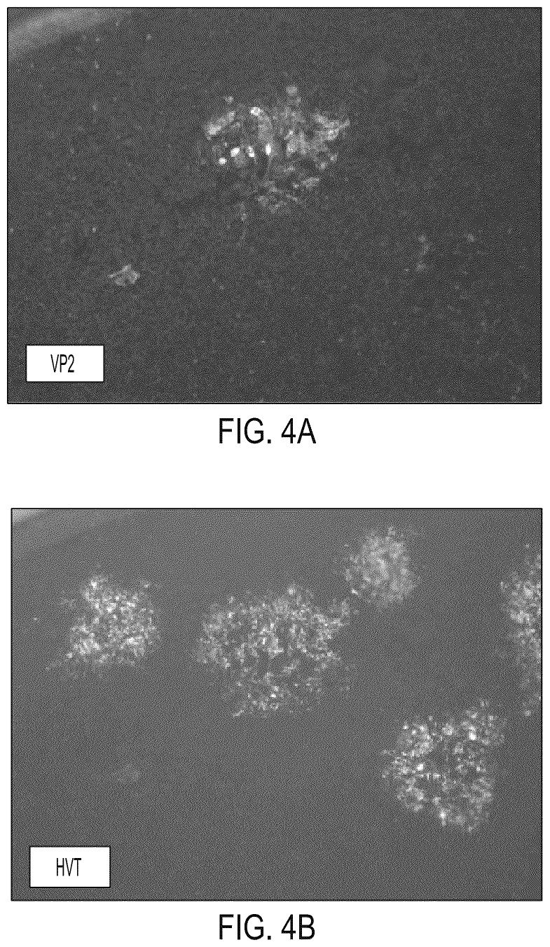

PCR analysis of the purified clone (left lane) using primers just outside of integration site of UL35-UL36 (upper primer: SEQ ID NO. 44; lower primer: SEQ ID NO. 45) gave a 1.922 kb band as predicted (FIG. 4). The DNA of modified transfer plasmid HVT-gfp-A was used as control (right lane). Please refer to FIG. 2.

### Example 2

### HVT-IBD Construction

### Construction of HVT-IBD #1

HVT-IBD #1 transfer plasmid (SEQ ID NO. 20) was chemically synthesized by GeneArt, ThermoFisher). 2.5 ug of the plasmid was transfected into secondary CEF cells using LTX transfection reagent (Invitrogen) in 6-well plate. Approximately 4-6 hours later, the transfected cells were infected with HVT at 0.055 moi. Three days later, the cells were passaged 1:7.5 to T75 with fresh CEF (1×10⁷ cells/T75). The cells were then plated onto 10 of 96 well-plates and duplicate plates were made three days later. One set of plates were fixed and stained with anti-IBDV chicken serum. Two wells that contained foci that positively stained for IBD were identified. The corresponding wells that contain positive staining foci were purified three rounds by limiting dilution method using 96-well plates. The purified virus was expanded using CEF cells and frozen stock made. It was designated as “HVT-IBD #1”.

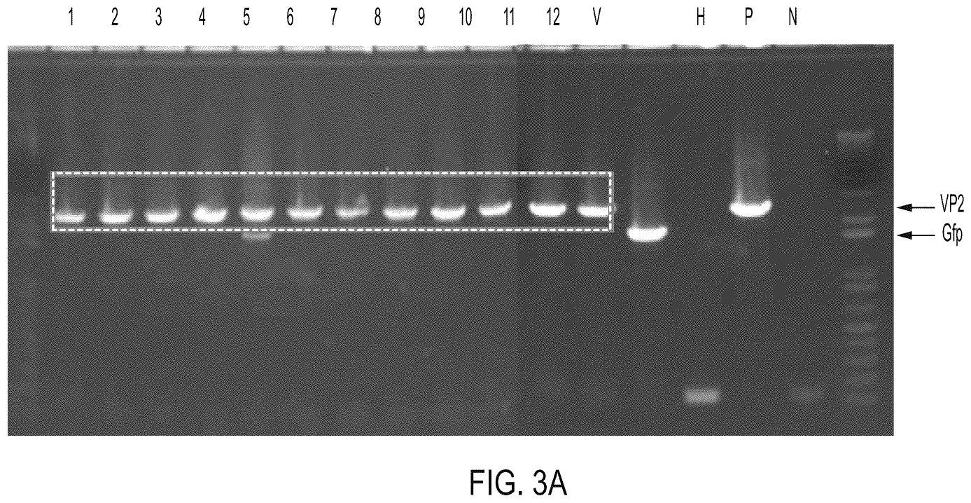

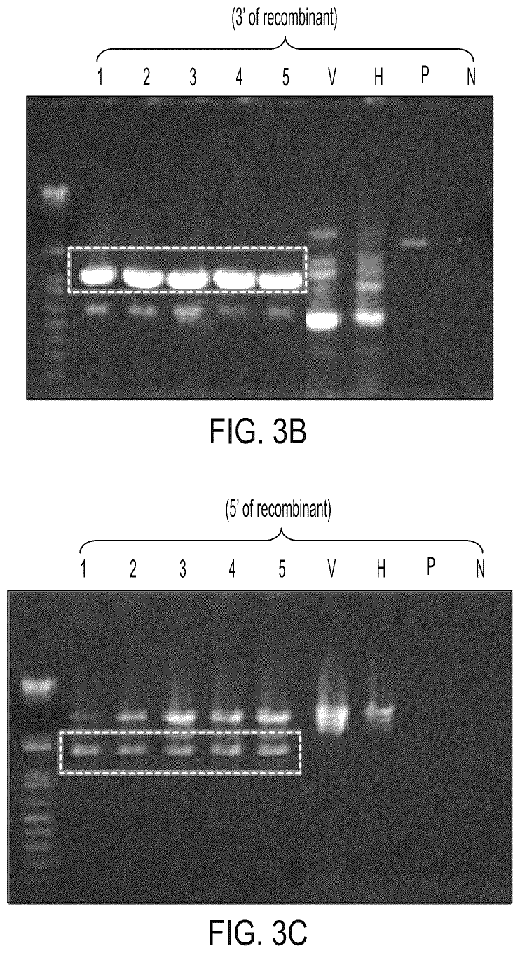

PCR analysis of the different clones using primers just outside of integration site of UL55-Gene3 (upper primer: SEQ ID NO. 46; lower primer: SEQ ID NO. 47, panel A) gave a band of 2.414 kb, while the PCR band of original vector is 1.922 kb. The correct integration was further confirmed by using primers surrounding the downstream junction of the insertion (upper primer SEQ ID NO. 48 that localized within IBDV VP2 coding region; lower primer SEQ ID NO.49 that localized downstream of the transfer plasmid. Panel B). A PCR band of 1.118 kb was obtained as expected. The correct integration for upstream integration site was performed using primers surrounding the upstream junction of the insertion (upper primer SEQ ID NO.50 that localized upstream of the transfer plasmid; lower primer SEQ ID NO.51 that within IBDV VP2 coding region, Panel C). A PCR band of 1.428 kb was obtained as expected. Please refer to FIGS. 3A, B and C.

### Construction of HVT-IBD #5

HVT-IBD #5 transfer plasmid (SEQ ID NO. 21) was chemically synthesized by GeneArt, ThermoFisher). JBJ-1 cells (a chicken fibroblast cell line) in a 6 well plate was transfected with 2.5 ug of the plasmid using LTX transfection reagent (Invitrogen). The transfected cells were infected with HVT at an moi of 0.05 approximately 5 hours post transfection. The transfected/infected cells were amplified via serial passage (1:4-1:10) and a portion subsequently seeded in 96 well plates in limiting dilutions. IBDV VP2 antigen expression was assessed by staining live cells the monolayers with antibody without fixation. Please see FIGS. 4A and 4B. Stained foci were harvested via trypsinization of the cells with cloning cylinders placed around the positive foci. This “live staining” followed by cloning cylinder passage was repeated 4 times and yielded pure VP2 positive cultures. The cultures were amplified via serial passage on JBJ-1 cells and before a final amplification on primary CEF cells in roller bottles. The harvested CEF cells were used to make a frozen cell stock and designated as, “HVT-IBD #5”.

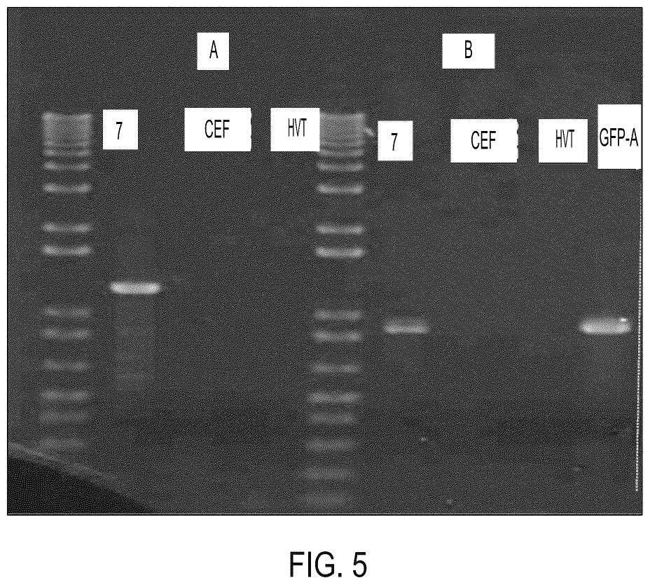

PCR analysis of clone #7 using 2 sets of primers to confirm integration of the insert across both insertion sites. In PCR A, the upper primer (SEQ ID NO.52) binds to the IBDV VP2 coding region, while the lower primer (SEQ ID NO.53) binds downstream of integration site of UL35-UL36. This set of primers yielded a PCR band of 1.244 kb as expected. In PCR B, the upper primer (SEQ ID NO. 54) binds upstream of the UL35-UL36 insertion site, and the lower primer (SEQ ID NO.55) binds within the human CMV promoter of the insert and yielded a PCR band of 0.926 kb as expected. Please refer to FIG. 5.

### Construction of HVT-IBD #6a

HVT-IBD #6a transfer plasmid (SEQ ID NO.22) was chemically synthesized by BioBasic Inc. 0.1 ug and 0.01 ug of linearized transfer plasmid (by digestion with EcoR1 and HindIII) was co-transfected with 2.5 ug of the HVT-gfp-A that was digested with Sbf1 into secondary CEF cells using PEI (Polyethylenimine) transfection reagent in 6-well plate. 4 days post transfection, 4 non-green foci were seen for 0.01 ug transfer plasmid transfection and 3 non-green foci were seen for 0.1 ug transfer plasmid transfection, while no foci were seen with HVT-gfp-A digested with Sbf1 alone. 2 non-green foci were purified 3 times by limiting dilution method using 96-well plate. The purified virus was expanded using CEF cells and frozen stock made. It was designated as “HVT-IBD #6a”.

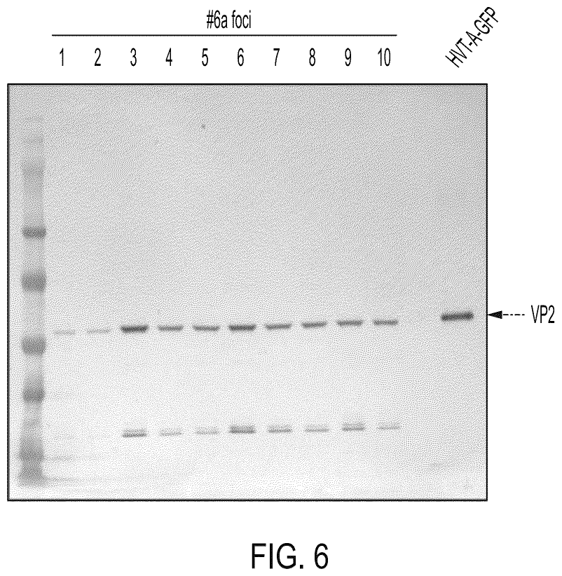

Infected cell lysate was prepared, and Western Blot analysis was performed using monoclonal antibody against IBDV R63. A protein band of about 50 KD was seen in all lanes, except the lane that contain the lysate of HVT-gfp-A vector. Please refer to FIG. 6.

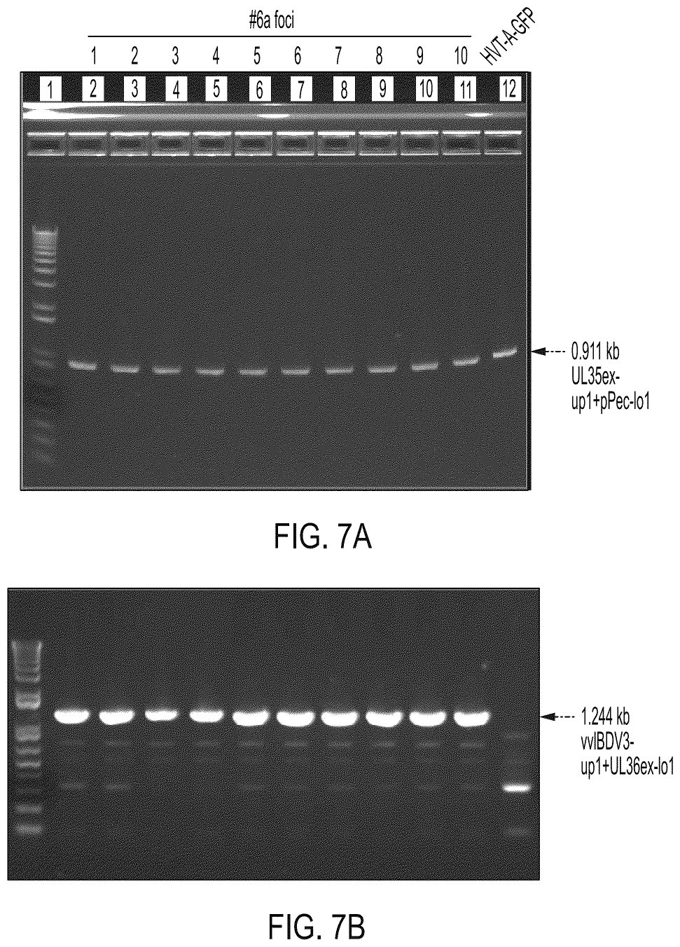

PCR analysis of the clones using 2 sets of primers to confirm the correct integration. The first primer set targeting the upstream integration site: upper primer 5′-SEQ ID NO.56 that localized upstream of UL35-UL36 integration site: lower primer SEQ ID NO. 57 that localized within the Pec promoter. This set of primers gave a PCR band of 0.911 kb as expected. The second primer set targeting downstream integration site: upper primer 5′-SEQ ID NO. 58 that localized within the IBDV VP2 coding region; lower primer 5′-SEQ ID NO. 59 that localized downstream of the UL35-UL36 insertion site. A PCR band of 1.244 kb was obtained as expected. Please refer to FIG. 7A and 7B.

### Construction of HVT-IBD #9

HVT-IBD #9 transfer plasmid (SEQ ID NO.23) was chemically synthesized by GeneArt, ThermoFisher). 2.5 ug of the plasmid was transfected into secondary CEF cells using LTX transfection reagent (Invitrogen) in 6-well plate. About 4-6 hours later, the transfected cells were infected with HVT-gfp-B at 0.075 moi. Three days later, the cells were passaged 1:10 to T75 with fresh CEF (1×10\hat{ }7 cells/T75) 3 times. The cells were then plated onto 10 of 96 well-plates and 90 non-green foci were obtained. Three of those were stained positive with anti-IBDV chicken serum. Two clones were purified 3 rounds by limiting dilution method using 96-well plates. The purified virus was expanded using CEF cells and frozen stock made. It was designated as “HVT-IBD #9”.

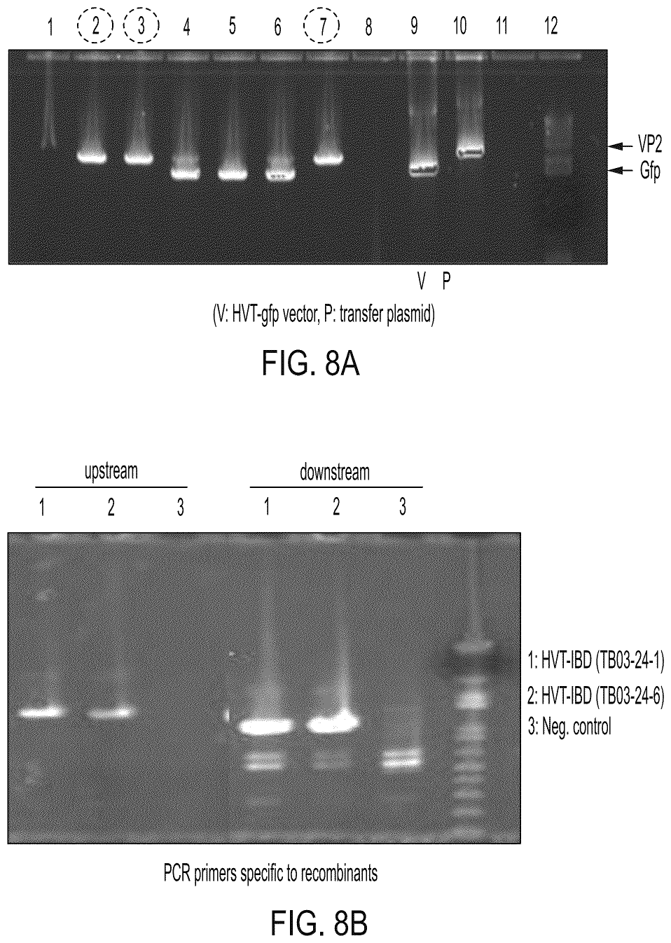

PCR analysis of the different clones using primers just outside of integration site of UL55-Gene3 (upper primer: SEQ ID NO.60; lower primer: SEQ ID NO. 61, panel A) gave a band of 2.536 kb, while the PCR band of original vector HVT-gfp-B is 1.922 kb. The correct integration was further confirmed by using primers surrounding the upstream junction of the insertion (upper primer SEQ ID NO. 62 that localized upstream of UL55-Gene3 insertion site; lower primer SEQ ID NO. 63 localized within IBDV VP2 coding region. A PCR band of 1.482 kb was obtained as expected. The correct integration for downstream site was performed using primers surrounding the downstream junction of the insertion (upper primer SEQ ID NO. 64 that localized within the IBDV VP2 sequence; lower primer SEQ ID NO. 65 that localize downstream of the UL55-Gene3 insertion site. A PCR band of 1.166 kb was obtained as expected. Please refer to FIGS. 8A and B.

### Construction of HVT-IBD #30

HVT-IBD #30 transfer plasmid (SEQ ID NO.24) was chemically synthesized by GeneArt, ThermoFisher. Secondary CEF cells were co-transfected with 0.1 ug of the plasmid and 2.5 ug of HVT using PEI (Polyethylenimine) transfection reagent in 6-well plate. Three days later, the cells were passaged 1:12 onto fresh CEF cells. Foci expressing IBD VP2 were visualized by staining unfixed cultures with chicken polyclonal serum against IBDV, and these foci marked with the aid of a fluorescent microscope. A total of 16 positive foci were passaged onto fresh CEF cells via trypsinization using cloning cylinders to segregate the foci from non VP2 expressing foci. Four of these cultures were clone three times following the same procedure before being amplified on primary CEF cells in roller bottles. A frozen stock of cells was put down and designated as “HVT-IBD #30”.

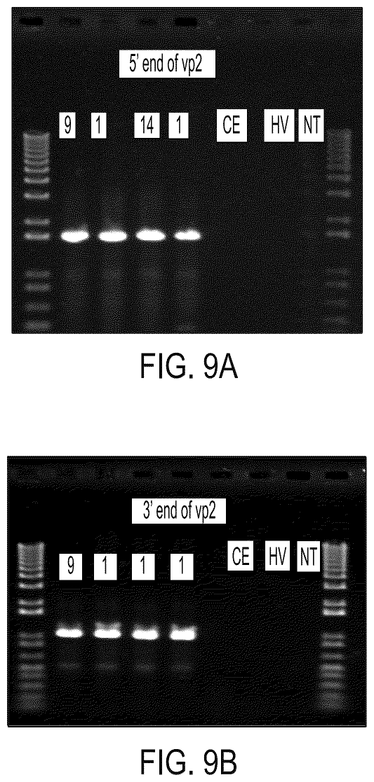

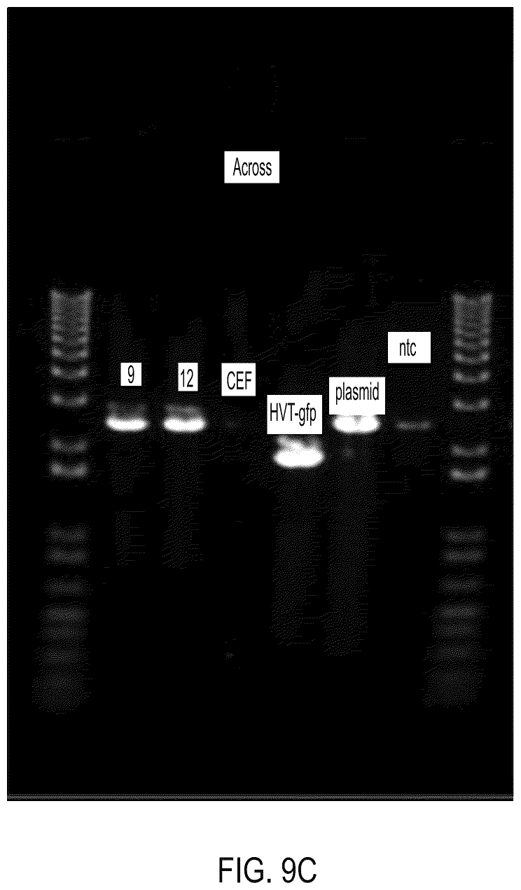

PCR analysis of 4 different clones using primers for upstream region of integration site of UL55-Gene3 (upper primer: SEQ ID NO.66; lower primer: SEQ ID NO.67, panel A) gave a band of 1.673 kb. The correct integration was further confirmed by using primers surrounding the 3′ junction of the insertion (upper primer SEQ ID NO. 68 that localized within IBDV VP2 coding region; lower primer SEQ ID NO. 69 that localized downstream of UL55-Gene3 insertion site, panel B). A PCR band of 1.082 kb was obtained as expected. The correct integration for downstream site was further confirmed by using primers outside of the expression cassette (upper primer SEQ ID NO.70; lower primer SEQ ID NO. 71 (panel C). A PCR band of 2.558 kb was obtained as expected. Please refer to FIGS. 9A-C.

### Construction of HVT-IBD #31

HVT-IBD #31 transfer plasmid (SEQ ID NO. 25) was chemically synthesized by GeneArt, ThermoFisher. 0.01 ug of linearized transfer plasmid (by digestion with EcoR1 and HindIII) was co-transfected with 2.5 ug of the HVT-gfp-A that was digested with Sbf1 into secondary CEF cells using PEI (Polyethylenimine) transfection reagent in 6-well plate. 4 days post transfection, 1 non-green foci were seen while no foci were seen with HVT-gfp-A digested with Sbf1 alone. After passage, 2 non-green foci were purified 3 times by limiting dilution method using 96-well plate. The purified virus was expanded using CEF cells and frozen stock made. It was designated as “HVT-IBD #31”.

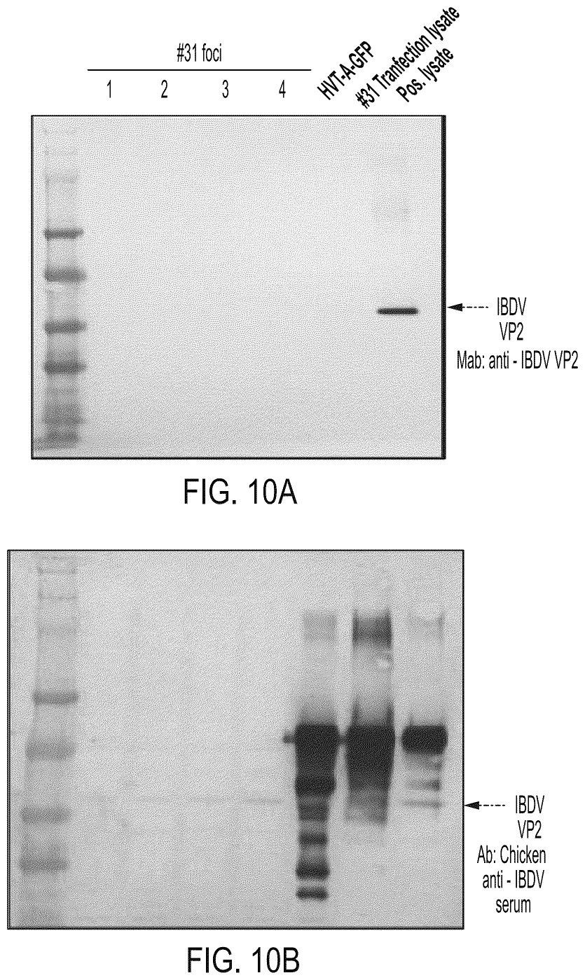

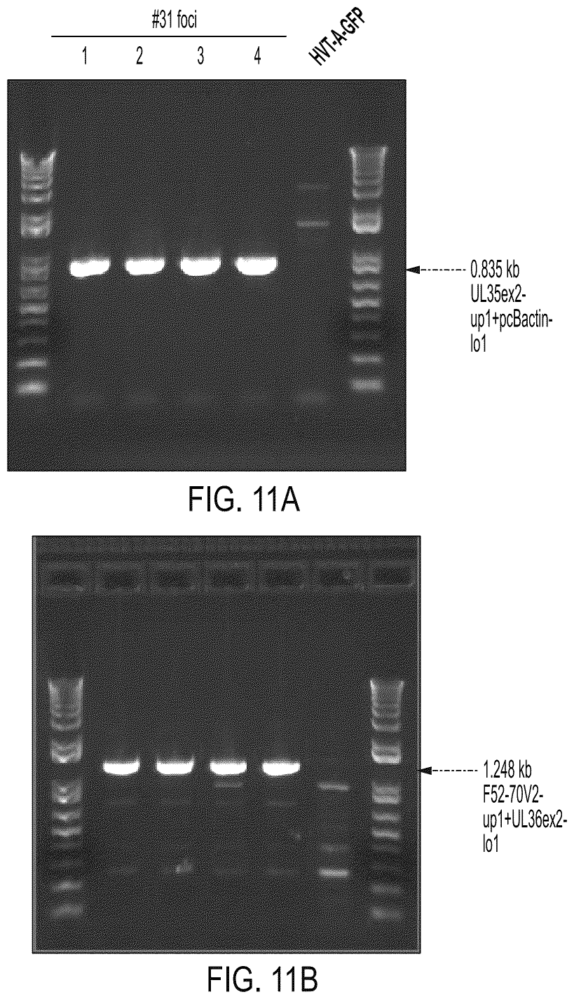

Infected cell lysate was prepared, and Western Blot analysis was performed using monoclonal antibody against IBDV R63. A protein band of about 50 KD was seen in all lanes with anti-IBDV chicken serum as probe only, not with mAb for IBDV R63. Please see FIGS. 10A and B.

PCR analysis of the clones using 2 sets of primers to confirm the correct integration. The first primer set targeting upstream integration site: upper primer SEQ ID NO. 72 that localized upstream of UL35-UL36 integration site; lower primer SEQ ID NO. 73 that localized within the chicken beta-actin promoter. This set of primers gave a PCR band of 0.835 kb as expected. The second primer set targeting the downstream integration site: upper primer SEQ ID NO.76 that localized within the IBDV VP2 coding region; lower primer SEQ ID NO.77 that localized downstream of the UL35-UL36 insertion site. A PCR band of 1.248 kb was obtained as expected. Please refer to FIG. 11A and B.

### Construction of HVT-IBD #34

HVT-IBD #34 transfer plasmid (SEQ ID NO. 28) was chemically synthesized by GeneArt, ThermoFisher. 2.5 ug of the plasmid was transfected into secondary CEF cells using LTX transfection reagent (Invitrogen) in 6-well plate. About 4-6 hours later, the transfected cells were infected with HVT-gfp-B at 0.05 moi. Three days later, the cells were passaged 1:15 to T75 with fresh CEF (1×10⁷ cells/T75). The infected cells were plated onto 10×96 well plates, grown for 3 days, then passage into replicate 96 well plates. One replicate was fixed and stained with anti-IBDV chicken serum and 3 wells containing foci which stained positive for IBDV were identified. The corresponding wells from the live replicate plate were purified by 3 rounds of limiting dilution cloning using 96 well plates. One of the purified viruses was expanded using CEF cells and frozen stock made. It was designated as “HVT-IBD #34”.

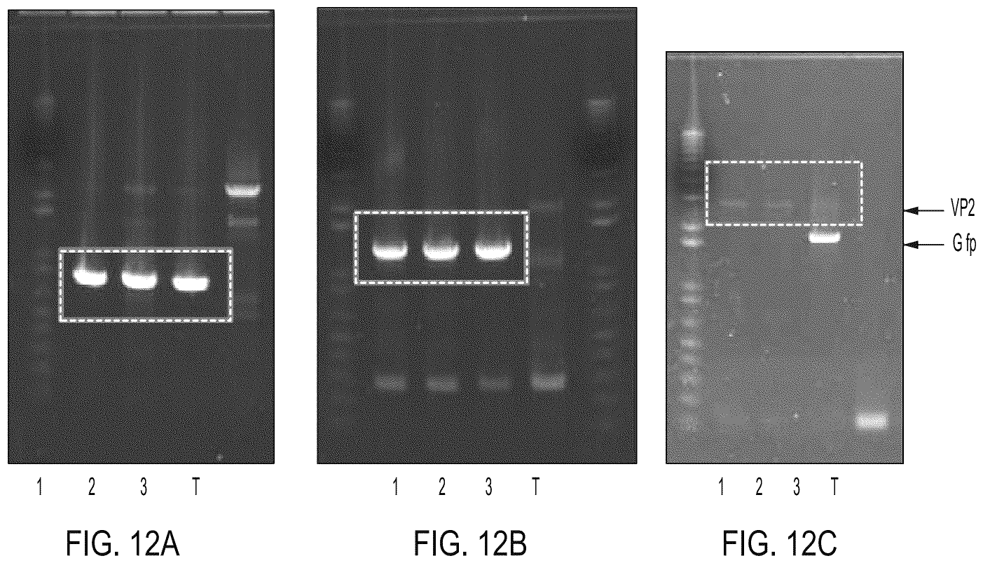

PCR analysis of 3 different clones using primers for upstream region of integration site of Gene3-UL55 (upper primer: SEQ ID NO.78; lower primer: SEQ ID NO.79 that localized within chicken beta-actin promoter, panel A) gave a band of 0.815 kb as expected. The correct integration was further confirmed by using primers surrounding the downstream junction of the insertion (upper primer SEQ ID NO.80 that localized within IBDV VP2 coding region; lower primer SEQ ID NO.81 that localized downstream of Gene3-UL55 insertion site, panel B). A PCR band of 1.296 kb was obtained as expected. The correct construct was further confirmed by using primers outside of the expression cassette (upper primer SEQ ID NO.82; lower primer SEQ ID NO.83 (panel C). A PCR band of 3.001 kb was obtained as expected. Please refer to FIGS. 12 A-C.

### Construction of HVT-ND #38

HVT-IBD #38 transfer plasmid (SEQ ID NO.29) was chemically synthesized by BioBasic, Inc. HindIII and ApoI digested transfer plasmid for HVT-ND #38 was co-transfected with Sbf1 digested HVT-gfp-A DNA using PEI (Polyethylenimine, 7.5 uL) in 6-well plate with secondary CEF cells. 6 days post transfection, the transfected cells were plated onto 96-well plate and live stained with NDV chicken serum. Seven wells containing the foci with positive staining were purified 3 times by limiting dilution. One of the purified viruses was expanded using CEF cells and frozen stock made. It was designated as “HVT-ND #38”.

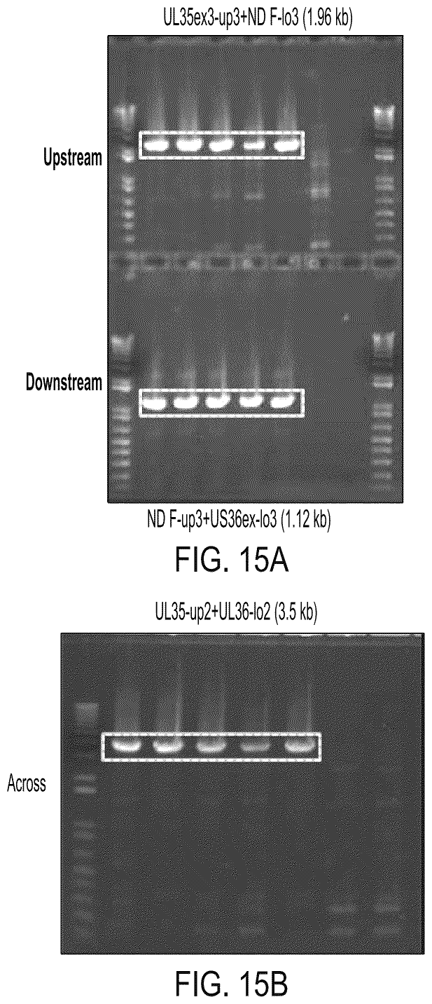

PCR analysis of 5 different clones using primers for upstream region of integration site of UL35-UL36 (upper primer: SEQ ID NO. 84; lower primer: SEQ ID NO.85 that localized within NDV F coding region, panel A) gave a band of 2.122 kb. The correct integration was further confirmed by using primers surrounding the 3′ junction of the insertion (upper primer SEQ ID NO.86 that localized within NDV F coding region; lower primer SEQ ID NO.87 that localized downstream of UL35-UL36 insertion site, panel B). A PCR band of 1.127 kb was obtained as expected. The correct construct was further confirmed by using primers outside of the expression cassette (upper primer SEQ ID NO. 88; lower primer SEQ ID NO.89 (panel C). A PCR band of 3.657 kb was obtained as expected. Please refer to FIGS. 15A and B.

### Construction of HVT-ND #39

HVT-IBD #39 transfer plasmid SEQ ID NO. 30) was chemically synthesized by BioBasic, Inc. Secondary CEF cells were co-transfected with 0.01 ug of the plasmid and 2.5 ug of HVT using PEI (Polyethylenimine) transfection reagent in a 6-well plate. Six days later, the cells were passaged 1:24 onto fresh CEF cells. Three days post passage, foci expressing NDV F protein were visualized by staining unfixed cultures with anti NDV specific chicken polyclonal serum, and these foci marked with the aid of a fluorescent microscope. A total of 4 positive foci were passaged onto fresh CEF cells via trypsinization using cloning cylinders to segregate the foci from non-F protein expressing foci. Four of these cultures were cloned three times following the same procedure before being amplified on primary CEFs in roller bottles. A frozen stock of cells (clone 2 in the figures) was put down and it was designated as “HVT-ND #39”.

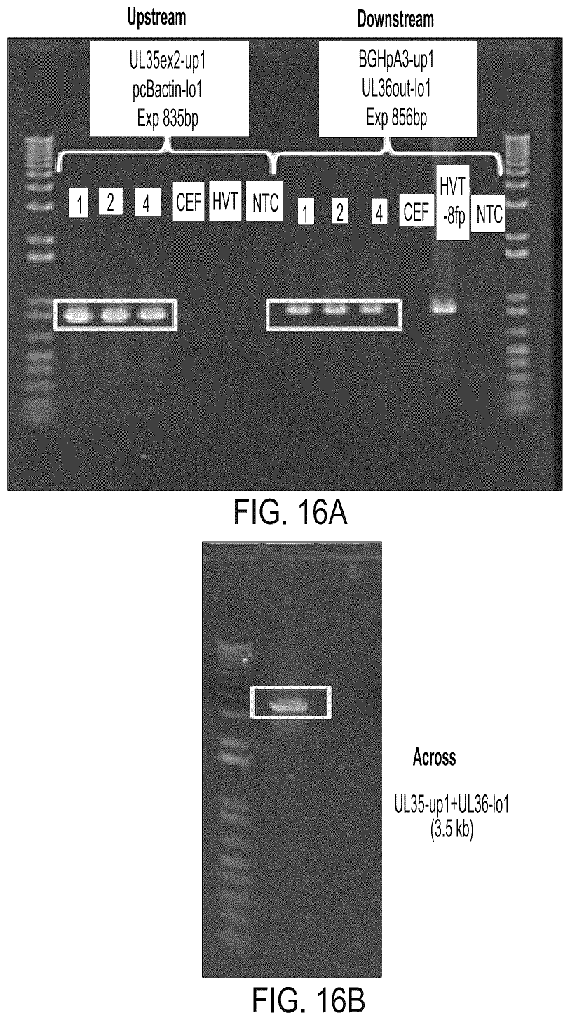

PCR analysis of 3 different clones using primers for upstream region of integration site of UL35-UL36 (upper primer: SEQ ID NO 90; lower primer: SEQ ID NO. 91 that localized within chicken beta-actin promoter, panel A) gave a band of 0.835 kb. The correct integration was further confirmed by using primers surrounding the downstream junction of the insertion (upper primer SEQ ID NO.92 that localized within poly A region; lower primer SEQ ID NO.93 that localized downstream of UL35-UL36 insertion site, panel B). A PCR band of 0.856 kb was obtained as expected. The correct construct was further confirmed by using primers outside of the expression cassette (upper primer SEQ ID NO. 94; lower primer SEQ ID NO. 95 (panel C). A PCR band of 3.449 kb was obtained as expected. Please refer to FIG. 16A and B.

### Construction of HVT-ND #40

HVT-IBD #40 transfer plasmid (SEQ ID NO.31) was chemically synthesized by BioBasic, Inc. HindIII digested transfer plasmid for HVT-ND #40 was co-transfected with Sbf1 digested HVT-gfp-A DNA using PEI (Polyethylenimine, 7.5 uL) in 6-well plate with secondary CEF cells. 7 days post-transfection, the transfected cells were plated onto 24-well plates and live stained with NDV chicken serum Four wells containing the foci with positive staining were purified 3 times by limiting dilution. One of the purified viruses was expanded using CEF cells and frozen stock made. It was designated as “HVT-ND #40”.

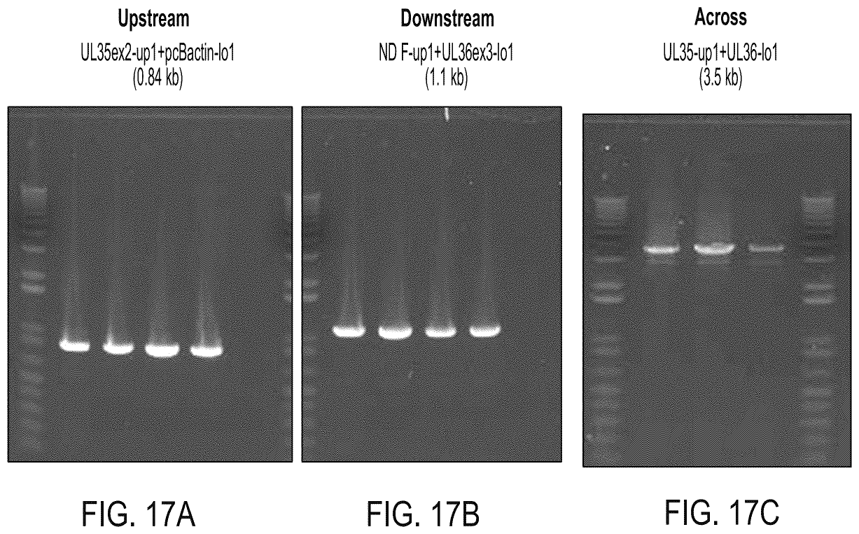

PCR analysis of 4 different clones using primers for upstream region of integration site of UL35-UL36 (upper primer: SEQ ID NO.96; lower primer: SEQ ID NO.97 that localized within chicken beta-actin promoter, panel A) gave a band of 0.835 kb. The correct integration was further confirmed by using primers surrounding the downstream junction of the insertion (upper primer SEQ ID NO.98 that localized within NDV F coding region; lower primer SEQ ID NO. 99 that localized downstream of UL35-UL36 insertion site, panel B). A PCR band of 0.856 kb was obtained as expected. The correct construct was further confirmed by using primers outside of the expression cassette (upper primer SEQ ID NO.100; lower primer SEQ ID NO.101 (panel C). A PCR band of 3.449 kb was obtained as expected. Please refer to FIGS. 17A-C.

### Construction of HVT-ND #42

Initial transfer plasmid HVT-ND #42 (SEQ ID NO. 33) was chemically synthesized by BioBasic, Inc. Cloning plasmid was chemically synthesized by DNA2.0.PCR amplification of NDV F gene expression cassette of HVT-ND #42 transfer plasmid by using the following primers: upper primer, SEQ ID NO.102; lower primer, 5′-SEQ ID NO. 103. The amplified PCR fragment was cloned into the AscI and NheI sites UL55/gene3 to make final transfer plasmid, HVT ND #42 (SEQ ID NO.35)

Transfer plasmid was transfected into CEF cells that was infected with HVT, using Lipofectamine LTX in 6-well plate. 3 days post transfection, the transfected/infected cells were plated onto duplicate 6 well plate and then 96-well plate for screening ND expressing foci by staining with NDV antiserum. Wells corresponding to containing the ND expression foci were purified 3 times by limiting dilution. One of the purified viruses was expanded using CEF cells and frozen stock made. It was designated as “HVT-ND #42”.

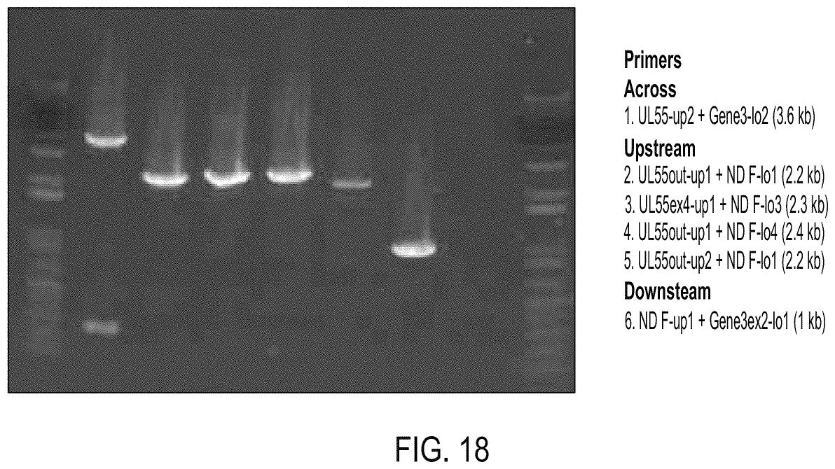

PCR analysis of one final clone using primers outside of the expression cassette (primer set 1: upper primer SEQ ID NO. 104; lower primer SEQ ID NO. 105) gave a band of 3.597 kb as expected. Four sets of primers for upstream integration region of UL55-Gene 3 with all upper primers located upstream and outside of the expression cassette, all lower primers located within ND F coding region. The primer set 2: upper primer: SEQ ID NO. 106, lower primer: SEQ ID NO. 107 that gave a band of 2.243 kb; The primer set 3: upper primer: SEQ ID NO.108; lower primer: SEQ ID NO. 109 that gave a PCR band of 2.356 kb. The primer set 4: upper primer: SEQ ID NO. 110; lower primer: SEQ ID NO. 111 that gave a PCR band of 2.424 kb. The primer set 5: upper primer: SEQ ID NO. 112; lower primer: SEQ ID NO. 113 that gave a PCR band of 2.170 kb. The correct integration was further confirmed by using primers surrounding the downstream junction of the insertion (primer set 6: upper primer SEQ ID NO. 114 that localized within NDV F gene coding sequence; lower primer SEQ ID NO. 115 that localized downstream of UL55-Gene3 insertion site, panel C). A PCR band of 0.971 kb was obtained as expected. Please refer to FIG. 18.

### Construction of HVT-ND #44

HVT-IBD #44 transfer plasmid (SEQ ID NO.36) was chemically synthesized by BioBasic, Inc. Transfer plasmid number 44 was digested with restriction enzymes EcoRI and HindIII to release the insert from plasmid sequences and the resultant digested DNA (10 ng) was used along with 2.5 μg of HVT-gfpB DNA to co-transfect secondary cells in using PEI (polyethylenimine). Four days post transfection the transfected cells were passed 1:6 with fresh secondary cells and live stained with chicken anti NDV polyclonal serum to identify NDV expressing foci three to four days post passage. Three positively staining foci were harvested via trypsinization using cloning. The harvested cells were serially diluted and plated on fresh secondary CEF cells. This process was repeated every three to four days until the NDV staining showed homogeneity, and then four subsequent clonings were performed. The cloned culture was then amplified, and frozen stock prepared. The frozen stock was designated as “HVT-ND #44”.

PCR analysis of 1 final clone using primers for upstream region of integration site of UL55-Gene 3 (upper primer: SEQ ID NO. 116 that localized upstream of UL55; lower primer: SEQ ID NO. 117 that localized within chicken beta-actin promoter, panel A) gave a band of 0.71 kb; A similarly localized primer pair: upper primer: SEQ ID NO. 118 lower primer: SEQ ID NO. 119 (Panel B that gave a PCR band of 0.965 kb The correct integration was further confirmed by using primers surrounding the downstream junction of the insertion (upper primer SEQ ID NO. 120 that localized within NDV F gene coding sequence; lower primer SEQ ID NO. 121 that localized downstream of UL55-Gene3 insertion site, panel C). A PCR band of 0.971 kb was obtained as expected. The correct construct was further confirmed by using primers outside of the expression cassette (upper primer SEQ ID NO.122; lower primer SEQ ID NO.123 (panel D). A PCR band of 3.438 kb was obtained as expected.

### Construction of HVT-ND #45

HVT-IBD #45 transfer plasmid (SEQ ID) was chemically synthesized by BioBasic, Inc. This plasmid was transfected into CEF cells that was infected with HVT-GFP-B, using Lipofectamine LTX in 6-well plate. 3 days post transfection, the transfected/infected cells were plated onto 96-well plate for screening of GFP negative foci. Wells containing the GFP negative foci were purified 3 times by limiting dilution. The purified viruses were IFA stained with chicken NDV serum to confirm the NDV F gene expression. One of the purified viruses was expanded using CEF cells and frozen stock made. It was designated as “HVT-ND #45”.

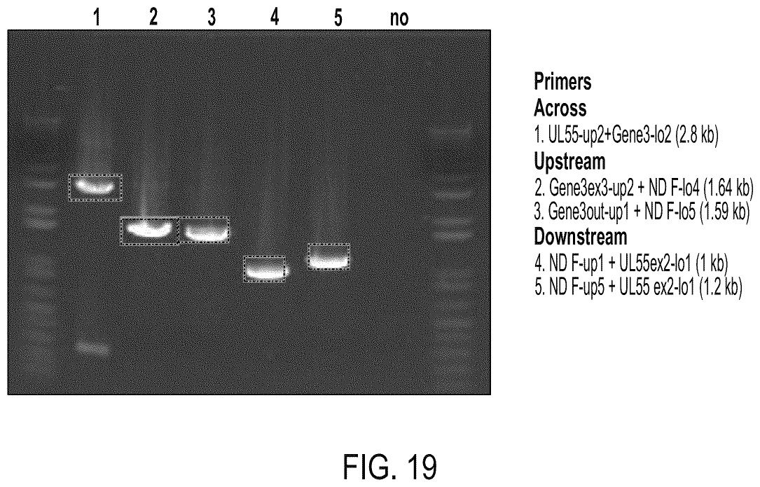

PCR analysis of 1 final clone using primers outside of the expression cassette (primer set 1: upper primer SEQ ID NO.124; lower primer SEQ ID NO.125) gave a band of 2.830 kb as expected. Two sets of primers for upstream integration region of Gene 3-UL55 with both upper primers located upstream and outside of the expression cassette, both lower primers located within ND F coding region. The primer set 2: upper primer: SEQ ID NO.126, lower primer: SEQ ID NO. 127 that gave a band of 1.635 kb; The primer set 3: upper primer SEQ ID NO.128; lower primer: SEQ ID NO. 129 that gave a PCR band of 1.588 kb. The correct integration was further confirmed by using 2 sets of primers surrounding the downstream junction of the insertion: primer set 4: upper primer SEQ ID NO. 130 that localized within NDV F gene coding sequence; lower primer SEQ ID NO. 131 that localized downstream of Gene3-UL55 insertion site. A PCR band of 0.993 kb was obtained as expected. Primer set 5: upper primer: SEQ ID NO.132, lower primer: SEQ ID NO.133, a PCR band of 1.137 kb was obtained as expected. Please refer to FIG. 19.

### Construction of HVT-ND #46

HVT-ND #46 transfer plasmid (SEQ ID NO.38) was chemically synthesized by BioBasic, Inc. This plasmid was transfected together with HVT-GFP-B viral DNA into CEF cells using PEI (Polyethylenimine, 7.5 uL) in 6-well plate. 4 days post transfection, the transfected cells were plated onto 96-well plate for screening of GFP negative foci. Wells containing the GFP negative foci were purified 3 times by limiting dilution. The purified viruses were IFA stained with chicken NDV serum to confirm the NDV F gene expression. One of the purified viruses was expanded using CEF cells and frozen stock made. It was designated as “HVT-ND #46”.

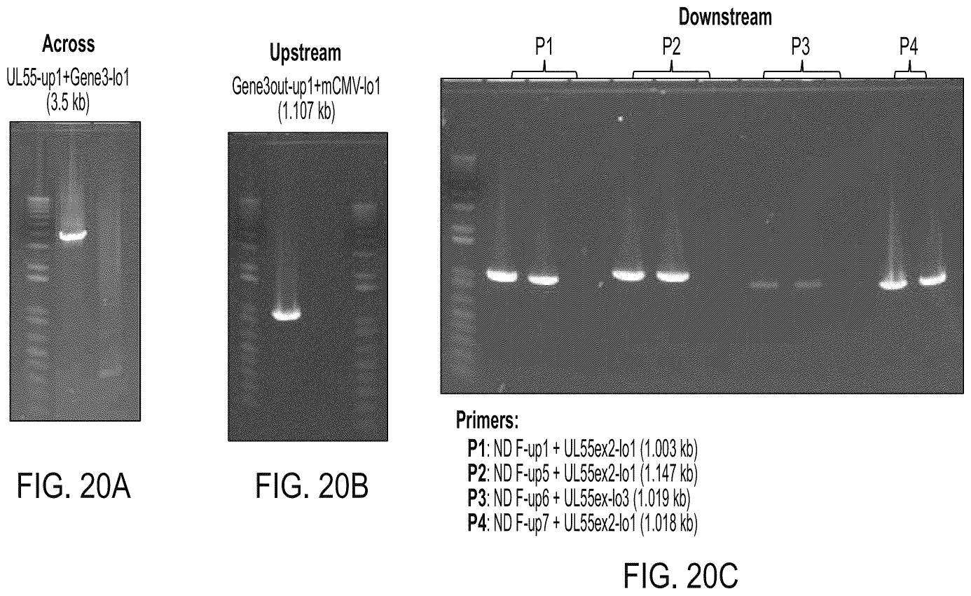

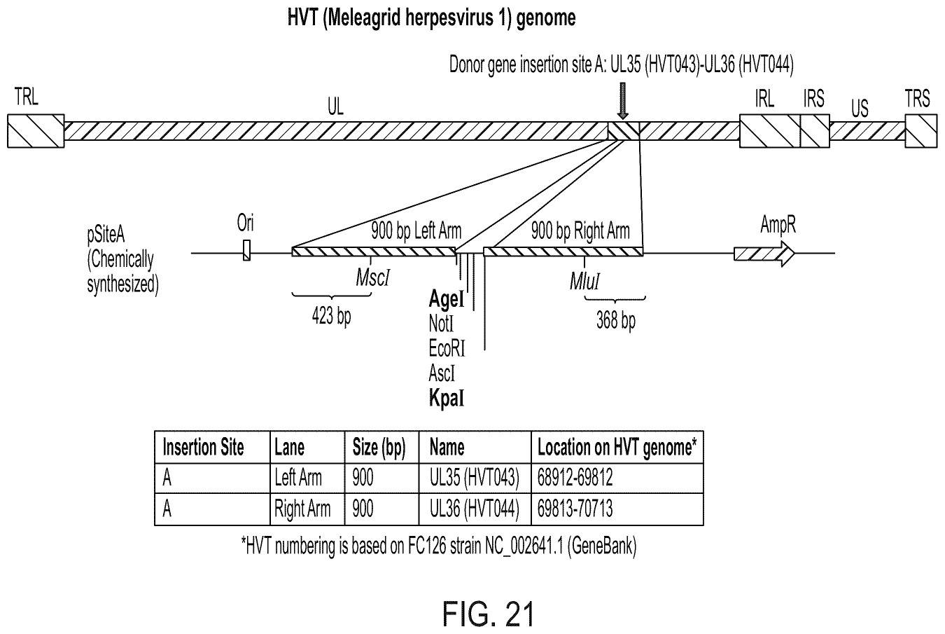

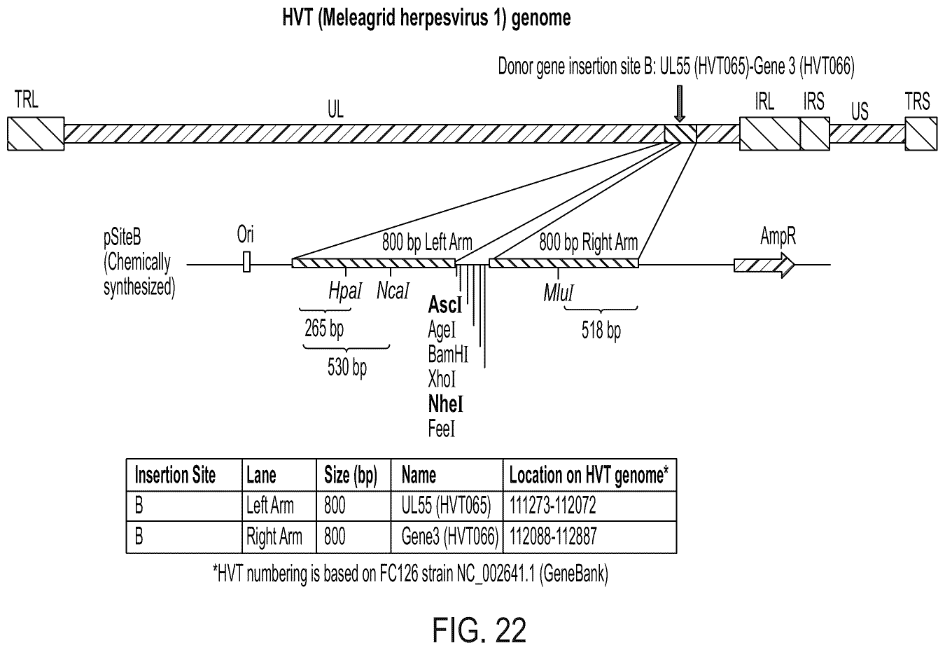

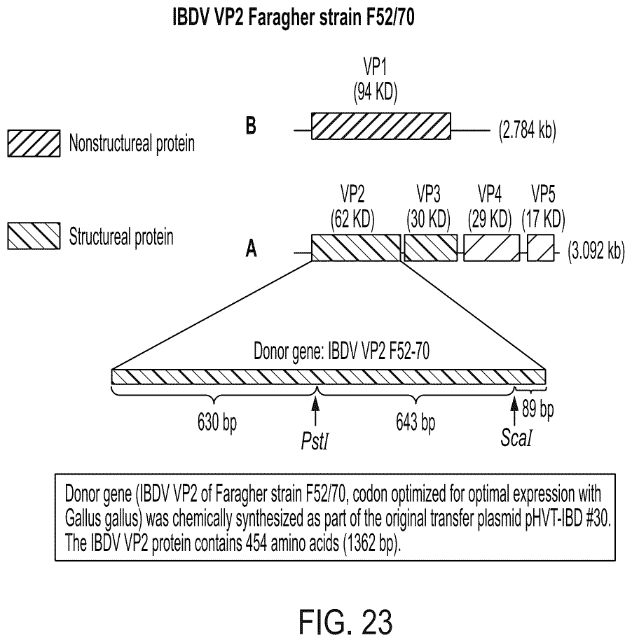

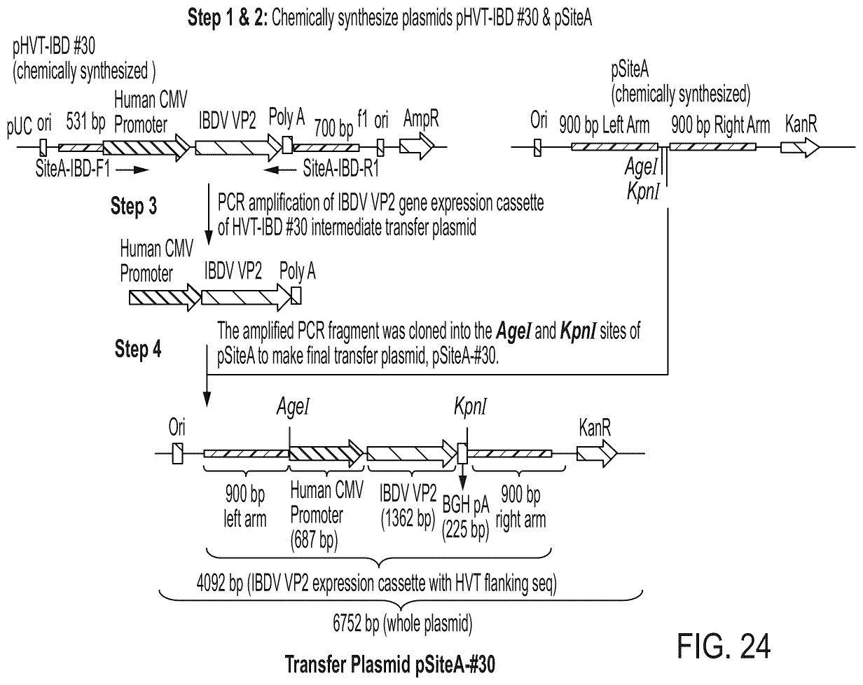

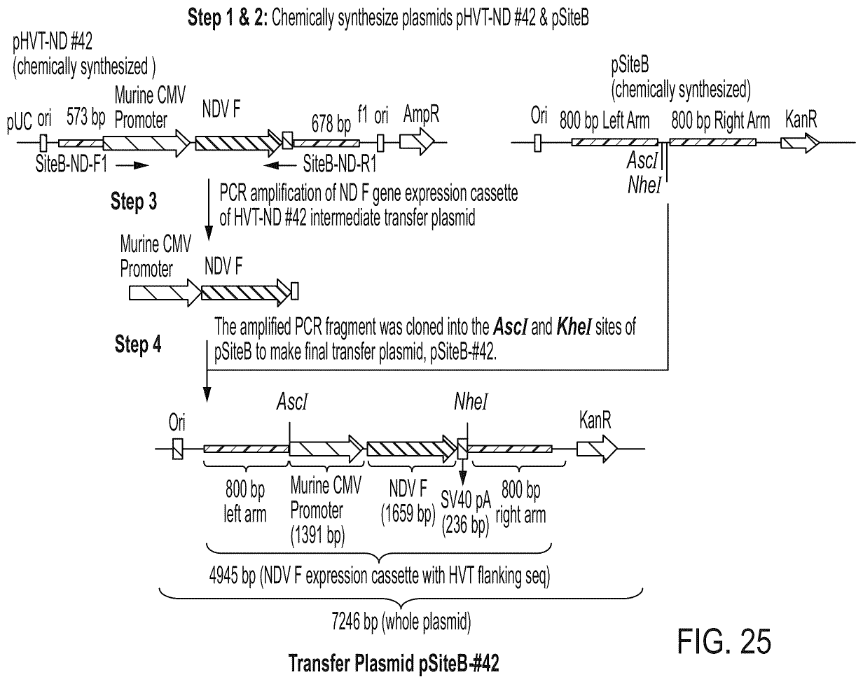

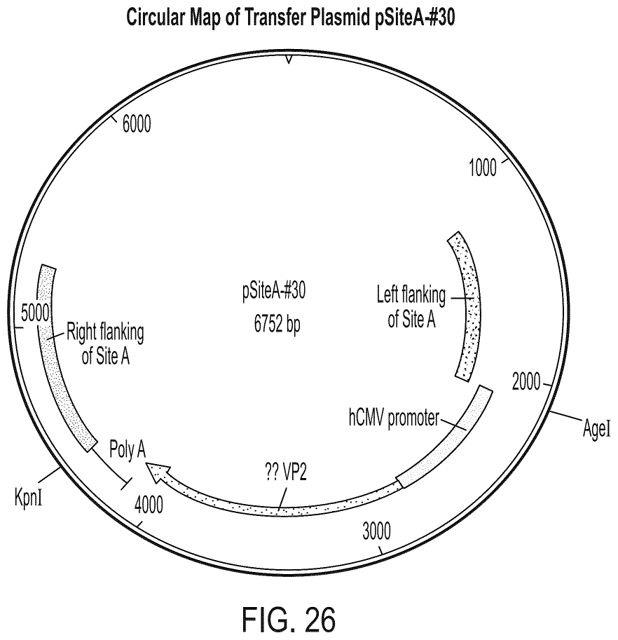

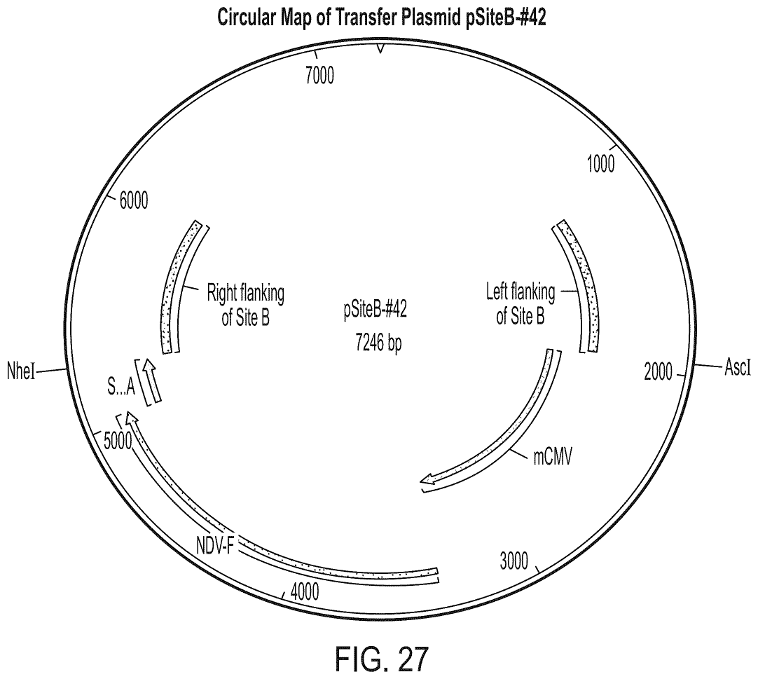

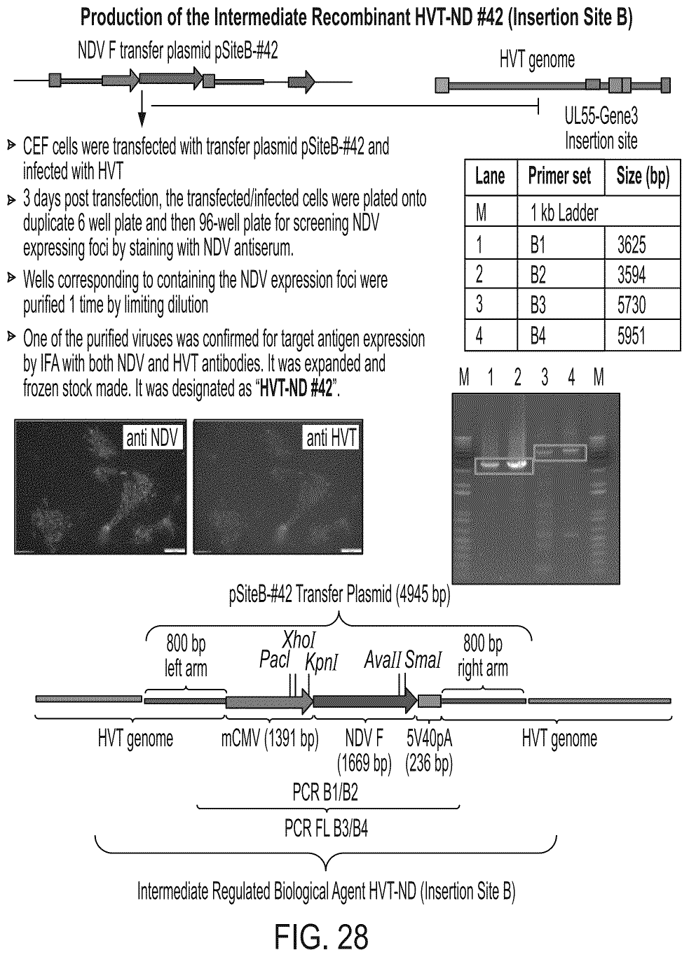

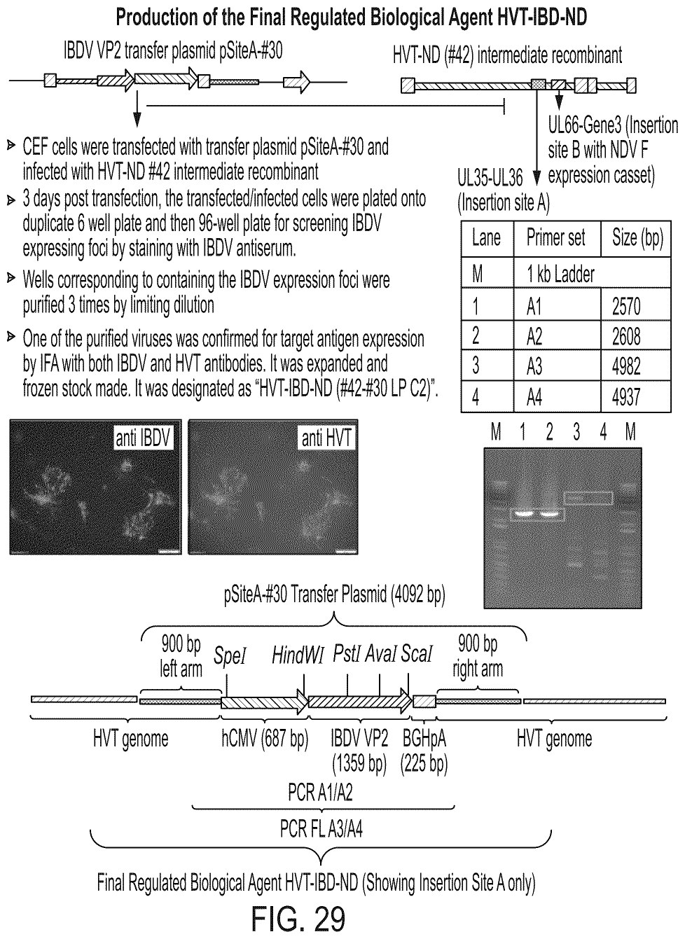

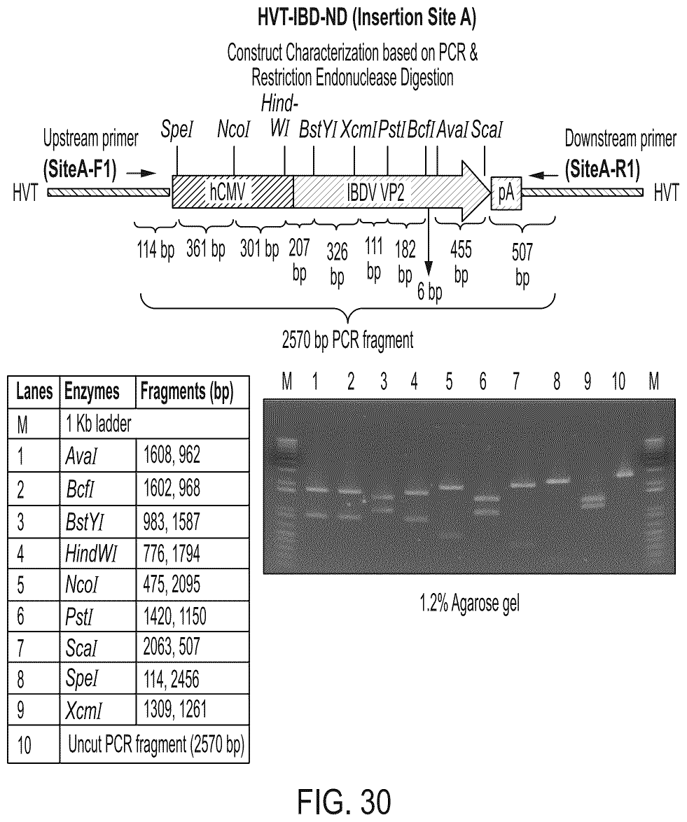

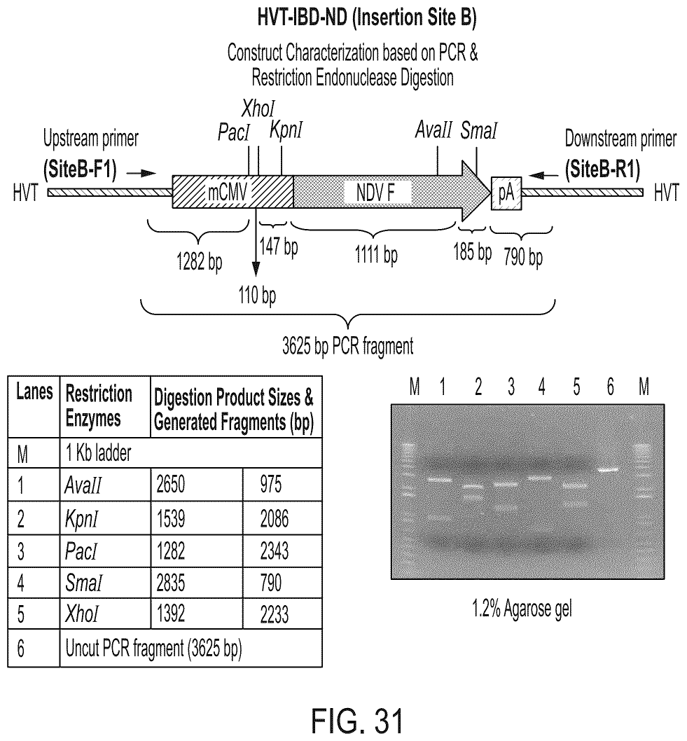

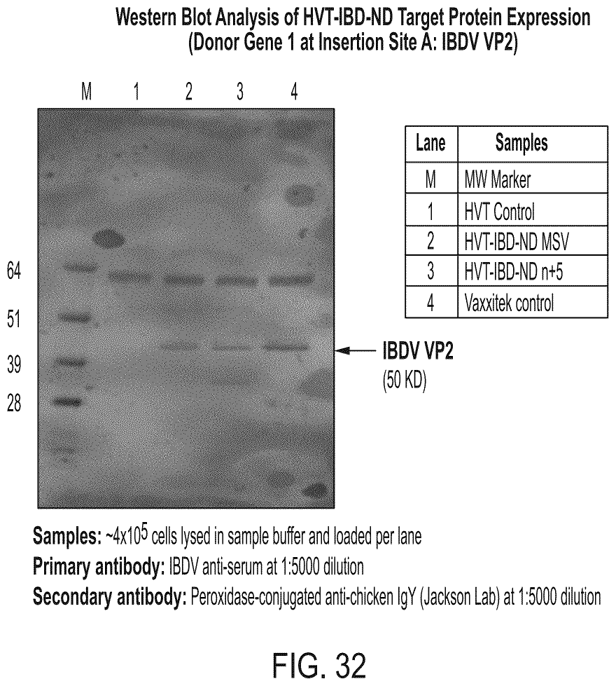

PCR analysis of 1 final clone using primers outside of the expression cassette (upper primer SEQ ID NO. 134; lower primer SEQ ID NO. 135) gave a band of 3.597 kb as expected. One sets of primers for upstream integration region of Gene 3-UL55 with upper primers located upstream and outside of the expression cassette (SEQ ID NO. 136), lower primers located within the murine CMV promoter (upper primer SEQ ID NO. 137) that gave a PCR band of 1.107 kb as expected. The correct integration was further confirmed by using 4 sets of primers surrounding the downstream junction of the insertion: P1: upper primer SEQ ID NO. 138 that localized within NDV F gene coding sequence; lower primer SEQ ID NO. 139 that localized downstream and outside of expression cassette. A PCR band of 1.003 kb was obtained as expected. P2: upper primer: SEQ ID NO. 140, lower primer: SEQ ID NO. 141, a PCR band of 1.147 kb was obtained as expected. P3: upper primer: SEQ ID NO. 142, lower primer: SEQ ID NO. 143, a PCR band of 1.019 kb was obtained as expected. P4: upper primer: SEQ ID NO. 144, lower primer: SEQ ID NO. 145, a PCR band of 1.018 kb was obtained as expected. Please refer to FIGS. 20A-C.

### Construction of HVT-ND #48

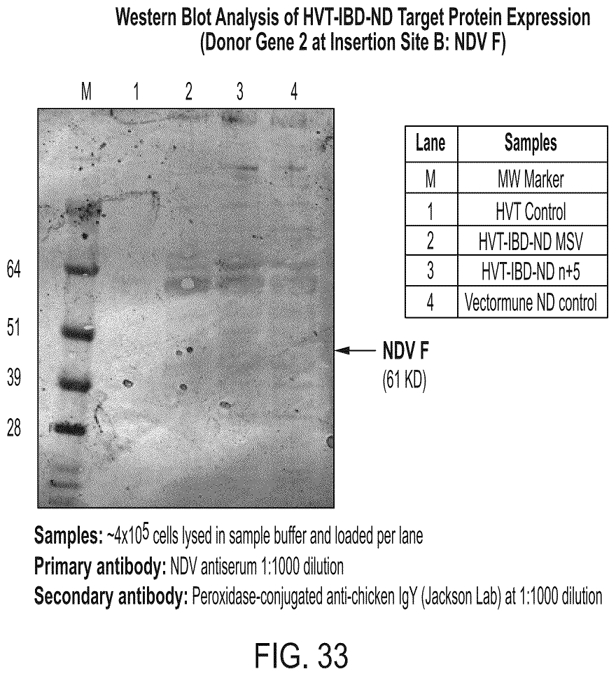

Linearized transfer plasmid for HVT-ND #48 (SEQ ID NO.39) was co-transfected with HVT-gfp-B DNA using PEI (Polyethylenimine, 7.5 uL) in 6-well plate with secondary CEF cells. 4 days post transfection, the transfected cells were plated onto 96-well plate and live stained with NDV chicken serum. Four non-green foci were found and 2 were stained positive with positive using NDV chicken serum. Please refer to FIG. 33A and B. The two clones were purified 3 times by limiting dilution. The purified virus was expanded using CEF cells and frozen stock made. It was designated as “HVT-ND #48”.

HVT-IBD #48 transfer plasmid was chemically synthesized by BioBasic, Inc. The transfer plasmid was digested with EcoRI and HindIII to release the insert from plasmid sequences and the resultant digested DNA (10 ng) was used along with 2.5 μg of HVT-gfp-B DNA to co-transfect secondary cells in using PEI (polyethylenimine). Three days post transfection the transfected cells were passed 1:6 with fresh secondary cells and live stained with NDV chicken polyclonal serum to identify NDV expressing foci four days post passage. Three positively staining foci were harvested via trypsinization using cloning cylinders. The harvested cells were serially diluted and plated on fresh secondary CEF cells. This process was repeated every three to four days until the NDV staining showed homogeneity, and then four subsequent cloning were performed. The cloned culture was then amplified from six well plate to 75 cm2 flask, to 225 cm2 flask before a final amplification in an 850 cm2 roller bottle using primary CEF cells. The final culture was harvested and designated “HVT-ND #48”.

PCR analysis of 1 final clone using primers for upstream region of integration site of Gene 3-UL55 (upper primer: SEQ ID NO.146; lower primer: SEQ ID NO.147 that localized within chicken beta-actin promoter, panel A) gave a band of 0.815 kb as expected; The correct integration was further confirmed by using primers surrounding the downstream junction of the insertion (upper primer SEQ ID NO. 148 that localized within NDV F coding region; lower primer SEQ ID NO.149 that localized downstream of Gene 3-UL55 insertion site, panel B). A PCR band of 1.003 kb was obtained as expected. Another similarly localized primers: upper primer: SEQ ID NO.150; lower primer: SEQ ID NO. 151 (Panel C) that gave a PCR band of 1.147 kb band as expected. The correct construct was further confirmed by using primers outside of the expression cassette (upper primer SEQ ID NO. 152; lower primer SEQ ID NO.153 (panel D). A PCR band of 3.430 kb was obtained as expected.

### Example 3

### In Vivo IBDV Efficacy Test of HVT-IBD #9, #34 of SFP Birds

Two HVT-IBD recombinants, HVT-IBD #9, #34 were tested for their in vivo efficacy against virulent IBDV challenge (STD strain, provided by USDA) in SPF birds. A positive control of a commercial vaccine Vaxxitek (Merial) was used in this study. 1500 pfu of each recombinant virus was injected in ovo at E18. The back-titer of each vaccine virus was also determined for each recombinant after vaccination. While 100% of HVT-IBD #9 express IBDV VP2 antigen, we found only 96% of HVT-IBD #34 express the antigen. IBDV STD challenge was carried out at Day 28 per USDA instruction. All birds were necropsied at 5 days post challenge. We observed 100% protection for HVT-IBD #9, and 90% protection for HVT-IBD #34, while our positive control Vaxxitek gave 97% protection.

TABLE 1
IBDV Efficacy Test in SPF Birds

| | | Dose (Pfu) | | | | Prevented |
|---|---|---|---|---|---|---|
| | | Target/ | | #Edema/ | | Fraction |
| Group | Treatment | Backtiter | Route | #challenged | | (PF) |
| T01 | None | — | — | 0/29 | (0%) | — |
| T02 | None | — | — | 22/22 | (100%) | — |
| T03 | Vaxxitek | 1500/1053 | In ovo | 1/30 | (3%) | 97% |
| T04 | HVT-IBD | 1500/1322 | In ovo | 0/30 | (0%) | 100% |
| | #9 | | | | | |
| T06 | HVT-IBD | 1500/1353* | In ovo | 3/29 | (10%) | 90% |
| | #34 | | | | | |

### Example 4

### In Vivo IBDV Efficacy Test of HVT-IBD #1, #5, #6a, #9, #30, #34 of SFP Birds

Six HVT-IBD recombinants, HVT-IBD #1, #5, #6a, #9, #30, #34 were tested for their in vivo efficacy against virulent IBDV challenge (STD strain, provided by USDA) in SPF birds. A positive control of a commercial vaccine Vaxxitek (Merial) was used in this study. 1500 pfu of each recombinant virus was injected in ovo at E18. The back-titer of each vaccine virus was also determined for each recombinant after vaccination. All recombinants were found to have 100% expression for IBDV VP2 antigen. IBDV STD challenge was carried out at Day 28 per USDA instruction. All birds were necropsied at 5 days post challenge. We observed 100% protection for HVT-IBD #9, and 96% protection for HVT-IBD #1, #30, #34, and 92% protection for HVT-IBD #6a, while our positive control Vaxxitek gave 92% protection.

TABLE 2
IBDV Efficacy Test in SPF Birds

| Treatment Description | | | | | | | | | |
|---|---|---|---|---|---|---|---|---|---|
| | | Dose (pfu) | | | | | % susceptible | | Prevented |
| | | target/ | Necropsy results | | | | (lesions + | | Fraction |
| Trt | Vaccine | backtiter | % Normal (#) | | % Edema (#) | | mortality) | | (PF) |
| T01 | None | NA | 100.0 | (24/24) | 0 | | 0 | | |
| T02 | None | NA | 0 | | 100.0 | (16/16) | 100.0 | (24/24) | 0 |
| T03 | Vaxxitek | 1500/1459 | 95.7 | (22/23) | 4.3 | (1/23) | 8.3 | (2/24) | 92 |
| T04 | HVT-IBD #9 | 1500/1519 | 100.0 | (24/24) | 0 | | 0 | | 100 |
| T06 | HVT-IBD #1 | 1500/1319 | 100.0 | (23/23) | 0 | | 4.2 | (1/24) | 96 |
| T07 | HVT-IBD #5 | 1500/1537 | 72.7 | (16/22) | 27.3 | (6/22) | 33.3 | (8/24) | 67 |
| T08 | HVT-IBD #6a | 1500/1105 | 91.7 | (22/24) | 8.3 | (2/24) | 8.3 | (2/24) | 92 |
| T09 | HVT-IBD #30 | 1500/1324 | 100.0 | (23/23) | 0 | | 4.2 | (1/24) | 96 |
| T10 | HVT-IBD #31 | 1500/1255 | 95.8 | (23/24) | 4.2 | (1/24) | 4.2 | (1/24) | 96 |

### Example 5

### IBDV Serology Responses of HVT-IBD #1, #5, #9, #15 of Commercial Broiler Birds

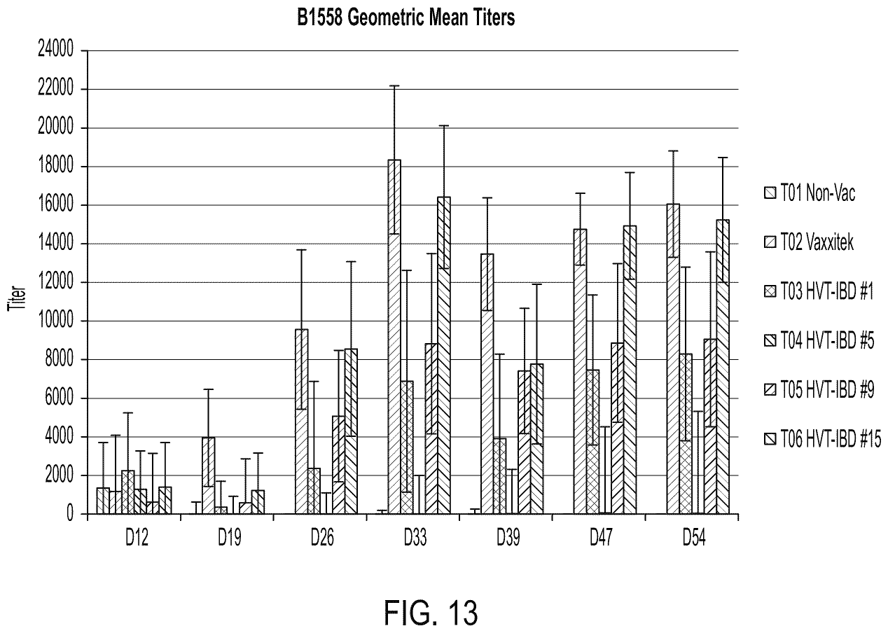

The serology responses against IBDV antigen was measured by using a commercial Elisa kit ProFlok ND plus. 1500 pfu (0.2 mL) of each recombinant (HVT-IBD #1, #5, #9, #15 were injected Subcutaneously (SC) for 1-day old chicks. Serum samples were isolated on days 12, 19, 26, 33, 39, 47 and 54 and can be seen in Table 3 below. The percentage of positive sample for each construct during the time course are shown in FIG. 13.

TABLE 3

| | | IBDV Titers (% Pos.) | | | | | | | |
|---|---|---|---|---|---|---|---|---|---|
| Trt | Vaccines | D0 | D12 | D19 | D26 | D33 | D39 | D47 | D54 |

| | | | | | | | | |
|---|---|---|---|---|---|---|---|---|
| T01 | Control | 90 | 20 | 0 | 3 | 3 | 0 | 0 |
| T03 | HVT-IBD #1 | 95 | 78 | 90 | 95 | 90 | 95 | 95 |
| T04 | HVT-IBD #5 | 90 | 35 | 8 | 20 | 43 | 50 | 43 |
| T05 | HVT-IBD #9 | 80 | 80 | 95 | 95 | 95 | 95 | 95 |
| T06 | HVT-IBD #15 | 90 | 90 | 95 | 95 | 97 | 100 | 100 |

### Example 6

### IBDV Serology Responses of HVT-IBD #6a, #30, #31 of Commercial Broiler Birds

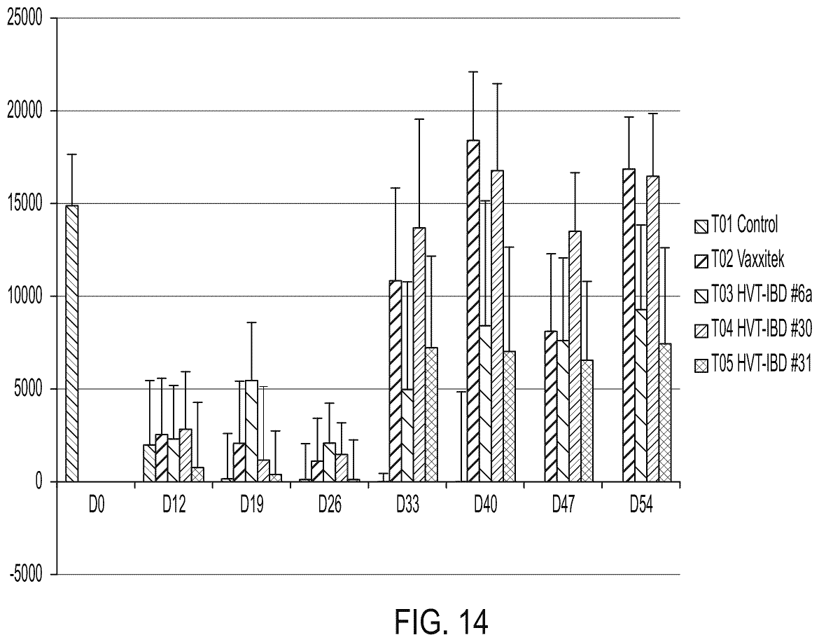

The serology responses against IBDV antigen was measured by using a commercial ELISA kit ProFlok IBD plus. 1500 pfu (0.2 mL) of each recombinant (HVT-IBD #61, #30, #31) were injected Subcutaneously (SC) for 1-day old chicks. Serum samples were isolated on days 12, 19, 26, 33, 39, 47 and 54 are shown in Table 4 below. The percentage of positive samples for each construct during the time course are shown in FIG. 14.

TABLE 4

| | B1583 | IBDV Titers (Geomean) | | | | | | | |
|---|---|---|---|---|---|---|---|---|---|
| Results | Vaccines | D0 | D12 | D19 | D26 | D33 | D40 | D47 | D54 |
| T01 | Control | 14883 | 1984 | 162 | 125 | 2 | 3 | 0 | 0 |
| T03 | HVT-IBD #6a | | 2305 | 5457 | 2083 | 4970 | 8414 | 7616 | 9281 |
| T04 | HVT-IBD #30 | | 2828 | 1171 | 1471 | 13686 | 16773 | 13505 | 16476 |
| T05 | HVT-IBD #31 | | 766 | 388 | 122 | 7227 | 7027 | 6553 | 7439 |

### Example 7

### In Vivo Efficacy Test of HVT-ND #38, #39, #44, #48 in SFP Birds

Four HVT-ND recombinants, HVT-IBD #38, #39, #44, #48 were tested for their in vivo efficacy against virulent NDV challenge (Texas GB strain, provided by USDA) in SPF birds. A positive control of a commercial vaccine Vectormune ND (Ceva) was used in this study. 1500 pfu of each recombinant virus was injected in ovo at E18. The back-titer of vaccine virus was also determined for each recombinant after vaccination. While 100% of HVT-ND #38 & #48 express NDV F antigen, we found only 95-96% of HVT-IBD #39 & #44 express the antigen. NDV Texas GB challenge was carried out at Day 28 per USDA instruction. All birds were observed for 2 weeks post challenge. We observed 90% protection for HVT-ND #38 and #48, and 50% protection for HVT-ND #39 and 60% protection for HVT-ND #44, while our positive control Vectormune ND gave 90% protection. Please refer to Table 5 below.

TABLE 5
NDV Efficacy Test in SPF Birds

| | | Dose (pfu) | % | % | | Stability |
|---|---|---|---|---|---|---|
| | | Target/ | Protected | Affected | | IFA by |
| Trt | Description | back-titer | (alive) | (mortality) | | Titration |
| T01 | Negative | — | NA | NA | | NA |
| T02 | Challenge control | — | 0 | 100 | (20/20) | NA |
| T03 | Vectormune ND | 1500/1427 | 90 | 10 | (2/20) | 100% |
| T06 | HVT-ND #38 | 1500/1215 | 90 | 10 | (2/20) | 100% |
| T07 | HVT-ND #39 | 1500/1637 | 50 | 50 | (10/20) | 95% |
| T08 | HVT-ND #44 | 1500/1377 | 60 | 40 | (8/20) | 96% |
| T09 | HVT-ND #48 | 1500/1328 | 90 | 10 | (2/20) | 100% |

The antibody response to various HVT-ND vaccine candidates were assayed by using ProFlok ND plus kit (Zoetis LLC). All titers were included without using the cut-off value (345) recommended by the kit. The percentage of birds with positive ND titer is shown in Table 6 below.

TABLE 6

| | | % Pos NDV ELISA Titer (GMT) | | | | | | | |
|---|---|---|---|---|---|---|---|---|---|
| Trt | Description | D 11 | | D 14 | | D 21 | | D 27 | |
| T01 | Negative | 0 | (0.4) | 0 | (0) | 0 | (0) | 0 | (0) |
| T02 | Challenge control | 0 | (0) | 0 | (0) | 0 | (0) | 0 | (0) |
| T06 | HVT-ND #38 | 0 | (0) | 44 | (6) | 72 | (21) | 65 | (22) |
| T07 | HVT-ND #39 | 0 | (0) | 13 | (0.5) | 30 | (2) | 33 | (3) |
| T08 | HVT-ND #44 | 0 | (0) | 31 | (2) | 41 | (8) | 45 | (11) |
| T09 | HVT-ND #48 | 33 | (4) | 42 | (5) | 80 | (51) | 91 | (104) |

### Example 8

### In Vivo NDV Efficacy Test of HVT-ND #40, #42, #45, #46 in SFP Birds

Four HVT-ND recombinants, HVT-IBD #40, #42, #45, #46 were tested for their in vivo efficacy against virulent NDV challenge (Texas GB strain, provided by USDA) in SPF birds. A positive control of a commercial vaccine Vectormune ND (Ceva) was used in this study. 1500 pfu of each recombinant virus was injected in ovo at E18. The back-titer of vaccine virus was determined for each recombinant after vaccination. While 100% of HVT-ND #42, #45, #46 express NDV F antigen, we found only 94-99% of HVT-IBD #40 express the antigen. NDV Texas GB challenge was carried out at Day 28 per USDA instruction. All birds were observed for 2 weeks post challenge. We observed 95% protection for HVT-ND #42 and #45, and 80% protection for HVT-ND #46, 55% protection for HVT-ND #40, while the positive control Vectormune ND gave 90% protection. Please see Table 7 below.

TABLE 7
NDV Efficacy in SPF Birds

| | | Dose (pfu) | | % | | Stability | |
|---|---|---|---|---|---|---|---|
| | | Target/ | % | Affected | | IFA by | |
| Trt | Description | Backtiter | Protected | (Mortality) | | Titration | |
| T01 | Negative | — | NA | 0 | (0/20) | | |
| T02 | Challenge control | — | 0 | 100 | (20/20) | | |
| T03 | Vectormune ND | 1500/1220 | 90 | 10 | (2/20) | | |
| T08 | HVT-ND #40 | 1500/1230 | 55 | 45 | (9/20) | 94-99% | ND |
| T09 | HVT-ND #42 | 1500/1870 | 95 | 5 | (1/20) | 100% | ND |
| T10 | HVT-ND #45 | 1500/1230 | 95 | 5 | (1/20) | 100% | ND |
| T11 | HVT-ND #46 | 1500/1820 | 80 | 20 | (4/20) | 100% | ND |

The antibody response to various HVT-ND vaccine candidates were assayed by using ProFlok ND plus kit (Zoetis LLC). All titers were included without using the cut-off value (345) recommended by the kit. The percentage of birds with positive ND titers is shown in the Table 8 below.

TABLE 8

| | | % Pos NDV ELISA Titer (GMT) | | | | | | |
|---|---|---|---|---|---|---|---|---|
| | | D 10 | D 13 | | D 21 | | D 27 | |
| T01 | Negative | — | — | | — | | — | |
| T02 | Challenge control | 0 (0) | 0 | (0) | 0 | (0) | 2 | (0.1) |
| T08 | HVT-ND #40 | 0 (0) | 15 | (0.9) | 26 | (4) | 31 | (4) |
| T09 | HVT-ND #42 | 0 (0) | 17 | (1.4) | 81 | (108) | 80 | (128) |
| T10 | HVT-ND #45 | 0 (0) | 0 | (0) | 42 | (5) | 79 | (50) |
| T11 | HVT-ND #46 | 0 (0) | 31 | (4) | 80 | (493) | 75 | (68) |

### Example 9

### In Vivo MDV Efficacy Test of HVT-ND #38, #42, #45 in SFP Birds

Three HVT-ND recombinants, HVT-IBD #38, #42, #45 were tested for their in vivo efficacy against virulent MDV challenge (GA22) in SPF birds. A positive control of a commercial vaccine Vectormune ND (Ceva) was used in this study. 1500 pfu of each recombinant virus was injected in ovo at E18. The back-titer of vaccine virus was determined for each recombinant after vaccination. MDV GA22 challenge was carried out at Day 5″ per USDA instruction. All birds were observed for 54 days post challenge. We observed 69% protection for HVT-ND #42 and #45, and 46% protection for HVT-ND #38, while the positive control Vectormune ND gave 62% protection. Please see Table 9 below.

TABLE 9
MDV Efficacy in SPF Birds

| | | PFU/dose | | | Prevented |
|---|---|---|---|---|---|
| | | (Target/ | # MD positive/ | | Fraction |
| Groups | Vaccine | Back-titer) | # challenged | | (PF %) |
| T01 | Control | — | 1/15 | (7%) | |
| T02 | Challenge control | — | 13/15 | (87%) | 0 |
| T03 | Vetormune ND | 1500/2024 | 5/15 | (33%) | 62 |
| T04 | HVT-ND #38 | 1500/1540 | 7/15 | (47%) | 46 |
| T05 | HVT-ND #42 | 1500/1689 | 4/15 | (27%) | 69 |
| T06 | HVT-ND #45 | 1500/1435 | 4/15 | (27%) | 69 |

### Example 10

### In Vivo ND Efficacy Test of HVT-ND #38, #42, #45 in Broiler Birds

Three HVT-ND recombinants, HVT-IBD #38, #42, #45 were tested for their in vivo efficacy against virulent NDV challenge (Texas GB strain, provided by USDA) in broiler birds. A positive control of a commercial vaccine Vectormune ND (Ceva) was used in this study. 4000 pfu of each recombinant virus were injected in ovo at E18. The back-titer of vaccine virus was determined for each recombinant after vaccination. NDV Texas GB challenge was carried out at Day 28 per USDA instruction. All birds were observed for 2 weeks post challenge. We observed 100% protection for HVT-ND #42 and #45, and 37% protection for HVT-ND #38, while our positive control Vectormune ND gave 50% protection. The back-titer of Vectormune ND was 0.

TABLE 10
NDV Efficacy in Broiler Birds

| | | Dose (pfu) | | | | ND Elisa Titer | | | | |
|---|---|---|---|---|---|---|---|---|---|---|
| | | Target/ | | | PF | (% Pos. w/o cut-off) | | | | |
| Trt | Description | Backtiter | % Mortality | | (%) | D0 | D10 | D17 | D25 | D32 |
| T01 | Negative | — | NA | | NA | 100 | 100 | 50 | 14 | 25 |
| T02 | Challenge | — | 92 | (11/12) | 0 | | 100 | 42 | 21 | 21 |
| | control | | | | | | | | | |
| T03 | Vectormune ND | 4000/0* | 46 | (5/11) | 50 | | 100 | 68 | 65 | 81 |
| T04 | HVT-ND #38 | 4000/6907 | 58 | (7/12) | 37 | | 100 | 79 | 43 | 18 |
| T05 | HVT-ND #42 | 4000/5350 | 0 | (0/12) | 100 | | 100 | 71 | 82 | 86 |
| T06 | HVT-ND #45 | 4000/5057 | 0 | (0/12) | 100 | | 100 | 68 | 71 | 93 |

The antibody response to various HVT-ND vaccine candidates were assayed by using ProFlok ND plus kit (Zoetis LLC). All titers were included without using the cut-off value (345) recommended by the kit.

### Example 11

### In Vivo NDV Efficacy Test of HVT-ND #38, #42, #45 in SPF Birds with Day 20 Challenge

Three HVT-ND recombinants, HVT-IBD #38, #42, #45 were tested for their in vivo efficacy against virulent NDV challenge (Texas GB strain, provided by USDA) in SPF birds with Day 20 challenge. A positive control of commercial vaccine Vectormune ND (Ceva) was used in this study. 2000 pfu of each recombinant virus were injected in ovo at E18. The back-titer of vaccine virus was determined for each recombinant after vaccination. NDV Texas GB challenge was carried out at Day 20 per USDA instruction. All birds were observed for 2 weeks post challenge. We observed 87.5% protection for HVT-ND #42, and 70% protection for HVT-ND #45, 65% protection for HVT-ND #38, while our positive control Vectormune ND gave 87.5% protection. Please see Table 11 below.

TABLE 11
NDV Efficacy in SPF Birds with D 20 Challenge

| | | Dose (pfu) | % | | PF |
|---|---|---|---|---|---|
| | | Target/ | Affected | | (Prevented |
| Trt | Description | Backtiter | (Mortality) | | Fraction) |
| T01 | Negative | — | NA | | NA |
| T02 | Challenge control | — | 100% | (40/40) | 0 |
| T03 | Vectormune ND | 2000/2880 | 12.5% | (5/40) | 87.5 |
| T04 | HVT-ND #38 | 2000/1069 | 35% | (14/40) | 65 |
| T05 | HVT-ND #42 | 2000/3032 | 12.5% | (5/40) | 87.5 |
| T06 | HVT-ND #45 | 2000/2371 | 30% | (12/40) | 70 |

### Example 12

### In Vivo NDV Efficacy Test of HVT-ND (#42, MSV+5) in SPF Birds with Day 17, 18 and 19 Challenge

Three HVT-ND recombinant, HVT-ND (#42, MSV+5) was tested for its in vivo efficacy against virulent NDV challenge (Texas GB strain) in SPF birds with Day 17, 18 and 19 challenge. All birds were observed for 2 weeks post challenge. We observed 100% ( 40/40), 88% ( 35/40), 98% ( 39/40) of protection were observed for NDV challenges at Day 17, 18, 19, respectively, for in ovo vaccination. 75% ( 30/40), 88% ( 35/40), 93% ( 37/40) of protection were observed for subcutaneous vaccination on day of hatch. Please see Table 12 below.

TABLE 12

| | | | Challenge | % NDV Efficacy | | | | | |
|---|---|---|---|---|---|---|---|---|---|
| | | | (D17, D18, | D31 | | D32 | | D33 | |
| Trt | Vaccine | Route | D19) | (D17) | | (D18) | | (D19) | |
| T01 | Non- | — | No | NA | (40/40) | NA | (40/40) | NA | (40/40) |
| | vaccinated | | | | | | | | |
| T02 | Chall control | — | Yes | 0 | (0/40) | 0 | (0/40) | 0 | (0/40) |
| T03 | HVT-ND | Sub- | Yes | 75 | (30/40) | 88 | (35/40) | 93 | (37/40) |
| | | cutaneous | | | | | | | |
| T04 | | In ovo | Yes | 100 | (40/40) | 88 | (35/40) | 98 | (39/40) |

### Example 13

### In Vivo NDV Efficacy Test of HVT-ND (#42, MSV+5) in SPF Birds with Day 16 and 19 Challenge

Three HVT-ND recombinant, HVT-ND (#42, MSV+5) was tested for its in vivo efficacy against virulent NDV challenge (Texas GB strain) in SPF birds with Day 16 and 19 challenge. All birds were observed for 2 weeks post challenge. We observed 85% ( 37/40) and 93% ( 37/40) of protection were observed for Day 16 and Day 19 NDV challenge for in ovo vaccination. 70% ( 28/40) and 95% ( 38/40) of protection were observed for subcutaneous vaccination on day of hatch. Please see Table 13 below.

TABLE 13

| | | | Challenge | % NDV Efficacy | | | |
|---|---|---|---|---|---|---|---|
| Trt | Vaccine | Route | (D 16, D 19) | D 30 (D 16) | | D 33 (D 19) | |
| T01 | Non- | — | Yes | 0 | (0/40) | 0 | (0/40) |
| | vaccinated | | | | | | |
| T02 | HVT-ND | Sub- | Yes | 70 | (28/40) | 95 | (38/40) |
| | | cutaneous | | | | | |
| T03 | | In ovo | Yes | 85 | (34/40) | 93 | (37/40) |

### Example 14

### Duration of Immunity Test of HVT-ND (#42, MSV+5) in SPF Birds by Day 63 Challenge

Three HVT-ND recombinant, HVT-ND (#42, MSV+5) was tested for duration of immunity against virulent NDV challenge (Texas GB strain) in SPF birds with Day 63 challenge. All birds were observed for 2 weeks post challenge. We observed 100% ( 30/30) of protection for both in ovo vaccination or subcutaneous vaccination on day of hatch. Please see Table 14 below.

TABLE 14

| | | | NDV | % NDV Efficacy | | | | | |
|---|---|---|---|---|---|---|---|---|---|
| | | | Challenge | % Clinical | | | | | |
| Trt | Vaccine | Route | (D63) | Signs | | % Mortality | | % Protection | |
| T01 | Non- | — | No | 0 | (0/30) | 0 | (0/30) | NA | (30/30) |
| | vaccinated | | | | | | | | |
| T02 | Chall control | — | Yes | 80 | (24/30) | 100 | (30/30) | 0 | (0/30) |
| T03 | HVT-ND | Sub- | Yes | 0 | (0/30) | 0 | (0/30) | 100 | (30/30) |
| | | cutaneous | | | | | | | |
| T04 | | In ovo | Yes | 0 | (0/30) | 0 | (0/30) | 100 | (30/30) |

### Example 15

### ND Immunogenicity Test of HVT-ND (#42, MSV+5) in SPF Birds

Three HVT-ND recombinant, HVT-ND (#42 MSV+5) was tested for immunogenicity against virulent NDV challenge (Texas GB strain, provided by USDA) on day 28 in SPF birds. All birds were observed for 2 weeks post challenge. We observed 100% ( 30/30) of protection for both in ovo vaccination or subcutaneous vaccination on day of hatch. Please see Table 15 below.

TABLE 15

| Trt | Description | Route | % Susceptible | | % Protection | Route | % Susceptible | | % Protection |
|---|---|---|---|---|---|---|---|---|---|
| T01 | Poulvac Diluent | SC | 0 | (0/40) | 100 | In ovo | 0 | (0/40) | 100 |
| T02 | HVT Placebo | | 100 | (40/40) | 0 | | 100 | (40/40) | 0 |
| | (CEF) | | | | | | | | |
| T04 | HVT-ND | | 0 | (0/40) | 100 | | 7.5 | (3/40) | 92.5 |

### Example 16

### MD Immunogenicity Test of HVT-ND (#42, MSV+5) in SPF Birds

Three HVT-ND recombinant, HVT-ND (#42, MSV+5) was tested for immunogenicity against virulent MDV challenge (GA22 strain) on day 5 in SPF birds. All birds were observed for 54 days post challenge. We observed 100% ( 30/30) of protection for both in ovo vaccination or subcutaneous vaccination on day of hatch. Please see Table 16 below.

TABLE 16

| | | | % MD | | | | % MD | | |
|---|---|---|---|---|---|---|---|---|---|
| Trt | Description | Route | lesions | | % Protection | Route | lesions | | % Protection |
| T01 | Poulvac Diluent | In ovo | 0 | (0/30) | 100 | SC | 0 | (0/30) | 100 |
| T02 | HVT Placebo | | 90 | (27/30) | 10 | | 100 | (30/30) | 0 |
| | (CEF) | | | | | | | | |
| T03 | HVT-ND X + 5 | | 17 | (5/30) | 83 | | 20 | (6/30) | 80 |

### Example 17

### In Vitro Growth experiment

In vitro growth experiment was carried out for HVT-ND #38, #42, #45. Roller bottles of 490 cm² were seeded with 5×108 primary CEF cells. HVT-ND #38, #42, #45 were inoculated into each roller bottle at three different MOI: 0.001, 0.003, 0.008. Infected cells were harvested at 48-hour post infection and titrated on CEF cells. Both HVT-ND #42 and #45 grow well and has titer of 2.86×106 and 2.97×106 pfu/mL, respectively. HVT-ND #38 had titer of 1.67×106 pfu/mL. Please see Table 17 below.

TABLE 17

| Growth Experiment for HVT-ND preMS viruses | | | | | | | | | | |
|---|---|---|---|---|---|---|---|---|---|---|
| | | | | Virus/ | Reps/Trt | Total | Total | Total | | |
| | | Cells/ | | 490RB | (490 | Cells/ | Virus/ | Virus mL/ | | Pfu/mL |
| Treatment | Virus | 490RB | MOI | (PFU) | RB) | Trt | Trt | Candidate | Pfu/mL | (Ave) |
| T01 | HVT- | 5.00E+08 | 0.001 | 5.00E+05 | 2 | 1.00E+09 | 1.00E+06 | 10.00 | 2.17E+05 | 1.67E+06 |
| T02 | ND #38 | 5.00E+08 | 0.003 | 1.50E+06 | 2 | 1.00E+09 | 3.00E+06 | | 1.13E+06 | |
| T03 | | 5.00E+08 | 0.008 | 4.00E+06 | 2 | 1.00E+09 | 8.00E+06 | | 1.71E+06 | |
| T04 | HVT- | 5.00E+08 | 0.001 | 5.00E+05 | 2 | 1.00E+09 | 1.00E+06 | 1.36 | 1.37E+06 | 2.86E+06 |
| T05 | ND #42 | 5.00E+08 | 0.003 | 1.50E+06 | 2 | 1.00E+09 | 3.00E+06 | | 2.88E+06 | |
| T06 | | 5.00E+08 | 0.008 | 4.00E+06 | 2 | 1.00E+09 | 8.00E+06 | | 4.32E+06 | |
| T07 | HVT- | 5.00E+08 | 0.001 | 5.00E+05 | 2 | 1.00E+09 | 1.00E+06 | 1.35 | 1.05E+06 | 2.97E+06 |
| T08 | ND #45 | 5.00E+08 | 0.003 | 1.50E+06 | 2 | 1.00E+09 | 3.00E+06 | | 3.15E+06 | |
| T09 | | 5.00E+08 | 0.008 | 4.00E+06 | 2 | 1.00E+09 | 8.00E+06 | | 4.70E+06 | |

### Example 18

### Construction of HVT-IBD-ND #42-#30 LP C2

### Generation of Transfer Plasmid-#42

Initial transfer plasmid HVT-ND #42 was chemically synthesized by BioBasic, Inc. Cloning plasmid UL55/gene 3 was chemically synthesized by DNA2.0 as described above. PCR amplification of NDV F gene expression cassette of HVT-ND #42 transfer plasmid by using the following primers: upper primer SEQ ID NO. 154; lower primer SEQ ID NO. 155.

The amplified PCR product was cloned into the AscI and NheI sites of UL55/gene3 to make final transfer plasmid #42. This plasmid was used for transfection/infection to make HVT-ND #42.

### Generation of Transfer Plasmid-#30

Initial transfer plasmid HVT-IBD #30 was chemically synthesized by BioBasic, Inc. Cloning plasmid was chemically synthesized by DNA2.0. PCR amplification of IBD gene expression cassette of plasmid #30 plasmid by using the following primers: upper primer SEQ ID NO. 156, lower primer and SEQ ID NO. 157. The amplified PCR product was cloned into the Agel and KpnI sites of UL35/36 to make final transfer plasmid #30. This plasmid was used for transfection/infection to make HVT-IBD-ND #42-#30 LP C2.

### Construction of HVT-ND #42

Co-infection/transfection: Seed CEF cell in 6 well plate, next day perform HVT working seed infection (140 ul)+plasmid #42 (linearized by SpeI+SbfI digestion) transfection using Lipofectamine™ LTX Reagent (ThermoFisher). Harvested the transfected cells on day 2 post-transfection. Screened positive foci in 6 well plate by IFA with chicken anti-NDV polyclonal antibody (live stain, ˜1:250 dilution), then further purified one time (by live stain) in 96 well plate by limited dilution to obtain the single clones. The purified clone was passed two times in 6 well plate in duplicate and the purity of the clone was confirmed by IFA (by fix and stain). The 6 well harvest was used for construction of HVT-IBD-ND #42-#30.

### Construction of HVT-IBD-ND #42-#30

Co-infection/transfection: Seed CEF cell in 6 well plate, next day perform HVT-ND #42 infection+plasmid #30 (linearized by SbfI digestion) transfection using Lipofectamine™ LTX Reagent (ThermoFisher). Harvested the transfected cells on day 3 post-transfection. Screened positive foci in 6 well plate by IFA with chicken anti-IBD polyclonal antibody (live stain, ˜1:250 dilution), then further purified one time in 96 well plate (by live stain) by limited dilution to obtain the single clones. Two purified clones were picked and passed in 6 well plate in duplicate and the purity of the clones were confirmed by IFA (by fix and stain). The clones were scaled up sequentially in T-75 flask, T-150 flask, T-225 flask, 850 ml roller bottle. The recombinant virus was harvested and aliquoted 1 ml/vial, froze at −80 C overnight then transferred into LN tank.

### Example 19

### In Vivo NDV Efficacy Test of HVT-IBD-ND #42-#30, #42-#32, #104 in SFP Birds

Seven HVT-IBD-ND recombinants, HVT-IBD-ND #42-#30 (3 clones), #42-#32 (2 clones), #104 (2 clones), were tested for their in vivo efficacy against virulent NDV challenge in SPF birds. NDV Texas GB challenge was carried out at Day 28. About 1500 PFU of each recombinant virus was injected in ovo at E18. All birds were observed for 2 weeks post challenge. Please see Table 18 below.

TABLE 18

| | | % ND Efficacy | | | |
|---|---|---|---|---|---|
| Trt | Description | D 14 | | D 21 | |
| T01 | Negative | — | (20/20) | — | (20/20) |
| T02 | Challenge | 0 | (0/20) | 0 | (0/20) |
| | control | | | | |
| T05 | HVT-IBD-ND #42-#30 LP C1 | 70 | (14/20) | 100 | (20/20) |
| | p17 | | | | |
| T06 | HVT-IBD-ND #42-#30 LP C2 | 75 | (15/20) | 95 | (19/20) |
| | p17 | | | | |
| T07 | HVT-IBD-ND #42-#32 LP C1 | 70 | (14/20) | 100 | (20/20) |
| | p17 | | | | |
| T08 | HVT-IBD-ND #42-#32 LP C2 | 65 | (13/20) | 100 | (20/20) |
| | p17 | | | | |
| T09 | HVT-IBD-ND #104 C1 | 70 | (14/20) | 100 | (20/20) |
| T10 | HVT-IBD-ND #104 C7 | 80 | (16/20) | 100 | (20/20) |

| | | | | |
|---|---|---|---|---|
| T11 | HVT-IBD-ND #42-#32 preMSV | 35 | (7/20) | — [90 (36/40), |
| | p36 | | | B1943] |

### Example 20

### In Vivo IBD Efficacy Test of HVT-IBD-ND #42-#30, #42-#32, #104 in SFP Birds

Seven HVT-IBD-ND recombinants, HVT-IBD-ND #42-#30 (3 clones), #42-#32 (2 clones), #104 (2 clones), were tested for their in vivo efficacy against virulent IBDV challenge in SPF birds on Day 14 and Day 21, respectively. About 2000 PFU of each recombinant virus was injected in ovo at E18. All birds were necropsied at 5 days post challenge. Please see Table 19 below.

TABLE 19

| | | % IBD Efficacy | | | |
|---|---|---|---|---|---|
| Trt | Description | D 14 | | D 21 | |
| T01 | Negative | — | (20/20) | — | (20/20) |
| T02 | Challenge | 0 | (0/20) | 5 | (1/20) |
| | control | | | | |
| T05 | HVT-IBD-ND #42-#30 LP C1 | 85 | (17/20) | 95 | (19/20) |
| | p17 | | | | |
| T06 | HVT-IBD-ND #42-#30 LP C2 | 90 | (18/20) | 90 | (18/20) |
| | p17 | | | | |
| T07 | HVT-IBD-ND #42-#32 LP C1 | 85 | (17/20) | 100 | (20/20) |
| | p17 | | | | |
| T08 | HVT-IBD-ND #42-#32 LP C2 | 75 | (15/20) | 85 | (17/20) |
| | p17 | | | | |
| T09 | HVT-IBD-ND #104 C1 | 60 | (12/20) | 100 | (20/20) |
| T10 | HVT-IBD-ND #104 C7 | 70 | (14/20) | 100 | (20/20) |
| T11 | HVT-IBD-ND #42-#32 preMSV | 75 | (15/20) | 95 | (19/20) |

### Example 21

### In Vivo MDV Efficacy Test of HVT-IBD-ND #42-#30 (4 Clones) in SFP Birds

Three HVT-IBD-ND recombinants #42-#30 (4 clones) were tested for their in vivo efficacy against virulent MDV challenge (GA22) in SPF birds. About 1500 PFU of each recombinant virus was injected in ovo at E18. MDV GA22 challenge was carried out at Day 5. All birds were observed for 54 days post challenge. Please see Table 20 below.

TABLE 20

| | | MD Efficacy | | |
|---|---|---|---|---|
| Groups | Vaccine | % Pos. (#) | | % Protection |
| T01 | Negative | 0 | (0/30) | 100 |
| T02 | Challenge | 93 | (28/30) | 7 |
| | control | | | |
| T04 | HVT-IBD-ND #42-#30 preMSV | 30 | (9/29) | 69 |
| | p33 | | | |
| T05 | HVT-IBD-ND #42-#30 LP C1 | 30 | (9/29) | 69 |
| | p17 | | | |
| T06 | HVT-IBD-ND #42-#30 LP C2 | 13 | (4/30) | 87 |
| | p17 | | | |
| T07 | HVT-IBD-ND #46-#30a preMSV | 30 | (9/30) | 70 |
| | p26 | | | |

### Example 22

### In Vivo vvIBD Efficacy Test of HVT-IBD-ND #42-#30, #42-#32, #104 in SPF Birds

Three HVT-IBD-ND recombinants, #42-#30 (2 clones), #42-#32 (2 clones), #104 were tested for their in vivo efficacy against very virulent IBDV challenge in SPF birds. About 1500 PFU of each recombinant virus were injected in ovo at E18. vvIBDV challenge was carried out at Day 14 and Day 21. All birds were observed for 10 days post challenge. Histology examination of bursa was conducted for each bird at end of study. Please see Table 21 below.

TABLE 21

| | | Day 14 vvIBD Efficacy | | | | | Day 21 vvIBD Efficacy | | | | |
|---|---|---|---|---|---|---|---|---|---|---|---|
| | | | | Mean | | | | | Mean | | |
| Trt | Description | % Mortality | | BF score | % Protection | | % Mortality | | BF score | % Protection | |
| T01 | Negative | 0 | (0/19) | 0.42 | — | (19/19) | 0 | (0/20) | 0.11 | — | (20/20) |
| T02 | Challenge control | 95 | (19/20) | 5.00 | 0 | (0/20) | 100 | (19/19) | 0.00 | 0 | (0/19) |
| T06 | HVT-IBD-ND #42-#30 LP | 0 | (0/20) | 2.35 | 65 | (13/20) | 0 | (0/20) | 1.20 | 85 | (17/20) |
| | C1 | | | | | | | | | | |
| T07 | HVT-IBD-ND #42-#30 LP | 0 | (0/20) | 2.80 | 60 | (12/20) | 5 | (1/20) | 0.90 | 85 | (17/20) |
| | C2 | | | | | | | | | | |
| T08 | HVT-IBD-ND #42-#32 LP | 0 | (0/19) | 2.89 | 53 | (10/19) | 0 | (0/20) | 0.90 | 85 | (17/20) |
| | C1 | | | | | | | | | | |
| T09 | HVT-IBD-ND #42-#32 LP | 5 | (1/20) | 2.58 | 50 | (10/20) | 5 | (1/20) | 0.68 | 95 | (19/20) |
| | C2 | | | | | | | | | | |
| T10 | HVT-IBD-ND #104 C7 | 0 | (0/20) | 3.00 | 50 | (10/20) | 10 | (2/20) | 1.94 | 55 | (11/20) |

### Example 23

### IBD Duration of Immunity Test of HVT-IBD-ND (#42-#30, X+5) in SPF Birds by Day 63 Challenge

The HVT-IBD-ND recombinant, #42-#30 (MSV+5) was tested for duration of immunity against virulent classic IBDV challenge in SPF birds with Day 63 challenge. All birds were observed for four days post challenge and followed by necropsy. Please see Table 22

TABLE 22

| | | | % Post-chall | % IBD | |
|---|---|---|---|---|---|
| | Vaccine | Route | Mortality | Protection | |
| T01 | Diluent | In ovo | 0 (0/30) | NA | (30/30) |
| T02 | Placebo | In ovo | 7 (2/30) | 7 | (2/30) |
| T03 | HVT-IBD-ND | SC | 0 (0/30) | 100 | (30/30) |
| T04 | HVT-IBD-ND | In ovo | 0 (0/30) | 100 | (30/30) |

### Example 24

### ND Immunogenicity Test of HVT-IBD-ND (#42-#30, MSV+5) in SPF Birds

The HVT-IBD-ND recombinant, #42-#30 (MSV+5) was tested for immunogenicity against virulent NDV challenge (Texas GB strain) on day 28 in SPF birds. All birds were observed for 2 weeks post challenge. Please see Table 23.

TABLE 23

| Trt | Description | Route | % Susceptible | | % Protection | Route | % Susceptible | | % Protection |
|---|---|---|---|---|---|---|---|---|---|
| T01 | Diluent | SC | 0 | (0/40) | NA | In ovo | 0 | (0/40) | NA |
| T02 | Placebo (CEF) | | 100 | (40/40) | 0 | | 100 | (40/40) | 0 |
| T03 | HVT-IBD-ND | | 0 | (0/40) | 100 | | 5 | (2/40) | 95 |
| | X + 5 | | | | | | | | |

### Example 25

### IBD Immunogenicity Test of HVT-IBD-ND (#42-#30, MSV+5) in SPF Birds

The HVT-IBD-ND recombinant, #42-#30 (MSV+5) was tested for immunogenicity against virulent IBDV challenge on day 34 in SPF birds. All birds were observed for 4 days post challenge and followed by necropsy for bursal lesions. Please see Table 24 below.

TABLE 24

| | | | | | % IBD | | | | | | % IBD | | |
|---|---|---|---|---|---|---|---|---|---|---|---|---|---|
| Trt | Description | Route | % Mortality | | Lesions | | % Protection | Route | % Mortality | | Lesions | | % Protection |
| T01 | Diluent | SC | 0 | (0/30) | 0 | (0/30) | NA | In ovo | 0 | (0/30) | 0 | (0/30) | NA |
| T02 | Placebo | | 100 | (30/30) | 100 | (30/30) | 0 | | 43 | (13/30) | 93 | (28/30) | 7 |
| | (CEF) | | | | | | | | | | | | |
| T03 | HVT-IBD- | | 0 | (0/30) | 0 | (0/30) | 100 | | 0 | (0/30) | 0 | (0/30) | 100 |
| | ND | | | | | | | | | | | | |

### Example 26

### MD Immunogenicity Test of HVT-IBD-ND (#42-#30, MSV+5) in SPF Birds

Three HVT-IBD-ND recombinant, #42-#30 (MSV+5) was tested for immunogenicity against virulent MDV challenge (GA22 strain) on day 5 in SPF birds. All birds were observed for 54 days post challenge. Please see Table 25 below.

TABLE 25

| | | | | | % MD | | % MD |
|---|---|---|---|---|---|---|---|
| Trt | Description | Route | % Mortality | | lesions | | Protection |
| T01 | Poulvac Diluent | SC | 0 | (0/30) | 0 | (0/30) | NA |
| T02 | Placebo (CEF) | | 40 | (12/30) | 83 | (25/30) | 17 |
| T04 | HVT-ND-IBD | | 3 | (1/30) | 17 | (5/30) | 83 |
| | X + 5 | | | | | | |

### Example 27

### ND Immunogenicity Test of HVT-IBD-ND (#42-#30, MSV+5) in SPF Birds Against an EU Challenge Strain

The HVT-IBD-ND recombinant, #42-#30 (MSV+5) was tested for immunogenicity against a virulent NDV Europe challenge (Herts Weybridge 33/56) on day 21 in SPF birds. All birds were observed for 2 weeks post challenge. Please see Table 26 below.

TABLE 26

| Trt | Description | Route | % Mortality | | % Protection |
|---|---|---|---|---|---|
| T01 | Control | — | 100 | (15/15) | NA |
| T03 | HVT-IBD-ND | SC | 4 | (1/26) | 96 |
| T04 | HVT-IBD-ND | In ovo | 4 | (1/26) | 96 |

### Example 28

### Compatibility of HVT-ND with Bursaplex

### IBDV Efficacy

BURSAPLEX™ (Zoetis, U.S. Pat. No. 5,871,748, herein incorporated by reference) is a vaccine against Infectious Bursal Disease (IBD) that comprises a vaccine conjugate consisting of a live, attenuated IBD strain 2512 and neutralizing antibody, BDA, bound to the virus. Bursaplex produces active immunity against IBD in poultry. particularly chickens. On E18 eggs were in ovo injected with either control or test vaccine (HVT-ND with Bursaplex at 1:1 ratio) and transferred to an allotted hatcher as designated by Biometrics along with eggs which were not injected. On the day of hatch, T04 birds were subcutaneously vaccinated. Blood samples were collected on Days 5, 12, 19, 26 and 33 for IBDV serology. On Day 34, designated birds were challenged with classic virulent IBDV and on Day 38 all birds were necropsied for presence of bursal lesions. No chicken in T01 negative group developed grossly observable lesions and 100 percent of the chickens in T02 challenge control group developed grossly observable lesions. T03 (HVT-ND+Bursaplex, in ovo) and T04 (HVT-ND+Bursaplex, SC) were both protected at 100%. It can be concluded that Poulvac Procerta HVT-ND and Poulvac Bursaplex are compatible when administered together and remain efficacious against an IBDV challenge when administered either in ovo or subcutaneously.

### NDV Efficacy

On E18 eggs were in ovo injected with either control or test vaccine (HVT-ND with Bursaplex at 1:1 ratio) and transferred to an allotted hatcher as designated by Biometrics along with eggs which were not injected. On the day of hatch, T04 birds were subcutaneously vaccinated. Blood samples were collected on Days 6, 13, 20 and 27 for NDV serology. On Day 28, designated birds were challenged with a velogenic NDV and on Day 42 all surviving birds were terminated. No chicken in T01 negative group developed clinical signs and 100 percent of the chickens in T02 challenge control group developed developed clinical signs of Newcastle disease, including mortality. T03 (HVT-ND+Bursaplex, in ovo) and T04 (HVT-ND+Bursaplex, SC) were protected at 92.5% and 95%, respectively. It can be concluded that Poulvac Procerta HVT-ND and Poulvac Bursaplex are compatible when administered together and remain efficacious against an NDV challenge when administered either in ovo or subcutaneously.

### Example 29

### Compatibility of HVT-ND with Magniplex

### IBD Efficacy

MAGNIPLEX™ (Zoetis) is a vaccine against Infectious Bursal Disease (IBD) that comprises a vaccine conjugate consisting of a live, attenuated IBD strain V877 and neutralizing antibody, BDA, bound to the virus. Bursaplex produces active immunity against IBD in poultry, particularly chickens. On E18 eggs were in ovo injected with either control or test vaccine (HVT-ND Pre-license serial with Magniplex at 1:1 ratio) and transferred to an allotted hatcher as designated by Biometrics along with eggs which were not injected. On the day of hatch, T04 birds were subcutaneously vaccinated. Blood samples were collected on Days 5, 12, 19, 26 and 33 for IBDV serology. On Day 34, designated birds were challenged with classic virulent IBDV and on Day 38 all birds were necropsied for presence of bursal lesions. No chicken in T01 negative group developed grossly observable lesions and 100 percent of the chickens in T02 challenge control group developed grossly observable lesions. T03 (HVT-ND+Magniplex, in ovo) and T04 (HVT-ND+Magniplex, SC) were both protected at 100%. It can be concluded that Poulvac Procerta HVT-ND and Poulvac Magniplex are compatible when administered together and remain efficacious against an IBDV challenge when administered either in ovo or subcutaneously.

TABLE 27

| | | | | % IBD Efficacy | | | | | |
|---|---|---|---|---|---|---|---|---|---|
| | | | Challenge | % Neg Bursal | | % Neg | | | |
| Trt. | Vaccine | Route | (D34) | Lesions | | Mortality | | % Protection | |
| T01 | Diluent | In ovo | No | 100 | (30/30) | 100 | (30/30) | NA | (30/30) |
| T02 | CEF cells | In ovo | Yes | 0 | (0/30) | 70 | (21/30) | 0 | (0/30) |
| T03 | HVT-ND + | In ovo | Yes | 100 | (30/30) | 100 | (30/30) | 100 | (30/30) |
| T04 | Magniplex | SC | Yes | 100 | (30/30) | 100 | (30/30) | 100 | (30/30) |

TABLE 28

| | | | % Positive - | | | | IBDV ELISA | | | | | | |
|---|---|---|---|---|---|---|---|---|---|---|---|---|---|
| | | | IBDV ELISA | | | | GMT (sdev) | | | | | | |
| | | Route | D6 | D13 | D20 | D27 | D6 | D13 | | D20 | | D27 | |
| T01 | Diluent | In ovo | 0 | 0 | 0 | 0 | 0 | 0 | (0) | 0 | (0) | 0 | (0) |
| T02 | CEF cells | In ovo | 0 | 0 | 0 | 0 | 0 | 0 | (0) | 0 | (0) | 0 | (0) |
| T03 | HVT-ND + | In ovo | 0 | 61 | 100 | 100 | 0 | 84 | (739) | 5790 | (2295) | 9510 | (3453) |
| T04 | Magniplex | SC | 0 | 33 | 100 | 100 | 0 | 11 | (1109) | 5813 | (2246) | 11531 | (2090) |

### ND Efficacy

On E18 eggs were in ovo injected with either control or test vaccine (HVT-ND with Magniplex at 1:1 ratio) and transferred to an allotted hatcher as designated by Biometrics along with eggs which were not injected. On the day of hatch, T04 birds were subcutaneously vaccinated. Blood samples were collected on Days 6, 13, 20 and 27 for NDV serology. On Day 28, designated birds were challenged with a velogenic NDV and on Day 42 all surviving birds were terminated. No chicken in T01 negative group developed clinical signs and 100 percent of the chickens in T02 challenge control group developed developed clinical signs of Newcastle disease, including mortality. T03 (HVT-ND+Magniplex, in ovo) and T04 (HVT-ND+Magniplex, SC) were protected at 92.5% and 95%, respectively. It can be concluded that Poulvac Procerta HVT-ND and Poulvac Magniplex are compatible when administered together and remain efficacious against an NDV challenge when administered either in ovo or subcutaneously.

TABLE 29

| | | | | % ND Efficacy | | | | | |
|---|---|---|---|---|---|---|---|---|---|
| | | | Challenge | % Clinical | | | | | |
| Trt. | Vaccines | Route | (D28) | Signs | | % Live Birds | | % Protection | |
| T01 | Diluent | In ovo | No | 0 | (0/40) | 100 | (40/40) | NA | (40/40) |
| T02 | CEF cells | In ovo | Yes | 75 | (30/40) | 0 | (0/40) | 0 | (0/40) |
| T03 | HVT-ND + | In ovo | Yes | 0 | (0/40) | 98 | (1/40) | 98 | (39/40) |
| T04 | Magniplex | SC | Yes | 0 | (0/40) | 100 | (40/40) | 100 | (40/40) |

TABLE 30

| | | | % Signal Positive - | | | | NDV ELISA | | | | | |
|---|---|---|---|---|---|---|---|---|---|---|---|---|
| | | | NDV ELISA | | | | GMT (sdev) | | | | | |
| Trt. | Vaccines | Route | D6 | D13 | D20 | D27 | D6 | D13 | D20 | | D27 | |
| T01 | Diluent | In ovo | 0 | 0 | 0 | 0 | 0 (0) | 0 (0) | 0 | (0) | 0 | (0) |
| T02 | CEF cells | In ovo | 0 | 0 | 0 | 0 | 0 (0) | 0 (0) | 0 | (0) | 0 | (0) |
| T03 | HVT-ND + | In ovo | 0 | 0 | 3 | 47 | 0 (0) | 0 (0) | 0.2 | (86) | 18 | (300) |
| T04 | Magniplex | SC | 0 | 0 | 14 | 47 | 0 (0) | 0 (0) | 1.3 | (150) | 22 | (451) |

1-21. (canceled)

22. A composition comprising a combination comprising (a) a recombinant Herpesvirus of Turkey (HVT) and (b) a live, attenuated Infectious Bursal Disease (IBD) Virus bound to a neutralizing antibody.

23. The composition of claim 22 wherein the recombinant HVT comprises a recombinant HVT genome comprising a nucleic acid sequence that encodes a heterologous antigen from a Newcastle Disease Virus (NDV).

24. The composition of claim 23 wherein the nucleotide sequence that encodes a heterologous antigen from a Newcastle Disease Virus is selected from the group consisting of: the nucleotide sequence coding for the F/HN chimera protein; the nucleotide sequence coding for the F protein; the nucleotide sequence coding for the NP protein; the nucleotide sequence coding for the P protein; the nucleotide sequence coding for the M protein; the nucleotide sequence coding for the HN protein; and the nucleotide sequence coding for the L protein of the Newcastle Disease Virus.

25. The composition of claim 24 wherein the recombinant Herpesvirus of Turkey (HVT) genome of claim 24 wherein the nucleotide sequence coding for a heterologous antigen from a Newcastle Disease Virus comprises the nucleotide sequence coding for the F protein.

26. The recombinant Herpesvirus of Turkey (HVT) genome of claim 25 wherein the nucleotide sequence coding for the F protein from a Newcastle Disease Virus comprises a nucleotide sequence comprising at least about 80% sequence identity to the nucleotide sequence that comprises SEQ ID NO. 3.

27. The recombinant Herpesvirus of Turkey (HVT) genome of claim 23 further comprising the nucleotide sequence of one or more promoters.

28. The recombinant Herpesvirus of Turkey (HVT) genome of claim 27 wherein the one or more promoters are selected from the group consisting of: immediate early cytomegalovirus human (hCMV) promoter: guinea pig immediate early CMV promoter; murine immediate early CMV promoter; Pec promoter; β-chicken actin promoter; SV40 promoter; Pseudorabies Virus promoters of glycoprotein X promoter; Herpes Simplex Virus-1 alpha 4 promoter; Marek's Disease Virus promoters of glycoproteins gA, gC, gB, gE, or gl promoter; Infectious Laryngotracheitis Virus promoters of glycoprotein gB, gE, gl, gD promoter; and Bovine Herpesvirus 1/1 VP8 promoter.

29. The recombinant Herpesvirus of Turkey (HVT) genome of claim 28 wherein the nucleotide sequences for one or more promoters comprise the nucleotide sequences for the immediate early cytomegalovirus human promoter (hCMV) comprising the nucleotide sequence that comprises SEQ ID NO. 1 and the murine immediate early CMV promoter comprising SEQ ID NO.2.

30. The recombinant Herpesvirus of Turkey (HVT) genome of claim 23 further comprising one or more nucleotide sequences that encode a poly A signal.

31. The recombinant Herpesvirus of Turkey (HVT) genome of claim 30 wherein the one or more nucleotide sequences that encode a poly A signal comprise either the SV40 poly A tail comprising the nucleotide sequence comprising SEQ ID NO. 12, or the Bovine Growth Hormone (BGH) poly A tail comprising the nucleotide sequence comprising SEQ ID NO. 6.

32. The infectious bursal disease virus of claim 22 wherein said virus is selected from either IBD strain 2512 or IBD strain V877.

33. A vaccine composition comprising the composition of claim 22 further comprising a pharmaceutically acceptable carrier, excipient, or adjuvant.

34. A kit comprising the vaccine of claim 33.
